SEQUENCE LISTING (105 sequences)

SEQ ID NO: 1 - protein, 791 aa, synthetic construct
  REGION 1..791: note = Synthetic Sequence
MIKKPSNRHALPKVIISEVNSEKILEFKIKYEKLARLDRFEVKAMHYEGKEIVFDEVLVNGGLIDVEYEDEHKTLFVKVGEKSYSIRGQKVGGKQRLLENRVSKTKVLLELSDGVPDKNAKLRKSRTERELIVVENIKLYSQIVGKEVSTTKEIYLTKRFLSYRSDLLFYYSFVDNFFKVAGNEKELWKINFDDASSAQFMGYVPFMVNDNLKNDNAYLKDYVSDNEQIKDDLKKVQTMFSTLRHALLHFNYEFLNLLIENIDKLNIDAKKEFIDEEKIKLFGENLSLAKVYRLYSDICVNRVGFNKFINSMLIKDGVENQALKAEFDRKFFGKAYTIDIHSNQAYKRIYNEHKKLVIKVSTLKDGQAIRRGNKKISELKEQMKSMTKKNSLARLECKMRLAFGFLYGEYNNYNTFKNNFDTNIKNSQFDVNDVEKSKAYFLSTYERRKPRTSEKLEKVAKNIERLELKTVIANDPLLKFILLMFAFMPQELKGEFLGFVKKYYHDVHSIDDDTKEQEQDVVEAMSTSLKLKILGRNIRSLTLFKYALSSQVNYNSTYNLFYVEGNRYGKIYKKLGISHNQEEFDKTLVVPLLRYYSALFKLMNDFEIYSLAKANPTAVSLQELVDDERSPYKQGRFYNFREVLKQVYLLSDNELTHCKIRITRNKIAHFITEDLLGKPLLGEIKLNLQRKDMVSFMEARGNIKELLDYDAINDFRMKVIHLRTKMRVNSDKLQTMMDLLSNPKTPNDFYNVYKVKGVEIINKHLLEVLAQTAEERSIEKQIREGNEKYAL

SEQ ID NO: 2 - protein, 809 aa, synthetic construct
  REGION 1..809: note = Synthetic Sequence
MIKKPSNRHALPKVIISEVNSEKILEFKIKYEKLARLDRFEVKAMHYEGKEIVFDEVLVNGGLIDVEYHDEHKTLFVKVGEKSYSIRGQKVGGKQRLLENRVSKTKVLLELSDGVEDNKGNLRKSKTERELIVAENIKLYSQIVGREVSTTKEIYLTKRFLSYRSDLLFYYSFVDNFFKVAGNEKELWKINFDDASSAQFMGYVPFMVNDNLKNDNAYLKDYVSDNEQIKDDLKKVQTMFSTLRHALLHFNYEFFEKLFNGEDVGFDFDIGFLNLLIENIDKLNIDAKKEFIDDEKIKLFGENLSLAKVYRLYSDICVNRVGFNKFINSMLIKDGVENQVLKAEFDRKFGGKAYTVDIHSNQAYKRIYNEHKKLVIKVSTLKDGQAIRRGNKKISELKEQMKSMTKKNSLARLECKMRLAFGFLYGEYNNYNAFKNNFDTHIKNSQFDVNDVEKSKAYFLSTYERRKPRTSEKLEKVAKNIESLELKTVIANDPLLKFILLMFVFMPQELKGEFLGFVKKYYHDVHSIDDDTKEQEEDVVEAMSTSLKLKILGRNIRSLTLFKYALSSQVNYNSTDNLFYVEGNRYGKIYKKLGISHNQEEFDKTLTVPLFRYYSALFKLMNDFEIYSLAKANPMAVSLQELVDDETSPYKQGDYFNFNKMLREIYGLTNDEIKSGKVVFMRNKIAHFDTEVLLSKPLLGQTKMNLQRKDIVSFIEARGNIKELLGYDAINDFRMKVIHLRTKMRVYSDKLQTMMDLLRSAKTPNDFYNVYKVKGVESINKYLLEVLAQTAEERSIEKQIKEGNEKYDL

SEQ ID NO: 3 - protein, 855 aa, synthetic construct
  REGION 1..855: note = Synthetic Sequence
MDKHPSNRYALPKVIISEVDHERILEFKVKYEKLARLDRFEVKAMHYDGAEIVFDEVVANGGLIEVEYQDNNKTITINLNGKKYTINGRKVGGKRRLLEDRISRGKVCLELHDKIPDEKGNLRSSRTERELITFDSTKLYSQIIGRDVASTKEIYLIKRFLAYRSDLLFYYGFIDNFFKVAGNKRELWKIDFSGDKNQELIKYFNFTINDKLKNDKGYLKEYTANDEQIKKDLQNTKEVFTALRHALMHFEYDFFEKLFNNEEIETLSKIHDIELLNTMINKLDKLNIDTRKEYIDDEKITVFGEEISLKTLYGLYAHTAINRVAFNKLINRFMVENGTENEALKKYFNSKAEGGIAYEIDIHQNSEYKQLYIQHKDLVSKLSALSDGDEIADTNKKISELKVKMKAITKANSLKRLEHKLRLTFGFIYTEYQDYNAFKNNFDTDIKSGRFIPKDSEGKRRGFDHRELDQLKRYYDATFADKKPQTKETFDEIDKQIDQLSLKNLIGDDTLLKVILLIYIFLPREIKGEFLGFVKKYYHDTKHIEEDTKDKDEGFDDTFPVGLKLKVLDKNIRALSVLKHSLSYQAKYNKKEEKKEQFYEAGNRHGRFYKKLGISHNQEEFDKSVYAPLLRYHAALFKLLNDFEIYSLAQHIEGKETLAQQIEKPQFSQYEHYNFRKMLSKTYPKSAERGALDNDAFDTVINMRNDIAHLSHEPLFECPLDGKKSYKLKQGKRTNTINVKPLPISRKMIVDFISSQSDMKKTLGYDAVNDLTMKIIQLRTRLKVYADKSETIKTLVDAAKTPNDFYHIYKVKGVEAINRHLLEVIGETKDEKRIRKRIESGNAIAGRTPADSQEN

SEQ ID NO: 4 - protein, 809 aa, synthetic construct
  REGION 1..809: note = Synthetic Sequence
MIKKPSNRHALPKVIISEVNSDNILEFKIKYEKLARLDKIEVKAMHYDNRNIVFDEVIVNDGLIELEYRDDHKRLFVKVGDKSYGITGQKVGGKQRLLENRVSKTKVQLELTDGVLDNKGKHRISRTERELIVATNIALYNQIIGREVKTTKEIYLIKRFLGYRSDLLFYYAFVDNFFKVADNEKELWKIDFDANNSTQLIKYISYIVNDNLKNDNAYLKEYVSNVEQIKEDLKKVQTIFSKLRHALLHFNYDFFEKLFNGKDVGFDFDIDFLNLLIENIDKLNIDAKKEFIDDEKIKLFGENLSLAKVYRLYSDICVNRVGFNKFINAMLIKDGVENQALKEAFDNKLGRKAYTIDIHSNQEYKGLYNRHKKLVIELSTLKNGQAIRKKNAEIAKLKEQMNEMTKKNSLSRLEHKLRLAFGFMYAEYNNHKAFKNNFDTDIKNSKFSENDVEKFKAYFLSTYEGRKRRTSEKLEKVAKNIESLKLKTLIANDPLLKFILLMFVFMPQELKGEFLGFVKKYYHDIHSIDEDSKEQENTVLELMPTSLKLKILGRNIRSLTLFKYALSSQVNYNSSDELFYVEGNRYGKIYKKLGISHNQEEFDKTLVVPLFRYYSALFKLMNDFEIYSLAQANPRVLSLQELVDDTTSPYKQGDYYNFKKMLTEIYGVTNDEVNEGKVVFMRNKIAHFETKILLSKPLLGQTKLNLQRKDIVSFIEARGDIKELLGYDAINDFRMKVIHLRTKMKVYADKLQTMMDLLRNVKTPNDFYNVYKVKGVESINKHLLEVLAQTDQERTIEKQMVEGNKKYKL

SEQ ID NO: 5 - RNA, 66 nt, synthetic construct
  variation 37..66: note = n is a, c, g, or u
  source 1..66: mol_type = other RNA
gttggaatataaccccgtttgtaggggtaataaaacnnnnnnnnnnnnnnnnnnnnnnnnnnnnnn

SEQ ID NO: 6 - protein, 775 aa, synthetic construct
  REGION 1..775: note = Synthetic Sequence
MAQVSKQTSKKRELSIDEYQGARKWCFTIAFNKALVNRDKNDGLFVESLLRHEKYSKHDWYDEDTRALIKCSTQAANAKAEALRNYFSHYRHSPGCLTFTAEDELRTIMERAYERAIFECRRRETEVIIEFPSLFEGDRITTAGVVFFVSFFVERRVLDRLYGAVSGLKKNEGQYKLTRKALSMYCLKDSRFTKAWDKRVLLFRDILAQLGRIPAEAYEYYHGEQGDKKRANDNEGTNPKRHKDKFIEFALHYLEAQHSEICFGRRHIVREEAGAGDEHKKHRTKGKVVVDFSKKDEDQSYYISKNNVIVRIDKNAGPRSYRMGLNELKYLVLLSLQGKGDDAIAKLYRYRQHVENILDVVKVTDKDNHVFLPRFVLEQHGIGRKAFKQRIDGRVKHVRGVWEKKKAATNEMTLHEKARDILQYVNENCTRSFNPGEYNRLLVCLVGKDVENFQAGLKRLQLAERIDGRVYSIFAQTSTINEMHQVVCDQILNRLCRIGDQKLYDYVGLGKKDEIDYKQKVAWFKEHISIRRGFLRKKFWYDSKKGFAKLVEEHLESGGGQRDVGLDKKYYHIDAIGRFEGANPALYETLARDRLCLMMAQYFLGSVRKELGNKIVWSNDSIELPVEGSVGNEKSIVFSVSDYGKLYVLDDAEFLGRICEYFMPHEKGKIRYHTVYEKGFRAYNDLQKKCVEAVLAFEEKVVKAKKMSEKEGAHYIDFREILAQTMCKEAEKTAVNKVRRAFFHHHLKFVIDEFGLFSDVMKKYGIEKEWKFPVK

SEQ ID NO: 7 - protein, 777 aa, synthetic construct
  REGION 1..777: note = Synthetic Sequence
GGMAQVSKQTSKKRELSIDEYQGARKWCFTIAFNKALVNRDKNDGLFVESLLRHEKYSKHDWYDEDTRALIKCSTQAANAKAEALANYFSAYRHSPGCLTFTAEDELRTIMERAYERAIFECRRRETEVIIEFPSLFEGDRITTAGVVFFVSFFVERRVLDRLYGAVSGLKKNEGQYKLTRKALSMYCLKDSRFTKAWDKRVLLFRDILAQLGRIPAEAYEYYHGEQGDKKRANDNEGTNPKRHKDKFIEFALHYLEAQHSEICFGRRHIVREEAGAGDEHKKHRTKGKVVVDFSKKDEDQSYYISKNNVIVRIDKNAGPRSYRMGLNELKYLVLLSLQGKGDDAIAKLYRYRQHVENILDVVKVTDKDNHVFLPRFVLEQHGIGRKAFKQRIDGRVKHVRGVWEKKKAATNEMTLHEKARDILQYVNENCTRSFNPGEYNRLLVCLVGKDVENFQAGLKRLQLAERIDGRVYSIFAQTSTINEMHQVVCDQILNRLCRIGDQKLYDYVGLGKKDEIDYKQKVAWFKEHISIRRGFLRKKFWYDSKKGFAKLVEEHLESGGGQRDVGLDKKYYHIDAIGRFEGANPALYETLARDRLCLMMAQYFLGSVRKELGNKIVWSNDSIELPVEGSVGNEKSIVFSVSDYGKLYVLDDAEFLGRICEYFMPHEKGKIRYHTVYEKGFRAYNDLQKKCVEAVLAFEEKVVKAKKMSEKEGAHYIDFREILAQTMCKEAEKTAVNKVARAFFAHHLKFVIDEFGLFSDVMKKYGIEKEWKFPVK

SEQ ID NO: 8 - protein, 802 aa, synthetic construct
  REGION 1..802: note = Synthetic Sequence
MQVENIKKGSSQGMYSIEQYEGAKKWCFAIVLNRAQTNLQGNPKLFEETLTRFERIRKEDWFDQETKKLIYAKQEQNEVEEEIQKAADEKLRDLRNYFSHYFHTPDCLIFTQNDPVRIIMEKAYEKARFEQAKKEQEDISIEFGELFEENGRITSAGVVFFASFFAERRFLNRLMGYVQGFTRTEGEYKITRDVFSTYCLRDSYSVKTPDHDAVMFRDILGYLSRVPSESYQRIKESQMRSETQLSERKTDKFILFALNYLEDYGLEDLADYTACFARTRIKREQDENTDGKEQKPHRKKPRVEIHFERAEGDPFYIKHNNVILRTQKKGAQTYIFRMGVYELKYLVLLSLLGKGAEAVKRIDRYVHSLRNQLPHIEKKSTEEIEGYVRFLPRFVRSHLGLLGVDDEKKIKARVDYVKAKWLEKKEKSRELQLHRKGRDILRYINERCERPLNIDEYNRILELLVTKHLDGFYRELEELKKTRRIDKNIVCNLSRHKSVNALHEKVCDLVVQELESLGREELKEYVGLIPKEEKEVSFEEKTDRVVKQPVIYKGFLRNEFFRESRKSFARLVEEAVREKGEVYDVPLGGEYYEIVSLDTFDKDNKRLYETLAMDRLLLMIARQYHLSLNKELAKRAQQIEWKKEDGEEVIIFTLKNPAQPEQSCSVRFSLRDYTKLYVMDDAEFLARLCDYFLPKDEEQIDYHRLYTQGMNRYTNLQREGIEAILELEKKTIGPEQPRPPKNYIPFSEIMDKSAYNEDDQKALRRVRNALLHHNLNFARADFKRFCGIMKREGIEKRWSLAV

SEQ ID NO: 9 - protein, 804 aa, synthetic construct
  REGION 1..804: note = Synthetic Sequence
MEFENIKKTSNKEVYSIEQYEGEKKWCFAIVLNRAQTNLEENPKLFEQTLTRFEKIMKQDWFNEETKKLIYEKEEENKVKEEIQIAASERLKNLRNYFSHYLHAPDCLIFNRNDTIRIIMEKAYEKSRFEAKKKQQEDISIEFPELFEEEDKITSAGVVFFVSFFIERRFLNRLMGYVQGFRKTEGEYNITRQVFSKYCLKDSYSVQAQDHDAVMFRDILGYLSRVPTEIYQHIKLTRKRSQDQLSERKTDKFILFALKYLEDYGLKDLADYTACFARSKIKRENEDTKETDGNKHKFHREKPVVEIHFDKEKQDQFYIKRNNVILKAQKKGGQSNVFRMGVYELKYLVLLSLLGKAEEAIQRIDRYISSLKKQLPYLDKISNEEIQKSINFLPRFVRSRLGLLQVDDEKRLKTRLEYVKAKWTDKKEGSRKLELHRKGRDILRYINERCDRPLSRKEYNNILKFIVNKDFAGFYNELEELKRTRRLDKNIIQKLSGHTTLNALHERVCDLVLQELGSLQSENLKEYIGLIPKEEKEVTFREKVDRILEQPVVYKGFLRYEFFKEDKKSFARLVEEAIKTKWSDFDIPLGEEYYNIPSLDRFDRTNKKLYETLAMDRLCLMMARQYYLRLNEKLAEKAQHIYWKKEDGREVIIFKFQNPKEQKKSFSIRFSILDYTKMYVMDDPEFLSRLWEYFIPKEAKEIDYHKHYARAFDKYTNLQKEGIDAILKLEGRIIERRKIKPAKNYIEFQEIMNRSGYNNDQQVALKRVRNALLHYNLNFEREHLKRFYGVVKREGIEKKWSLIV

SEQ ID NO: 10 - protein, 805 aa, synthetic construct
  REGION 1..805: note = Synthetic Sequence
MKVENIKEKSKKAMYLINHYEGPKKWCFAIVLNRACDNYEDNPHLFSKSLLEFEKTSRKDWFDEETRELVEQADTEIQPNPNLKPNTTANRKLKDIRNYFSHHYHKNECLYFKNDDPIRCIMEAAYEKSKIYIKGKQIEQSDIPLPELFESSGWITPAGILLLASFFVERGILHRLMGNIGGFKDNRGEYGLTHDIFTTYCLKGSYSIRAQDHDAVMFRDILGYLSRVPTESFQRIKQPQIRKEGQLSERKTDKFITFALNYLEDYGLKDLEGCKACFARSKIVREQENVESINDKEYKPHENKKKVEIHFDQSKEDRFYINRNNVILKIQKKDGHSNIVRMGVYELKYLVLMSLVGKAKEAVEKIDNYIQDLRDQLPYIEGKNKEEIKEYVRFFPRFIRSHLGLLQINDEEKIKARLDYVKTKWLDKKEKSKELELHKKGRDILRYINERCDRELNRNVYNRILELLVSKDLTGFYRELEELKRTRRIDKNIVQNLSGQKTINALHEKVCDLVLKEIESLDTENLRKYLGLIPKEEKEVTFKEKVDRILKQPVIYKGFLRYQFFKDDKKSFVLLVEDALKEKGGGCDVPLGKEYYKIVSLDKYDKENKTLCETLAMDRLCLMMARQYYLSLNAKLAQEAQQIEWKKEDSIELIIFTLKNPDQSKQSFSIRFSVRDFTKLYVTDDPEFLARLCSYFFPVEKEIEYHKLYSEGINKYTNLQKEGIEAILELEKKLIERNRIQSAKNYLSFNEIMNKSGYNKDEQDDLKKVRNSLLHYKLIFEKEHLKKFYEVMRGEGIEKKWSLIV

SEQ ID NO: 11 - protein, 805 aa, synthetic construct; identical to SEQ ID NO: 10
  REGION 1..805: note = Synthetic Sequence

SEQ ID NO: 12 - protein, 838 aa, synthetic construct
  REGION 1..838: note = Synthetic Sequence
MQFENIKDTGQKPIYSIDQYEGAKKWCFAIVLNRACDNYEDNPQLFSESLLRFEEVNRRDWFDKDIRDLIKKADTEDQIEPKRKPNTPVNRRLHDIRNYFSHSRHQDDCLYFKNDDPMRCIMEAAYEKAKIHIKGRQTEQSDIPLPELFDANNKITSAGVLFLASFFVERGILHRLMGNIGGFKDNRGKYGLTHDIFTTYCLKDSYSIHASDPKVVLFRDIAGYLSLVACEYYPTYLSKIPKENAGGKSSDEEKYAERKTDKFILFALKYLEEFVLPSLKDDYLVDIGRIDIIREESKETEEKDEQYKPHPNQGKVKVVFDSINKELPYYINHNTVILRIQKNGVMAYSCKIGVNDLKYLLLLCLQGKTDKALDAIYNYLHSMQDPPEVVKIGATDKLFQGLPEFILKQSGIKVQDKNKEKAARIKYIRDKWEKKKSESADIELHRKGRDILRYVNWHCETPLGTEKYDQLLVLLVNKNFAGFGDELNQLKRTEIISKDIFEKLSGFKTINTLHQKVCNLVLEELSFFEKSNPEKLEEYIGLIRKPAPENNPPPEYKEKVRRFVEQPMIYKGFLRDQFFVNKDQDGKKLKEQKTFAKLVEETLGQNADVPLGKDFYYVPNIEKDEKKNRFHKDNAVLYETLALDRLCAMMARKCLTQINKNLAEKSEEIDWRNEDGKDFIYLKLVKSDRPQETFKIRFKVNDFAKLYVMDDPDFLGGLMKHFFPQEHSIEYHKLYRNGIERYTDRQKDGIEAILRLEDSVIRQKGMKPKPAKNYISFSEIMAQTDYPEHDQKVLNKVRRALLHYHLKFEPADYNRFVDIMKKDKFWDGERKNEESRGK

SEQ ID NO: 13 - protein, 838 aa, synthetic construct
  REGION 1..838: note = Synthetic Sequence
MQFENIKDTGQKPIYSIDQYEGAKKWCFAIVLNRACDNYEDNPQLFSESLLRFEEVNRRDWFDKDIRDLIKKADTEDQIEPKRKPNTPVNRRLHDIRNYFSHSRHQDDCLYFKNDDPMRCIMEAAYEKAKIHIKGRQTEQSDIPLPELFDANNKITSAGVLFLASFFVERGILHRLMGNIGGFKDNRGKYGLTHDIFTTYCLKDSYSIHASDPKVVLFRDIAGYLSLVACEYYPTYLSKIPKENAGEKSSDEEKYAERKTDKFILFALKYLEEFVLPSLKDDYLVDIGRIDIIREESKETEEKDEQYKPHPNQGKVKVVFDSINKELPYYINHNTVILRIQKNGVMAYSCKIGVNDLKYLLLLCLQGKTDKALDAIYNYLHSMQDPPEVVKIGATDKLFQGLPEFILKQSGIKVQDKNKEKAARIKYIRDKWEKKKSESADMELHRKGRDILRYVNWHCETPLGTEKYDQLLVLLVNKNFVVFGDELNQLKRTEIISKDILEKLSGFQTINTLHQKVCNLVLEELSSLEKNDPGKLAEHIGLVRKPAPENNPPPEYKEKVRRFVEQPMIYKGFLRDQFFVNKDQDGKKLKEQKTFAKLVEETLGQNADVPLGKDFYYVPNIEKDEKKNRFHKDNAVLYETLALDRLCAMMARKCLTQINKNLAEKSEEIDWRNEDGKDFIYLKLVKSDRPQETFKIRFKVNDFAKLYVMDDPDFLGGLMKHFFPQEHSIEYHKLYRNGIERYTDRQKDGIEAILRLEDSVIRQKGMKPKPAKNYISFSEIMAQTDYPEHDQKVLNKVRRAVLHYHLKFEPADYNRFVDIMKKNKFWDGERKNKESRGR

SEQ ID NO: 14 - protein, 905 aa, synthetic construct
  REGION 1..905: note = Synthetic Sequence
MNPVDIKEASKKAVYYIDQYKGDKKWCFAIVLNRAYDNLEDNPNLLNESLLRFENISLMSWFDFQEDEKQMRDSVFQGYENINKAEQKKEIKLKPDIQKAVVKRLEDLRNYFSHRLHTDDCLTFLKEDPIRFILEKAYEKAKLVHLGRETQESKDFSVPLFEDDKITTAGVILFASFFVEMRILIRVLGLKDVRGFTLTEGKFNLTRKAICHYALPDSYSIKTPRDTKLFRDILGYLSRMPSESYDHYFPEAKKKAEEGEKDKEGEGKDKENKEPQWSKRNTDKFMVFAMRYLEDFQPDVFRICFARQDIQQPQREKDQERKPHKQKGKRKLQFPENLQEARDNPAVRYFIRQNNIDIQIQKNNQKIHCRMGLNELKYLILLCLKGQGGEAIEAIYNEAGQVGNKLPHLAKMRKEGFQDYQKWMPGFVLNHHGLMPEGPNRKEPVAARVGYIRQKWERKRDGSKEARLDSKARDILQYINECGQEYALQQDRERKGTLNIDKYRKIHDFLVNKNIEAFRQELDGLKVDEKVREDLKKRQTVNDLHQRVCNLMIRKLEDLQQSGDLEQLKCYIGLAPAWEYRDEDQTGKEQKEQRFENKVKNLKPMLYRGFLRERFFAEYRSKENKRNFADLVEDVRQKKGEGDVPVDLIYYQIEGDTQEIQVANKKLTETLARDRLCLLIGREYLEQLNRTLSQNAIEERHGPKRKYFIKTEWSKEPVQMGDGKERMRDVIICRIRENPEDENPLCSIRFAVKHWTKLYVMDDPFFLSDVHSYFLSKSNEIDYHTLNQKGICQYTNLQADCMVNILKLEKKVFERVTKRDIDKEKDIKKLINEINNKLQHLPAKKEDNRVPFLLVCEAALEKGILKEKSEIDVVSMVRNSAFHYQLYFSKSEKETFDRIMRREGI

SEQ ID NO: 15 - protein, 900 aa, synthetic construct
  REGION 1..900: note = Synthetic Sequence
MQTATQEQKQKQSIYSILNYQGQRKWCFAIVLNRALDNINPKRETETGKYKNKELFYKSLLRFEGIKKQPWFDETKAEKENVTAKEIIDSKDKAAELLLNLRNYFSHNYHTEKCLYFGTESQHKQIRLIMEAAYERAKAELTGRRTGQEISAEAEKDKDGNIKKYKLSDVPWPPLFDEKDIITTAGVVFFASFFTEAGQIFRLMNWINGLKRNDDKFNITRRALSFYSLPDSYAEAIAEYEVEEDGASRTIRYKAKIFKDILNYLRRIPSETYKLYHSGEENKISGKKEEKGEDENTPVERKTDKFAEFAMRYLEDFEGVRFARYRINTKTRENEVFFDEDELKKLIDKKGVPEQEKDKKFEDYRYYYVKNNAILKTEKGSIRIGINELKYFVLLSLDKMGQQAKEKINSFLSKFTGDNLGNREFIKANIEELPPFILKKFDPLAEDKEKRIEKRVDYLIRKWKRKKEDYEKMRINDKVGGILRYVNENLKPGKKLNAEGYKYLQKLLTMERFSEFEKEINKFEDERESRLKRGALSEIERLKSIDKMFLKVCSIVLKKLESLEGDELAGYIGLKKQPLTDEKAAENQEYDNVLQRYIETKIALPKGTLRDLYLKTGGKNNFSDAVEDVLEKKKLDFDIELDKKYYDYEIDKRKEAPKDREGLKTARKLRETMAKDRLCLLTGMKFYENIREDLNIRWERRVGKNVIYADIYKKGDKTKKLFTLKFSEKDYVKMYVIDNTDFLRQVWEKFVKGKEGQEVNYHEFYQKGIQKGLGDFQRDVALKVLKFEETVVEKNAGTGLPVSGINNKLSGIIKNRGREMKSDFNKHRDVFVSNDTICKAFFDDADDIERVKKIRNSAFHYNADFEDEDYTGFNKIMDREGIKIKEIDGKKQEGKQRKRF

SEQ ID NO: 16 - protein, 866 aa, synthetic construct
  REGION 1..866: note = Synthetic Sequence
MAVDYSLKNEWYREINKSCFTVALNVAYDNCKAKGHENLLREAQRSKGGITNEQIKNVQTEIKTRLEDIRSHFSHFYHDEKSLIFEKDNIVKDFLESAYEKAQSSVIGSTRQSDYKGVVPPLFEPHDGMITAAGVVFLASFFCHRSNVYRMLGAVKGFKHTGKEELSDGAKRDYGFTRRLMAHYSLRDSYVIKAEETKSFRDLLGYLSRVPQQAVDWLNEHNQLSEDEKKEFLNQKPSDEESQEQSKTENTDRQADRMPRRSLRKTDKFILFAAKFIEDWAQKEKMDVTFARYQKTVTEDENKNQDGKQVRDVQLKYEKDTKKLNPDFDYKWTYYIRNNHAIIQIKPDEYKQAVSARISENELKYLVLLIFQGKGWEAIKKIGDYIFHIGNKIKIGRFDHNEERRMPSFLKNPPADIIGEMVENRLKYIRDELNKVIETIKKEEPQNNKWLLYKGKKISIILKFISDSISDIKKRPDVNEYNTLRDMLQKLDFDNFYERLKSYVSEGRIEQTLYDEIKGIKDISTLCIKICELRLAALEELEKEGGDDLNKYIGLAVQEKHKNYDDSNTPQKKAERFLESQFSVGKNFLRETFYDEYIKNRKSLYEIIKEKITGITPLNENRWYLMDKNPKEFESKDSKIIRGLCNIYIQDILCMKIALWYYENLSPSYKNKLKWDFIGQGFGYDRYKLSYKTDCGITIEFKLADLNRLDIIEKPKMIENICHSFILEKDVKKQTISWHEFRQDGIAKYRKLQKEVVEAVFEFENSLKIPDKNWLTQGYVPFNKNKRFEDKGFSTFILEEAVRKGKIKSDDKEPLRKVRTDFFHEQFDSTDAERRIFDKYMPAKHDGKNKGGKMQEKQEKSYTRRI

SEQ ID NO: 17 - protein, 871 aa, synthetic construct
  REGION 1..871: note = Synthetic Sequence
MEKYLIKNFEGINKSKFTVALNIANDNCKNKGIQELLKEAQRSKGGITDTQITEVQEHIKERLNSVRNYFSHCYHEKKPLYFEANDPVKIFLEETFAKAVENLQGRFLSDKYKLTVPPLFEPNQNNTITAAGVIFLASFFCHRSYVYRMLGGIPGFKRSDKKKWGDGQKIDYGFTRKLMSFYSLRDSYSVNVQENKELTAFRDILGYLARVPGQAIDWLIEKGKLTKEEGKQFYLGEQSEEREEKAKKEEIKYALRKTDKFMLFAVRFIEDWAEQERIKVEFARYEKMTIVNENKKQDEKEERKVKFVSDEPTAAGWTYYIRNNHAIIKIIPDDKKKKAVSARISENELKYLVLTIIDGNGKNAIAYIGDYIFRTARQIENKSYNAESEKYAPAFVRGGQKKSVDKRIKYIRDEIQQVINDIEAEQEKQKNEQDAPAENRTWLIYKGKKISIILRYVNDNIAEYKKRLSVTEYNELRGYLQQLDFINFHRKLAEYQHHGRLPNGFAESINKFQDLSKLCIEVCERQKKKLQEMAAKGGIELEQYIGLAPKEENQEQNKYATKANNFIKVWLSIPENFLRQKFYDKFCKQQECKNKGSDKPDNTSVPQRKYFIAIIREKNIRPIHADKYYLLGQNPKDYERPDGKIIRQLCDVYCKDGLCMAMAKWYYENRLGKFKDLIEWQTGDDKQQHGYAGHTLEYQATEKIKIRFKLADFTRLDIIEPPERVKNICRQWETELLKKTRDGTISWYDFKLNGLEPYRQWQGYAVADIFWFEESLKINETQWQGRTHMPFNFEKDKPLWCNILDEAVKQNKIEKQDTQALRRVRHDCFHEEFLANYEQLKIFKNLISDKAKDAKPKDKKSRKNEQKYGKR

SEQ ID NO: 18 - protein, 789 aa, synthetic construct
  REGION 1..789: note = Synthetic Sequence
MENIKLEKQKAAFYFNQAELNLKAIEGNIFDKGRRKTLFDNPKILSKVENFIFNFKDVTKNAKGEIDCLLSKLMELRNFYSHYVHKPDVKELSKGEKPLLERYYQIAIEATGSENVKLEIIENDKWLTDAGVLLFLCMFLKKSQANKLISGISGFKRNDTFGQPRRNLFNYFSVRERYKVVPDMQKHFLLFVLVNHLSEQDDYIEKAQQPYNIGEGLFFHRIASTFLNVSGILRNMEFYTYQSKRLKEQRGELKREKDIFTWEEPFQGNSYFEINGHKGVIGEDELKELCYALLSYNKSKYAVEQIEKFLKGFGEVKSEQEIRDSDILNESYFPTNYFAESNIGSIKEKILNRLGKTDDSYKKTGTKIKPYDMMKEVMEFINNSLPADEKLKRKDYRRYLKMVRIWDSEKDNIKREFESKEWSKYFSSNFWMAKNLERVYGLAREKNAELFNKLKAVVEKMDEREFEKYRQINSAEDLASLRRLANDYGVKWEEKDWQEYSGQIKKQISDRQKLTIMKQRITAELKKKHGIENLNLRITIDSNKSRKAVLNRIAVPRGFVKEHILGWQGSEKVSKKTREAKCKILLSKEYEELSKQFFQTRNYDKMTQVNSLYEKNKLIAFMAVYLMGQLNIRFDKPTRLNELEKAEVDFKISDKVTAKIPFSQYPSLVYAMSSKYADSVGSYKFENDEKNKPFLGKIDIIEKQRMEFIKEVLGFEEYLFEKKIIDKSKFADTATHISFREICDELIQKGWDENKLTNLKDARNAALHGEIPAETSFREAKPLINGLKK

SEQ ID NO: 19 - protein, 792 aa, synthetic construct
  REGION 1..792: note = Synthetic Sequence
MSPDFIKLEKQEAAFYFNQTELNLKAIESNIFDKQQRVILLNNPQILAKVGDFIFNFRDVTKNAKGEIDCLLLKLRELRNFYSHYVYTDDVKILSNGERPLLEKYYQFAIEATGSENVKLEIIESNNRLTEAGVLFFLCMFLKKSQANKLISGISGFKRNDPTGQPRRNLFTYFSVREGYKVVPDMQKHFLLFVLVNHLSGQDDYIEKAQKPYDIGEGLFFHRIASTFLNISGILRNMEFYIYQSKRLKEQQGELKREKDIFPWIEPFQGNSYFEINGNKGIIGEDELKELCYALLVAGKDVRAVEGKITQFLEKFKNADNAQQVEKDEMLDRNNFPANYFAESNIGSIKEKILNRLGKTDDSYNKTGTKIKPYDMMKEVMEFINNSLPADEKLKRKDYRRYLKMVRIWDSEKDNIKREFESKEWSKYFSSDFWMAKNLERVYGLAREKNAELFNKLKAVVEKMDEREFEKYRLINSAEDLASLRRLAKDFGLKWEEKDWQEYSGQIKKQISDRQKLTIMKQRITAELKKKHGIENLNLRITIDSNKSRKAVLNRIAVPRGFVKEHILGWQGSEKVSKKTREAKCKILLSKEYEELSKQFFQTRNYDKMTQVNGLYEKNKLLAFMVVYLMERLNILLNKPTELNELEKAEVDFKISDKVMAKIPFSQYPSLVYAMSSKYADSVGSYKFENDEKNKPFLGKIDTIEKQRMEFIKEVLGFEEYLFEKKIIDKSEFADTATHISFDEICNELIKKGWDKDKLTKLKDARNAALHGEIPAETSFREAKPLINGLKK

SEQ ID NO: 20 - protein, 792 aa, synthetic construct; identical to SEQ ID NO: 19
  REGION 1..792: note = Synthetic Sequence

SEQ ID NO: 21 - protein, 790 aa, synthetic construct
  REGION 1..790: note = Synthetic Sequence
MNGIELKKEEAAFYFNQAELNLKAIEDNIFDKERRKTLLNNPQILAKMENFIFNFRDVTKNAKGEIDCLLLKLRELRNFYSHYVHKRDVRELSKGEKPILEKYYQFAIESTGSENVKLEIIENDAWLADAGVLFFLCIFLKKSQANKLISGISGFKRNDDTGQPRRNLFTYFSIREGYKVVPEMQKHFLLFSLVNHLSNQDDYIEKAHQPYDIGEGLFFHRIASTFLNISGILRNMKFYTYQSKRLVEQRGELKREKDIFAWEEPFQGNSYFEINGHKGVIGEDELKELCYAFLIGNQDANKVEGRITQFLEKFRNANSVQQVKDDEMLKPEYFPANYFAESGVGRIKDRVLNRLNKAIKSNKAKKGEIIAYDKMREVMAFINNSLPVDEKLKPKDYKRYLGMVRFWDREKDNIKREFETKEWSKYLPSNFWTAKNLERVYGLAREKNAELFNKLKADVEKMDERELEKYQKINDAKDLANLRRLASDFGVKWEEKDWDEYSGQIKKQITDSQKLTIMKQRITAGLKKKHGIENLNLRITIDINKSRKAVLNRIAIPRGFVKRHILGWQESEKVSKKIREAECEILLSKEYEELSKQFFQSKDYDKMTRINGLYEKNKLIALMAVYLMGQLRILFKEHTKLDDITKTTVDFKISDKVTVKIPFSNYPSLVYTMSSKYVDNIGNYGFSNKDKDKPILGKIDVIEKQRMEFIKEVLGFEKYLFDDKIIDKSKFADTATHISFAEIVEELVEKGWDKDRLTKLKDARNKALHGEILTGTSFDETKSLINELKK

SEQ ID NO: 22 - protein, 790 aa, synthetic construct; identical to SEQ ID NO: 21
  REGION 1..790: note = Synthetic Sequence

SEQ ID NO: 23 - protein, 797 aa, synthetic construct
  REGION 1..797: note = Synthetic Sequence
MSGIELKKEEAAFYFNQAELNLKAIEVSIFDEGRRKTLLNNPKILAKVENFIFNSEDVTKNAKGEIDCLLSKLMELRNFYSHYVHKPDVKELSKGEKPILEKYYQFAIDATASADVKLEIIENDTWLTDAGVLLLLCMFLKKSQANKLIGGISGFKRNDPTGQPRRNLFTYYSVREGYKVVPEMQKHFLLFALVNHLSNQDDYIEKAQQPYDIGEGLFFHRIASTFLDISGILRNMKFYTYQSKRLKEQRGELKREKDSFEWIEPFQGNSYFSVDGQKGVIGEDELKELCYALLIGKQDANKVEGRITQFLKKFKNADDAQKVSDDEMLDRGNFPASYFAERRVGSIKDKILSSLEQAIKSYKTSGADVKAYNKMKEVMEFINNSLPVDEKLKRKDYKRYLGMVRLWGSERDNIKREFEAKGWSKYFTSGFWMAKNLERVYGLAREKNAELFNKLKTAVEKMDEREFVKYQQINDAKDLASLRQLANDFGVNWEEKDWEKYSGQIKKQITDSQKLTIMKQRITAGLKRKHGIENLNLRITIDSSKSRKAVLNRIAIPRGFVKKHILDWQGSEKVPKKIREAKCKILLSKEYEELSRQFYKVKDYDKMTQINSLYEKNKLIALMAVYLMEQLRIQLKEHTELRNLDKTTVDFRISDKVTEKIPFSQYPSLVYAMSREYADNVDNYKFSEEDKKKLDKIKKNLFLGKIDIIEKQRMEFIKEVLGFEEYLFDDKIIDRSKFADTATHISFGEIVGELIGKGWDKDKLTKLEYARNKALHGEIPEATSFNEAKQLINELKK

SEQ ID NO: 24 - protein, 792 aa, synthetic construct
  REGION 1..792: note = Synthetic Sequence
MSPDFIKLEKQEAAFYFNQTELNLKAIESNILDKQQRMILLNNPRILAKVGNFIFNFRDVTKNAKGEIDCLLFKLEELRNFYSHYVHTDNVKELSNGEKPLLERYYQIAIQATRSEDVKFELFETRNENKITDAGVLFFLCMFLKKSQANKLISGISGFKRNDPTGQPRRNLFTYFSAREGYKALPDMQKHFLLFTLVNYLSNQDEYISELKQYGEIGQGAFFNRIASTFLNISGISGNTKFYSYQSKRIKEQRGELNSEKDSFEWIEPFQGNSYFEINGHKGVIGEDELKELCYALLVAKQDINAVEGKIMQFLKKFRNTGNLQQVKDDEMLEIEYFPASYFNESKKEDIKKEILGRLDKKIRSCSAKAEKAYDKMKEVMEFINNSLPAEEKLKRKDYRRYLKMVRFWSREKGNIEREFRTKEWSKYFSSDFWRKNNLEDVYKLATQKNAELFKNLKAAAEKMGETEFEKYQQINDVKDLASLRRLTQDFGLKWEEKDWEEYSEQIKKQITDRQKLTIMKQRVTAELKKKHGIENLNLRITIDSNKSRKAVLNRIAIPRGFVKKHILGWQGSEKISKNIREAECKILLSKKYEELSRQFFEAGNFDKLTQINGLYEKNKLTAFMSVYLMGRLNIQLNKHTELGNLKKTEVDFKISDKVTEKIPFSQYPSLVYAMSRKYVDNVDKYKFSHQDKKKPFLGKIDSIEKERIEFIKEVLDFEEYLFKNKVIDKSKFSDTATHISFKEICDEMGKKGCNRNKLTELNNARNAALHGEIPSETSFREAKPLINELKK

SEQ ID NO: 25 - protein, 803 aa, synthetic construct
  REGION 1..803: note = Synthetic Sequence
MNAIELKKEEAAFYFNQARLNISGLDEIIEKQLPHIGSNRENAKKTVDMILDNPEVLKKMENYVFNSRDIAKNARGELEALLLKLVELRNFYSHYVHKDDVKTLSYGEKPLLDKYYEIAIEATGSKDVRLEIIDDKNKLTDAGVLFLLCMFLKKSEANKLISSIRGFKRNDKEGQPRRNLFTYYSVREGYKVVPDMQKHFLLFTLVNHLSNQDEYISNLRPNQEIGQGGFFHRIASKFLSDSGILHSMKFYTYRSKRLTEQRGELKPKKDHFTWIEPFQGNSYFSVQGQKGVIGEEQLKELCYVLLVAREDFRAVEGKVTQFLKKFQNANNVQQVEKDEVLEKEYFPANYFENRDVGRVKDKILNRLKKITESYKAKGREVKAYDKMKEVMEFINNCLPTDENLKLKDYRRYLKMVRFWGREKENIKREFDSKKWERFLPRELWQKRNLEDAYQLAKEKNTELFNKLKTTVERMNELEFEKYQQINDAKDLANLRQLARDFGVKWEEKDWQEYSGQIKKQITDRQKLTIMKQRITAALKKKQGIENLNLRITTDTNKSRKVVLNRIALPKGFVRKHILKTDIKISKQIRQSQCPIILSNNYMKLAKEFFEERNFDKMTQINGLFEKNVLIAFMIVYLMEQLNLRLGKNTELSNLKKTEVNFTITDKVTEKVQISQYPSLVFAINREYVDGISGYKLPPKKPKEPPYTFFEKIDAIEKERMEFIKQVLGFEEHLFEKNVIDKTRFTDTATHISFNEICDELIKKGWDENKIIKLKDARNAALHGKIPEDTSFDEAKVLINELKK

SEQ ID NO: 26 - protein, 811 aa, synthetic construct
  REGION 1..811: note = Synthetic Sequence
MTKSLLLWRYLIMNIIKLKKEEAAFYFNQTILNLSGLDEIIEKQIPHIISNKENAKKVIDKIFNNRLLLKSVENYIYNFKDVAKNARTEIEAILLKLVELRNFYSHYVHNDTVKILSNGEKPILEKYYQIAIEATGSKNVKLVIIENNNCLTDSGVLFLLCMFLKKSQANKLISSVSGFKRNDKEGQPRRNLFTYYSVREGYKVVPDMQKHFLLFALVNHLSEQDDHIEKQQQSDELGKGLFFHRIASTFLNESGIFNKMQFYTYQSNRLKEKRGELKHEKDTFTWIEPFQGNSYFTLNGHKGVISEDQLKELCYTILIEKQNVDSLEGKIIQFLKKFQNVSSKQQVDEDELLKREYFPANYFGRAGTGTLKEKILNRLDKRMDPTSKVTDKAYDKMIEVMEFINMCLPSDEKLRQKDYRRYLKMVRFWNKEKHNIKREFDSKKWTRFLPTELWNKRNLEEAYQLARKENKKKLEDMRNQVRSLKENDLEKYQQINYVNDLENLRLLSQELGVKWQEKDWVEYSGQIKKQISDNQKLTIMKQRITAELKKMHGIENLNLRISIDTNKSRQTVMNRIALPKGFVKNHIQQNSSEKISKRIREDYCKIELSGKYEELSRQFFDKKNFDKMTLINGLCEKNKLIAFMVIYLLERLGFELKEKTKLGELKQTRMTYKISDKVKEDIPLSYYPKLVYAMNRKYVDNIDSYAFAAYESKKAILDKVDIIEKQRMEFIKQVLCFEEYIFENRIIEKSKFNDEETHISFTQIHDELIKKGRDTEKLSKLKHARNKALHGEIPDGTSFEKAKLLINEIKK

SEQ ID NO: 27 - protein, 1000 aa, synthetic construct
  REGION 1..1000: note = Synthetic Sequence
MSPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYA

SEQ ID NO: 28 - DNA, 36 bp, synthetic construct
  source 1..36: mol_type = other DNA
gttagaatataaccctgtttgtaggggtaataaaac

SEQ ID NO: 29 - DNA, 36 bp, synthetic construct; identical to SEQ ID NO: 28
  source 1..36: mol_type = other DNA

SEQ ID NO: 30 - DNA, 36 bp, synthetic construct
  source 1..36: mol_type = other DNA
gttggaatataaccccgtttgtaggggtaataaaac

SEQ ID NO: 31 - DNA, 36 bp, synthetic construct; identical to SEQ ID NO: 28
  source 1..36: mol_type = other DNA

SEQ ID NO: 32 - protein, 6 aa, synthetic construct
  VARIANT 4: note = X is any naturally occurring amino acid
KKRXKR

SEQ ID NO: 33 - protein, 7 aa, synthetic construct
  VARIANT 5: note = X is any naturally occurring amino acid
KRKRXKR

SEQ ID NO: 34 - protein, 9 aa, synthetic construct
  REGION 1..9: note = Synthetic Sequence
PAAKRVKLD

SEQ ID NO: 35 - protein, 16 aa, synthetic construct
  REGION 1..16: note = Synthetic Sequence
KRPAATKKAGQAKKKK

SEQ ID NO: 36 - protein, 7 aa, synthetic construct
PKKKRKV

SEQ ID NO: 37 - protein, 24 aa, synthetic construct
  REGION 1..24: note = Synthetic Sequence
DPKKKRKVDPKKKRKVDPKKKRKV

SEQ ID NO: 38 - protein, 16 aa, synthetic construct
  REGION 1..16: note = Synthetic Sequence
RQIKIWFQNRRMKWKK

SEQ ID NO: 39 - protein, 187 aa, synthetic construct
  REGION 1..187: note = Synthetic Sequence
MDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 40 - protein, 190 aa, synthetic construct
  REGION 1..190: note = Synthetic Sequence
MDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 41 - protein, 236 aa, synthetic construct
  REGION 1..236: note = Synthetic Sequence
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 42 - protein, 190 aa, synthetic construct
  REGION 1..190: note = Synthetic Sequence
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 43 - protein, 386 aa, synthetic construct
  REGION 1..386: note = Synthetic Sequence
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 44 - protein, 373 aa, synthetic construct
  REGION 1..373: note = Synthetic Sequence
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 45 - protein, 384 aa, synthetic construct
  REGION 1..384: note = Synthetic Sequence
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 46 - protein, 183 aa, synthetic construct
  REGION 1..183: note = Synthetic Sequence
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPEFADCWENFVDHEKPLSFNPYKMLEELDKNSRAIKRRLERIKQS

SEQ ID NO: 47 - protein, 16 aa, synthetic construct
  VARIANT 5: note = X is any naturally occurring amino acid
SGGSXTENSGGSSGGS

SEQ ID NO: 48 - protein, 166 aa, synthetic construct
  REGION 1..166: note = Synthetic Sequence
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 49 - protein, 167 aa, synthetic construct
  REGION 1..167: note = Synthetic Sequence
MSEVEFSHEYWMRHALTLAKRAWDEREVPVGAVLVLNNRVIGEGWNRSIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRQVFNAQKKAQSSTD

SEQ ID NO: 50 - protein, 167 aa, synthetic construct
  REGION 1..167: note = Synthetic Sequence
MSEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMRRQVFNAQKKAQSSTD

SEQ ID NO: 51 - protein, 167 aa, synthetic construct
  REGION 1..167: note = Synthetic Sequence
MSEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMPRQVFNAQKKAQSSTD

SEQ ID NO: 52 - protein, 167 aa, synthetic construct
  REGION 1..167: note = Synthetic Sequence
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 53 - protein, 1226 aa, synthetic construct
  REGION 1..1226: note = Synthetic Sequence
MNPRQGYSLSGYYTHPFQGYEHRQLRYQQPGPGSSPSSFLLKQIEFLKGQLPEAPVIGKQTPSLPPSLPGLRPRFPVLLASSTRGRQVDIRGVPRGVHLRSQGLQRGFQHPSPRGRSLPQRGVDCLSSHFQELSIYQDQEQRILKFLEELGEGKATTAHDLSGKLGTPKKEINRVLYSLAKKGKLQKEAGTPPLWKIAVSTQAWNQHSGVVRPDGHSQGAPNSDPSLEPEDRNSTSVSEDLLEPFIAVSAQAWNQHSGVVRPDSHSQGSPNSDPGLEPEDSNSTSALEDPLEFLDMAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 54 - protein, 701 aa, synthetic construct
  REGION 1..701: note = Synthetic Sequence
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 55 - protein, 677 aa, synthetic construct
  REGION 1..677: note = Synthetic Sequence
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 56 - protein, 560 aa, synthetic construct
  VARIANT 229: note = X is any naturally occurring amino acid
AFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLGRLEDLKSKTLGDLVREAEKIFNKRETPEEREERIRRETEEKEERRRTVDEQKEKERDRRRHREMSKLLATVVIGQEQDRQEGERKRPQLDKDQCAYCKEKGHWAKDCPKKPRGPRGPRPQTSLLTLGDXGGQGQDPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVVGPMGQPLQVLTLNIEDEYRLHETSKEPDVSLGFTWLSDFPQAWAESGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFR

SEQ ID NO: 57 - protein, 215 aa, synthetic construct
  REGION 1..215: note = Synthetic Sequence
MYRRHLKHSRVKNLFKFVSAKMNTVFTVESALEFDTCFHLEYSPSVKFYEAQPEGFYYEFAGRQCPYTPDFRLVDQNDSVSFLEIKPSDKVADPDFLHRFPLKQQRAIELSSPLKLVTEKQIRIDPILGNLKLLHRYSGFQSFTPLHMQLLGLVQKLGRVSLLRLSDSIDAPPEEVLASALSLIARGIMQSDLTVQKIGISSFVWAGGHSGIDHG

SEQ ID NO: 58 - protein, 624 aa, synthetic construct
  REGION 1..624: note = Synthetic Sequence
MDKHNGGLFEDEFVIPQPSTSTSPIDAIQAVLPATVDSFPYVLKVEALHRRDYILWVEKNLAGGWTEKNLTPLLADAALVLPPPTPNWRTLARWRKIYIQHGRKLVSLIPKHQAKGNARSRLPPSDELFFEQAVHRYLVGEQPSIASAFQLYSDSIRIENLGVVENSIKTISYMAFYNRIKKLPAYQVMKSRKGSYIADVEFKAIASHKPPSRIMERVEIDHTPLDLLLLDDDLLVPLGRPSLTLLIDAYSHCVVGFNLNFNQPSYESVRNALLSSISKKDYVKNKYPSIEHEWPCYGKPETLVVDNGVEFWSASLAQSCLELGINIQYNPVRKPWLKPMIERMFGIINRKLLEPIPGKTFSNIQEKGDYDPQKDAVMRFSTFLEIFHHWVIDVYHYEPDSRYRYIPIISWQHGNKDAPPAPIIGDDLTKLEVILSLSLHCTHRRGGIQRYHLRYDSDELASYRMNYPDQTRGKRKVLVKLNPRDISYVYVFLEDLGSYIRVPCIDPIGYTKGLSLQEHQINVKLHRDFINEQMDVVSLSKARIYLNDRIKNELIEVRRNIRQRNVKGVNKIAKYRNVGSHAETSIVHELNHPATNEVISKMESASQPEHCDDWDNFTSGLEPY

SEQ ID NO: 59 - protein, 335 aa, synthetic construct
  REGION 1..335: note = Synthetic Sequence
MDLSCHDADKLRSFIECYVETPLLRAIQEDFDRLRFNKQFAGEPQCMLLTGDTGTGKSSLIRHYAAKHPEQVRHGFIHKPLLVSRIPSRPTLESTMVELLKDLGQFGSSDRIHKSSAESLTEALIKCLKRCETELIIIDAFQELIENKTREKRNQIANRLKYISETAKIPIVLVGMPWATKIAEEPQWSSRLLIRRSIPYFKLSDDRENFIRLIMGLANRMPFETQARLETKHTIYALFAACYGSLRALKQLLDESVKQALAAHAETLKHEHIAVAYALFYPDQVNPFLQPIDEIKACEVKQYSRYEIDAAGKEEVLNPLQFTDKIPISQLLKKR

SEQ ID NO: 60 - protein, 167 aa, synthetic construct
  REGION 1..167: note = Synthetic Sequence
MIEAPDVKPWLFLIKPYEGESLSHFLGRFRRANHLSASGLGTLAGIGAIVARWERFHFNPRPSQQELEAIASVVEVDAQRLAQMLPPAGVGMQHEPIRLCGACYAESPCHRIEWQYKSVWKCDRHQLKILAKCPNCQAPFKMPALWEDGCCHRCRMPFAEMAKLQKV

SEQ ID NO: 61 - protein, 10 aa, synthetic construct
  REGION 1..10: note = Synthetic Sequence
GGSGGSGGSG

SEQ ID NO: 62 - protein, 16 aa, synthetic construct
  REGION 1..16: note = Synthetic Sequence
GGSGGSGGGGSGGGGS

SEQ ID NO: 63 - protein, 5 aa, synthetic construct
  REGION 1..5: note = Synthetic Sequence
GGGGS

SEQ ID NO: 64 - skipped

SEQ ID NO: 65 - protein, 5 aa, synthetic construct
  REGION 1..5: note = Synthetic Sequence
GGGGS

SEQ ID NO: 66 - protein, 8 aa, synthetic construct
GGGGGGGG

SEQ ID NO: 67 - protein, 6 aa, synthetic construct
  REGION 1..6: note = Synthetic Sequence
GGGGGG

SEQ ID NO: 68 - protein, 5 aa, synthetic construct
  REGION 1..5: note = Synthetic Sequence
EAAAK

SEQ ID NO: 69 - protein, 7 aa, synthetic construct
  REGION 1..7: note = Synthetic Sequence
AEAAAKA

SEQ ID NO: 70 - protein, 12 aa, synthetic construct
  REGION 1..12: note = Synthetic Sequence
AEAAAKEAAAKA

SEQ ID NO: 71 - protein, 16 aa, synthetic construct
  REGION 1..16: note = Synthetic Sequence
AEAAAKALEAEAAAKA

SEQ ID NO: 72 - protein, 5 aa, synthetic construct
  REGION 1..5: note = Synthetic Sequence
PAPAP

SEQ ID NO: 73 - protein, 18 aa, synthetic construct
  REGION 1..18: note = Synthetic Sequence
KESGSVSSEQLAQFRSLD

SEQ ID NO: 74 - protein, 14 aa, synthetic construct
  REGION 1..14: note = Synthetic Sequence
EGKSSGSGSESKST

SEQ ID NO: 75 - protein, 12 aa, synthetic construct
  REGION 1..12: note = Synthetic Sequence
GSAGSAAGSGEF

SEQ ID NO: 76 - DNA, 82 bp, synthetic construct
  source 1..82: mol_type = other DNA
cgccccattgacgcaaatgggcggtaggcgtgtacggtgggaggtctatataagcagagctggtttagtgaaccgtcagatc

SEQ ID NO: 77 - protein, 22 aa, synthetic construct
  REGION 1..22: note = Synthetic Sequence
GSGPKKKRKVAAAYPYDVPDYA

SEQ ID NO: 78 - protein, 12 aa, synthetic construct
  REGION 1..12: note = Synthetic Sequence
MSPKKKRKVEAS

SEQ ID NO: 79 - protein, 234 aa, synthetic construct
  REGION 1..234: note = Synthetic Sequence
CAGCTCACAGACATGATAAGATACATTGATGAGTTTGGACAAACCACAACTAGAATGCAGTGAAAAAAATGCTTTATTTGTGAAATTTGTGATGCTATTGCTTTATTTGTAACCATTATAAGCTGCAATAAACAAGTTAACAACAACAATTGCATTCATTTTATGTTTCAGGTTCAGGGGGAGGTGTGGGAGGTTTTTTAAAGCAAGTAAAACCTCTACAAATGTGGTATTGGC

SEQ ID NO: 80 - protein, 782 aa, synthetic construct
MESNKNPSNRHSLPKVIISDVDKDNILEFKVKYEKLGRLDKFKIVSMKYEDRDIVFRDIVSSDKSLEFSLANSNREIIVNLDNKKYTIRGQRVDNNEEKAKKVQLILTDNIKDENGAIRETLTERELIDNSDSIYSKIAGRKINSSKDIYLIKRYLAYRSNLQFFYNFIDKFFKIVDNKELWNIEFGNKHIEYFKFLINDNIKNANGYLYSYLQDNRRVKNDLYKTKDIFSKLRHALMHFDYEFFDKLFNNENLELDLNIEFLNLTIQNIDKLNIDTKKSYIGNQKIKIYSEEIKLDELYNLYNTISINRLGFNRLINSFFMQDGLENRKLKEFFNEEANSEEIYFVDIHQNRDYKKLYIKHKNFVAKLYGNRDGKSIAKLNRDISNIKKQMQEITDKNSTLRLEYKLRVAFGFIYTNYKNYRHFKNSFDNDLKSGRFNNIDLSQIIEYYKNSYTNKDVLIRVTIKKIDKLNLNALIKDDNLLKIILLIFTFIPNELKGKFLGFIKRYYHDIKHIDEDSKEELEFNDGLSTSLKLKILHKNIRKLTILKYSLATESKYNKKDNYYYEDGHKTKRFLSSLGVSHNIEEFDKTIYTPFFKYYSAMYKLINDFEIFALTQFDSSANLKEITMKEELKQDNEYNFKILLRETNLYDENIVKLRNKISHIDGEFLFSNPLNRRINISSMREKITNFIDSKNIKKILGYDALNDLSMKIIQQKTKLEANANKDEKINELIKNAQKANDYYSIYKLKAIEGINKRLLKIIGETKQEKYIKDKIIKGNNK

SEQ ID NO: 81 - protein, 782 aa, synthetic construct
MESNKNPSNRHSLPKVIISDVDKDNILEFKVKYEKLGRLDKFKIVSMKYEDRDIVFRDIVSSDKSLEFSLANSNREIIVNLDNKKYTIRGQRVDNNEEKAKKVQLILTDNIKDENGAIRETLTERELIDNSDSIYSKIAGRKINSSKDIYLIKRYLAYRSNLQFFYNFIDKFFKIVDNKELWNIEFGNKHIEYFKFLINDNIKNANGYLYSYLQDNRRVKNDLYKTKDIFSKLRHALMHFDYEFFDKLFNNENLELDLNIEFLNLTIQNIDKLNIDTKKSYIGNQKIKIYSEEIKLDELYNLYNTISINRLGFNRLINSFFMQDGLENRKLKEFFNEEANSEEIYFVDIHQNRDYKKLYIKHKNFVAKLYGNRDGKTIARLNRDISNIKKQMQEITDKNSTLRLEYKLRVAFGFIYTNYKNYMHFENSFDNDLKSGRFNNIDLSKIIEYYKNSYTNKDVRIRVTIKKIDKLNLNALIKDDNLLKIILLIFTFIPNELKGEFLGFIKRYYHDIKHIDEDSKEELEFNDGLSTSLKLKILHKNIRKLTILKYSLATESKYNKKDNYYYEDGHKTKRFLSSLGVSHNIEEFDKTIYTPFFKYYSAMYKLINDFEIFALTQFDSSANLKEITMKEELKQDNEYNFKILLRETNLYDENIVKLRNKISHIDGEFLFSNPLNRRINISSMREKITNFIDSKNIKKILGYDALNDLSMKIIQQKTKLEANANKDEKINELIKNAQKANDYYSIYKLKAIEGINKRLLKIIGETKQEKYIKDKIIKGNNK

SEQ ID NO: 82 - protein, 782 aa, synthetic construct
MESNKNPSNRHSLPKVIISDVDKDNILEFKVKYEKLGRLDKFKIVSMKYEDRDIVFRDIVSSDKSLEFSLANSNREIIVNLDNKKYTIRGQRVDNNEEKAKKVQLILTDNIKDENGAIRETLTERELIDNSDSIYSKIAGRKINSSKDIYLIKRYLAYRSNLQFFYNFIDKFFKIVDNKELWNIEFDNKHIEYFKFLINDNIKNANGYLYSYLQDNRRVKNDLYKTKDIFSKLRHALMHFDYEFFDKLFNNENLELDLNIEFLNFTIQNIDKLNIDTKKSYIGNQKIKIYSEEIKLDELYNLYNTISINRLGFNRLINSFFMQDGLENRELKKFFNEEANSEEIYFVDIHQNRDYKKLYIKHKNFVAKLYGNRDGKSIAKLNRDISNIKKQMQEITDKNSTLRLEYKLRVAFGFIYTNYKNYRHFKNSFDNDLKSGRFNNIDLSKIIEYYKNSCTNKDVRIRVTIKKIDKLNLNALIKDDNLLKIILLIFTFIPNELKGEFLGFIKRYYHDIKHIDEDSKEELEFNDGLSKSLKLKILHKNIRKLTILKYSLATESKYNKKDNYYYEDGHKTKRFLSSLGVSHNIEEFDKTIYTPFFKYYSAMYKLINDFEIFALTQFDSSANLKEITMKEELKQDNEYNFKILLRETNLYDENIVKLRNKISHIDGEFLFSNPLNRRINISSMREKITNFIDSKNIKKILGYDALNDLSMKIIQQKTKLEANANKDEKINELIKNAQKANDYYSIYKLKAIEGINKRLLKIIGETKQEKYIKDKIIKGNNK

SEQ ID NO: 83 - protein, 804 aa, synthetic construct
MTKKPANRHALPKVIISEVDSEKILEFKIKYEKLARLDRVEVKAMHYEGKSIVFDEVVVNGGLIDVEYQDDHKTLFVKVGEKSYSIRGQKVGGKQRLREERVSQVKVQLELTDGSSERVSRTERELIVADNIKLYSQIVGREVKTTKEIYLAKRFLGYRSDLLFYYGFVDNFFKVAGNEKELWKIDFEASESSQLLAYIPYMVNDNLKNNDAYLKDYIANEEQIKSDLKKVQTIFSELRHALLHFNYDFFEKLFNGEDVGFDFDIEFLNLLIANIDKLNIDAKKEFITDEKIKLFGENLSLAKVYKLYSDICVNRVGFNKFINSMLIKDGLENQALKSEFDRKQGHKAYYIDIHSNEEYKRLYNRHKALVIKVSTLRDGQKIRKGNAEISEFKKQMNSMTTKNSLSHLEHKMRLAFGFMYGEYNHYNAFKNGFDTDVKNRKFDETDVSKSKAYFLSTYERQKPRTREKLERVAKDIESLKLETLIAHDPLLKFILLMFAFMPREIKGEFLGFVKKYYHDVHSIEVDIIEQELDVVESMSTSLKLKNLGRNIRSLTLFKYALSAKVNYNGSDESFYEEGNRYGKIYKKLGISHNQEEFDKTLVVPLFRYYSALFKLMNDFEIYSLAKANPTALNLQMLVDDETSPYKQGNYYNFNKMLREVHGVTNDEIKNGQAVFMRNKIAHFDTEVLLSKPLLGQTKMNLQRKIIIEFIKARGEMREILGYDAINDFRMKVVHLRTKMKVYSDKLQTMMDLLRSAKTPNDFYNVYKVKGVESINKQLLEVLAETAEERSIEKQICEGNMKYNS

SEQ ID NO: 84 - protein, 806 aa, synthetic construct
MSKNPSNRNSLPKVIINKVDENIILEFKIKYEKLARLDRFEVRSMRYDGDGRIIFDEVVANGGLLDVGYEDDNKTIVVKIENKAYKIYGKKVGGKKRLNGKISKAKVQLILTDNIRKNANDTHRQSLTERELIDKNEIDLYSKIAEREISSTKDIYLVKRFLAYRSDLLLYYAFVNDYVKVKGNKEEFWKTPIDDKIIDYFIYTINDTLKNKEGYLEKYIVDRDQIKKDLEKTKRIFSHLRHKLMHYDFRFFTDLFDGKDVDIKVDNSTQKISELLDIKFLNIVIEELEKLNIDAKKEFIDDEKIPLFRQEIELKKLYSIYAHTAINRVAFNKLINSFLIKDGIENKELKEYFNAQNQGKESYYIDIHQNKEYKKLYIEHKDLLAKLSATKNGKEIAKINRELADKKEQMKQITKANSLKRLEYKLRLAFGFIYTEYKDYETFKNSFDTDTKNQKFDAIDNAKIIEYFEATNKAKKIEKLEEILKGIDKLSLKTLIQDDILLKFLLLFFTFLPQEIKGEFLGFIRKYYHDITSLDEDTKDKDDEITELSRSLKLKIFAKNIRKLSILKHSLSYQIKYNKKESSYYEVGNAFNKMFKKQAISHNLEEFGKSIYLPMLKYYSALYKLINDFEIYALYKDMDTSETLSQQVDKQEYERNEYFNFETLLRKKFGNDIEKVLVTYRNKIAHLDFNFLYDKPINKFISLYKSRDKIVNYIKNHDTQAVLKYDAVNDFVMKVIQQRSKLKVYADKEQTIESMIQNAQNPNDFYNIYKVKAVENINQHLLKVIGYTDSEKAIEEKIRAGNISKS

SEQ ID NO: 85 - protein, 843 aa, synthetic construct
MLKKPVNRYALPKVIISEVNHEDILEFKIKYEKLGRLDRVAVKKMHYEKENIVFDEVDVNGGLIEVMYKDEHQILLVQAGGKSYSIRGKKIGGKQRKREDRVSQVKIQLELTDGVLDKNEKYRVSQTERELIVNDNIKIYSQIVGKEVKTTKEIYLIKRFLGYRSDLLFYYGFVDNFFKVVGNKTELWKINFQDTKNEKLIEYFKFSINDKLKNDETYLKVYSSDNQNIEEDLTKVKNNFSKLRRALMHFDYGFFEKLFNDEDVGFDLDIMFLNVIIKNLDKLNIDTRKEFIDDEKIKIFGEELSLKHLYGMYAHIAINRVAFNKLINSFMMQDGVENRSLKEYFNKRAKDGVAYEVDIHQNSQYKELYKQHKNLVSKVSALSDGVAIAKMNDEIYTLKEKMKQITKPNSLKRLEHKLRLAFGFIYSEYKDYDDFKNNFNDHIIDGRFVPKDEEGKRRAFDSRELARLQGYYDVTLQNKKPQTKEKLGEVSKKIDSLSLATLIDDDKLLKFILLMFTFMPQELKGEFLGFVKKYYHDTKHIEEDSKDKDKDFADGLSVGLRLKVLDKNIRNLSILKHSLSLQTKYNKKDNYFYEDGNVHGRFFKSLGISHNQEEFSKSVYAPLLKYYSALYKLINDFEIYTLAQYITTEYPTLSKVIDSEKFHLRWDNRSKELVPSDDYVFSTLTNKTYDHEKVKELNFIRNKISHFNSKELFEIPLQGYQMKGKKKLPFFLSKKREEIIDEIELQKDIQKTLGYDAINDFNMKMVQLYTKLKVYANKEETIEKMLEEATTPNDFYNVYKVKGVETINKHLLDVIGETEREKFIRIQIEVNNKRVSNENLDKL

SEQ ID NO: 86 - protein, 876 aa, synthetic construct
MSKKPANRHALPKVIISEVDSEKILEFKIKYEKLARLDRVEVKAMHYEGKSIVFDEVVVNGGLIDVEYQDNHKTLFVKVGEKSYSIRGKKVGGKQRLREERVSQVKVQLELSDGSSERVSRTERELIVNENIKLYSQIVGREVKTTKEIYLAKRFLGYRSDLLFYYGFVDNFFKEAKLFNARKNPIELWSEEFYVNDKLSNYTKFMFNDNLKNSESYLKEYIKNNEKENQKIKNDLESARDIFATLRHNLMHFNYSFFERLFKGKDVKIKNLQTKKFESLSNVLRNIEFLNKVIQSIDKLNIDTRKEFIDKEKIKLFNEDLDLQQLYGFFAHTAINRVAFNKLINSFIIKDGIENEQLKEYFNQRVDGTAYEIDIHQNREYKELYKKHKNLVSKVSTLSDGKEIAKGNSEISALKEQMNKITKANSLKRLEYKLRLAFGFIYTEYGSYKAFVSRFNEDTKRKKIKNVEFEKIGFEKQKEYFKSTFEPLKSKKKDNLEKLIQEYEKLSLNDLIENDTFLKVILLLFIFMPKEVKGDFLGFIKKYYHDTKYIEEDTKEKDEGFTNTLPIGLKLKIVERNIAKLSVLKHSLSLKVKYNRGQYEEDNTYRKVFKKLNISHNQEEFHKSMFSPLLRYYASLYKLINDFEIYTLSHYITDKYSTLNKVIASKQFHYRYVWNRKENKGELVKTDNYTFSTLLSKKYEHKNSQEISEMRNKISHFDEKILFKFPLEEVNSFFKGKGKNKKEEPVKSLVEKREEIISLMEKQTDMQKILGYDAINDFRMKTVQFQTKLKEDSKKKEETIKKMIAEAKIPNDFYNIYKVKGVESINKHLLKIIGQTDKEWKIEGDILDGNFKIACKNQRLEEKQQRAKNKQNLDKL

SEQ ID NO: 87 - protein, 919 aa, synthetic construct
MIKNPSNRHSLPKVIISEVDHEKILEFKIKYEKLARLDRFEVKAMHYEGKEIVFDEVLVNGGLIEVEYQDDNKTLFVKVGEKSYRICGERIGGNYIVTEYKDKNEPKSKKHFRLIEKDGKYFKPNGEEVTKNIRKSSVKVLLTLTDGVEDNNGKLRKSRTERELIVADNIKLYSQIVGREVTTTKEIYLVKRFLGYRSDLLFYYGFVNNFFHVAGKREELWKIDFDTLPSNSPLLEYFKFTINDEKYLKSYSSDIQQIKKDLQNNKYIFLVNGEDIEIKAENYNIKPLSELLNIEFLNIQIKKDLQNSKYIFSALRHALMHFDYDFFVRLFNGEDIEIKAKNGNKKPLSELLNIEFLNIMIENIDKLNIDTRKEFIDDDDVPIKLFGEEMKPKNLYGLYAHTAINRVAFNKLINSFMMENGVENQALKSYFDQKAGGVAYEVDIHQNSNYKKLYVKHKNLVSKVSTLSDGQEIAKVNAKISELKEQMKKITKANSLKRLEHKFRLAFGFVYSEYKDYEAFKNNFDTDIKKGKFVPKDKEGKRRAFDHRELEQLKGYFDSTFKSKKPNTKEKLGELSKSTGKLSLKALIGDDMFLKFILLMFTFMPQELKGEFLGFVKKYYHDTKHIEEDTKDRDDGFSNERPMGLKLKVLDKNIRSLSILKHSLSFQTKYNKKDKSFYEDGNVHGKFYKKLGISHNQEEFNKSVYAPLFKYYSALSKLINDFEIYSLTQHVVGSETLAQQVRKRKFIKKGYYNFGNLLKKTDSIIRSSRDNDIFYAVIDMRNTISHLSVEPMFDYPLNGKKFYKLYENKVICVDPLKSRKMIIDFIKRQTEMKKTLGYDAVNDFTMKMVQLQTKLKVYANKEKTIEKMKEEAQTPNDYYNIYKVKGVEAINQYLLEIIGETDDEALIRKLINRGNSINP

SEQ ID NO: 88 - protein, 846 aa, synthetic construct
MKKIKNPSNRNSLPSIIISRFDDKNIYELKVKYEKLARLDKLEIEDMSLDEESTLLFKKVKFNGIEIEIKNQKLLEFDSYIISGKKQTNTTGKTIISLLKEGKKVTYNVTKKDGKYYKNGKEFIIPQNANKLPDRLINDKFIITIEDKVRDEDTKRKKETQRDILSDDTIETYKRISSYKSIKSEDIYTIKRYITFKSDMMFFYTFVDDFFNPIKKQDLWKVKFGEVENLGKFIEFTLNDTLKNPKGILETYCKDLKTVQADFAKINTIFSKIRHSLVHFDFVFIDKLLSNQKIEEFDFDIKLLNDVIDKTQDLYYEAKKEFIEDEKITILDEKDMEIKKLYTFFSKIDIKQPAFNKLINSFIIKDGIENIELKTYIKEKYKSEYFIDIHANKEYKKIYNEHKKLVGENQFLQLNPKENGQKIKELNDQVEEYKKQMKTITEANSLKRLEFKLRLAFGFIKVEYGRFDTFKNSFDEDIKKGKFKEISFEKIKGYLDKTYAKEQFFNYGSNKKTKKPYSILDDIENETLKELVQNDNLLKVILLFYIFTPKELKGEFLGFIKKFYHDTKNITKDTKDEEKELENLKLETPLKLKILEKNLKKITIFNYSIFSNINFDTTNKRFYAEGNRFNRIYKKLNISHNQDEFDKSLFAPLLQYYMNLYKLIGDFEIYLLLKFDNKKDLSELSNDERLKFRGYYNFTTLLSKWFQFDPKRDKKYEKVLRLRNTISHQDINNMIINFEKSTILSQRENIVQLIEEQNDLKEILKYDAVNDFTMKTIQLLKSIEIQSDKSKTINELLSNKDISANDFYNIYKVKGVEMIKKELFNRLGKREIEKKIEEEIAKSTIC

SEQ ID NO: 89 - protein, 822 aa, synthetic construct
MEKIKKPSNRNSIPSIIISDYDASKIKEIKVKYLKLARLDKITIQDMEIVDNIVEFKKILLNGTEHTIIDNKKIEFDNYEITGCIKPSNKRRDGKISQAKYVVTITDKYLRDNEKEKRFKSTERELPNDILLSRYKQISGFDTLTSKDIYKIKRYIDFKNEMLFYFQFIEEFFNPLLPKGKNFYDLNIEQNKDKVAKFIVYRLNDDFKNQSLNSYIQKTDTIKYDFIKVQKILNDFRHALAHFDFEFIQKFFDNQLDKTRFDINTISLIKTLLQKKEGKHYQEKNNYIDDNDTLTIFDDKDSKFSKLHNFYTKISQKKPAFNKLINSFLSQDGIPNEEFKRYLATKKLDFFEDIHSNKEYKEIYINHKNQVIEKQKEESQEKPDGQKLKNFNDELQKLKDKMNTITKQNSLNRLEVKLRLAFGFIANEYNYNFKNFNDNFTLDVKNEQKIKAFKNSSNEKLKEYFESTFEAKQFFYYGKNKNIFNSIENETLEELVKESPLLQIITFLYLFIPKELQGEFVGFILEIYHHTKNISSDTKEDEISIEDAQNSFSLKLKILAKNLRGLQLFNYSLSHNTLYNNKQDFFYEKGNRWQNIYKNFQISHNQDEFDIHLVIPVIKYYINLNKLIGDFEIYALLKYADQNSITEKLSDITKRDDLKFKGHYNFSTLLNRTFGISVYSDKNPISIQNIKQIRNDIAHQNIENMLKAFENSEIFAQREEIVNYLQTEHQMQEILHYNPINDFTMKTVQYLKSLSVHSQKEGKIADIHKKDNLVPNDYYLIYKLKAIEILKQKVIEAIGETKDEKKIKNAIAKEEQIKKGNN

SEQ ID NO: 90 - DNA, 36 bp, synthetic construct
  source 1..36: mol_type = other DNA
gttagaatataccccattttgtatggggattaaaac

SEQ ID NO: 91 - DNA, 36 bp, synthetic construct
  source 1..36: mol_type = other DNA
gttggaatatagccctgtttgtaggggtaataaaac

SEQ ID NO: 92 - DNA, 36 bp, synthetic construct
  source 1..36: mol_type = other DNA
gatggaatataaccccgtttgtaggggtaataaaac

SEQ ID NO: 93 - DNA, 39 bp, synthetic construct
  source 1..39: mol_type = other DNA
agtagaatataaccctgatattagtaggggtaataaaac

SEQ ID NO: 94 - DNA, 36 bp, synthetic construct
  source 1..36: mol_type = other DNA
agtagaatataaccctgttagtaggggtaataaaac

SEQ ID NO: 95 - DNA, 37 bp, synthetic construct
  source 1..37: mol_type = other DNA
tattgactatacccctgatttgtaggggtaaaagaga

SEQ ID NO: 96 - DNA, 37 bp, synthetic construct
  source 1..37: mol_type = other DNA
tattgactatacccctgatttgtaggggtaaaaaaac

SEQ ID NO: 97 - DNA, 36 bp, synthetic construct
  source 1..36: mol_type = other DNA
gtcagactataccctcgtttgtagggggaataaaac

SEQ ID NO: 98-101 - skipped (4 entries)

SEQ ID NO: 102 - protein, 12 aa, synthetic construct
MSPKKKRKVEAS

SEQ ID NO: 103 - protein, 22 aa, synthetic construct
GSGPKKKRKVAAAYPYDVPDYA

SEQ ID NO: 104 - DNA, 82 bp, synthetic construct; identical to SEQ ID NO: 76
  source 1..82: mol_type = other DNA

SEQ ID NO: 105 - DNA, 234 bp, synthetic construct
  source 1..234: mol_type = other DNA
cagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggc

(19) United States
(12) Patent Application Publication
(10) Pub. No.: US 2024/0344087 A1
(43) Pub. Date: Oct. 17, 2024

(54) GENE-MODIFYING ENDONUCLEASES

(71) Applicant: Amber Bio Inc., San Francisco, CA (US)

(72) Inventors: Basem AL-SHAYEB, San Francisco, CA (US); Jacob BORRAJO, San Francisco, CA (US); Mohammad Kamyab JAVANMARDI, San Francisco, CA (US); Kushagra SHARMA, San Francisco, CA (US)

(21) Appl. No.: 18/518,831

(22) Filed: Nov. 24, 2023

Related U.S. Application Data
(60) Provisional application No. 63/504,721, filed on May 26, 2023.
(60) Provisional application No. 63/500,779, filed on May 8, 2023.
(60) Provisional application No. 63/386,784, filed on Dec. 9, 2022.
(60) Provisional application No. 63/384,937, filed on Nov. 23, 2022.

(51) Int. Cl. C12N 15/90; C12N 9/22; C12N 15/11

(52) U.S. Cl. CPC C12N 15/907; C12N 9/22; C12N 15/11; C12N 15/111; C12N 2310/20

(57) ABSTRACT

The disclosure relates to compositions and methods that modify target nucleic acids, as well as methods of detecting nucleic acids. Various compositions are described herein, including compositions comprising endonucleases, endonuclease systems, and chimeric proteins having the endonuclease and a nucleic-acid modulating domain or a nucleic acid modifying domain.

### CROSS-REFERENCE TO RELATED APPLICATIONS

This application claims priority to U.S. Provisional Application No. 63/384,937, filed on Nov. 23, 2022, U.S. Provisional Application No. 63/386,784, filed Dec. 9, 2022, U.S. Provisional Application No. 63/500,779, filed May 8, 2023, and U.S. Provisional Application No. 63/504,721, filed May 26, 2023, the entire contents which are incorporated herein in their entireties.

### FIELD

The disclosure relates to compositions, systems, and methods that modify target RNA, as well as methods of detecting a nucleic acid.

### DESCRIPTION OF THE TEXT FILE SUBMITTED ELECTRONICALLY

The instant application contains a sequence listing, which has been submitted in XML format via EFS-Web. The contents of the XML copy named “AMR-006US/134241-5006_Sequence Listing,” which was created on Nov. 24, 2023 and is 150,000 bytes in size, the contents of which are incorporated herein by reference in their entirety.

### BACKGROUND

Bacterial adaptive immune systems employ CRISPRs (clustered regularly interspaced short palindromic repeats) and CRISPR-associated (Cas) proteins for RNA-guided nucleic acid cleavage. The CRISPR-Cas systems thereby confer adaptive immunity in bacteria and archaea via RNA-guided nucleic acid interference. To provide anti-viral immunity, processed CRISPR array transcripts (crRNAs) assemble with Cas protein-containing surveillance complexes that recognize nucleic acids bearing sequence complementarity to the virus derived segment of the crRNAs, known as the spacer.

CRISPR-Cas tools have been widely used for gene editing, gene activation, gene inactivation, protein imaging, and beyond. For example, the RNA-guided endonucleases of the CRISPR-Cas9 system, including the most widely used Cas9 from Streptococcus pyogenes (SpCas9), can be used as a gene-editing tool in certain organisms. Although many current Cas9 polypeptides are capable of high-efficiency gene modifications, limitations remain due to off-target activities, such as the undesirable production of modifications within the genome at sites other than the desired target. Further, current endonuclease may be restricted in use due to protospacer adjacent motif (PAM) specificities and packaging constraints for delivery of system components.

Accordingly, there is a need for new gene engineering technologies, e.g., for therapy and/or diagnosis.

### SUMMARY

Therefore, the present disclosure provides, in aspects, a composition comprising an endonuclease comprising a sequence, optionally comprising a higher eukaryotes and prokaryotes nucleotide-binding domain (HEPN) domain, or a fragment or variant thereof, having at least about 70% (or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%) identity to any one of SEQ ID NOs: 1-4, and/or SEQ ID NOs: 80-89, or having about 1 to about 20 amino acid modifications (e.g. about 1, or about 2, or about 3, or about 4, or about 5, or about 6, or about 7, or about 8, or about 9, or about 10, or about 11, or about 12, or about 13, or about 14, or about 15, or about 16, or about 17, or about 18, or about 19, or about 20 modifications). In embodiments, the sequence comprises at least two HEPN domains, or fragments or variants thereof.

Additionally, the present disclosure provides, in aspects, a composition comprising an endonuclease comprising a sequence, optionally comprising one or more higher eukaryotes and prokaryotes nucleotide-binding domain (HEPN) domains, or a fragment or variant thereof, and having at least about 70% (or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%) identity to any one of SEQ ID NOs: 1-4, and/or SEQ ID NOs: 80-89, or having about 1 to about 20 amino acid modifications (e.g. about 1, or about 2, or about 3, or about 4, or about 5, or about 6, or about 7, or about 8, or about 9, or about 10, or about 11, or about 12, or about 13, or about 14, or about 15, or about 16, or about 17, or about 18, or about 19, or about 20 modifications). In embodiments, the sequence comprises a fragment or variant of a HEPN domain. In embodiments, the sequence comprises at least two HEPN domains, or fragments or variants thereof.

In aspects, the present disclosure provides a composition comprising a nucleic acid encoding an endonuclease comprising a sequence, optionally comprising one or more HEPN domains, or a fragment or variant thereof, and having at least about 70% (or at least about 75%, or at least about 80%, or at least about 85%, or at least about 90%, or at least about 91%, or at least about 92%, or at least about 93%, or at least about 94%, or at least about 95%, or at least about 96%%, or at least about 97%, or at least about 98%, or at least about 99%) identity to one or more of SEQ ID NOs: 1-4, and/or SEQ ID NOs: 80-89, or having about 1 to about 20 amino acid modifications (e.g. about 1, or about 2, or about 3, or about 4, or about 5, or about 6, or about 7, or about 8, or about 9, or about 10, or about 11, or about 12, or about 13, or about 14, or about 15, or about 16, or about 17, or about 18, or about 19, or about 20 modifications). In embodiments, the sequence comprises at least one HEPN domain, or fragments or variants thereof. In embodiments, the sequence comprises at least two HEPN domains, or fragments or variants thereof.

In aspects, the present disclosure provides a composition comprising a nuclease system, comprising (a) an endonuclease comprising a sequence, optionally comprising a HEPN domain, or a fragment or variant thereof, and having at least about 70% (or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%) to one or more of SEQ ID NOs: 1-4, and/or SEQ ID NOs: 80-89, or having about 1 to about 20 amino acid modifications (e.g. about 1, or about 2, or about 3, or about 4, or about 5, or about 6, or about 7, or about 8, or about 9, or about 10, or about 11, or about 12, or about 13, or about 14, or about 15, or about 16, or about 17, or about 18, or about 19, or about 20 modifications); and (b) an RNA molecule comprising a sequence complementary to one strand of a target nucleic acid molecule. In embodiments, the sequence comprises at least one HEPN domain, or fragments or variants thereof. In embodiments, the sequence comprises at least two HEPN domains, or fragments or variants thereof.

In embodiments, the composition further comprises one or more donor polynucleotides and/or is suitable for introducing one or more donor polynucleotides into a target nucleic acid molecule.

In embodiments, the endonuclease is suitable for introducing one or more excisions into a target nucleic acid molecule.

In aspects, the present disclosure provides a composition comprising a chimeric protein comprising: an endonuclease comprising a sequence, optionally comprising a HEPN domain, or a fragment or variant thereof, and having at least about 70% (or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%) to one or more of SEQ ID NOs: 1-4, and/or SEQ ID NOs: 80-89, or having about 1 to about 20 amino acid modifications (e.g. about 1, or about 2, or about 3, or about 4, or about 5, or about 6, or about 7, or about 8, or about 9, or about 10, or about 11, or about 12, or about 13, or about 14, or about 15, or about 16, or about 17, or about 18, or about 19, or about 20 modifications); and a nucleic acid-modulating domain or a nucleic acid-modifying domain, or nucleic acid-interacting/binding domain, comprising a sequence comprising a catalytic domain, or a fragment or variant thereof, wherein (a) and (b) do not naturally occur together in a same reading frame. In embodiments, the sequence comprises at least one HEPN domain, or fragments or variants thereof. In embodiments, the sequence comprises at least two HEPN domains, or fragments or variants thereof. In embodiments, the nucleic acid-modulating domain or a nucleic acid-modifying domain is a nucleic acid-interacting domain, e.g. selected from MCP, lambdaN, PP7, QBeta, SLBP, and TBP/TAR. In embodiments, the endonuclease reduces or enhances collateral activity for nucleic acid detection.

In aspects, the present disclosure provides a composition comprising a complex comprising chimeric protein and an RNA molecule, wherein the chimeric protein comprises an endonuclease comprising a sequence, optionally comprising one or more HEPN domains, or a fragment or variant thereof, and having at least about 70% (or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%) to one or more of SEQ ID NOs: 1-4, and/or SEQ ID NOs: 80-89, or having about 1 to about 20 amino acid modifications (e.g. about 1, or about 2, or about 3, or about 4, or about 5, or about 6, or about 7, or about 8, or about 9, or about 10, or about 11, or about 12, or about 13, or about 14, or about 15, or about 16, or about 17, or about 18, or about 19, or about 20 modifications); and a nucleic acid-modulating domain or a nucleic acid-modifying domain comprising a sequence comprising a catalytic domain, or a fragment or variant thereof, wherein (a) and (b) do not naturally occur together in a same reading frame and the RNA molecule comprises a sequence complementary to one strand of a target nucleic acid molecule.

In embodiments, the sequence comprises at least one HEPN domain, or fragments or variants thereof. In embodiments, the sequence comprises at least two HEPN domains, or fragments or variants thereof. In embodiments, the nucleic acid-modulating domain or the nucleic acid-modifying domain has one or more of nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, debranching activity, transesterification activity, photolyase activity and glycosylase activity. In embodiments, the nucleic acid-modulating domain or the nucleic acid-modifying domain is a Methyltransferase-like Protein 3 (METTL3) methyltransferase domain, a METTL3: Methyltransferase-like Protein 1 (METTL1) fusion, or a fragment or variant thereof.

In embodiments, the nucleic acid-modulating domain or the nucleic acid-modifying domain is selected from a deaminase, reverse transcriptase, transposase, integrase, and recombinase. In embodiments, the deaminase is a cytidine or cytosine deaminase, or a fragment or variant thereof. In embodiments, the cytidine or cytosine deaminase is selected from activation-induced cytidine deaminase (AID), cytidine deaminase 1 (CDA1), and apolipoprotein B mRNA-editing complex (APOBEC), or a fragment or variant thereof. In embodiments, the APOBEC is selected from A3A, AB3, APOBEC1, APOBEC3C, APOBEC3D, APOBEC3F, APOBEC3G, and APOBEC3H, or a fragment or variant thereof. In embodiments, the APOBEC has an amino acid sequence of one of SEQ ID NO: 39 [A3A], SEQ ID NO: 40 [AB3], SEQ ID NO: 41 [APOBEC1], SEQ ID NO: 42 [APOBEC3C], SEQ ID NO: 43 [APOBEC3D], SEQ ID NO: 44 [APOBEC3F], SEQ ID NO: 45 [APOBEC3G], and SEQ ID NO: 46 [APOBEC3H], or a fragment or variant thereof, or an amino acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto.

In embodiments, the deaminase is a DNA-specific adenine or adenosine deaminase, or a fragment or variant thereof. In embodiments, the DNA-specific adenine or adenosine deaminase is selected from tRNA-specific adenosine deaminase 7.10 (TadA 7.10), tRNA-specific adenosine deaminase 6.3 (TadA 6.3), tRNA-specific adenosine deaminase 7.8 (TadA 7.8), tRNA-specific adenosine deaminase 7.9 (TadA 7.9), and tRNA-specific adenosine deaminase 8e (TadA8e (TadA-8e V106W)) or a fragment or variant thereof. In embodiments, the TadA has an amino acid sequence of one of SEQ ID NO: 48 [TadA 7.10], SEQ ID NO: 49 [TadA 6.3], SEQ ID NO: 50 [TadA 7.8], SEQ ID NO: 51 [TadA 7.9], and SEQ ID NO: 52 [TadA 8e], or a fragment or variant thereof, or an amino acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto.

In embodiments, the deaminase is a RNA-specific adenine or adenosine deaminase, or a fragment or variant thereof. In embodiments, the RNA-specific adenine or adenosine deaminase is an adenosine deaminases acting on RNA (ADAR) enzyme, or a fragment or variant thereof. In embodiments, the ADAR is selected from ADAR1, ADAR2, and ADAR3, or a fragment or variant thereof. In embodiments, the ADAR has an amino acid sequence of one of SEQ ID NO: 53 [ADAR1] and SEQ ID NO: 54 [ADAR2] or a fragment or variant thereof, or an amino acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto. In embodiments, and as a non-limiting example, the catalytic deaminase domain of ADAR1 comprises amino acids 833-1226 of SEQ ID NO: 53. As another non-limiting example the catalytic deaminase domain of ADAR2 comprises amino acids 299-701 of SEQ ID NO: 54.

In embodiments, the deaminase further comprises a nuclear localization signal. In embodiments, the endonuclease further comprises a uracil glycosylase inhibitor (UGI), or a fragment or variant thereof. In embodiments, the RNA molecule is a guide RNA (gRNA). In embodiments, the gRNA comprises a sequence that interacts with the endonuclease. In embodiments, the endonuclease forms a complex with the gRNA.

In embodiments, the composition is suitable for base editing. In embodiments, the composition is suitable for DNA base editing. In embodiments, the composition is suitable for RNA base editing. In embodiments, the composition is suitable for catalyzing C>T nucleotide conversions or A>G nucleotide conversions in a target nucleic acid.

In embodiments, the composition comprises both an adenosine deaminase and a cytidine deaminase.

In embodiments, the composition is suitable for dual base editing.

In embodiments, the reverse transcriptase is Moloney murine leukemia virus reverse transcriptase (M-MLV RT) or M-MLV RT(D200N/L603W/T330P/T306K/W313F), or a fragment or variant thereof. In embodiments, the M-MLV RT has an amino acid sequence of SEQ ID NO: 55 [M-MLV RT] or SEQ ID NO 56 [M-MLV RT(D200N/L603W/T330P/T306K/W313F)] or a fragment or variant thereof, or an amino acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto.

In embodiments, the composition further comprises a dominant negative human MutL homolog (MLH1). In embodiments, the composition is suitable for use with a dominant negative MLH1.

In embodiments, the RNA molecule is or comprises a prime editing guide RNA (pegRNA). In embodiments, the endonuclease forms a complex with the pegRNA. In embodiments, the pegRNA serves as a template for transcription of a new DNA sequence. In embodiments, the pegRNA binds to a DNA strand opposite from a typical gRNA binding site. In embodiments, the pegRNA comprises a gRNA containing a primer binding site (PBS) and a reverse transcriptase (RT) template sequence. In embodiments, the RNA molecule is or comprises a gRNA. In embodiments, the gRNA comprises a sequence that interacts with the endonuclease.

In embodiments, the endonuclease forms a complex with the gRNA. In embodiments, the composition comprises both a gRNA and a pegRNA.

In embodiments, the composition is suitable for prime editing.

In embodiments, the transposase is selected from Tn1, Tn2, Tn3, Tn5, Tn7, Tn9, Tn10, Tn552, Tn903, Tn1000/Gamma-delta, Tn/O, tnsA, tnsB, tnsC, tniQ, IS10, ISS, IS911, Minos, Sleeping beauty, piggyBac, Tol2, Mos1, Himarl, Hermes, Tol2, Minos, Tel, P-element, MuA, Ty1, Chapaev, transib, Tc1/mariner, and Tc3 donor DNA system.

In embodiments, the transposase is a transposon 7-like (Tn7-like) transposon system, or a fragment or variant thereof. In embodiments, the transposase is one or more of transposon 7 protein A (TnsA), transposon 7 protein B (Tns B), transposon 7 protein C (Tns C), and transposition of integron protein Q (TniQ), or a fragment or variant thereof. In embodiments, the Tn7-like transposon system is derived from Vibrio cholerae Tn6677.

In embodiments, the transposase has an amino acid sequence of one or more of SEQ ID NO: 57 [TnsA], SEQ ID NO: 58 [TnsB], SEQ ID NO: 59 [TnsC], and SEQ ID NO: 60 [TniQ], or a fragment or variant thereof, or an amino acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto.

In embodiments, the integrase is a serine-recombinase, or a fragment or variant thereof.

In embodiments, the serine-recombinase is Bxb1, or a fragment or variant thereof. In embodiments, the recombinase is a Gin invertase or Tn3 resolvase, or a fragment or variant thereof.

In embodiments, the nucleic acid-modulating domain or the nucleic acid-modifying domain comprises one or more modifications, (e.g., without limitation, mutations) to reduce activity relative to an unmutated form.

In embodiments, the nucleic acid-modulating domain or the nucleic acid-modifying domain comprises one or more modifications, (e.g., without limitation, mutations) to increase activity relative to an unmutated form.

In embodiments, the sequence of (a) is disposed at the N-terminus of the chimeric protein and the sequence of (b) is disposed at the C-terminus of the chimeric protein.

In embodiments, the sequence of (a) is disposed at the C-terminus of the chimeric protein and the sequence of (b) is disposed at the N-terminus of the chimeric protein.

In embodiments, the composition further comprises a linker that joins the sequence of (a) and the sequence of (b). In embodiments, the linker is between about 4 and about 40 amino acids, or about 10 and about 40 amino acids, or about 20 and about 40 amino acids, or about 30 and about 40 amino acids, or about 4 and about 30 amino acids, or about 4 and about 20 amino acids, or about 4 and about 10 amino acids, or about 5 amino acids, or about 10 amino acids, or about 15 amino acids, or about 20 amino acids, or about 25 amino acids, or about 30 amino acids, or about 35 amino acids, or about 40 amino acids. In embodiments, the linker is substantially comprised of glycine and serine residues. In embodiments, the linker is (GGS)ₙ, wherein n is 1, or 2, or 3, or 4, or 5. In embodiments, the linker is GGSGGSGGSG (SEQ ID NO: 61), GGSGGSGGGGSGGGGS (SEQ ID NO: 62), GGGGS (SEQ ID NO: 63), GGS (SEQ ID NO: 64), (GGGGS)ₙ(n=1-4) (SEQ ID NO: 65), (Gly)₈ (SEQ ID NO: 66), (Gly)₆ (SEQ ID NO: 67), (EAAAK)ₙ(n=1-3) (SEQ ID NO: 68), A(EAAAK)ₙA (n=2-5) (SEQ ID NO: 69), AEAAAKEAAAKA (SEQ ID NO: 70), A(EAAAK)₄ALEA(EAAAK)₄A (SEQ ID NO: 71), PAPAP (SEQ ID NO: 72), KESGSVSSEQLAQFRSLD (SEQ ID NO: 73), EGKSSGSGSESKST (SEQ ID NO: 74), and GSAGSAAGSGEF (SEQ ID NO: 75), or a variant thereof, wherein the variant comprises about 1, or about 2, or about 3, or about 4, or about 5 mutations, the mutations selected from substitutions or deletions.

In embodiments, the endonuclease is suitable for creating a double stranded break in a nucleic acid. In embodiments, the endonuclease is suitable for creating a nick in a nucleic acid. In embodiments, the endonuclease is suitable for nucleic acid modification by homology-directed repair (HDR). In embodiments, the endonuclease is suitable for nucleic acid modification by non-homologous end joining (NHEJ). In embodiments, the endonuclease recognizes a PAM. In embodiments, the endonuclease recognizes a plurality of PAMs. In embodiments, the endonuclease comprises one or more modifications, (e.g., without limitation, mutations) to reduce catalytic activity relative to an unmutated form. In embodiments, the endonuclease comprises one or more modifications, (e.g., without limitation, mutations) to render the endonuclease substantially catalytically inactive relative to an unmutated form. In embodiments, the endonuclease comprises one or more modifications, (e.g., without limitation, modifications, (e.g., without limitation, mutations) to increase catalytic activity relative to an unmutated form. In embodiments, the endonuclease comprises one or more modifications, (e.g., without limitation, mutations) to render the endonuclease substantially catalytically hyperactive relative to an unmutated form. In embodiments, the endonuclease has nickase activity. In embodiments, the endonuclease comprises one or more modifications, (e.g., without limitation, mutations) to produce nickase activity. In embodiments, the endonuclease has collateral cleavage activity. In embodiments, the endonuclease comprises one or more modifications, (e.g., without limitation, mutations) to produce collateral cleavage activity. In embodiments, the endonuclease has at least about 75% identity to one or more of SEQ ID NOs: 1-4, and/or SEQ ID NOs: 80-89. In embodiments, the endonuclease has at least about 80% identity to one or more of SEQ ID NOs: 1-4, and/or SEQ ID NOs: 80-89. In embodiments, the endonuclease has at least about 85% identity to one or more of SEQ ID NOs: 1-4, and/or SEQ ID NOs: 80-89.

In embodiments, the endonuclease has at least about 90% identity to one or more of SEQ ID NOs: 1-4, and/or SEQ ID NOs: 80-89. In embodiments, the endonuclease has at least about 95% identity to one or more of SEQ ID NOs: 1-4, and/or SEQ ID NOs: 80-89. In embodiments, the endonuclease has at least about 97% identity to one or more of SEQ ID NOs: 1-4, and/or SEQ ID NOs: 80-89. In embodiments, the endonuclease has at least about 99% identity to one or more of SEQ ID NOs: 1-4, and/or SEQ ID NOs: 80-89.

In embodiments, the endonuclease has about 1 to about 15 amino acid modifications. In embodiments, the endonuclease has about 1 to about 10 amino acid modifications. In embodiments, the endonuclease has about 1 to about 5 amino acid modifications. In embodiments, the endonuclease has about 1, or about 2, or about 3, or about 4, or about 5, or about 10, or about 15, or about 20 amino acid modifications. In embodiments, the amino acid modifications are selected from substitutions and deletions.

In embodiments, the endonuclease (or chimeric protein) comprises a domain from a different endonuclease. In embodiments, the different endonuclease is a Cas endonuclease. In embodiments, the domain is a PAM-interacting domain. In embodiments, the target nucleic acid is or comprises single-stranded RNA (ssRNA). In embodiments, the target nucleic acid is or comprises double-stranded RNA (dsRNA). In embodiments, the target nucleic acid is or comprises single-stranded DNA (ssDNA). In embodiments, the target nucleic acid is or comprises double-stranded DNA (dsDNA). In embodiments, the target nucleic acid is about 2 to about 6 nucleotides upstream of a PAM sequence. In embodiments, the RNA molecule is or comprises a guide ribonucleic structure configured to form a complex with the endonuclease.

In embodiments, the guide ribonucleic structure (i) comprises (a) a CRISPR RNA (crRNA) suitable for hybridizing to a target nucleic acid molecule and/or (b) a transactivating CRISPR RNA (tracrRNA) suitable for interacting with the endonuclease or (ii) lacks a (a) a crRNA suitable for hybridizing to a target nucleic acid molecule and/or (b) a tracrRNA suitable for interacting with the endonuclease.

In embodiments, the RNA molecule is or comprises a gRNA. In embodiments, the gRNA comprises a sequence that interacts with the endonuclease. In embodiments, the endonuclease forms a complex with the gRNA.

In embodiments, the RNA molecule is or comprises the nucleic acid sequence of SEQ ID NOs: 1-4, and/or SEQ ID NOs: 80-89, or a fragment or variant thereof, or a nucleic acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto.

In embodiments, the RNA molecule has perfect sequence complementarity to one strand of a target nucleic acid molecule. In embodiments, the RNA molecule has partial sequence complementarity to one strand of a target nucleic acid molecule.

In embodiments, the composition further comprises a viral vector. In embodiments, the viral vector is or comprises an AAV. In embodiments, the AAV is or comprises one or more of AAV1, AAV2, AAV3, AAV4, AAV5, AAV6, AAV7, AAV8, AAV9, AAV10, AAV11, AAV12, AAV13, AAV2/1, AAV2/5, AAV2/8, AAV2/9, AAV3/1, AAV3/5, AAV3/8, and AAV3/9. In embodiments, the composition further comprises a non-viral vector. In embodiments, the composition further comprises a lipid nanoparticle (LNP) liposomes, lipoplexes or polymeric nanoparticle. In embodiments, the LNP comprises one or more of ionizable lipids, amino lipids, anionic lipids, neutral lipids, amphipathic lipids, helper lipids, structural lipids, PEG lipids, and lipoids. In embodiments, the composition further comprises a virus-like particle (VLP).

In aspects, the present disclosure provides a nucleic acid encoding the endonuclease or chimeric protein of any one of the embodiments and/or aspects disclosed herein. In embodiments, the nucleic acid is or comprises a DNA molecule or an RNA molecule. In embodiments, the RNA is or comprises mRNA or modified mRNA (mmRNA). In embodiments, the DNA is or comprises a vector or plasmid. In embodiments, the nucleic acid comprises a codon optimized sequence. In embodiments, the nucleic acid comprises one or more modifications. In embodiments, the modifications are one or more of base modifications and backbone modifications.

In aspects, the present disclosure provides a viral vector comprising the nucleic acid of any one of the embodiments and/or aspects disclosed herein. In embodiments, the viral vector is or comprises an AAV. In embodiments, the AAV is or comprises one or more of AAV1, AAV2, AAV3, AAV4, AAV5, AAV6, AAV7, AAV8, AAV9, AAV10, AAV11, AAV12, AAV13, AAV2/1, AAV2/5, AAV2/8, AAV2/9, AAV3/1, AAV3/5, AAV3/8, and AAV3/9.

In aspects, the present disclosure provides a viral vector comprising the nucleic acid of any one of the embodiments and/or aspects disclosed herein. In embodiments, the viral vector is or comprises a VLP.

In embodiments, the endonuclease mediates a trans-splicing event.

In embodiments, the endonuclease mediates an exon skipping or exon inclusion event.

In aspects, the present disclosure provides a lipid nanoparticle comprising the nucleic acid of any one of the embodiments and/or aspects disclosed herein.

In aspects, the present disclosure provides a cell comprising a nucleic acid of any one of the embodiments and/or aspects disclosed herein, the viral vector of any one of the embodiments and/or aspects disclosed herein, or the lipid nanoparticle of any one of the embodiments and/or aspects disclosed herein.

In embodiments, the cell is a prokaryotic cell. In embodiments, the cell is a eukaryotic cell. In embodiments, the cell is a mammalian cell. In embodiments, the cell is a human cell. In embodiments, the cell is an immortalized cell. In embodiments, the cell is harvested from a subject.

In aspects, the present disclosure provides a pharmaceutical composition comprising the composition of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, or the cell of any one of the embodiments and/or aspects disclosed herein, and a pharmaceutically acceptable carrier.

In aspects, the present disclosure provides a composition comprising an RNA molecule comprising a nucleic acid sequence of SEQ ID NOs: 1-4, and/or SEQ ID NOs: 80-89, or a fragment or variant thereof, or a nucleic acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto.

In embodiments, the RNA molecule interacts with an endonuclease comprising a sequence comprising, optionally a HEPN domain, or a fragment or variant thereof, and having at least about 70% (or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%) to one or more of SEQ ID NOs: 1-4, and/or SEQ ID NOs: 80-89, or having about 1 to about 20 amino acid modifications (e.g. about 1, or about 2, or about 3, or about 4, or about 5, or about 6, or about 7, or about 8, or about 9, or about 10, or about 11, or about 12, or about 13, or about 14, or about 15, or about 16, or about 17, or about 18, or about 19, or about 20 modifications). In embodiments, the sequence comprises at least one HEPN domain, or fragments or variants thereof. In embodiments, the sequence comprises at least two HEPN domains, or fragments or variants thereof.

In embodiments, the RNA molecule comprises one or more modifications. In embodiments, the modifications are one or more of base modifications and backbone modifications. In embodiments, the RNA molecule comprises a sequence complementary to one strand of a target nucleic acid molecule. In embodiments, the RNA molecule has perfect sequence complementarity to one strand of a target nucleic acid molecule.

In embodiments, the RNA molecule has partial sequence complementarity to one strand of a target nucleic acid molecule.

In aspects, the present disclosure provides a composition comprising a nucleic acid encoding an endonuclease comprising a sequence, optionally comprising a HEPN domain, or a fragment or variant thereof, in conjunction with an RNA containing a repeat having at least about 70% identity to one or more of SEQ ID NOs: 28-31, and/or SEQ ID NOs: 90-97. In embodiments, the composition has least about 75%, or at least about 80%, or at least about 85%, or at least about 90%, or at least about 91%, or at least about 92%, or at least about 93%, or at least about 94%, or at least about 95%, or at least about 96%%, or at least about 97%, or at least about 98%, or at least about 99%) identity to SEQ ID NOs: 28-31, and/or SEQ ID NOs: 90-97, or has about 1 to about 20 nucleotide modifications (e.g. about 1, or about 2, or about 3, or about 4, or about 5, or about 6, or about 7, or about 8, or about 9, or about 10, or about 11, or about 12, or about 13, or about 14, or about 15, or about 16, or about 17, or about 18, or about 19, or about 20 modifications). In embodiments, the sequence comprises at least one HEPN domain, or fragments or variants thereof. In embodiments, the sequence comprises at least two HEPN domains, or fragments or variants thereof.

In aspects, the present disclosure provides a kit comprising a container comprising the composition of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, the cell of any one of the embodiments and/or aspects disclosed herein, or the pharmaceutical composition of any one of the embodiments and/or aspects disclosed herein and with instructions for use in modulating and/or modifying a nucleic acid.

In aspects, the present disclosure provides a method of modulating and/or modifying a nucleic acid in a cell, comprising contacting the cell with the composition of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, the cell of any one of the embodiments and/or aspects disclosed herein, or the pharmaceutical composition of any one of the embodiments and/or aspects disclosed herein.

In aspects, the present disclosure provides a method of modulating and/or modifying a nucleic acid in a subject in need thereof, comprising administering an effective amount of the cell with the composition of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, the cell of any one of the embodiments and/or aspects disclosed herein, or the pharmaceutical composition of any one of the embodiments and/or aspects disclosed herein to the subject.

In embodiments, the modulating and/or modifying is selected from one or more of cleaving, nicking, methylating, labeling, and mutating the nucleic acid. In embodiments, the modulating and/or modifying is selected from one or more of cleaving the nucleic acid; inserting a nucleic acid, editing the nucleic acid; modulating transcription from the nucleic acid; isolating the nucleic acid, binding the nucleic acid, and imaging the nucleic acid.

In aspects, the present disclosure provides a method of disrupting, correcting, and/or replacing a gene in a cell, comprising contacting the cell with the composition of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, the cell of any one of the embodiments and/or aspects disclosed herein, or the pharmaceutical composition of any one of the embodiments and/or aspects disclosed herein.

In aspects, the present disclosure provides a method of disrupting, correcting, and/or replacing a gene in a subject in need thereof, comprising administering an effective amount of the composition of any one of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, the cell of any one of the embodiments and/or aspects disclosed herein, or the pharmaceutical composition of any one of the embodiments and/or aspects disclosed herein to the subject.

In aspects, the present disclosure provides a method of treating, ameliorating or preventing a disease or disorder in a subject, comprising (a) contacting a cell with the composition of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, the cell of any one of the embodiments and/or aspects disclosed herein, or the pharmaceutical composition of any one of the embodiments and/or aspects disclosed herein, and (b) administering an effective amount of the cell to the subject.

In aspects, the present disclosure provides a method of treating, ameliorating or preventing a disease or disorder in a subject, comprising administering an effective amount of the composition of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, the cell of any one of the embodiments and/or aspects disclosed herein, or the pharmaceutical composition of any one of the embodiments and/or aspects disclosed herein to the subject.

In embodiments, composition of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, the cell of any one of the embodiments and/or aspects disclosed herein, or the pharmaceutical composition of any one of the embodiments and/or aspects disclosed herein for use in treating, ameliorating or preventing a patient with a disease or disorder.

In aspects, the present disclosure provides use of the composition of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, the cell of any one of the embodiments and/or aspects disclosed herein, or the pharmaceutical composition of any one of the embodiments and/or aspects disclosed herein in the manufacture of a medicament for the treating, ameliorating or preventing of a disease or disorder.

In aspects, the present disclosure provides a method of detecting and/or quantifying a nucleic acid in a sample, comprising contacting the sample with a composition of any one of the embodiments and/or aspects disclosed herein.

In embodiments, the nucleic acid is a target and/or reporter nucleic acid. In embodiments, the method comprises detection of a reporter signal, the reporter signal being generated upon endonuclease cleavage. In embodiments, the reporter signal is a fluorescent signal. In embodiments, the endonuclease has collateral cleavage activity.

In various embodiments, the composition disclosed herein, or the trans-splicing system disclosed herein, further comprises a repair RNA (repRNA) sequence, comprising: (a) one or more exons and/or introns; (b) a splice donor and/or splice acceptor, wherein the repRNA is suitable for trans-splicing. In embodiments, the trans-splicing system comprises a splice donor, a splice acceptor, and replaces an internal exon. In embodiments, the repRNA is operably linked to the RNA molecule comprising a sequence complementary to one strand of a target nucleic acid molecule or the gRNA.

In aspects, the present disclosure provides a system for targeting a nucleic acid for trans-splicing, the system comprising: (a) an endonuclease of any one of the embodiments disclosed herein, and optionally an RNA molecule comprising a sequence complementary to one strand of a target nucleic acid molecule; (b) an RNA-binding polypeptide that associates with the endonuclease; and (c) a repair RNA (repRNA) sequence, comprising: (i) one or more exons and/or introns; (ii) a splice donor and/or splice acceptor.

In embodiments, the RNA molecule is a gRNA.

In embodiments, the endonuclease is not linked, associated, and/or fused with an RNA binding protein.

In embodiments, the repRNA is not operably linked to one or more gRNAs. In embodiments, the repRNA is provided in trans to one or more gRNAs.

In embodiments, the repRNA further comprises a ribozyme site. In embodiments, the ribozyme site is a hairpin, hammerhead, hepatitis delta virus (HDV), Varkud satellite (VS), or glmS ribozyme site, or a variant thereof. In embodiments, the ribozyme site is a HDV ribozyme site. In embodiments, the ribozyme site is upstream of the one or more exons and/or introns of the repRNA.

In aspects, the present disclosure provides a system for targeting a nucleic acid for trans-splicing, the system comprising: (a) an endonuclease of any one of claims 1-108 and an RNA molecule comprising a sequence complementary to one strand of a target nucleic acid molecule; and (b) a repair RNA (repRNA) sequence, comprising: (i) one or more exons and/or introns; (ii) a splice donor and/or splice acceptor.

In embodiments, the RNA molecule is a gRNA. In embodiments, the endonuclease is not linked, associated, and/or fused with an RNA binding protein. In embodiments, the repRNA is operably linked to one or more gRNAs.

In embodiments, the composition comprises a gRNA, repRNA, and a Cas endonuclease operably linked to a single promoter or a bidirectional promoter.

In embodiments, the gRNA and repRNA are located on a first side of the bidirectional promoter, and the Cas endonuclease is located on a second side of the bidirectional promoter.

In embodiments, disclosed herein is a composition comprising an endonuclease and having an amino acid sequence of least 90%, or at least 95%, or at least 98%, or at least 99% identity to SEQ ID NO: 3. In embodiments, substitutions are made to SEQ ID NO: 3:

Met Asp Lys His Pro Ser Asn Arg Tyr Ala Leu Pro Lys Val Ile Ile Ser Glu Val
Asp His Glu Arg Ile Leu Glu Phe Lys Val Lys Tyr Glu Lys Leu Ala Arg Leu
Asp Arg Phe Glu Val Lys Ala Met His Tyr Asp Gly Ala Glu Ile Val Phe Asp
Glu Val Val Ala Asn Gly Gly Leu Ile Glu Val Glu Tyr Gln Asp Asn Asn Lys
Thr Ile Thr Ile Asn Leu Asn Gly Lys Lys Tyr Thr Ile Asn Gly Arg Lys Val Gly
Gly Lys Arg Arg Leu Leu Glu Asp Arg Ile Ser Arg Gly Lys Val Cys Leu Glu
Leu His Asp Lys Ile Pro Asp Glu Lys Gly Asn Leu Arg Ser Ser Arg Thr Glu
Arg Glu Leu Ile Thr Phe Asp Ser Thr Lys Leu Tyr Ser Gln Ile Ile Gly Arg Asp
Val Ala Ser Thr Lys Glu Ile Tyr Leu Ile Lys Arg Phe Leu Ala Tyr Arg Ser Asp
Leu Leu Phe Tyr Tyr Gly Phe Ile Asp Asn Phe Phe Lys Val Ala Gly Asn Lys
Arg Glu Leu Trp Lys Ile Asp Phe Ser Gly Asp Lys Asn Gln Glu Leu Ile Lys
Tyr Phe Asn Phe Thr Ile Asn Asp Lys Leu Lys Asn Asp Lys Gly Tyr Leu Lys
Glu Tyr Thr Ala Asn Asp Glu Gln Ile Lys Lys Asp Leu Gln Asn Thr Lys Glu
Val Phe Thr Ala Leu Arg His Ala Leu Met His Phe Glu Tyr Asp Phe Phe Glu
Lys Leu Phe Asn Asn Glu Glu Ile Glu Thr Leu Ser Lys Ile His Asp Ile Glu Leu
Leu Asn Thr Met Ile Asn Lys Leu Asp Lys Leu Asn Ile Asp Thr Arg Lys Glu
Tyr Ile Asp Asp Glu Lys Ile Thr Val Phe Gly Glu Glu Ile Ser Leu Lys Thr Leu
Tyr Gly Leu Tyr Ala His Thr Ala Ile Asn Arg Val Ala Phe Asn Lys Leu Ile Asn
Arg Phe Met Val Glu Asn Gly Thr Glu Asn Glu Ala Leu Lys Lys Tyr Phe Asn
Ser Lys Ala Glu Gly Gly Ile Ala Tyr Glu Ile Asp Ile His Gln Asn Ser Glu Tyr
Lys Gln Leu Tyr Ile Gln His Lys Asp Leu Val Ser Lys Leu Ser Ala Leu Ser Asp
Gly Asp Glu Ile Ala Asp Thr Asn Lys Lys Ile Ser Glu Leu Lys Val Lys Met
Lys Ala Ile Thr Lys Ala Asn Ser Leu Lys Arg Leu Glu His Lys Leu Arg Leu
Thr Phe Gly Phe Ile Tyr Thr Glu Tyr Gln Asp Tyr Asn Ala Phe Lys Asn Asn
Phe Asp Thr Asp Ile Lys Ser Gly Arg Phe Ile Pro Lys Asp Ser Glu Gly Lys Arg
Arg Gly Phe Asp His Arg Glu Leu Asp Gln Leu Lys Arg Tyr Tyr Asp Ala Thr
Phe Ala Asp Lys Lys Pro Gln Thr Lys Glu Thr Phe Asp Glu Ile Asp Lys Gln
Ile Asp Gln Leu Ser Leu Lys Asn Leu Ile Gly Asp Asp Thr Leu Leu Lys Val
Ile Leu Leu Ile Tyr Ile Phe Leu Pro Arg Glu Ile Lys Gly Glu Phe Leu Gly Phe
Val Lys Lys Tyr Tyr His Asp Thr Lys His Ile Glu Glu Asp Thr Lys Asp Lys
Asp Glu Gly Phe Asp Asp Thr Phe Pro Val Gly Leu Lys Leu Lys Val Leu Asp
Lys Asn Ile Arg Ala Leu Ser Val Leu Lys His Ser Leu Ser Tyr Gln Ala Lys Tyr
Asn Lys Lys Glu Glu Lys Lys Glu Gln Phe Tyr Glu Ala Gly Asn Arg His Gly
Arg Phe Tyr Lys Lys Leu Gly Ile Ser His Asn Gln Glu Glu Phe Asp Lys Ser
Val Tyr Ala Pro Leu Leu Arg Tyr His Ala Ala Leu Phe Lys Leu Leu Asn Asp
Phe Glu Ile Tyr Ser Leu Ala Gln His Ile Glu Gly Lys Glu Thr Leu Ala Gln Gln
Ile Glu Lys Pro Gln Phe Ser Gln Tyr Glu His Tyr Asn Phe Arg Lys Met Leu
Ser Lys Thr Tyr Pro Lys Ser Ala Glu Arg Gly Ala Leu Asp Asn Asp Ala Phe
Asp Thr Val Ile Asn Met Arg Asn Asp Ile Ala His Leu Ser His Glu Pro Leu Phe
Glu Cys Pro Leu Asp Gly Lys Lys Ser Tyr Lys Leu Lys Gln Gly Lys Arg Thr
Asn Thr Ile Asn Val Lys Pro Leu Pro Ile Ser Arg Lys Met Ile Val Asp Phe Ile
Ser Ser Gln Ser Asp Met Lys Lys Thr Leu Gly Tyr Asp Ala Val Asn Asp Leu
Thr Met Lys Ile Ile Gln Leu Arg Thr Arg Leu Lys Val Tyr Ala Asp Lys Ser Glu
Thr Ile Lys Thr Leu Val Asp Ala Ala Lys Thr Pro Asn Asp Phe Tyr His Ile Tyr
Lys Val Lys Gly Val Glu Ala Ile Asn Arg His Leu Leu Glu Val Ile Gly Glu Thr
Lys Asp Glu Lys Arg Ile Arg Lys Arg Ile Glu Ser Gly Asn Ala Ile Ala Gly Arg
Thr Pro Ala Asp Ser Gln Glu Asn

As described herein, substitutions may be made to this sequence to generate the inventive endonuclease (including, taking into account degeneracy of the genetic code).

In some embodiments, the endonuclease has one or more substitutions at positions corresponding to D38X, A59X, G172X, T236X, T319X, H375X, H419X, T424X, E529X, T541X, G562X, K564X, D569X, A586X, N641X, D642X, S647X, D721X, R779X, K13X, K566X, G554X, A35X, E110X, G314X, K114X, D498X, I86X, V57X, H249X, R704X of SEQ ID NO: 3, wherein the substitution is defined by X and wherein X is any amino acid. In some embodiments, X is an essential or non-essential amino acid.

In some embodiments, X is a hydrophilic or hydrophobic amino acid.

In some embodiments, X is a hydrophilic amino acid.

In some embodiments, X is a polar and positively charged hydrophilic amino acid. In some embodiments, X is selected from arginine (R) or lysine (K).

In some embodiments, X is a polar and neutral charged hydrophilic amino acid. In some embodiments, X is selected from asparagine (N), glutamine (Q), serine (S), threonine (T), proline (P), and cysteine (C).

In some embodiments, X is a polar and negatively charged hydrophilic amino acid. In some embodiments, X is selected from aspartate (D) or glutamate (E).

In some embodiments, wherein X is an aromatic, polar and positively charged hydrophilic amino acid. In some embodiments, wherein X is histidine (H).

In some embodiments, X is a hydrophobic amino acid.

In some embodiments, X is a hydrophobic, aliphatic amino acid. In some embodiments, X is selected from glycine (G), alanine (A), leucine (L), isoleucine (I), methionine (M), or valine (V).

In some embodiments, X is a hydrophobic, aromatic amino acid. In some embodiments, wherein X is selected from phenylalanine (F), tryptophan (W), or tyrosine (Y).

In some embodiments, the endonuclease of SEQ ID NO: 3 comprises one or more of the following substitutions:
a hydrophilic residue other than aspartate (D) at a position corresponding to 38;
a hydrophobic residue other than alanine (A) at a position corresponding to 59;
a hydrophobic residue other than glycine (G) at a position corresponding to 172;
a hydrophilic residue other than threonine (T) at a position corresponding to 236;
a hydrophilic residue other than threonine (T) at a position corresponding to 319;
a hydrophilic residue other than histidine (H) at a position corresponding to 375;
a hydrophilic residue other than histidine (H) at a position corresponding to 419;
a hydrophilic residue other than threonine (T) at a position corresponding to 424;
a hydrophilic residue other than glutamate (E) at a position corresponding to 529;
a hydrophilic residue other than threonine (T) at a position corresponding to 541;
a hydrophobic residue other than glycine (G) at a position corresponding to 562;
a hydrophilic residue other than lysine (K) at a position corresponding to 564;
a hydrophilic residue other than aspartate (D) at a position corresponding to 569;
a hydrophobic residue other than alanine (A) at a position corresponding to 586;
a hydrophilic residue other than asparagine (N) at a position corresponding to 641;
a hydrophilic residue other than aspartate (D) at a position corresponding to 642;
a hydrophilic residue other than serine (S) at a position corresponding to 647;
a hydrophilic residue other than aspartate (D) at a position corresponding to 721;
a hydrophilic residue other than arginine (R) at a position corresponding to 779;
a hydrophilic residue other than lysine (K) at a position corresponding to 13;
a hydrophilic residue other than lysine (K) at a position corresponding to 566;
a hydrophobic residue other than glycine (G) at a position corresponding to 554;
a hydrophobic residue other than alanine (A) at a position corresponding to 35;
a hydrophilic residue other than glutamate (E) at a position corresponding to 110;
a hydrophobic residue other than glycine (G) at a position corresponding to 314;
a hydrophilic residue other than lysine (K) at a position corresponding to 114;
a hydrophilic residue other than aspartate (D) at a position corresponding to 498;
a hydrophobic residue other than isoleucine (I) at a position corresponding to 86;
a hydrophobic residue other than valine (V) at a position corresponding to 57;
a hydrophilic residue other than histidine (H) at a position corresponding to 249; and
a hydrophilic residue other than arginine (R) at a position corresponding to 704.

In some embodiments, the endonuclease of SEQ ID NO: 3 comprises D38F. In some embodiments, the endonuclease of SEQ ID NO: 3 comprises A59V. In some embodiments, the endonuclease of SEQ ID NO: 3 comprises G172L. In some embodiments, the endonuclease of SEQ ID NO: 3 comprises T236L. In some embodiments, the endonuclease of SEQ ID NO: 3 comprises T319I. In some embodiments, the endonuclease of SEQ ID NO: 3 comprises H375L. In some embodiments, the endonuclease of SEQ ID NO: 3 comprises H419Y. In some embodiments, the endonuclease of SEQ ID NO: 3 comprises T424F. In some embodiments, the endonuclease of SEQ ID NO: 3 comprises E529L. In some embodiments, the endonuclease of SEQ ID NO: 3 comprises T541L. In some embodiments, the endonuclease of SEQ ID NO: 3 comprises G562Y. In some embodiments, the endonuclease of SEQ ID NO: 3 comprises K564M. In some embodiments, the endonuclease of SEQ ID NO: 3 comprises D569L. In some embodiments, the endonuclease of SEQ ID NO: 3 comprises A586I. In some embodiments, the endonuclease of SEQ ID NO: 3 comprises N641F. In some embodiments, the endonuclease of SEQ ID NO: 3 comprises D642L. In some embodiments, the endonuclease of SEQ ID NO: 3 comprises S647L. In some embodiments, the endonuclease of SEQ ID NO: 3 comprises D721L. In some embodiments, the endonuclease of SEQ ID NO: 3 comprises R779I. In some embodiments, the endonuclease of SEQ ID NO: 3 comprises K13R. In some embodiments, the endonuclease of SEQ ID NO: 3 comprises K566R. In some embodiments, the endonuclease of SEQ ID NO: 3 comprises G554H. In some embodiments, the endonuclease of SEQ ID NO: 3 comprises A35N. In some embodiments, the endonuclease of SEQ ID NO: 3 comprises E110T. In some embodiments, the endonuclease of SEQ ID NO: 3 comprises G314Q. In some embodiments, the endonuclease of SEQ ID NO: 3 comprises K114P. In some embodiments, the endonuclease of SEQ ID NO: 3 comprises D498P. In some embodiments, the endonuclease of SEQ ID NO: 3 comprises I86P. In some embodiments, the endonuclease of SEQ ID NO: 3 comprises V57E. In some embodiments, the endonuclease of SEQ ID NO: 3 comprises H249W. In some embodiments, the endonuclease of SEQ ID NO: 3 comprises R704F.

In some embodiments, the endonuclease of SEQ ID NO: 3 comprises D38F and A59V. In some embodiments, the endonuclease of SEQ ID NO: 3 comprises D38F, A59V, and G172L. In some embodiments, the endonuclease of SEQ ID NO: 3 comprises D38F, A59V, G172L, and T236L. In some embodiments, the endonuclease of SEQ ID NO: 3 comprises D38F, A59V, G172L, T236L, and T319I. In some embodiments, the endonuclease of SEQ ID NO: 3 comprises D38F, A59V, G172L, T236L, T319I, and H375L. In some embodiments, the endonuclease of SEQ ID NO: 3 comprises D38F, A59V, G172L, T236L, T319I, H375L, and H419Y. In some embodiments, the endonuclease of SEQ ID NO: 3 comprises D38F, A59V, G172L, T236L, T319I, H375L, H419Y, and T424F. In some embodiments, the endonuclease of SEQ ID NO: 3 comprises D38F, A59V, G172L, T236L, T319I, H375L, H419Y, T424F, and E529L. In some embodiments, the endonuclease of SEQ ID NO: 3 comprises D38F, A59V, G172L, T236L, T319I, H375L, H419Y, T424F, E529L, and T541L. In some embodiments, the endonuclease of SEQ ID NO: 3 comprises D38F, A59V, G172L, T236L, T319I, H375L, H419Y, T424F, E529L, T541L, and G562X. In some embodiments, the endonuclease of SEQ ID NO: 3 comprises D38F, A59V, G172L, T236L, T319I, H375L, H419Y, T424F, E529L, T541L, G562X, and K564M. In some embodiments, the endonuclease of SEQ ID NO: 3 comprises D38F, A59V, G172L, T236L, T319I, H375L, H419Y, T424F, E529L, T541L, G562X, K564M, and D569L. In some embodiments, the endonuclease of SEQ ID NO: 3 comprises D38F, A59V, G172L, T236L, T319I, H375L, H419Y, T424F, E529L, T541L, G562X, K564M, D569L, and A586I. In some embodiments, the endonuclease of SEQ ID NO: 3 comprises D38F, A59V, G172L, T236L, T319I, H375L, H419Y, T424F, E529L, T541L, G562X, K564M, D569L, A586I, and N641F. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, and D642L. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, and S647L. In some embodiments, the endonuclease of SEQ ID NO: 3 comprises D38F, A59V, G172L, T236L, T319I, H375L, H419Y, T424F, E529L, T541L, G562X, aK564M, D569L, A586I, N641F, D642L, S647L, and D721L. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, and R779I. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, and K13R. In some embodiments, the endonuclease of SEQ ID NO: 3 comprises D38F, A59V, G172L, T236L, T319I, H375L, H419Y, T424F, E529L, T541L, G562X, aK564M, D569L, A586I, N641F, D642L, S647L, D721L, R779I, K13R, and K566R. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, K13R, K566R, and G554H. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, K13R, K566R, G554H, and A35N. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, K13R, K566R, G554H, and A35N. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, K13R, K566R, G554H, A35N, and E110T. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, K13R, K566R, G554H, A35N, E110T, and G314Q. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, K13R, K566R, G554H, A35N, E110T, G314Q, and K114P. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, K13R, K566R, G554H, A35N, E110T, G314Q, K114P, and D498P. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, K13R, K566R, G554H, A35N, E110T, G314Q, K114P, D498P, and 186P. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, K13R, K566R, G554H, A35N, E110T, G314Q, K114P, D498P, I86P, and V57E. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, K13R, K566R, G554H, A35N, E110T, G314Q, K114P, D498P, I86P, V57E, and H249W. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, K13R, K566R, G554H, A35N, E110T, G314Q, K114P, D498P, I86P, V57E, H249W, and R704F.

In some embodiments, the disclosed herein are one or more (e.g. about 1, or about 2, or about 3, or about 4, or about 5, or about 6, or about 7, or about 8, or about 9, or about 10, or about 15, or about 20, or about 30) substitutions to SEQ ID NO: 3 or a sequence with at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, 99.8, 99.9% identity to SEQ ID NO: 3 (or about 70%, or about 75%, or about 80%, or about 85%, or about 90, or about 95%, or about 96%, or about 97%, or about 98%, or about 99% identity to SEQ ID NO: 3). In various embodiments, one or more amino acids of SEQ ID NO: 3 is substituted with a naturally occurring amino acid, such as a hydrophilic amino acid (e.g. a polar and positively charged hydrophilic amino acid, such as arginine (R) or lysine (K); a polar and neutral of charge hydrophilic amino acid, such as asparagine (N), glutamine (Q), serine (S), threonine (T), proline (P), and cysteine (C), a polar and negatively charged hydrophilic amino acid, such as aspartate (D) or glutamate (E), or an aromatic, polar and positively charged hydrophilic amino acid, such as histidine (H)) or a hydrophobic amino acid (e.g. a hydrophobic, aliphatic amino acid such as glycine (G), alanine (A), leucine (L), isoleucine (I), methionine (M), or valine (V), a hydrophobic, aromatic amino acid, such as phenylalanine (F), tryptophan (W), or tyrosine (Y) or a non-classical amino acid (e.g. selenocysteine, pyrrolysine, N-formylmethionine β-alanine, GABA and δ-Aminolevulinic acid. 4-Aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

In illustrative embodiments, inventive substitutions include, but are not limited to one or more (e.g. about 1, or about 2, or about 3, or about 4, or about 5, or about 6, or about 7, or about 8, or about 9, or about 10, or about 15, or about 20, or about 30) substitutions to SEQ ID NO: 3, or a sequence with at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, 99.8, 99.9% identity to SEQ ID NO: 3: D38F, A59V, G172L, T236L, T319I, H375L, H419Y, T424F, E529L, T541L, G562X, K564M, D569L, A586I, N641F, D642L, S647L, D721L, R779I, K13R, K566R, G554H, A35N, E110T, G314Q, K114P, D498P, I86P, V57E, H249W, and R704F.

The details of one or more examples of the disclosure are set forth in the description below. Other features or advantages of the present disclosure will be apparent from the following drawings, detailed description of several examples, and also from the appended claims. The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs.

### DESCRIPTION OF THE DRAWINGS

FIG. 1A, FIG. 1B, FIG. 1C, and FIG. 1D are images showing the protein sizes of the Cas13K2F system for SEQ ID NO: 1 (Cas13K2F1), SEQ ID NO: 2 (Cas13K2F2), SEQ ID NO: 3 (Cas13K2F3), SEQ ID NO: 4 (Cas13K2F5), SEQ ID NO: 80 (Cas13K2F7), SEQ ID NO: 81 (Cas13K2F8), SEQ ID NO: 82 (Cas13K2F9), SEQ ID NO: 83 (Cas13K2F10), SEQ ID NO: 84 (Cas13K2F11), SEQ ID NO: 85 (Cas13K2F12), SEQ ID NO: 86 (Cas13K2F13), and SEQ ID NO: 87 (Cas13K2F14). The red or black arrows in FIG. 1 indicate the placement of the higher eukaryotes and prokaryotes nucleotide-binding (HEPN) domain in each protein.

FIG. 2 is a non-limiting image showing a representative guide RNA structure from the Cas13K2F system.

FIG. 3 is an image showing the design of guide RNAs (gRNAs) to target multiple sites across the coding sequence of eGFP in HEK293T cells.

FIG. 4 is an image showing the gRNA structure for the Cas13K2F system (SEQ ID NO: 5).

FIG. 5 is a percent identity matrix of SEQ ID NO: 6 (Cas13X.1), SEQ ID NO: 7 (Cas13bt3), SEQ ID NO: 8 (Cas13bt2), SEQ ID NO: 9 (Cas13bt1), SEQ ID NO: 10 (Cas13bt8), SEQ ID NO: 11 (Cas13X.2), SEQ ID NO: 12 (Cas13bt9), SEQ ID NO: 13 (Cas13bt11), SEQ ID NO: 14 (Cas13bt5), SEQ ID NO: 15 (Cas13bt10), SEQ ID NO: 16 (Cas13bt15), SEQ ID NO: 17 (Cas13bt7), SEQ ID NO: 18 (Cas13bt6), SEQ ID NO: 19 (Cas13bt14), SEQ ID NO: 20 (Cas13Y.3), SEQ ID NO: 21 (Cas13bt12), SEQ ID NO: 22 (Cas13Y.1), SEQ ID NO: 23 (Cas13bt4), SEQ ID NO: 24 (Cas13bt16), SEQ ID NO: 25 (Cas13Y.5), and SEQ ID NO: 26 (Cas13Y.4).

FIG. 6 is an image showing a maximum likelihood phylogenetic tree of SEQ ID NO: 6 (Cas13X.1), SEQ ID NO: 7 (Cas13bt3), SEQ ID NO: 8 (Cas13bt2), SEQ ID NO: 9 (Cas13bt1), SEQ ID NO: 10 (Cas13bt8), SEQ ID NO: 11 (Cas13X.2), SEQ ID NO: 12 (Cas13bt9), SEQ ID NO: 13 (Cas13bt11), SEQ ID NO: 14 (Cas13bt5), SEQ ID NO: 15 (Cas13bt10), SEQ ID NO: 16 (Cas13bt15), SEQ ID NO: 17 (Cas13bt7), SEQ ID NO: 18 (Cas13bt6), SEQ ID NO: 19 (Cas13bt14), SEQ ID NO: 20 (Cas13Y.3), SEQ ID NO: 21 (Cas13bt12), SEQ ID NO: 22 (Cas13Y.1), SEQ ID NO: 23 (Cas13bt4), SEQ ID NO: 24 (Cas13bt16), SEQ ID NO: 25 (Cas13Y.5), and SEQ ID NO: 26 (Cas13Y.4).

FIGS. 7A-7C each show results of RNA cleavage experiments for SEQ ID NO: 2 (FIG. 7A), SEQ ID NO: 3 (FIG. 7B), and SEQ ID NO: 27 (FIG. 7C).

FIG. 8A is an image showing a maximum likelihood phylogenetic tree of Cas13, which has high divergence of Cas13, a clade which includes the Cas13K2F system, which is a fringe CRISPR-Cas13 family with <7% identity to previously characterized RNA-targeting systems.

FIG. 8B is a graph showing the percent of GFP positive cells following transfection with an SD reporter encoding the 5′end of GFP, an SA reporter having MS2 stem loops and encoding the 3′end of GFP, and/or (i) a catalytically inactive Cas13K2F fused to a MS2 coat protein (“dCas13K2F-MS2”) and gRNA targeting the SD reporter (Cas13K2F gRNA 1 or 2), or (ii) a catalytically inactive PspCas13 fused to a MS2 coat protein (dPspCas13b-MS2) and gRNA targeting the SD reporter (PspCas13b gRNA). Control cells were transfected with a non-targeting (NT) gRNA.

FIG. 9 is a graph showing the percent of GFP positive cells following transfection with (i) a target containing a sequence encoding the 5′end of GFP, a splice donor, a gene A intron, a splice acceptor, and a gene A exon, (ii) a template having two MS2 stem loops and a sequence encoding the 3′end of GFP, (iii) dCas13K2F fused to a MS2 coat protein (dCas13K2F-MS2) and gRNA directed to the target (gRNA12, gRNA2, gRNA18, or gRNA19). Control cells were transfected with a non-targeting (NT) gRNA or target only (“no RepRNA”).

FIG. 10A is an image showing ColabFold-based predicted protein structures and domain organizations of Cas13e (used herein interchangeably with “Cas13K2F”) and Cas13c representatives relative to EsiCas13d (PDB:6E9F). FIG. 10B is an image showing an RNA knockdown strategy to test the trans-splicing activity of Cas13e in mammalian cells. FIG. 10C is a graph showing the relative Cas13e GFP fluorescence (=MFI targeting crRNA/MFI nontargeting crRNA) of HEK293T-GFP cells transfected with plasmids expressing Cas13e or RfxCas13d, and GFP-targeting crRNA, measured by flow cytometry to show trans-splicing. Percent GFP detected in mammalian cells relative to non-targeting negative controls. In FIG. 10C, plasmids expressing Cas13e1 (used herein interchangeably with “Cas13K2F1”), Cas13e2 (used herein interchangeably with “Cas13K2F2”), Cas13e3 (used herein interchangeably with “Cas13K2F3”), Cas13e4 (used herein interchangeably with “Cas13K2F4”), and Cas13e5 (used herein interchangeably with “Cas13K2F5”) are shown on the x-axis. FIG. 10D is a graph showing the validation of dCas13e activity as capable of trans-splicing in mammalian cells.

FIG. 11A is an image showing v1 SE3 AAVs and the strategy for RNA replacement at the 3′ end. FIG. 11B is an image of experimental workflow for evaluating the performance of SE3 as AAV plasmids in alternative cell types. FIG. 11C is an image showing editing performance of SE3 with targeting (T) and non-targeting (NT) guides in HepG2 cell lines. FIG. 11D is a graph showing the number of pathogenic mutations, which are plotted in maroon (dark) per position in the USH2A gene, with exons shown in blue (grey color) and introns shown in light cream color (white color). Over 700 pathogenic variants occur throughout the full length. FIG. 11E is an image showing a non-limiting strategy for the correction of the 5′ end of a target RNA. FIG. 11F is a graph showing 5′ editing as applied to a USH2A reporter, with gRNAs targeting intron 12, vs a non-targeting guide where activity is driven by the presence of the repair RNA.

FIG. 12 is an image showing ColabFold-based predicted protein structures and domain organizations of different Cas13 family members. Cas13e is used herein interchangeably with “Cas13K2F”.

FIG. 13A and FIG. 13B are graphs showing Cas13K2F trans-splicing across two different RNA targets (MMP9 (FIG. 13A) and USH2A (FIG. 13B)) using PP7 (PCP) as the RNA binding partner (RBP) partner.

FIG. 14 is a graph showing the percent of GFP positive cells following transfection with an SD reporter encoding the 5′end of GFP and (i) an SA reporter having a sequence motif for the indicated RNA binding protein (RBP) and encoding the 3′end of GFP (“SA Reporter only” bars); or (ii) an SA reporter and a splice editor (“SE”) that was dPspCas13b fused to the indicated RBP and PspCas13b gRNA targeting the SD reporter (“SE+SA Reporter” bars). Control cells were transfected with SD reporter only or SD reporter and SA reporter only.

FIG. 15A is an image and FIG. 15B is a graph showing AAV delivery of dCas13K2F3 with a targeting (T) or non-targeting (NT) gRNA and a repRNA to facilitate trans-splicing in HEK293T cells.

FIG. 16 is a graph showing amino acid substitutions in dCas13K2F3 that enable either improvement or reduction of trans-splicing efficacy in human cells compared to the original (WT) sequence.

FIG. 17 is an image showing, without wishing to be bound by theory, internal exon replacement using 2 nucleases with 2 separate gRNAs and RBPs, with a repRNA shown in green (directly to the left of the splice donor site in the image).

FIG. 18 is an image showing, without wishing to be bound by theory, how internal exon replacement is achieved with a single gRNA, nuclease and an RBP in combination with a binding motif (BM) in the repRNA, shown in orange (and labeled BM2 in the image).

FIG. 19 is a graph showing targeting (T) in the first set of four bars on the left, or non-targeting (NT) in the second set of four bars on the right, of one gRNA, nuclease and RBP (PCP) in combination with a binding motif (BM) (see constructs in FIG. 18) to facilitate internal exon replacement.

FIG. 20 is an image showing, without wishing to be bound by theory, how internal exon replacement is achieved with two gRNAs, two nucleases and RBPs in combination with, or without, a binding motif (BM) in the repRNA, shown in orange (and labeled BM2 in the image).

FIG. 21A and FIG. 21B are graphs showing targeting (T) or non-targeting (NT) of two gRNAs, two nucleases and RBPs in combination with, or without, a binding motif (BM) in the repRNA (see constructs in FIG. 20) to facilitate internal exon replacement. In FIG. 21A, the first bar in each set is 3′ SE (T) (far left), the next bar in each set is 3′ SE (NT) (second from left), the next bar in each set is 5′ SE (T) (middle), the next bar in each set is 5′ SE (NT) (second from right), and the last bar in each set is 3′ and 5′ SE (T) (far right). In FIG. 21B, the first bar in each set is 3′ SE (T) (far left), the next bar in each set is 5′ SE (NT) (second from left), the next bar in each set is 3′ SE (T) (middle), the next bar in each set is 3′ SE (NT) (second from right), and the last bar in each set is 3′ and 5′ SE (T) (far right).

FIG. 22A is an image and FIG. 22B is a graph showing, without wish to be bound by theory, the design of a guide repair RNA (“grepRNA”), which are devoid of an RBP. FIG. 22B shows grepRNA only, or grepRNA and dCas13, for internal exon replacement across two different RNA targets (USH2A, left side of FIG. 22B; and MMP9, right side of FIG. 22B). In the top image of FIG. 22A, from 3′ to 5′, is the repRNA, gRNA, and dCas13-RBP. In the bottom image of FIG. 22B, from 3′ to 5′, is the grepRNA, and dCas13.

FIG. 23A is an image showing integrated USH2A target for 5′ replacement. FIG. 23B is a graph showing targeting (T) or non-targeting (NT) of an integrated 5′ USH2A target to facilitate internal exon replacement.

### DETAILED DESCRIPTION

The present disclosure provides, inter alia, compositions and methods related to new families of CRISPR-Cas effector proteins, including nucleic acids encoding CRISPR-Cas effector proteins, and RNA components to induce DNA targeting, and methods of use thereof.

The present disclosure is based, in part, on the discovery of compositions and methods related to type VI CRISPR-Cas effector proteins, optionally, complexed with a guide nucleic acid, that are capable of modifying a target nucleic acid. The present disclosure also provides methods of modifying a target nucleic acid using an endonuclease or chimeric protein of the present disclosure and, optionally, a guide RNA.

Belonging to type VI CRISPR-Cas enzymes, Cas13 enzymes were identified as RNA-guided RNA-targeting proteins. While Cas9 cleaves DNA to interrupt DNA replication, Cas13 digests RNA to abate transcription. The CRISPR-Cas13 system can be divided into six subtypes (a, b1, b2, c, d, X, Y). Each subtype carries Cas13, which is a single effector protein. All Cas13 proteins exhibit two distinct RNase activities. One is RNA-targeting degradation, the other is pre-crRNA processing. About six total Cas13 variants have been identified to date.

The present endonucleases (e.g., SEQ ID NO: 1, SEQ ID NO: 2, SEQ ID NO: 3, and SEQ ID NO: 4, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, or fragments or variants thereof), without wishing to be bound by theory, belong to a Cas13K2F system.

### Endonucleases (Protein, Nucleic Acid, and System)

The present disclosure provides, in aspects, a composition comprising an endonuclease comprising a sequence, or a fragment or variant thereof, having at least about 70% (or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%) to one or more of SEQ ID NOs: 1-4, and/or SEQ ID NOs: 80-89, or having about 1 to about 20 amino acid modifications (e.g. about 1, or about 2, or about 3, or about 4, or about 5, or about 6, or about 7, or about 8, or about 9, or about 10, or about 11, or about 12, or about 13, or about 14, or about 15, or about 16, or about 17, or about 18, or about 19, or about 20 modifications).

The present disclosure provides, in aspects, a composition comprising an endonuclease comprising a sequence, optionally comprising a HEPN domain, or a fragment or variant thereof, and having at least about 70% (or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%) to one or more of SEQ ID NOs: 1-4, and/or SEQ ID NOs: 80-89, or having about 1 to about 20 amino acid modifications (e.g. about 1, or about 2, or about 3, or about 4, or about 5, or about 6, or about 7, or about 8, or about 9, or about 10, or about 11, or about 12, or about 13, or about 14, or about 15, or about 16, or about 17, or about 18, or about 19, or about 20 modifications).

In embodiments, the sequence comprises at least one HEPN domain, or fragments or variants thereof. In embodiments, the sequence comprises at least two HEPN domains, or fragments or variants thereof.

In embodiments, the sequence comprises one or more truncated HEPN domains.

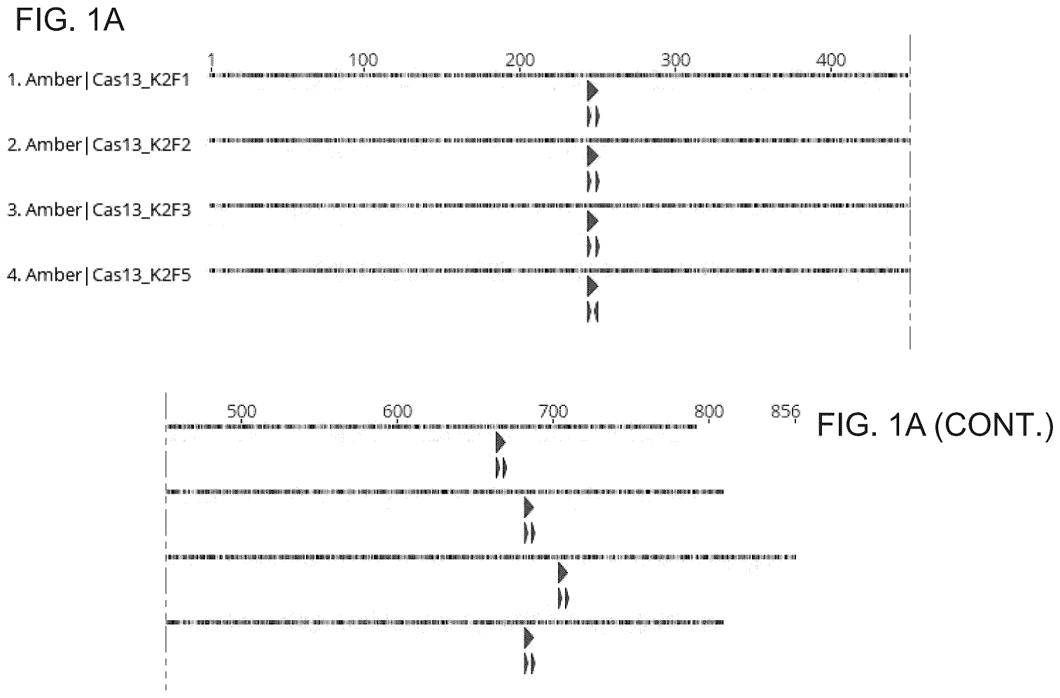

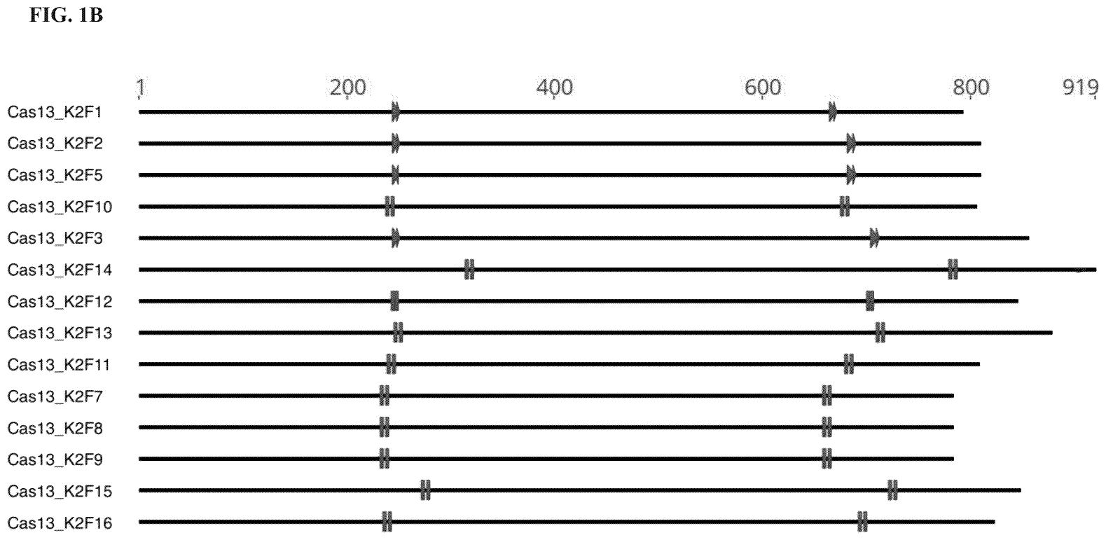

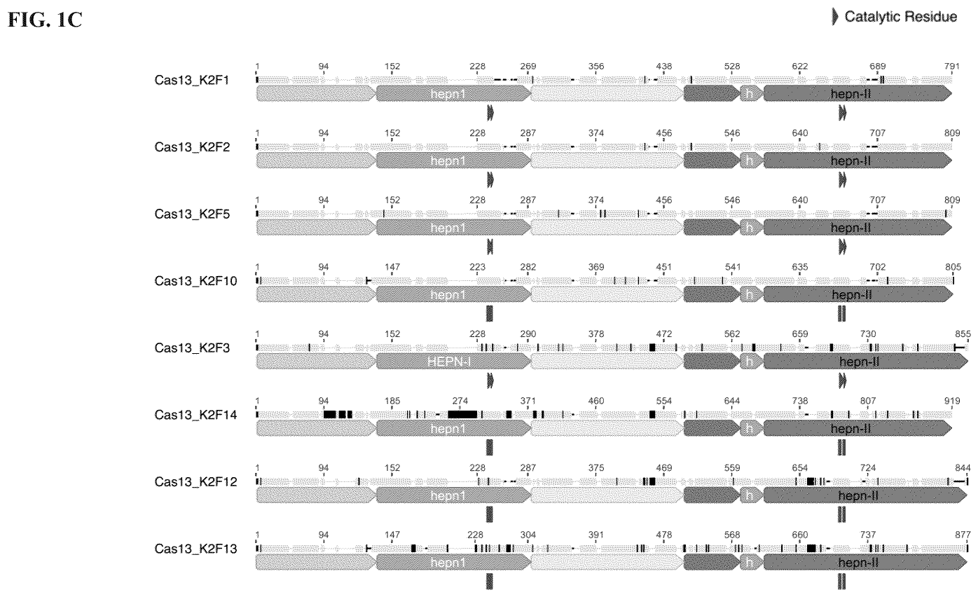

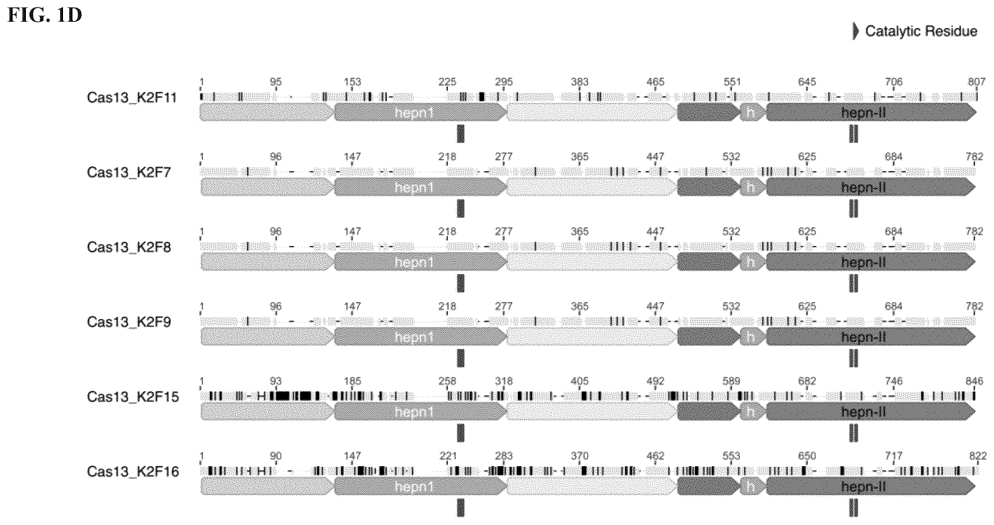

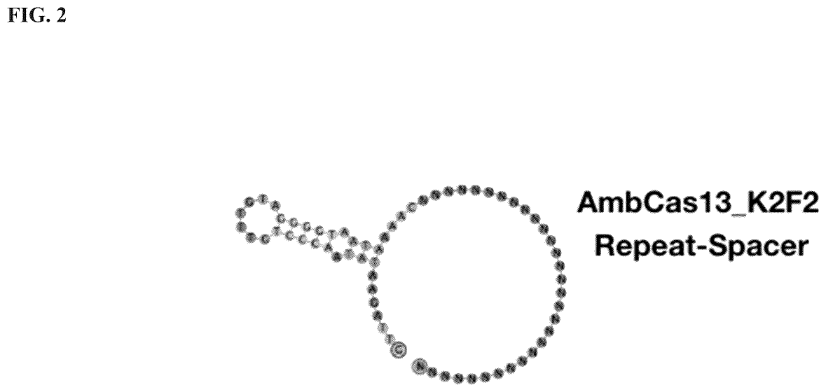

In embodiments, one or more HEPN domains are located according to the positions of the arrows in FIG. 1A, FIG. 1B, FIG. 1C, and FIG. 1D and/or by reference to Table 1 or Table 2.

In embodiments, a polypeptide of the present disclosure, e.g., endonuclease or chimeric protein, is provided as a nucleic acid (e.g., an mRNA, a DNA, a plasmid, an expression vector, a viral vector, and the like) that encodes the endonuclease or chimeric protein. In embodiments, the endonuclease or chimeric protein of the present disclosure is provided directly as a protein (e.g., without an associated guide RNA or with an associate guide RNA, i.e., as a ribonucleoprotein complex). An endonuclease or chimeric protein or nucleic acid of the present disclosure can be introduced into a cell (provided to the cell) by any convenient method; such methods are known to those of ordinary skill in the art.

In embodiments, the endonuclease is suitable for creating a double stranded break in a nucleic acid. In embodiments, the endonuclease is suitable for creating a nick in a nucleic acid. In embodiments, the endonuclease is suitable for nucleic acid modification by HDR. In embodiments, the endonuclease is suitable for nucleic acid modification by NHEJ.

In embodiments, the endonuclease recognizes a PAM. In embodiments, the endonuclease recognizes a plurality of PAMs (e.g., about 2, or about 3, or about 4, or about 5, or about 6, or about 8, or about 10 PAMs). In embodiments, the PAM sequence is about 1 to about 20, or about 2 to about 12, or about 2 to about 6, or about 2, or about 3, or about 4, or about 5, or about 6, or about 8, or about 10 nucleotides in length.

In embodiments, the endonuclease (or chimeric protein) comprises one or more mutations to reduce catalytic activity relative to an unmutated form. In embodiments, the one or more mutations to reduce catalytic activity relative to an unmutated form are in one or more HEPN domains of the present endonucleases. One of skill in the art may select the one or more mutations to reduce catalytic activity relative to an unmutated form by reference, e.g., to FIG. 1A, FIG. 1B, FIG. 1C, and FIG. 1D and/or by reference to Table 1 or Table 2 and/or by reference to structural information about other endonucleases known in the art, e.g. Slaymaker, et al., “High-Resolution Structure of Cas13b and Biochemical Characterization of RNA Targeting and Cleavage” 2019, Cell Reports 26, 3741-3751, Zhang et al., “Structural Basis for the RNA-Guided Ribonuclease Activity of CRISPR-Cas13d” Cell 175(1): 212-22, 2018 (each hereby incorporated by reference in their entireties), and the like.

In embodiments, the endonuclease (or chimeric protein) comprises one or more mutations to render the endonuclease substantially catalytically inactive relative to an unmutated form. In embodiments, the one or more mutations to render the endonuclease substantially catalytically inactive relative to an unmutated form are in one or more HEPN domains of the present endonucleases. One of skill in the art may select the one or more mutations to render the endonuclease substantially catalytically inactive relative to an unmutated form by reference, e.g., to FIG. 1A, FIG. 1B, FIG. 1C, and FIG. 1D and/or by reference to Table 1 or Table 2 and/or by reference to structural information about other endonucleases known in the art, e.g. Slaymaker, et al., “High-Resolution Structure of Cas13b and Biochemical Characterization of RNA Targeting and Cleavage” 2019, Cell Reports 26, 3741-3751, Zhang et al., “Structural Basis for the RNA-Guided Ribonuclease Activity of CRISPR-Cas13d” Cell 175(1): 212-22, 2018 (each hereby incorporated by reference in their entireties), and the like.

In embodiments, the endonuclease (or chimeric protein) comprises one or more mutations to increase catalytic activity relative to an unmutated form. In embodiments, the one or more mutations to increase catalytic activity relative to an unmutated form are in one or more HEPN domains of the present endonucleases. One of skill in the art may select the one or more mutations to increase catalytic activity relative to an unmutated form by reference, e.g., to FIG. 1 and/or by reference to Table 1 or Table 2 and/or by reference to structural information about other endonucleases known in the art, e.g. Slaymaker, et al., “High-Resolution Structure of Cas13b and Biochemical Characterization of RNA Targeting and Cleavage” 2019, Cell Reports 26, 3741-3751, Zhang et al., “Structural Basis for the RNA-Guided Ribonuclease Activity of CRISPR-Cas13d” Cell 175(1): 212-22, 2018 (each hereby incorporated by reference in their entireties), and the like.

In embodiments, the endonuclease (or chimeric protein) comprises one or more mutations to render the endonuclease substantially catalytically hyperactive relative to an unmutated form. In embodiments, the one or more mutations to render the endonuclease substantially catalytically hyperactive relative to an unmutated form are in one or more HEPN domains of the present endonucleases. One of skill in the art may select the one or more mutations to render the endonuclease substantially catalytically hyperactive relative to an unmutated form by reference, e.g., to FIG. 1A, FIG. 1B, FIG. 1C, and FIG. 1D and/or by reference to Table 1 or Table 2 and/or by reference to structural information about other endonucleases known in the art, e.g. Slaymaker, et al., “High-Resolution Structure of Cas13b and Biochemical Characterization of RNA Targeting and Cleavage” 2019, Cell Reports 26, 3741-3751, Zhang et al., “Structural Basis for the RNA-Guided Ribonuclease Activity of CRISPR-Cas13d” Cell 175(1): 212-22, 2018 (each hereby incorporated by reference in their entireties), and the like.

In embodiments, the endonuclease has nickase activity. In embodiments, the endonuclease (or chimeric protein) comprises one or more mutations to produce nickase activity. In embodiments, the one or more mutations to produce nickase activity relative to an unmutated form are in one or more HEPN domains of the present endonucleases. One of skill in the art may select the one or more mutations to produce nickase activity relative to an unmutated form by reference, e.g., to FIG. 1A, FIG. 1B, FIG. 1C, and FIG. 1D and/or by reference to Table 1 or Table 2 and/or by reference to structural information about other endonucleases known in the art, e.g. Slaymaker, et al., “High-Resolution Structure of Cas13b and Biochemical Characterization of RNA Targeting and Cleavage” 2019, Cell Reports 26, 3741-3751, Zhang et al., “Structural Basis for the RNA-Guided Ribonuclease Activity of CRISPR-Cas13d” Cell 175(1): 212-22, 2018 (each hereby incorporated by reference in their entireties), and the like.

In embodiments, the endonuclease has collateral cleavage activity. In embodiments, the endonuclease (or chimeric protein) comprises one or more mutations to produce, increase, remove, or decrease collateral cleavage activity. In embodiments, the one or more mutations to produce, increase, remove, or decrease collateral cleavage activity relative to an unmutated form are in one or more HEPN domains of the present endonucleases. One of skill in the art may select the one or more mutations to produce, increase, remove, or decrease collateral cleavage activity relative to an unmutated form by reference, e.g., to FIG. 1A, FIG. 1B, FIG. 1C, and FIG. 1D and/or by reference to Table 1 or Table 2 and/or by reference to structural information about other endonucleases known in the art, e.g. Slaymaker, et al., “High-Resolution Structure of Cas13b and Biochemical Characterization of RNA Targeting and Cleavage” 2019, Cell Reports 26, 3741-3751, Zhang et al., “Structural Basis for the RNA-Guided Ribonuclease Activity of CRISPR-Cas13d” Cell 175(1): 212-22, 2018 (each hereby incorporated by reference in their entireties), and the like.

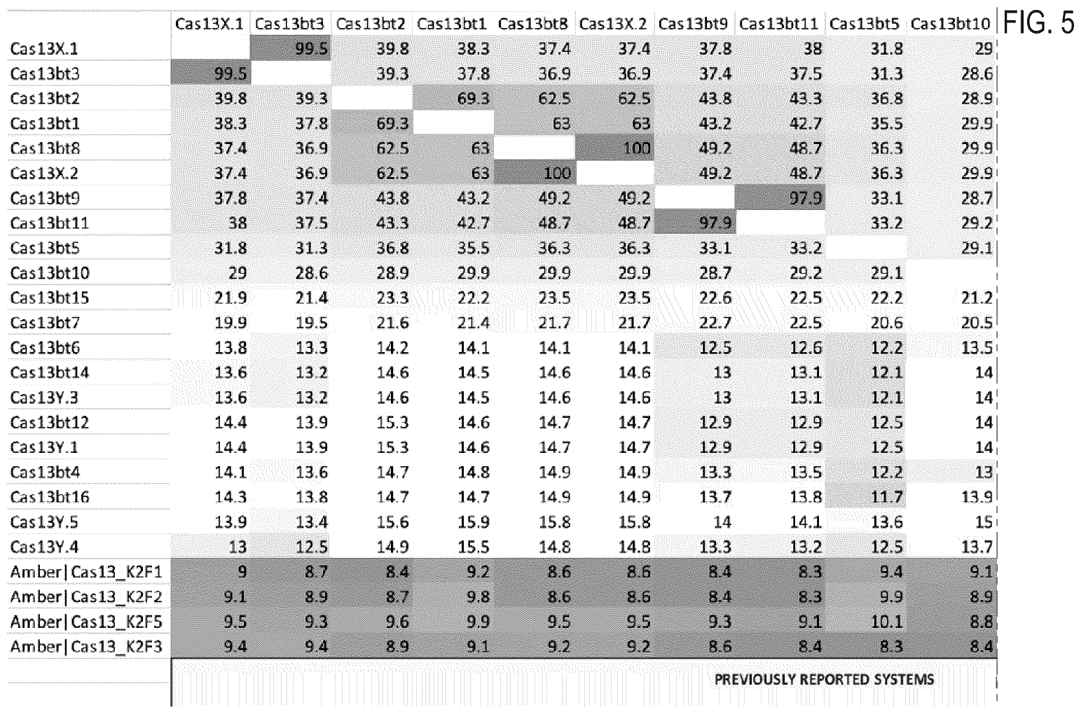

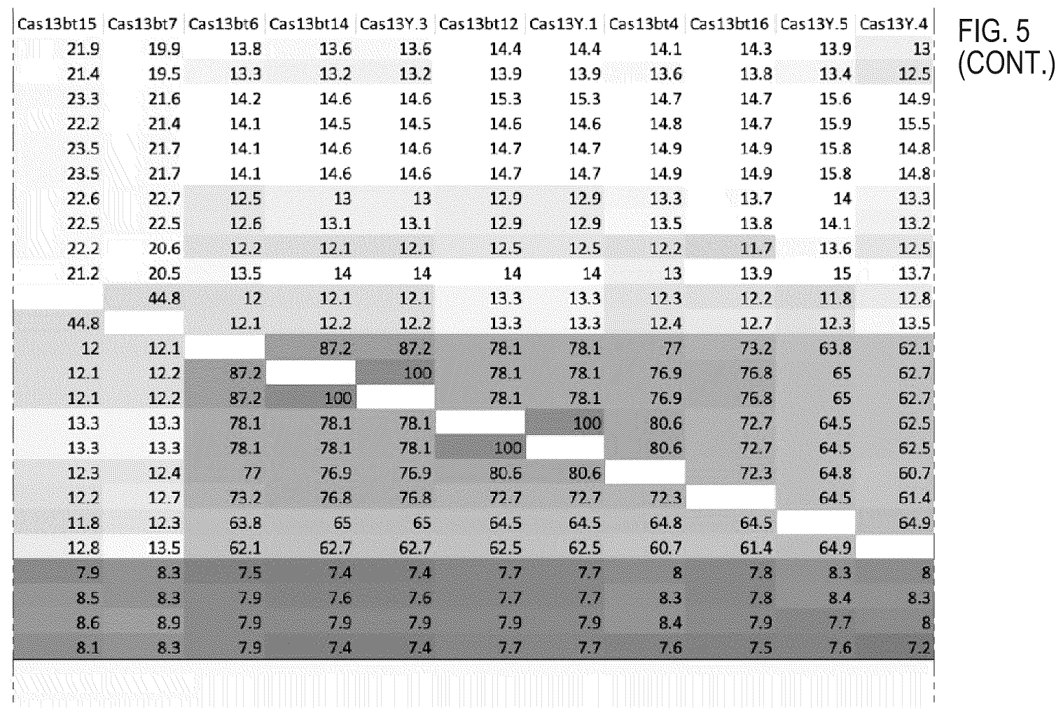

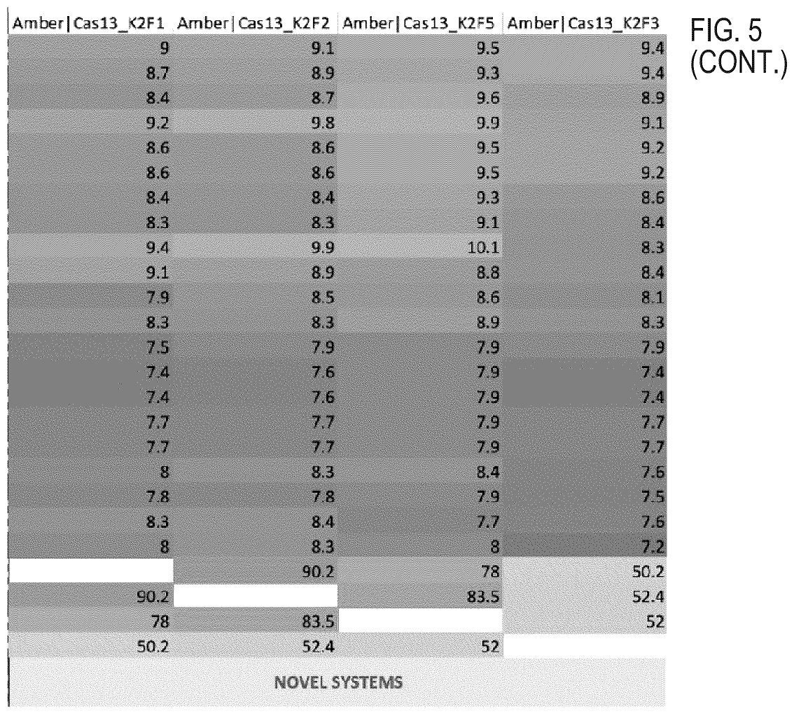

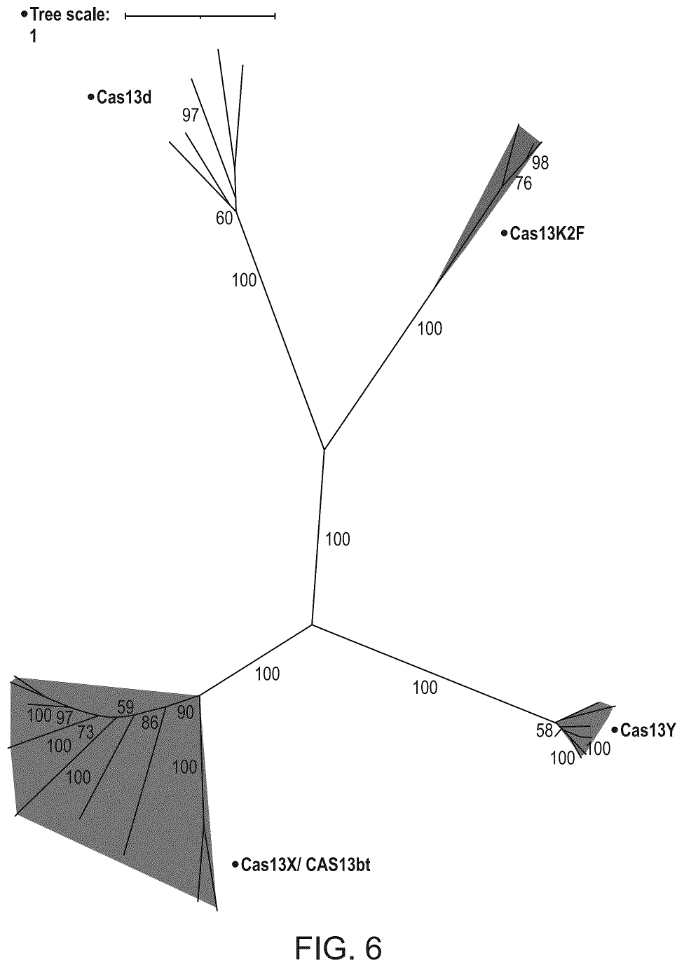

In embodiments, one of skill in the art may select residues to alter in light of a desired percent sequence identity and/or select amino acid modifications by reference to the domains of e.g., to FIG. 1A, FIG. 1B, FIG. 1C, and FIG. 1D and/or by reference to Table 1 or Table 2 and/or reference to the alignment information of FIG. 5 and/or the phylogenetic information of FIG. 6 and/or by reference to structural information about other endonucleases known in the art, e.g. Slaymaker, et al., “High-Resolution Structure of Cas13b and Biochemical Characterization of RNA Targeting and Cleavage” 2019, Cell Reports 26, 3741-3751, Zhang et al., “Structural Basis for the RNA-Guided Ribonuclease Activity of CRISPR-Cas13d” Cell 175(1): 212-22, 2018 (each hereby incorporated by reference in their entireties), and the like.

In embodiments, the amino acid modifications are amino acid mutations or amino acid substitutions. In embodiments, the amino acid substitutions are conservative and/or non-conservative substitutions. In embodiments, the amino acid modifications are amino acid truncations of two or more amino acids (e.g. about to about 100, or about 2 to about 90, or about 2 to about 80, or about 2 to about 70, or about 2 to about 60, or about 2 to about 50, or about 2 to about 40, or about 2 to about 30, or about 2 to about 20, or about 2 to about 10, or about 20 to about 100, or about 50 to about 100, or about 70 to about 100 amino acids).

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In embodiments, the substitutions may also include non-classical amino acids (e.g. selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

In embodiments, the percent sequence identity between a particular nucleic acid or amino acid sequence and a sequence referenced by a particular sequence identification number is determined as follows. A nucleic acid or amino acid sequence is compared to the sequence set forth in a particular sequence identification number using, e.g., the BLAST 2 Sequences (Bl2seq) program from the stand-alone version of BLASTZ containing BLASTN version 2.0.14 and BLASTP version 2.0.14 or the like. This stand-alone version of BLASTZ can be obtained online at or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options may be set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -l; -r is set to 2; and all other options are left at their default setting. For example, the following command can be used to generate an output file containing a comparison between two sequences: C:\Bl2seq -i c:\seq1.txt -j c:\seq2.txt -p blastn -o c:\output.txt -q -1 -r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences. Once aligned, the number of matches is determined by counting the number of positions where an identical nucleotide or amino acid residue is presented in both sequences. The percent sequence identity is determined by dividing the number of matches either by the length of the sequence set forth in the identified sequence (e.g., any of SEQ ID NOs: 1-4, and/or SEQ ID NOs: 80-89), or by an articulated length (e.g., 100 consecutive nucleotides or amino acid residues from a sequence set forth in an identified sequence), followed by multiplying the resulting value by 100. It is noted that the percent sequence identity value is rounded to the nearest tenth. For example, 75.11, 75.12, 75.13, and 75.14 is rounded down to 75.1, while 75.15, 75.16, 75.17, 75.18, and 75.19 is rounded up to 75.2. It also is noted that the length value will always be an integer.

In embodiments, the endonuclease has at least about 75% identity to one or more of SEQ ID NOs: 1-4, and/or SEQ ID NOs: 80-89. In embodiments, the endonuclease has at least about 80% identity to one or more of SEQ ID NOs: 1-4, and/or SEQ ID NOs: 80-89. In embodiments, the endonuclease has at least about 85% identity to one or more of SEQ ID NOs: 1-4, and/or SEQ ID NOs: 80-89. In embodiments, the endonuclease has at least about 90% identity to one or more of SEQ ID NOs: 1-4, and/or SEQ ID NOs: 80-89. In embodiments, the endonuclease has at least about 95% identity to one or more of SEQ ID NOs: 1-4, and/or SEQ ID NOs: 80-89. In embodiments, the endonuclease has at least about 97% identity to one or more of SEQ ID NOs: 1-4, and/or SEQ ID NOs: 80-89. In embodiments, the endonuclease has at least about 99% identity to one or more of SEQ ID NOs: 1-4, and/or SEQ ID NOs: 80-89.

In embodiments, the endonuclease has about 1 to about 15 amino acid modifications. In embodiments, the endonuclease has about 1 to about 10 amino acid modifications. In embodiments, the endonuclease has about 1 to about 5 amino acid modifications. In embodiments, the endonuclease has about 1, or about 2, or about 3, or about 4, or about 5, or about 10, or about 15, or about 20 amino acid modifications. In embodiments, the amino acid modifications are selected from substitutions and deletions.

In embodiments, the endonuclease is selected from Table 1 below.

In embodiments, the sequence comprises at least one HEPN domain, or fragments or variants thereof. In embodiments, the sequence comprises at least two HEPN domains, or fragments or variants thereof. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to one or more of SEQ ID NOs: 1-4, and/or SEQ ID NOs: 80-89. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 1. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 2. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 3. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 4. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 80. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 81. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 82. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 83. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 84. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 85. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 86. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 87. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 88. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 89. In embodiments, the endonuclease comprises about 1 to about 20 amino acid modifications (e.g. about 1, or about 2, or about 3, or about 4, or about 5, or about 6, or about 7, or about 8, or about 9, or about 10, or about 11, or about 12, or about 13, or about 14, or about 15, or about 16, or about 17, or about 18, or about 19, or about 20 modifications) to SEQ ID NOs: 1-4, and/or SEQ ID NOs: 80-89. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 1. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 2. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 3. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 4. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 80. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 81. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 82. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 83. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 84. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 85. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 86. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 87. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 88. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 89.

In aspects, the present disclosure provides a composition comprising a nucleic acid encoding an endonuclease comprising a sequence, optionally comprising a HEPN domain, or a fragment or variant thereof, and having at least about 70% identity to one or more of SEQ ID NOs: 1-4, and/or SEQ ID NOs: 80-89, or having about 1 to about 20 amino acid modifications (e.g. about 1, or about 2, or about 3, or about 4, or about 5, or about 6, or about 7, or about 8, or about 9, or about 10, or about 11, or about 12, or about 13, or about 14, or about 15, or about 16, or about 17, or about 18, or about 19, or about 20 modifications). In embodiments, the sequence comprises at least two HEPN domains, or fragments or variants thereof. In embodiments, the HEPN domain is located according to the positions of the arrows in FIG. 1A, FIG. 1B, FIG. 1C, and FIG. 1D. In embodiments, the endonuclease is selected from Table 1 below. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to one or more of SEQ ID NOs: 1-4, and/or SEQ ID NOs: 80-89. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 1. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 2. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 3. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 4. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 80. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 81. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 82. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 83. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 84. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 85. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 86. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 87. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 88. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 89. In embodiments, the endonuclease comprises about 1 to about 20 amino acid modifications (e.g. about 1, or about 2, or about 3, or about 4, or about 5, or about 6, or about 7, or about 8, or about 9, or about 10, or about 11, or about 12, or about 13, or about 14, or about 15, or about 16, or about 17, or about 18, or about 19, or about 20 modifications) to SEQ ID NOs: 1-4, and/or SEQ ID NOs: 80-89. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 1. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 2. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 3. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 4. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 80. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 81. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 82. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 83. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 84. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 85. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 86. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 87. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 88. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 89.

In aspects, the present disclosure provides a composition comprising a nuclease system, comprising (a) an endonuclease comprising a sequence, optionally comprising a HEPN domain, or a fragment or variant thereof, and having at least about 70% identity to one or more of SEQ ID NOs: 1-4, and/or SEQ ID NOs: 80-89, or having about 1 to about 20 amino acid modifications (e.g. about 1, or about 2, or about 3, or about 4, or about 5, or about 6, or about 7, or about 8, or about 9, or about 10, or about 11, or about 12, or about 13, or about 14, or about 15, or about 16, or about 17, or about 18, or about 19, or about 20 modifications); and (b) an RNA molecule comprising a sequence complementary to one strand of a target nucleic acid molecule. In embodiments, the HEPN domain is located according to the positions of the arrows in FIG. 1A, FIG. 1B, FIG. 1C, and FIG. 1D. In embodiments, the sequence comprises at least two HEPN domains, or fragments or variants thereof. In embodiments, the endonuclease is selected from Table 1 below. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to one or more of SEQ ID NOs: 1-4, and/or SEQ ID NOs: 80-89. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 1. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 2. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 3. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 4. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 80. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 81. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 82. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 83. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 84. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 85. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 86. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 87. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 88. In embodiments, the endonuclease comprises a sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identity to SEQ ID NO: 89. In embodiments, the endonuclease comprises about 1 to about 20 amino acid modifications (e.g. about 1, or about 2, or about 3, or about 4, or about 5, or about 6, or about 7, or about 8, or about 9, or about 10, or about 11, or about 12, or about 13, or about 14, or about 15, or about 16, or about 17, or about 18, or about 19, or about 20 modifications) to SEQ ID NOs: 1-4, and/or SEQ ID NOs: 80-89. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 1. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 2. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 3. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 4. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 80. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 81. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 82. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 83. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 84. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 85. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 86. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 87. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 88. In embodiments, the endonuclease comprises about 1, about 2, about 3, about 4, about 5, about 6, about 7, about 8, about 9, about 10, about 11, about 12, about 13, about 14, about 15, about 16, about 17, about 18, about 19, or about 20 amino acid modifications to SEQ ID NO: 89.

TABLE 1
Amino Acid Sequences of SEQ ID NOs: 1-4 and
SEQ ID NOs: 80-89

| | SEQ ID |
|---|---|
| Sequence | NO: |
| MIKKPSNRHALPKVIISEVNSEKILEFKIKYEKLARLDRFEVKAMHYE | 1 |
| GKEIVFDEVLVNGGLIDVEYEDEHKTLFVKVGEKSYSIRGQKVGGKQ | |
| RLLENRVSKTKVLLELSDGVPDKNAKLRKSRTERELIVVENIKLYSQI | |
| VGKEVSTTKEIYLTKRFLSYRSDLLFYYSFVDNFFKVAGNEKELWKIN | |
| FDDASSAQFMGYVPFMVNDNLKNDNAYLKDYVSDNEQIKDDLKKV | |
| QTMFSTLRHALLHFNYEFLNLLIENIDKLNIDAKKEFIDEEKIKLFGEN | |
| LSLAKVYRLYSDICVNRVGFNKFINSMLIKDGVENQALKAEFDRKFF | |
| GKAYTIDIHSNQAYKRIYNEHKKLVIKVSTLKDGQAIRRGNKKISELK | |
| EQMKSMTKKNSLARLECKMRLAFGFLYGEYNNYNTFKNNFDTNIKN | |
| SQFDVNDVEKSKAYFLSTYERRKPRTSEKLEKVAKNIERLELKTVIAN | |
| DPLLKFILLMFAFMPQELKGEFLGFVKKYYHDVHSIDDDTKEQEQDV | |
| VEAMSTSLKLKILGRNIRSLTLFKYALSSQVNYNSTYNLFYVEGNRYG | |
| KIYKKLGISHNQEEFDKTLVVPLLRYYSALFKLMNDFEIYSLAKANPT | |
| AVSLQELVDDERSPYKQGRFYNFREVLKQVYLLSDNELTHCKIRITRN | |
| KIAHFITEDLLGKPLLGEIKLNLQRKDMVSFMEARGNIKELLDYDAIN | |
| DFRMKVIHLRTKMRVNSDKLQTMMDLLSNPKTPNDFYNVYKVKGV | |
| EIINKHLLEVLAQTAEERSIEKQIREGNEKYAL | |
| MIKKPSNRHALPKVIISEVNSEKILEFKIKYEKLARLDRFEVKAMHYE | 2 |
| GKEIVFDEVLVNGGLIDVEYHDEHKTLFVKVGEKSYSIRGQKVGGKQ | |
| RLLENRVSKTKVLLELSDGVEDNKGNLRKSKTERELIVAENIKLYSQI | |
| VGREVSTTKEIYLTKRFLSYRSDLLFYYSFVDNFFKVAGNEKELWKIN | |
| FDDASSAQFMGYVPFMVNDNLKNDNAYLKDYVSDNEQIKDDLKKV | |
| QTMFSTLRHALLHFNYEFFEKLFNGEDVGFDFDIGFLNLLIENIDKLNI | |
| DAKKEFIDDEKIKLFGENLSLAKVYRLYSDICVNRVGFNKFINSMLIK | |
| DGVENQVLKAEFDRKFGGKAYTVDIHSNQAYKRIYNEHKKLVIKVST | |
| LKDGQAIRRGNKKISELKEQMKSMTKKNSLARLECKMRLAFGFLYG | |
| EYNNYNAFKNNFDTHIKNSQFDVNDVEKSKAYFLSTYERRKPRTSEK | |
| LEKVAKNIESLELKTVIANDPLLKFILLMFVFMPQELKGEFLGFVKKY | |
| YHDVHSIDDDTKEQEEDVVEAMSTSLKLKILGRNIRSLTLFKYALSSQ | |
| VNYNSTDNLFYVEGNRYGKIYKKLGISHNQEEFDKTLTVPLFRYYSA | |
| LFKLMNDFEIYSLAKANPMAVSLQELVDDETSPYKQGDYFNFNKML | |
| REIYGLTNDEIKSGKVVFMRNKIAHFDTEVLLSKPLLGQTKMNLQRK | |
| DIVSFIEARGNIKELLGYDAINDFRMKVIHLRTKMRVYSDKLQTMMD | |
| LLRSAKTPNDFYNVYKVKGVESINKYLLEVLAQTAEERSIEKQIKEGN | |
| EKYDL | |
| MDKHPSNRY ALPKVIISEVDHERILEFKVKYEKLARLDRFEVKAMHY | 3 |
| DGAEIVFDEVVANGGLIEVEYQDNNKTITINLNGKKYTINGRKVGGK | |
| RRLLEDRISRGKVCLELHDKIPDEKGNLRSSRTERELITFDSTKLYSQII | |
| GRDVASTKEIYLIKRFLAYRSDLLFYYGFIDNFFKVAGNKRELWKIDF | |
| SGDKNQELIKYFNFTINDKLKNDKGYLKEYTANDEQIKKDLQNTKEV | |
| FTALRHALMHFEYDFFEKLFNNEEIETLSKIHDIELLNTMINKLDKLNI | |
| DTRKEYIDDEKITVFGEEISLKTLYGLYAHTAINRVAFNKLINRFMVE | |
| NGTENEALKKYFNSKAEGGIAYEIDIHQNSEYKQLYIQHKDLVSKLSA | |
| LSDGDEIADTNKKISELKVKMKAITKANSLKRLEHKLRLTFGFIYTEY | |
| QDYNAFKNNFDTDIKSGRFIPKDSEGKRRGFDHRELDQLKRYYDATF | |
| ADKKPQTKETFDEIDKQIDQLSLKNLIGDDTLLKVILLIYIFLPREIKGE | |
| FLGFVKKYYHDTKHIEEDTKDKDEGFDDTFPVGLKLKVLDKNIRALS | |
| VLKHSLSYQAKYNKKEEKKEQFYEAGNRHGRFYKKLGISHNQEEFD | |
| KSVYAPLLRYHAALFKLLNDFEIYSLAQHIEGKETLAQQIEKPQFSQY | |
| EHYNFRKMLSKTYPKSAERGALDNDAFDTVINMRNDIAHLSHEPLFE | |
| CPLDGKKSYKLKQGKRTNTINVKPLPISRKMIVDFISSQSDMKKTLGY | |
| DAVNDLTMKIIQLRTRLKVYADKSETIKTLVDAAKTPNDFYHIYKVK | |
| GVEAINRHLLEVIGETKDEKRIRKRIESGNAIAGRTPADSQEN | |
| MIKKPSNRHALPKVIISEVNSDNILEFKIKYEKLARLDKIEVKAMHYD | 4 |
| NRNIVFDEVIVNDGLIELEYRDDHKRLFVKVGDKSYGITGQKVGGKQ | |
| RLLENRVSKTKVQLELTDGVLDNKGKHRISRTERELIVATNIALYNQII | |
| GREVKTTKEIYLIKRFLGYRSDLLFYYAFVDNFFKVADNEKELWKIDF | |
| DANNSTQLIKYISYIVNDNLKNDNAYLKEYVSNVEQIKEDLKKVQTIF | |
| SKLRHALLHFNYDFFEKLFNGKDVGFDFDIDFLNLLIENIDKLNIDAK | |
| KEFIDDEKIKLFGENLSLAKVYRLYSDICVNRVGFNKFINAMLIKDGV | |
| ENQALKEAFDNKLGRKAYTIDIHSNQEYKGLYNRHKKLVIELSTLKN | |
| GQAIRKKNAEIAKLKEQMNEMTKKNSLSRLEHKLRLAFGFMYAEYN | |
| NHKAFKNNFDTDIKNSKFSENDVEKFKAYFLSTYEGRKRRTSEKLEK | |
| VAKNIESLKLKTLIANDPLLKFILLMFVFMPQELKGEFLGFVKKYYHD | |
| IHSIDEDSKEQENTVLELMPTSLKLKILGRNIRSLTLFKYALSSQVNYN | |
| SSDELFYVEGNRYGKIYKKLGISHNQEEFDKTLVVPLFRYYSALFKLM | |
| NDFEIYSLAQANPRVLSLQELVDDTTSPYKQGDYYNFKKMLTEIYGV | |
| TNDEVNEGKVVFMRNKIAHFETKILLSKPLLGQTKLNLQRKDIVSFIE | |
| ARGDIKELLGYDAINDFRMKVIHLRTKMKVYADKLQTMMDLLRNV | |
| KTPNDFYNVYKVKGVESINKHLLEVLAQTDQERTIEKQMVEGNKKY | |
| KL | |
| MESNKNPSNRHSLPKVIISDVDKDNILEFKVKYEKLGRLDKFKIVSMK | 80 |
| YEDRDIVFRDIVSSDKSLEFSLANSNREIIVNLDNKKYTIRGQRVDNNE | |
| EKAKKVQLILTDNIKDENGAIRETLTERELIDNSDSIYSKIAGRKINSSK | |
| DIYLIKRYLAYRSNLQFFYNFIDKFFKIVDNKELWNIEFGNKHIEYFKF | |
| LINDNIKNANGYLYSYLQDNRRVKNDLYKTKDIFSKLRHALMHFDYE | |
| FFDKLFNNENLELDLNIEFLNLTIQNIDKLNIDTKKSYIGNQKIKIYSEEI | |
| KLDELYNLYNTISINRLGFNRLINSFFMQDGLENRKLKEFFNEEANSEE | |
| IYFVDIHQNRDYKKLYIKHKNFVAKLYGNRDGKSIAKLNRDISNIKKQ | |
| MQEITDKNSTLRLEYKLRVAFGFIYTNYKNYRHFKNSFDNDLKSGRF | |
| NNIDLSQIIEYYKNSYTNKDVLIRVTIKKIDKLNLNALIKDDNLLKIILLI | |
| FTFIPNELKGKFLGFIKRYYHDIKHIDEDSKEELEFNDGLSTSLKLKILH | |
| KNIRKLTILKYSLATESKYNKKDNYYYEDGHKTKRFLSSLGVSHNIEE | |
| FDKTIYTPFFKYYSAMYKLINDFEIFALTQFDSSANLKEITMKEELKQD | |
| NEYNFKILLRETNLYDENIVKLRNKISHIDGEFLFSNPLNRRINISSMRE | |
| KITNFIDSKNIKKILGYDALNDLSMKIIQQKTKLEANANKDEKINELIK | |
| NAQKANDYYSIYKLKAIEGINKRLLKIIGETKQEKYIKDKIIKGNNK | |
| MESNKNPSNRHSLPKVIISDVDKDNILEFKVKYEKLGRLDKFKIVSMK | 81 |
| YEDRDIVFRDIVSSDKSLEFSLANSNREIIVNLDNKKYTIRGQRVDNNE | |
| EKAKKVQLILTDNIKDENGAIRETLTERELIDNSDSIYSKIAGRKINSSK | |
| DIYLIKRYLAYRSNLQFFYNFIDKFFKIVDNKELWNIEFGNKHIEYFKF | |
| LINDNIKNANGYLYSYLQDNRRVKNDLYKTKDIFSKLRHALMHFDYE | |
| FFDKLFNNENLELDLNIEFLNLTIQNIDKLNIDTKKSYIGNQKIKIYSEEI | |
| KLDELYNLYNTISINRLGFNRLINSFFMQDGLENRKLKEFFNEEANSEE | |
| IYFVDIHQNRDYKKLYIKHKNFVAKLYGNRDGKTIARLNRDISNIKKQ | |
| MQEITDKNSTLRLEYKLRVAFGFIYTNYKNYMHFENSFDNDLKSGRF | |
| NNIDLSKIIEYYKNSYTNKDVRIRVTIKKIDKLNLNALIKDDNLLKIILLI | |
| FTFIPNELKGEFLGFIKRYYHDIKHIDEDSKEELEFNDGLSTSLKLKILH | |
| KNIRKLTILKYSLATESKYNKKDNYYYEDGHKTKRFLSSLGVSHNIEE | |
| FDKTIYTPFFKYYSAMYKLINDFEIFALTQFDSSANLKEITMKEELKQD | |
| NEYNFKILLRETNLYDENIVKLRNKISHIDGEFLFSNPLNRRINISSMRE | |
| KITNFIDSKNIKKILGYDALNDLSMKIIQQKTKLEANANKDEKINELIK | |
| NAQKANDYYSIYKLKAIEGINKRLLKIIGETKQEKYIKDKIIKGNNK | |
| MESNKNPSNRHSLPKVIISDVDKDNILEFKVKYEKLGRLDKFKIVSMK | 82 |
| YEDRDIVFRDIVSSDKSLEFSLANSNREIIVNLDNKKYTIRGQRVDNNE | |
| EKAKKVQLILTDNIKDENGAIRETLTERELIDNSDSIYSKIAGRKINSSK | |
| DIYLIKRYLAYRSNLQFFYNFIDKFFKIVDNKELWNIEFDNKHIEYFKF | |
| LINDNIKNANGYLYSYLQDNRRVKNDLYKTKDIFSKLRHALMHFDYEF | |
| FDKLFNNENLELDLNIEFLNFTIQNIDKLNIDTKKSYIGNQKIKIYSEEI | |
| KLDELYNLYNTISINRLGFNRLINSFFMQDGLENRELKKFFNEEANSEE | |
| IYFVDIHQNRDYKKLYIKHKNFVAKLYGNRDGKSIAKLNRDISNIKKQ | |
| MQEITDKNSTLRLEYKLRVAFGFIYTNYKNYRHFKNSFDNDLKSGRF | |
| NNIDLSKIIEYYKNSCTNKDVRIRVTIKKIDKLNLNALIKDDNLLKIILLI | |
| FTFIPNELKGEFLGFIKRYYHDIKHIDEDSKEELEFNDGLSKSLKLKILH | |
| KNIRKLTILKYSLATESKYNKKDNYYYEDGHKTKRFLSSLGVSHNIEE | |
| FDKTIYTPFFKYYSAMYKLINDFEIFALTQFDSSANLKEITMKEELKQD | |
| NEYNFKILLRETNLYDENIVKLRNKISHIDGEFLFSNPLNRRINISSMRE | |
| KITNFIDSKNIKKILGYDALNDLSMKIIQQKTKLEANANKDEKINELIK | |
| NAQKANDYYSIYKLKAIEGINKRLLKIIGETKQEKYIKDKIIKGNNK | |
| MTKKPANRHALPKVIISEVDSEKILEFKIKYEKLARLDRVEVKAMHYE | 83 |
| GKSIVFDEVVVNGGLIDVEYQDDHKTLFVKVGEKSYSIRGQKVGGKQ | |
| RLREERVSQVKVQLELTDGSSERVSRTERELIVADNIKLYSQIVGREV | |
| KTTKEIYLAKRFLGYRSDLLFYYGFVDNFFKVAGNEKELWKIDFEAS | |
| ESSQLLAYIPYMVNDNLKNNDAYLKDYIANEEQIKSDLKKVQTIFSEL | |
| RHALLHFNYDFFEKLFNGEDVGFDFDIEFLNLLIANIDKLNIDAKKEFI | |
| TDEKIKLFGENLSLAKVYKLYSDICVNRVGFNKFINSMLIKDGLENQA | |
| LKSEFDRKQGHKAYYIDIHSNEEYKRLYNRHKALVIKVSTLRDGQKIR | |
| KGNAEISEFKKQMNSMTTKNSLSHLEHKMRLAFGFMYGEYNHYNAF | |
| KNGFDTDVKNRKFDETDVSKSKAYFLSTYERQKPRTREKLERVAKDI | |
| ESLKLETLIAHDPLLKFILLMFAFMPREIKGEFLGFVKKYYHDVHSIEV | |
| DIIEQELDVVESMSTSLKLKNLGRNIRSLTLFKYALSAKVNYNGSDES | |
| FYEEGNRYGKIYKKLGISHNQEEFDKTLVVPLFRYYSALFKLMNDFEI | |
| YSLAKANPTALNLQMLVDDETSPYKQGNYYNFNKMLREVHGVTND | |
| EIKNGQAVFMRNKIAHFDTEVLLSKPLLGQTKMNLQRKIIIEFIKARGE | |
| MREILGYDAINDFRMKVVHLRTKMKVYSDKLQTMMDLLRSAKTPN | |
| DFYNVYKVKGVESINKQLLEVLAETAEERSIEKQICEGNMKYNS | |
| MSKNPSNRNSLPKVIINKVDENIILEFKIKYEKLARLDRFEVRSMRYDG | 84 |
| DGRIIFDEVVANGGLLDVGYEDDNKTIVVKIENKAYKIYGKKVGGKK | |
| RLNGKISKAKVQLILTDNIRKNANDTHRQSLTERELIDKNEIDLYSKIA | |
| EREISSTKDIYLVKRFLAYRSDLLLYYAFVNDYVKVKGNKEEFWKTPI | |
| DDKIIDYFIYTINDTLKNKEGYLEKYIVDRDQIKKDLEKTKRIFSHLRH | |
| KLMHYDFRFFTDLFDGKDVDIKVDNSTQKISELLDIKFLNIVIEELEKL | |
| NIDAKKEFIDDEKIPLFRQEIELKKLYSIYAHTAINRVAFNKLINSFLIK | |
| DGIENKELKEYFNAQNQGKESYYIDIHQNKEYKKLYIEHKDLLAKLS | |
| ATKNGKEIAKINRELADKKEQMKQITKANSLKRLEYKLRLAFGFIYTE | |
| YKDYETFKNSFDTDTKNQKFDAIDNAKIIEYFEATNKAKKIEKLEEILK | |
| GIDKLSLKTLIQDDILLKFLLLFFTFLPQEIKGEFLGFIRKYYHDITSLDE | |
| DTKDKDDEITELSRSLKLKIFAKNIRKLSILKHSLSYQIKYNKKESSYY | |
| EVGNAFNKMFKKQAISHNLEEFGKSIYLPMLKYYSALYKLINDFEIYA | |
| LYKDMDTSETLSQQVDKQEYERNEYFNFETLLRKKFGNDIEKVLVTY | |
| RNKIAHLDFNFLYDKPINKFISLYKSRDKIVNYIKNHDTQAVLKYDAV | |
| NDFVMKVIQQRSKLKVYADKEQTIESMIQNAQNPNDFYNIYKVKAVE | |
| NINQHLLKVIGYTDSEKAIEEKIRAGNISKS | |
| MLKKPVNRYALPKVIISEVNHEDILEFKIKYEKLGRLDRVAVKKMHY | 85 |
| EKENIVFDEVDVNGGLIEVMYKDEHQILLVQAGGKSYSIRGKKIGGK | |
| QRKREDRVSQVKIQLELTDGVLDKNEKYRVSQTERELIVNDNIKIYSQ | |
| IVGKEVKTTKEIYLIKRFLGYRSDLLFYYGFVDNFFKVVGNKTELWKI | |
| NFQDTKNEKLIEYFKFSINDKLKNDETYLKVYSSDNQNIEEDLTKVKN | |
| NFSKLRRALMHFDYGFFEKLFNDEDVGFDLDIMFLNVIIKNLDKLNID | |
| TRKEFIDDEKIKIFGEELSLKHLYGMYAHIAINRVAFNKLINSFMMQD | |
| GVENRSLKEYFNKRAKDGVAYEVDIHQNSQYKELYKQHKNLVSKVS | |
| ALSDGVAIAKMNDEIYTLKEKMKQITKPNSLKRLEHKLRLAFGFIYSE | |
| YKDYDDFKNNFNDHIIDGRFVPKDEEGKRRAFDSRELARLQGYYDVT | |
| LQNKKPQTKEKLGEVSKKIDSLSLATLIDDDKLLKFILLMFTFMPQEL | |
| KGEFLGFVKKYYHDTKHIEEDSKDKDKDFADGLSVGLRLKVLDKNIR | |
| NLSILKHSLSLQTKYNKKDNYFYEDGNVHGRFFKSLGISHNQEEFSKS | |
| VYAPLLKYYSALYKLINDFEIYTLAQYITTEYPTLSKVIDSEKFHLRWD | |
| NRSKELVPSDDYVFSTLTNKTYDHEKVKELNFIRNKISHFNSKELFEIP | |
| LQGYQMKGKKKLPFFLSKKREEIIDEIELQKDIQKTLGYDAINDFNMK | |
| MVQLYTKLKVYANKEETIEKMLEEATTPNDFYNVYKVKGVETINKH | |
| LLDVIGETEREKFIRIQIEVNNKRVSNENLDKL | |
| MSKKPANRHALPKVIISEVDSEKILEFKIKYEKLARLDRVEVKAMHYE | 86 |
| GKSIVFDEVVVNGGLIDVEYQDNHKTLFVKVGEKSYSIRGKKVGGKQ | |
| RLREERVSQVKVQLELSDGSSERVSRTERELIVNENIKLYSQIVGREVK | |
| TTKEIYLAKRFLGYRSDLLFYYGFVDNFFKEAKLFNARKNPIELWSEE | |
| FYVNDKLSNYTKFMFNDNLKNSESYLKEYIKNNEKENQKIKNDLESA | |
| RDIFATLRHNLMHFNYSFFERLFKGKDVKIKNLQTKKFESLSNVLRNI | |
| EFLNKVIQSIDKLNIDTRKEFIDKEKIKLFNEDLDLQQLYGFFAHTAIN | |
| RVAFNKLINSFIIKDGIENEQLKEYFNQRVDGTAYEIDIHONREYKELY | |
| KKHKNLVSKVSTLSDGKEIAKGNSEISALKEQMNKITKANSLKRLEY | |
| KLRLAFGFIYTEYGSYKAFVSRFNEDTKRKKIKNVEFEKIGFEKQKEY | |
| FKSTFEPLKSKKKDNLEKLIQEYEKLSLNDLIENDTFLKVILLLFIFMPK | |
| EVKGDFLGFIKKYYHDTKYIEEDTKEKDEGFTNTLPIGLKLKIVERNIA | |
| KLSVLKHSLSLKVKYNRGQYEEDNTYRKVFKKLNISHNQEEFHKSMF | |
| SPLLRYYASLYKLINDFEIYTLSHYITDKYSTLNKVIASKQFHYRYVW | |
| NRKENKGELVKTDNYTFSTLLSKKYEHKNSQEISEMRNKISHFDEKIL | |
| FKFPLEEVNSFFKGKGKNKKEEPVKSLVEKREEIISLMEKQTDMQKIL | |
| GYDAINDFRMKTVQFQTKLKEDSKKKEETIKKMIAEAKIPNDFYNIYK | |
| VKGVESINKHLLKIIGQTDKEWKIEGDILDGNFKIACKNQRLEEKQQR | |
| AKNKQNLDKL | |
| MIKNPSNRHSLPKVIISEVDHEKILEFKIKYEKLARLDRFEVKAMHYE | 87 |
| GKEIVFDEVLVNGGLIEVEYQDDNKTLFVKVGEKSYRICGERIGGNYI | |
| VTEYKDKNEPKSKKHFRLIEKDGKYFKPNGEEVTKNIRKSSVKVLLTL | |
| TDGVEDNNGKLRKSRTERELIVADNIKLYSQIVGREVTTTKEIYLVKR | |
| FLGYRSDLLFYYGFVNNFFHVAGKREELWKIDFDTLPSNSPLLEYFKF | |
| TINDEKYLKSYSSDIQQIKKDLQNNKYIFLVNGEDIEIKAENYNIKPLSE | |
| LLNIEFLNIQIKKDLQNSKYIFSALRHALMHFDYDFFVRLENGEDIEIK | |
| AKNGNKKPLSELLNIEFLNIMIENIDKLNIDTRKEFIDDDDVPIKLFGEE | |
| MKPKNLYGLYAHTAINRVAFNKLINSFMMENGVENQALKSYFDQKA | |
| GGVAYEVDIHQNSNYKKLYVKHKNLVSKVSTLSDGQEIAKVNAKISE | |
| LKEQMKKITKANSLKRLEHKFRLAFGFVYSEYKDYEAFKNNFDTDIK | |
| KGKFVPKDKEGKRRAFDHRELEQLKGYFDSTFKSKKPNTKEKLGELS | |
| KSTGKLSLKALIGDDMFLKFILLMFTFMPQELKGEFLGFVKKYYHDT | |
| KHIEEDTKDRDDGFSNERPMGLKLKVLDKNIRSLSILKHSLSFQTKYN | |
| KKDKSFYEDGNVHGKFYKKLGISHNQEEFNKSVYAPLFKYYSALSKL | |
| INDFEIYSLTQHVVGSETLAQQVRKRKFIKKGYYNFGNLLKKTDSIIRS | |
| SRDNDIFYAVIDMRNTISHLSVEPMFDYPLNGKKFYKLYENKVICVDP | |
| LKSRKMIIDFIKRQTEMKKTLGYDAVNDFTMKMVQLQTKLKVYANK | |
| EKTIEKMKEEAQTPNDYYNIYKVKGVEAINQYLLEIIGETDDEALIRKL | |
| INRGNSINP | |
| MKKIKNPSNRNSLPSIIISRFDDKNIYELKVKYEKLARLDKLEIEDMSL | 88 |
| DEESTLLFKKVKFNGIEIEIKNQKLLEFDSYIISGKKQTNTTGKTIISLLK | |
| EGKKVTYNVTKKDGKYYKNGKEFIIPQNANKLPDRLINDKFIITIEDK | |
| VRDEDTKRKKETQRDILSDDTIETYKRISSYKSIKSEDIYTIKRYITFKS | |
| DMMFFYTFVDDFFNPIKKQDLWKVKFGEVENLGKFIEFTLNDTLKNP | |
| KGILETYCKDLKTVQADFAKINTIFSKIRHSLVHFDFVFIDKLLSNQKIE | |
| EFDFDIKLLNDVIDKTQDLYYEAKKEFIEDEKITILDEKDMEIKKLYTF | |
| FSKIDIKQPAFNKLINSFIIKDGIENIELKTYIKEKYKSEYFIDIHANKEY | |
| KKIYNEHKKLVGENQFLQLNPKENGQKIKELNDQVEEYKKQMKTITE | |
| ANSLKRLEFKLRLAFGFIKVEYGRFDTFKNSFDEDIKKGKFKEISFEKI | |
| KGYLDKTYAKEQFFNYGSNKKTKKPYSILDDIENETLKELVQNDNLL | |
| KVILLFYIFTPKELKGEFLGFIKKFYHDTKNITKDTKDEEKELENLKLE | |
| TPLKLKILEKNLKKITIFNYSIFSNINFDTTNKRFYAEGNRFNRIYKKLN | |
| ISHNQDEFDKSLFAPLLQYYMNLYKLIGDFEIYLLLKFDNKKDLSELS | |
| NDERLKFRGYYNFTTLLSKWFQFDPKRDKKYEKVLRLRNTISHQDIN | |
| NMIINFEKSTILSQRENIVQLIEEQNDLKEILKYDAVNDFTMKTIQLLKS | |
| IEIQSDKSKTINELLSNKDISANDFYNIYKVKGVEMIKKELFNRLGKRE | |
| IEKKIEEEIAKSTIC | |
| MEKIKKPSNRNSIPSIIISDYDASKIKEIKVKYLKLARLDKITIQDMEIVD | 89 |
| NIVEFKKILLNGTEHTIIDNKKIEFDNYEITGCIKPSNKRRDGKISQAKY | |
| VVTITDKYLRDNEKEKRFKSTERELPNDILLSRYKQISGFDTLTSKDIY | |
| KIKRYIDFKNEMLFYFQFIEEFFNPLLPKGKNFYDLNIEQNKDKVAKFI | |
| VYRLNDDFKNQSLNSYIQKTDTIKYDFIKVQKILNDFRHALAHFDFEFI | |
| QKFFDNQLDKTRFDINTISLIKTLLQKKEGKHYQEKNNYIDDNDTLTIF | |
| DDKDSKFSKLHNFYTKISQKKPAFNKLINSFLSQDGIPNEEFKRYLATK | |
| KLDFFEDIHSNKEYKEIYINHKNQVIEKQKEESQEKPDGQKLKNFNDE | |
| LQKLKDKMNTITKQNSLNRLEVKLRLAFGFIANEYNYNFKNFNDNFT | |
| LDVKNEQKIKAFKNSSNEKLKEYFESTFEAKQFFYYGKNKNIFNSIEN | |
| ETLEELVKESPLLQIITFLYLFIPKELQGEFVGFILEIYHHTKNISSDTKE | |
| DEISIEDAQNSFSLKLKILAKNLRGLQLFNYSLSHNTLYNNKQDFFYE | |
| KGNRWQNIYKNFQISHNQDEFDIHLVIPVIKYYINLNKLIGDFEIYALL | |
| KYADQNSITEKLSDITKRDDLKFKGHYNFSTLLNRTFGISVYSDKNPIS | |
| IQNIKQIRNDIAHQNIENMLKAFENSEIFAQREEIVNYLQTEHQMQEIL | |
| HYNPINDFTMKTVQYLKSLSVHSQKEGKIADIHKKDNLVPNDYYLIY | |
| KLKAIEILKQKVIEAIGETKDEKKIKNAIAKEEQIKKGNN | |

In any aspects or embodiments herein, the present endonuclease, or a fragment or variant thereof, having at least about 70% identity to one or more of SEQ ID NOs: 1-4, and/or SEQ ID NOs: 80-89, has the N terminal M residue removed.

In any aspects or embodiments herein, the present endonuclease, or a fragment or variant thereof, having at least about 70% identity to one or more of SEQ ID NOs: 1-4, and/or SEQ ID NOs: 80-89, has the N terminal M residue removed and a SV40 NLS SV40 sequence is added to the N-terminus (MSPKKKRKVEAS (SEQ ID NO: 78)).

In any aspects or embodiments herein, the present endonuclease, or a fragment or variant thereof, having at least about 70% identity to one or more ofSEQ ID NOs: 1-4, and/or SEQ ID NOs: 80-89, has the N terminal M residue removed and a SV40 NLS SV40 sequence is added to the N-terminus (MSPKKKRKVEAS (SEQ ID NO: 78)) and a HA tag is added to the C-terminus (GSGPKKKRKVAAAYPYDVPDYA (SEQ ID NO: 77)).

In embodiments, the catalytic domain is selected from Table 2. In embodiments, a mutation of any of the present endonucleases is of one or more of the residues of Table 2.

TABLE 2
Illustrative Positioning of Catalytic Residues

| | Catalytic | | |
|---|---|---|---|
| | Domain | | Amino Acid |
| Protein | Type | Residue | Number |
| Cas13K2F1 | HEPN | R | 244 |
| (SEQ ID NO: 1) | | | |
| Cas13K2F1 | HEPN | H | 249 |
| (SEQ ID NO: 1) | | | |
| Cas13K2F1 | HEPN | H | 669 |
| (SEQ ID NO: 1) | | | |
| Cas13K2F1 | HEPN | R | 664 |
| (SEQ ID NO: 1) | | | |
| Cas13K2F2 | HEPN | R | 244 |
| (SEQ ID NO: 2) | | | |
| Cas13K2F2 | HEPN | H | 249 |
| (SEQ ID NO: 2) | | | |
| Cas13K2F2 | HEPN | H | 687 |
| (SEQ ID NO: 2) | | | |
| Cas13K2F2 | HEPN | R | 682 |
| (SEQ ID NO: 2) | | | |
| Cas13K2F3 | HEPN | R | 244 |
| (SEQ ID NO: 3) | | | |
| Cas13K2F3 | HEPN | H | 249 |
| (SEQ ID NO: 3) | | | |
| Cas13K2F3 | HEPN | R | 704 |
| (SEQ ID NO: 3) | | | |
| Cas13K2F3 | HEPN | H | 709 |
| (SEQ ID NO: 3) | | | |
| Cas13K2F5 | HEPN | R | 244 |
| (SEQ ID NO: 4) | | | |
| Cas13K2F5 | HEPN | H | 249 |
| (SEQ ID NO: 4) | | | |
| Cas13K2F5 | HEPN | H | 687 |
| (SEQ ID NO: 4) | | | |
| Cas13K2F5 | HEPN | R | 682 |
| (SEQ ID NO: 4) | | | |
| Cas13K2F7 | HEPN | R | 234 |
| (SEQ ID NO: 80) | | | |
| Cas13K2F7 | HEPN | H | 239 |
| (SEQ ID NO: 80) | | | |
| Cas13K2F7 | HEPN | R | 659 |
| (SEQ ID NO: 80) | | | |
| Cas13K2F7 | HEPN | H | 664 |
| (SEQ ID NO: 80) | | | |
| Cas13K2F8 | HEPN | R | 234 |
| (SEQ ID NO: 81) | | | |
| Cas13K2F8 | HEPN | H | 239 |
| (SEQ ID NO: 81) | | | |
| Cas13K2F8 | HEPN | R | 659 |
| (SEQ ID NO: 81) | | | |
| Cas13K2F8 | HEPN | H | 664 |
| (SEQ ID NO: 81) | | | |
| Cas13K2F9 | HEPN | R | 234 |
| (SEQ ID NO: 82) | | | |
| Cas13K2F9 | HEPN | H | 239 |
| (SEQ ID NO: 82) | | | |
| Cas13K2F9 | HEPN | R | 659 |
| (SEQ ID NO: 82) | | | |
| Cas13K2F9 | HEPN | H | 664 |
| (SEQ ID NO: 82) | | | |
| Cas13K2F10 | HEPN | R | 239 |
| (SEQ ID NO: 83) | | | |
| Cas13K2F10 | HEPN | H | 244 |
| (SEQ ID NO: 83) | | | |
| Cas13K2F10 | HEPN | R | 677 |
| (SEQ ID NO: 83) | | | |
| Cas13K2F10 | HEPN | H | 682 |
| (SEQ ID NO: 83) | | | |
| Cas13K2F11 | HEPN | R | 241 |
| (SEQ ID NO: 84) | | | |
| Cas13K2F11 | HEPN | H | 246 |
| (SEQ ID NO: 84) | | | |
| Cas13K2F11 | HEPN | R | 681 |
| (SEQ ID NO: 84) | | | |
| Cas13K2F11 | HEPN | H | 686 |
| (SEQ ID NO: 84) | | | |
| Cas13K2F12 | HEPN | R | 244 |
| (SEQ ID NO: 85) | | | |
| Cas13K2F12 | HEPN | H | 249 |
| (SEQ ID NO: 85) | | | |
| Cas13K2F12 | HEPN | R | 701 |
| (SEQ ID NO: 85) | | | |
| Cas13K2F12 | HEPN | H | 706 |
| (SEQ ID NO: 85) | | | |
| Cas13K2F13 | HEPN | R | 247 |
| (SEQ ID NO: 86) | | | |
| Cas13K2F13 | HEPN | H | 252 |
| (SEQ ID NO: 86) | | | |
| Cas13K2F13 | HEPN | R | 711 |
| (SEQ ID NO: 86) | | | |
| Cas13K2F13 | HEPN | H | 716 |
| (SEQ ID NO: 86) | | | |
| Cas13K2F14 | HEPN | R | 316 |
| (SEQ ID NO: 87) | | | |
| Cas13K2F14 | HEPN | H | 321 |
| (SEQ ID NO: 87) | | | |
| Cas13K2F14 | HEPN | R | 781 |
| (SEQ ID NO: 87) | | | |
| Cas13K2F14 | HEPN | H | 786 |
| (SEQ ID NO: 87) | | | |
| Cas13K2F15 | HEPN | R | 274 |
| (SEQ ID NO: 88) | | | |
| Cas13K2F15 | HEPN | H | 279 |
| (SEQ ID NO: 88) | | | |
| Cas13K2F15 | HEPN | R | 723 |
| (SEQ ID NO: 88) | | | |
| Cas13K2F15 | HEPN | H | 728 |
| (SEQ ID NO: 88) | | | |
| Cas13K2F16 | HEPN | R | 237 |
| (SEQ ID NO: 89) | | | |
| Cas13K2F16 | HEPN | H | 242 |
| (SEQ ID NO: 89) | | | |
| Cas13K2F16 | HEPN | R | 694 |
| (SEQ ID NO: 89) | | | |
| Cas13K2F16 | HEPN | H | 699 |
| (SEQ ID NO: 89) | | | |

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to one of SEQ ID NO: 1, SEQ ID NO: 2, SEQ ID NO: 3, SEQ ID NO: 4, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, and an amino acid modification one or more positions that have an R or H residue in the wild type sequence.

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to one of SEQ ID NO: 1, SEQ ID NO: 2, SEQ ID NO: 3, SEQ ID NO: 4, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, and an amino acid modification at one or more positions in a HEPN domain.

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to one of SEQ ID NO: 1, SEQ ID NO: 2, SEQ ID NO: 3, SEQ ID NO: 4, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, and an amino acid modification at one or more positions in a region of the endonuclease at about 100 to about 300 amino acids, or about 150 to about 300 amino acids, or about 150 to about 250 amino acids, or about 100 to about 300 amino acids, or about 200 to about 250 amino acids, or about 240 to about 250 amino acids from the N-terminus of the endonuclease.

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to one of SEQ ID NO: 1, SEQ ID NO: 2, SEQ ID NO: 3, SEQ ID NO: 4, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, and an amino acid modification at one or more positions in a region of the endonuclease at about 100 to about 300 amino acids, or about 150 to about 300 amino acids, or about 150 to about 250 amino acids, or about 100 to about 300 amino acids, or about 200 to about 250 amino acids, or about 240 to about 250 amino acids from the C-terminus of the endonuclease.

In embodiments, the amino acid modification is a hydrophilic or hydrophobic amino acid. In embodiments, the amino acid modification is amino acid modification is a hydrophilic amino acid. In embodiments, the amino acid modification is a polar and positively charged hydrophilic amino acid. In embodiments, the amino acid modification is arginine (R) or lysine (K). In embodiments, the amino acid modification is a polar and neutral charged hydrophilic amino acid. In embodiments, the amino acid modification is selected from asparagine (N), glutamine (Q), serine (S), threonine (T), proline (P), and cysteine (C). In embodiments, the amino acid modification is a polar and negatively charged hydrophilic amino acid. In embodiments, the amino acid modification is selected from aspartate (D) or glutamate (E). In embodiments, the amino acid modification is an aromatic, polar and positively charged hydrophilic amino acid. In embodiments, the amino acid modification is histidine (H). In embodiments, the amino acid modification is a hydrophobic amino acid. In embodiments, the amino acid modification is a hydrophobic, aliphatic amino acid. In embodiments, the amino acid modification is selected from glycine (G), alanine (A), leucine (L), isoleucine (I), methionine (M), or valine (V). In embodiments, the amino acid modification is a hydrophobic, aromatic amino acid. In embodiments, the amino acid modification is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 1 and an amino acid modification at position R244. In embodiments, the amino acid modifications are selected from substitutions and deletions. In embodiments, the amino acid modification to SEQ ID NO: 1 at R244 is an essential or non-essential amino acid. In embodiments, the amino acid modification at R244 is a hydrophilic or hydrophobic amino acid. In embodiments, the amino acid modification at R244 is amino acid modification is a hydrophilic amino acid. In embodiments, the amino acid modification at R244 is a polar and positively charged hydrophilic amino acid. In embodiments, the amino acid modification at R244 is lysine (K). In embodiments, the amino acid modification at R244 is a polar and neutral charged hydrophilic amino acid. In embodiments, the amino acid modification at R244 is selected from asparagine (N), glutamine (Q), serine (S), threonine (T), proline (P), and cysteine (C). In embodiments, the amino acid modification at R244 is a polar and negatively charged hydrophilic amino acid. In embodiments, the amino acid modification at R244 is selected from aspartate (D) or glutamate (E). In embodiments, the amino acid modification at R244 is an aromatic, polar and positively charged hydrophilic amino acid. In embodiments, the amino acid modification at R244 is histidine (H). In embodiments, the amino acid modification at R244 is a hydrophobic amino acid. In embodiments, the amino acid modification at R244 is a hydrophobic, aliphatic amino acid. In embodiments, the amino acid modification at R244 is selected from glycine (G), alanine (A), leucine (L), isoleucine (I), methionine (M), or valine (V). In embodiments, the amino acid modification at R244 is a hydrophobic, aromatic amino acid. In embodiments, the amino acid modification at R244 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 1 and an amino acid modification at position H249. In embodiments, the amino acid modifications are selected from substitutions and deletions. In embodiments, the amino acid modification to SEQ ID NO: 1 at H249 is an essential or non-essential amino acid. In embodiments, the amino acid modification at H249 is a hydrophilic or hydrophobic amino acid. In embodiments, the amino acid modification at H249 is amino acid modification is a hydrophilic amino acid. In embodiments, the amino acid modification at H249 is a polar and positively charged hydrophilic amino acid. In embodiments, the amino acid modification at H249 is selected from arginine (R) or lysine (K). In embodiments, the amino acid modification at H249 is a polar and neutral charged hydrophilic amino acid. In embodiments, the amino acid modification at H249 is selected from asparagine (N), glutamine (Q), serine (S), threonine (T), proline (P), and cysteine (C). In embodiments, the amino acid modification at H249 is a polar and negatively charged hydrophilic amino acid. In embodiments, the amino acid modification at H249 is selected from aspartate (D) or glutamate (E). In embodiments, the amino acid modification at H249 is an aromatic, polar and positively charged hydrophilic amino acid. In embodiments, the amino acid modification at H249 is a hydrophobic amino acid. In embodiments, the amino acid modification at H249 is a hydrophobic, aliphatic amino acid. In embodiments, the amino acid modification at H249 is selected from glycine (G), alanine (A), leucine (L), isoleucine (I), methionine (M), or valine (V). In embodiments, the amino acid modification at H249 is a hydrophobic, aromatic amino acid. In embodiments, the amino acid modification at H249 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 1 and an amino acid modification at position H669. In embodiments, the amino acid modifications are selected from substitutions and deletions. In embodiments, the amino acid modification to SEQ ID NO: 1 at H669 is an essential or non-essential amino acid. In embodiments, the amino acid modification at H669 is a hydrophilic or hydrophobic amino acid. In embodiments, the amino acid modification at H669 is amino acid modification is a hydrophilic amino acid. In embodiments, the amino acid modification at H669 is a polar and positively charged hydrophilic amino acid. In embodiments, the amino acid modification at H669 is selected from arginine (R) or lysine (K). In embodiments, the amino acid modification at H669 is a polar and neutral charged hydrophilic amino acid. In embodiments, the amino acid modification at H669 is selected from asparagine (N), glutamine (Q), serine (S), threonine (T), proline (P), and cysteine (C). In embodiments, the amino acid modification at H669 is a polar and negatively charged hydrophilic amino acid. In embodiments, the amino acid modification at H669 is selected from aspartate (D) or glutamate (E). In embodiments, the amino acid modification at H669 is an aromatic, polar and positively charged hydrophilic amino acid. In embodiments, the amino acid modification at H669 is a hydrophobic amino acid. In embodiments, the amino acid modification at H669 is a hydrophobic, aliphatic amino acid. In embodiments, the amino acid modification at H669 is selected from glycine (G), alanine (A), leucine (L), isoleucine (I), methionine (M), or valine (V). In embodiments, the amino acid modification at H669 is a hydrophobic, aromatic amino acid. In embodiments, the amino acid modification at H669 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 1 and an amino acid modification at position R664. In embodiments, the amino acid modifications are selected from substitutions and deletions. In embodiments, the amino acid modification to SEQ ID NO: 1 at R664 is an essential or non-essential amino acid. In embodiments, the amino acid modification at R664 is a hydrophilic or hydrophobic amino acid. In embodiments, the amino acid modification at R664 is amino acid modification is a hydrophilic amino acid. In embodiments, the amino acid modification at R664 is a polar and positively charged hydrophilic amino acid. In embodiments, the amino acid modification at R664 is lysine (K). In embodiments, the amino acid modification at R664 is a polar and neutral charged hydrophilic amino acid. In embodiments, the amino acid modification at R664 is selected from asparagine (N), glutamine (Q), serine (S), threonine (T), proline (P), and cysteine (C). In embodiments, the amino acid modification at R664 is a polar and negatively charged hydrophilic amino acid. In embodiments, the amino acid modification at R664 is selected from aspartate (D) or glutamate (E). In embodiments, the amino acid modification at R664 is an aromatic, polar and positively charged hydrophilic amino acid. In embodiments, the amino acid modification at R664 is histidine (H). In embodiments, the amino acid modification at R664 is a hydrophobic amino acid. In embodiments, the amino acid modification at R664 is a hydrophobic, aliphatic amino acid. In embodiments, the amino acid modification at R664 is selected from glycine (G), alanine (A), leucine (L), isoleucine (I), methionine (M), or valine (V). In embodiments, the amino acid modification at R664 is a hydrophobic, aromatic amino acid. In embodiments, the amino acid modification at R664 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 2 and an amino acid modification at position R244. In embodiments, the amino acid modifications are selected from substitutions and deletions. In embodiments, the amino acid modification to SEQ ID NO: 2 at R244 is an essential or non-essential amino acid. In embodiments, the amino acid modification at R244 is a hydrophilic or hydrophobic amino acid. In embodiments, the amino acid modification at R244 is amino acid modification is a hydrophilic amino acid. In embodiments, the amino acid modification at R244 is a polar and positively charged hydrophilic amino acid. In embodiments, the amino acid modification at R244 is lysine (K). In embodiments, the amino acid modification at R244 is a polar and neutral charged hydrophilic amino acid. In embodiments, the amino acid modification at R244 is selected from asparagine (N), glutamine (Q), serine (S), threonine (T), proline (P), and cysteine (C). In embodiments, the amino acid modification at R244 is a polar and negatively charged hydrophilic amino acid. In embodiments, the amino acid modification at R244 is selected from aspartate (D) or glutamate (E). In embodiments, the amino acid modification at R244 is an aromatic, polar and positively charged hydrophilic amino acid. In embodiments, the amino acid modification at R244 is histidine (H). In embodiments, the amino acid modification at R244 is a hydrophobic amino acid. In embodiments, the amino acid modification at R244 is a hydrophobic, aliphatic amino acid. In embodiments, the amino acid modification at R244 is selected from glycine (G), alanine (A), leucine (L), isoleucine (I), methionine (M), or valine (V). In embodiments, the amino acid modification at R244 is a hydrophobic, aromatic amino acid. In embodiments, the amino acid modification at R244 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 2 and an amino acid modification at position H249. In embodiments, the amino acid modifications are selected from substitutions and deletions. In embodiments, the amino acid modification to SEQ ID NO: 2 at H249 is an essential or non-essential amino acid. In embodiments, the amino acid modification at H249 is a hydrophilic or hydrophobic amino acid. In embodiments, the amino acid modification at H249 is amino acid modification is a hydrophilic amino acid. In embodiments, the amino acid modification at H249 is a polar and positively charged hydrophilic amino acid. In embodiments, the amino acid modification at H249 is selected from arginine (R) or lysine (K). In embodiments, the amino acid modification at H249 is a polar and neutral charged hydrophilic amino acid. In embodiments, the amino acid modification at H249 is selected from asparagine (N), glutamine (Q), serine (S), threonine (T), proline (P), and cysteine (C). In embodiments, the amino acid modification at H249 is a polar and negatively charged hydrophilic amino acid. In embodiments, the amino acid modification at H249 is selected from aspartate (D) or glutamate (E). In embodiments, the amino acid modification at H249 is an aromatic, polar and positively charged hydrophilic amino acid. In embodiments, the amino acid modification at H249 is a hydrophobic amino acid. In embodiments, the amino acid modification at H249 is a hydrophobic, aliphatic amino acid. In embodiments, the amino acid modification at H249 is selected from glycine (G), alanine (A), leucine (L), isoleucine (I), methionine (M), or valine (V). In embodiments, the amino acid modification at H249 is a hydrophobic, aromatic amino acid. In embodiments, the amino acid modification at H249 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 2 and an amino acid modification at position H687. In embodiments, the amino acid modifications are selected from substitutions and deletions. In embodiments, the amino acid modification to SEQ ID NO: 2 at H687 is an essential or non-essential amino acid. In embodiments, the amino acid modification at H687 is a hydrophilic or hydrophobic amino acid. In embodiments, the amino acid modification at H687 is amino acid modification is a hydrophilic amino acid. In embodiments, the amino acid modification at H687 is a polar and positively charged hydrophilic amino acid. In embodiments, the amino acid modification at H687 is selected from arginine (R) or lysine (K). In embodiments, the amino acid modification at H687 is a polar and neutral charged hydrophilic amino acid. In embodiments, the amino acid modification at H687 is selected from asparagine (N), glutamine (Q), serine (S), threonine (T), proline (P), and cysteine (C). In embodiments, the amino acid modification at H687 is a polar and negatively charged hydrophilic amino acid. In embodiments, the amino acid modification at H687 is selected from aspartate (D) or glutamate (E). In embodiments, the amino acid modification at H687 is an aromatic, polar and positively charged hydrophilic amino acid. In embodiments, the amino acid modification at H687 is a hydrophobic amino acid. In embodiments, the amino acid modification at H687 is a hydrophobic, aliphatic amino acid. In embodiments, the amino acid modification at H687 is selected from glycine (G), alanine (A), leucine (L), isoleucine (I), methionine (M), or valine (V). In embodiments, the amino acid modification at H687 is a hydrophobic, aromatic amino acid. In embodiments, the amino acid modification at H687 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 2 and an amino acid modification at position R682. In embodiments, the amino acid modifications are selected from substitutions and deletions. In embodiments, the amino acid modification to SEQ ID NO: 2 at R682 is an essential or non-essential amino acid. In embodiments, the amino acid modification at R682 is a hydrophilic or hydrophobic amino acid. In embodiments, the amino acid modification at R682 is amino acid modification is a hydrophilic amino acid. In embodiments, the amino acid modification at R682 is a polar and positively charged hydrophilic amino acid. In embodiments, the amino acid modification at R682 is lysine (K). In embodiments, the amino acid modification at R682 is a polar and neutral charged hydrophilic amino acid. In embodiments, the amino acid modification at R682 is selected from asparagine (N), glutamine (Q), serine (S), threonine (T), proline (P), and cysteine (C). In embodiments, the amino acid modification at R682 is a polar and negatively charged hydrophilic amino acid. In embodiments, the amino acid modification at R682 is selected from aspartate (D) or glutamate (E). In embodiments, the amino acid modification at R682 is an aromatic, polar and positively charged hydrophilic amino acid. In embodiments, the amino acid modification at R682 is histidine (H). In embodiments, the amino acid modification at R682 is a hydrophobic amino acid. In embodiments, the amino acid modification at R682 is a hydrophobic, aliphatic amino acid. In embodiments, the amino acid modification at R682 is selected from glycine (G), alanine (A), leucine (L), isoleucine (I), methionine (M), or valine (V). In embodiments, the amino acid modification at R682 is a hydrophobic, aromatic amino acid. In embodiments, the amino acid modification at R682 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 3 and an amino acid modification at position R244. In embodiments, the amino acid modifications are selected from substitutions and deletions. In embodiments, the amino acid modification to SEQ ID NO: 3 at R244 is an essential or non-essential amino acid. In embodiments, the amino acid modification at R244 is a hydrophilic or hydrophobic amino acid. In embodiments, the amino acid modification at R244 is amino acid modification is a hydrophilic amino acid. In embodiments, the amino acid modification at R244 is a polar and positively charged hydrophilic amino acid. In embodiments, the amino acid modification at R244 is lysine (K). In embodiments, the amino acid modification at R244 is a polar and neutral charged hydrophilic amino acid. In embodiments, the amino acid modification at R244 is selected from asparagine (N), glutamine (Q), serine (S), threonine (T), proline (P), and cysteine (C). In embodiments, the amino acid modification at R244 is a polar and negatively charged hydrophilic amino acid. In embodiments, the amino acid modification at R244 is selected from aspartate (D) or glutamate (E). In embodiments, the amino acid modification at R244 is an aromatic, polar and positively charged hydrophilic amino acid. In embodiments, the amino acid modification at R244 is histidine (H). In embodiments, the amino acid modification at R244 is a hydrophobic amino acid. In embodiments, the amino acid modification at R244 is a hydrophobic, aliphatic amino acid. In embodiments, the amino acid modification at R244 is selected from glycine (G), alanine (A), leucine (L), isoleucine (I), methionine (M), or valine (V). In embodiments, the amino acid modification at R244 is a hydrophobic, aromatic amino acid. In embodiments, the amino acid modification at R244 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 3 and an amino acid modification at position H249. In embodiments, the amino acid modifications are selected from substitutions and deletions. In embodiments, the amino acid modification to SEQ ID NO: 3 at H249 is an essential or non-essential amino acid. In embodiments, the amino acid modification at H249 is a hydrophilic or hydrophobic amino acid. In embodiments, the amino acid modification at H249 is amino acid modification is a hydrophilic amino acid. In embodiments, the amino acid modification at H249 is a polar and positively charged hydrophilic amino acid. In embodiments, the amino acid modification at H249 is selected from arginine (R) or lysine (K). In embodiments, the amino acid modification at H249 is a polar and neutral charged hydrophilic amino acid. In embodiments, the amino acid modification at H249 is selected from asparagine (N), glutamine (Q), serine (S), threonine (T), proline (P), and cysteine (C). In embodiments, the amino acid modification at H249 is a polar and negatively charged hydrophilic amino acid. In embodiments, the amino acid modification at H249 is selected from aspartate (D) or glutamate (E). In embodiments, the amino acid modification at H249 is an aromatic, polar and positively charged hydrophilic amino acid. In embodiments, the amino acid modification at H249 is a hydrophobic amino acid. In embodiments, the amino acid modification at H249 is a hydrophobic, aliphatic amino acid. In embodiments, the amino acid modification at H249 is selected from glycine (G), alanine (A), leucine (L), isoleucine (I), methionine (M), or valine (V). In embodiments, the amino acid modification at H249 is a hydrophobic, aromatic amino acid. In embodiments, the amino acid modification at H249 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 3 and an amino acid modification at position R704. In embodiments, the amino acid modifications are selected from substitutions and deletions. In embodiments, the amino acid modification to SEQ ID NO: 3 at R704 is an essential or non-essential amino acid. In embodiments, the amino acid modification at R704 is a hydrophilic or hydrophobic amino acid. In embodiments, the amino acid modification at R704 is amino acid modification is a hydrophilic amino acid. In embodiments, the amino acid modification at R704 is a polar and positively charged hydrophilic amino acid. In embodiments, the amino acid modification at R704 is lysine (K). In embodiments, the amino acid modification at R704 is a polar and neutral charged hydrophilic amino acid. In embodiments, the amino acid modification at R704 is selected from asparagine (N), glutamine (Q), serine (S), threonine (T), proline (P), and cysteine (C). In embodiments, the amino acid modification at R704 is a polar and negatively charged hydrophilic amino acid. In embodiments, the amino acid modification at R704 is selected from aspartate (D) or glutamate (E). In embodiments, the amino acid modification at R704 is an aromatic, polar and positively charged hydrophilic amino acid. In embodiments, the amino acid modification at R704 is histidine (H). In embodiments, the amino acid modification at R704 is a hydrophobic amino acid. In embodiments, the amino acid modification at R704 is a hydrophobic, aliphatic amino acid. In embodiments, the amino acid modification at R704 is selected from glycine (G), alanine (A), leucine (L), isoleucine (I), methionine (M), or valine (V). In embodiments, the amino acid modification at R704 is a hydrophobic, aromatic amino acid. In embodiments, the amino acid modification at R704 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 3 and an amino acid modification at position H709. In embodiments, the amino acid modifications are selected from substitutions and deletions. In embodiments, the amino acid modification to SEQ ID NO: 3 at H709 is an essential or non-essential amino acid. In embodiments, the amino acid modification at H709 is a hydrophilic or hydrophobic amino acid. In embodiments, the amino acid modification at H709 is amino acid modification is a hydrophilic amino acid. In embodiments, the amino acid modification at H709 is a polar and positively charged hydrophilic amino acid. In embodiments, the amino acid modification at H709 is selected from arginine (R) or lysine (K). In embodiments, the amino acid modification at H709 is a polar and neutral charged hydrophilic amino acid. In embodiments, the amino acid modification at H709 is selected from asparagine (N), glutamine (Q), serine (S), threonine (T), proline (P), and cysteine (C). In embodiments, the amino acid modification at H709 is a polar and negatively charged hydrophilic amino acid. In embodiments, the amino acid modification at H709 is selected from aspartate (D) or glutamate (E). In embodiments, the amino acid modification at H709 is an aromatic, polar and positively charged hydrophilic amino acid. In embodiments, the amino acid modification at H709 is a hydrophobic amino acid. In embodiments, the amino acid modification at H709 is a hydrophobic, aliphatic amino acid. In embodiments, the amino acid modification at H709 is selected from glycine (G), alanine (A), leucine (L), isoleucine (I), methionine (M), or valine (V). In embodiments, the amino acid modification at H709 is a hydrophobic, aromatic amino acid. In embodiments, the amino acid modification at H709 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 4 and an amino acid modification at position R244. In embodiments, the amino acid modifications are selected from substitutions and deletions. In embodiments, the amino acid modification to SEQ ID NO: 4 at R244 is an essential or non-essential amino acid. In embodiments, the amino acid modification at R244 is a hydrophilic or hydrophobic amino acid. In embodiments, the amino acid modification at R244 is amino acid modification is a hydrophilic amino acid. In embodiments, the amino acid modification at R244 is a polar and positively charged hydrophilic amino acid. In embodiments, the amino acid modification at R244 is lysine (K). In embodiments, the amino acid modification at R244 is a polar and neutral charged hydrophilic amino acid. In embodiments, the amino acid modification at R244 is selected from asparagine (N), glutamine (Q), serine (S), threonine (T), proline (P), and cysteine (C). In embodiments, the amino acid modification at R244 is a polar and negatively charged hydrophilic amino acid. In embodiments, the amino acid modification at R244 is selected from aspartate (D) or glutamate (E). In embodiments, the amino acid modification at R244 is an aromatic, polar and positively charged hydrophilic amino acid. In embodiments, the amino acid modification at R244 is histidine (H). In embodiments, the amino acid modification at R244 is a hydrophobic amino acid. In embodiments, the amino acid modification at R244 is a hydrophobic, aliphatic amino acid. In embodiments, the amino acid modification at R244 is selected from glycine (G), alanine (A), leucine (L), isoleucine (I), methionine (M), or valine (V). In embodiments, the amino acid modification at R244 is a hydrophobic, aromatic amino acid. In embodiments, the amino acid modification at R244 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 4 and an amino acid modification at position H249. In embodiments, the amino acid modifications are selected from substitutions and deletions. In embodiments, the amino acid modification to SEQ ID NO: 4 at H249 is an essential or non-essential amino acid. In embodiments, the amino acid modification at H249 is a hydrophilic or hydrophobic amino acid. In embodiments, the amino acid modification at H249 is amino acid modification is a hydrophilic amino acid. In embodiments, the amino acid modification at H249 is a polar and positively charged hydrophilic amino acid. In embodiments, the amino acid modification at H249 is selected from arginine (R) or lysine (K). In embodiments, the amino acid modification at H249 is a polar and neutral charged hydrophilic amino acid. In embodiments, the amino acid modification at H249 is selected from asparagine (N), glutamine (Q), serine (S), threonine (T), proline (P), and cysteine (C). In embodiments, the amino acid modification at H249 is a polar and negatively charged hydrophilic amino acid. In embodiments, the amino acid modification at H249 is selected from aspartate (D) or glutamate (E). In embodiments, the amino acid modification at H249 is an aromatic, polar and positively charged hydrophilic amino acid. In embodiments, the amino acid modification at H249 is a hydrophobic amino acid. In embodiments, the amino acid modification at H249 is a hydrophobic, aliphatic amino acid. In embodiments, the amino acid modification at H249 is selected from glycine (G), alanine (A), leucine (L), isoleucine (I), methionine (M), or valine (V). In embodiments, the amino acid modification at H249 is a hydrophobic, aromatic amino acid. In embodiments, the amino acid modification at H249 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 4 and an amino acid modification at position H687. In embodiments, the amino acid modifications are selected from substitutions and deletions. In embodiments, the amino acid modification to SEQ ID NO: 4 at H687 is an essential or non-essential amino acid. In embodiments, the amino acid modification at H687 is a hydrophilic or hydrophobic amino acid. In embodiments, the amino acid modification at H687 is amino acid modification is a hydrophilic amino acid. In embodiments, the amino acid modification at H687 is a polar and positively charged hydrophilic amino acid. In embodiments, the amino acid modification at H687 is selected from arginine (R) or lysine (K). In embodiments, the amino acid modification at H687 is a polar and neutral charged hydrophilic amino acid. In embodiments, the amino acid modification at H687 is selected from asparagine (N), glutamine (Q), serine (S), threonine (T), proline (P), and cysteine (C). In embodiments, the amino acid modification at H687 is a polar and negatively charged hydrophilic amino acid. In embodiments, the amino acid modification at H687 is selected from aspartate (D) or glutamate (E). In embodiments, the amino acid modification at H687 is an aromatic, polar and positively charged hydrophilic amino acid. In embodiments, the amino acid modification at H687 is a hydrophobic amino acid. In embodiments, the amino acid modification at H687 is a hydrophobic, aliphatic amino acid. In embodiments, the amino acid modification at H687 is selected from glycine (G), alanine (A), leucine (L), isoleucine (I), methionine (M), or valine (V). In embodiments, the amino acid modification at H687 is a hydrophobic, aromatic amino acid. In embodiments, the amino acid modification at H687 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 4 and an amino acid modification at position R682. In embodiments, the amino acid modifications are selected from substitutions and deletions. In embodiments, the amino acid modification to SEQ ID NO: 4 at R682 is an essential or non-essential amino acid. In embodiments, the amino acid modification at R682 is a hydrophilic or hydrophobic amino acid. In embodiments, the amino acid modification at R682 is amino acid modification is a hydrophilic amino acid. In embodiments, the amino acid modification at R682 is a polar and positively charged hydrophilic amino acid. In embodiments, the amino acid modification at R682 is lysine (K). In embodiments, the amino acid modification at R682 is a polar and neutral charged hydrophilic amino acid. In embodiments, the amino acid modification at R682 is selected from asparagine (N), glutamine (Q), serine (S), threonine (T), proline (P), and cysteine (C). In embodiments, the amino acid modification at R682 is a polar and negatively charged hydrophilic amino acid. In embodiments, the amino acid modification at R682 is selected from aspartate (D) or glutamate (E). In embodiments, the amino acid modification at R682 is an aromatic, polar and positively charged hydrophilic amino acid. In embodiments, the amino acid modification at R682 is histidine (H). In embodiments, the amino acid modification at R682 is a hydrophobic amino acid. In embodiments, the amino acid modification at R682 is a hydrophobic, aliphatic amino acid. In embodiments, the amino acid modification at R682 is selected from glycine (G), alanine (A), leucine (L), isoleucine (I), methionine (M), or valine (V). In embodiments, the amino acid modification at R682 is a hydrophobic, aromatic amino acid. In embodiments, the amino acid modification at R682 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 80 and an amino acid modification at position R234. In embodiments, the amino acid modifications are selected from substitutions and deletions. In embodiments, the amino acid modification to SEQ ID NO: 80 at R234 is an essential or non-essential amino acid. In embodiments, the amino acid modification at R234 is a hydrophilic or hydrophobic amino acid. In embodiments, the amino acid modification at R234 is amino acid modification is a hydrophilic amino acid. In embodiments, the amino acid modification at R234 is a polar and positively charged hydrophilic amino acid. In embodiments, the amino acid modification at R234 is lysine (K). In embodiments, the amino acid modification at R234 is a polar and neutral charged hydrophilic amino acid. In embodiments, the amino acid modification at R234 is selected from asparagine (N), glutamine (Q), serine (S), threonine (T), proline (P), and cysteine (C). In embodiments, the amino acid modification at R234 is a polar and negatively charged hydrophilic amino acid. In embodiments, the amino acid modification at R234 is selected from aspartate (D) or glutamate (E). In embodiments, the amino acid modification at R234 is an aromatic, polar and positively charged hydrophilic amino acid. In embodiments, the amino acid modification at R234 is histidine (H). In embodiments, the amino acid modification at R234 is a hydrophobic amino acid. In embodiments, the amino acid modification at R234 is a hydrophobic, aliphatic amino acid. In embodiments, the amino acid modification at R234 is selected from glycine (G), alanine (A), leucine (L), isoleucine (I), methionine (M), or valine (V). In embodiments, the amino acid modification at R234 is a hydrophobic, aromatic amino acid. In embodiments, the amino acid modification at R234 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 80 and an amino acid modification at position H239. In embodiments, the amino acid modifications are selected from substitutions and deletions. In embodiments, the amino acid modification to SEQ ID NO: 80 at H239 is an essential or non-essential amino acid. In embodiments, the amino acid modification at H239 is a hydrophilic or hydrophobic amino acid. In embodiments, the amino acid modification at H239 is amino acid modification is a hydrophilic amino acid. In embodiments, the amino acid modification at H239 is a polar and positively charged hydrophilic amino acid. In embodiments, the amino acid modification at H239 is selected from arginine (R) or lysine (K). In embodiments, the amino acid modification at H239 is a polar and neutral charged hydrophilic amino acid. In embodiments, the amino acid modification at H239 is selected from asparagine (N), glutamine (Q), serine (S), threonine (T), proline (P), and cysteine (C). In embodiments, the amino acid modification at H239 is a polar and negatively charged hydrophilic amino acid. In embodiments, the amino acid modification at H239 is selected from aspartate (D) or glutamate (E). In embodiments, the amino acid modification at H239 is an aromatic, polar and positively charged hydrophilic amino acid. In embodiments, the amino acid modification at H239 is a hydrophobic amino acid. In embodiments, the amino acid modification at H239 is a hydrophobic, aliphatic amino acid. In embodiments, the amino acid modification at H239 is selected from glycine (G), alanine (A), leucine (L), isoleucine (I), methionine (M), or valine (V). In embodiments, the amino acid modification at H239 is a hydrophobic, aromatic amino acid. In embodiments, the amino acid modification at H239 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 80 and an amino acid modification at position R659. In embodiments, the amino acid modifications are selected from substitutions and deletions. In embodiments, the amino acid modification to SEQ ID NO: 80 at R659 is an essential or non-essential amino acid. In embodiments, the amino acid modification at R659 is a hydrophilic or hydrophobic amino acid. In embodiments, the amino acid modification at R659 is amino acid modification is a hydrophilic amino acid. In embodiments, the amino acid modification at R659 is a polar and positively charged hydrophilic amino acid. In embodiments, the amino acid modification at R659 is lysine (K). In embodiments, the amino acid modification at R659 is a polar and neutral charged hydrophilic amino acid. In embodiments, the amino acid modification at R659 is selected from asparagine (N), glutamine (Q), serine (S), threonine (T), proline (P), and cysteine (C). In embodiments, the amino acid modification at R659 is a polar and negatively charged hydrophilic amino acid. In embodiments, the amino acid modification at R659 is selected from aspartate (D) or glutamate (E). In embodiments, the amino acid modification at R659 is an aromatic, polar and positively charged hydrophilic amino acid. In embodiments, the amino acid modification at R659 is histidine (H). In embodiments, the amino acid modification at R659 is a hydrophobic amino acid. In embodiments, the amino acid modification at R659 is a hydrophobic, aliphatic amino acid. In embodiments, the amino acid modification at R659 is selected from glycine (G), alanine (A), leucine (L), isoleucine (I), methionine (M), or valine (V). In embodiments, the amino acid modification at R659 is a hydrophobic, aromatic amino acid. In embodiments, the amino acid modification at R659 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 80 and an amino acid modification at position H664. In embodiments, the amino acid modifications are selected from substitutions and deletions. In embodiments, the amino acid modification to SEQ ID NO: 80 at H664 is an essential or non-essential amino acid. In embodiments, the amino acid modification at H664 is a hydrophilic or hydrophobic amino acid. In embodiments, the amino acid modification at H664 is amino acid modification is a hydrophilic amino acid. In embodiments, the amino acid modification at H664 is a polar and positively charged hydrophilic amino acid. In embodiments, the amino acid modification at H664 is selected from arginine (R) or lysine (K). In embodiments, the amino acid modification at H664 is a polar and neutral charged hydrophilic amino acid. In embodiments, the amino acid modification at H664 is selected from asparagine (N), glutamine (Q), serine (S), threonine (T), proline (P), and cysteine (C). In embodiments, the amino acid modification at H664 is a polar and negatively charged hydrophilic amino acid. In embodiments, the amino acid modification at H664 is selected from aspartate (D) or glutamate (E). In embodiments, the amino acid modification at H664 is an aromatic, polar and positively charged hydrophilic amino acid. In embodiments, the amino acid modification at H664 is a hydrophobic amino acid. In embodiments, the amino acid modification at H664 is a hydrophobic, aliphatic amino acid. In embodiments, the amino acid modification at H664 is selected from glycine (G), alanine (A), leucine (L), isoleucine (I), methionine (M), or valine (V). In embodiments, the amino acid modification at H664 is a hydrophobic, aromatic amino acid. In embodiments, the amino acid modification at H664 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 81 and an amino acid modification at position R234. In embodiments, the amino acid modifications are selected from substitutions and deletions. In embodiments, the amino acid modification to SEQ ID NO: 81 at R234 is an essential or non-essential amino acid. In embodiments, the amino acid modification at R234 is a hydrophilic or hydrophobic amino acid. In embodiments, the amino acid modification at R234 is amino acid modification is a hydrophilic amino acid. In embodiments, the amino acid modification at R234 is a polar and positively charged hydrophilic amino acid. In embodiments, the amino acid modification at R234 is lysine (K). In embodiments, the amino acid modification at R234 is a polar and neutral charged hydrophilic amino acid. In embodiments, the amino acid modification at R234 is selected from asparagine (N), glutamine (Q), serine (S), threonine (T), proline (P), and cysteine (C). In embodiments, the amino acid modification at R234 is a polar and negatively charged hydrophilic amino acid. In embodiments, the amino acid modification at R234 is selected from aspartate (D) or glutamate (E). In embodiments, the amino acid modification at R234 is an aromatic, polar and positively charged hydrophilic amino acid. In embodiments, the amino acid modification at R234 is histidine (H). In embodiments, the amino acid modification at R234 is a hydrophobic amino acid. In embodiments, the amino acid modification at R234 is a hydrophobic, aliphatic amino acid. In embodiments, the amino acid modification at R234 is selected from glycine (G), alanine (A), leucine (L), isoleucine (I), methionine (M), or valine (V). In embodiments, the amino acid modification at R234 is a hydrophobic, aromatic amino acid. In embodiments, the amino acid modification at R234 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 81 and an amino acid modification at position H239. In embodiments, the amino acid modifications are selected from substitutions and deletions. In embodiments, the amino acid modification to SEQ ID NO: 81 at H239 is an essential or non-essential amino acid. In embodiments, the amino acid modification at H239 is a hydrophilic or hydrophobic amino acid. In embodiments, the amino acid modification at H239 is amino acid modification is a hydrophilic amino acid. In embodiments, the amino acid modification at H239 is a polar and positively charged hydrophilic amino acid. In embodiments, the amino acid modification at H239 is selected from arginine (R) or lysine (K). In embodiments, the amino acid modification at H239 is a polar and neutral charged hydrophilic amino acid. In embodiments, the amino acid modification at H239 is selected from asparagine (N), glutamine (Q), serine (S), threonine (T), proline (P), and cysteine (C). In embodiments, the amino acid modification at H239 is a polar and negatively charged hydrophilic amino acid. In embodiments, the amino acid modification at H239 is selected from aspartate (D) or glutamate (E). In embodiments, the amino acid modification at H239 is an aromatic, polar and positively charged hydrophilic amino acid. In embodiments, the amino acid modification at H239 is a hydrophobic amino acid. In embodiments, the amino acid modification at H239 is a hydrophobic, aliphatic amino acid. In embodiments, the amino acid modification at H239 is selected from glycine (G), alanine (A), leucine (L), isoleucine (I), methionine (M), or valine (V). In embodiments, the amino acid modification at H239 is a hydrophobic, aromatic amino acid. In embodiments, the amino acid modification at H239 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 81 and an amino acid modification at position R659. In embodiments, the amino acid modifications are selected from substitutions and deletions. In embodiments, the amino acid modification to SEQ ID NO: 81 at R659 is an essential or non-essential amino acid. In embodiments, the amino acid modification at R659 is a hydrophilic or hydrophobic amino acid. In embodiments, the amino acid modification at R659 is amino acid modification is a hydrophilic amino acid. In embodiments, the amino acid modification at R659 is a polar and positively charged hydrophilic amino acid. In embodiments, the amino acid modification at R659 is lysine (K). In embodiments, the amino acid modification at R659 is a polar and neutral charged hydrophilic amino acid. In embodiments, the amino acid modification at R659 is selected from asparagine (N), glutamine (Q), serine (S), threonine (T), proline (P), and cysteine (C). In embodiments, the amino acid modification at R659 is a polar and negatively charged hydrophilic amino acid. In embodiments, the amino acid modification at R659 is selected from aspartate (D) or glutamate (E). In embodiments, the amino acid modification at R659 is an aromatic, polar and positively charged hydrophilic amino acid. In embodiments, the amino acid modification at R659 is histidine (H). In embodiments, the amino acid modification at R659 is a hydrophobic amino acid. In embodiments, the amino acid modification at R659 is a hydrophobic, aliphatic amino acid. In embodiments, the amino acid modification at R659 is selected from glycine (G), alanine (A), leucine (L), isoleucine (I), methionine (M), or valine (V). In embodiments, the amino acid modification at R659 is a hydrophobic, aromatic amino acid. In embodiments, the amino acid modification at R659 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 81 and an amino acid modification at position H664. In embodiments, the amino acid modifications are selected from substitutions and deletions. In embodiments, the amino acid modification to SEQ ID NO: 81 at H664 is an essential or non-essential amino acid. In embodiments, the amino acid modification at H664 is a hydrophilic or hydrophobic amino acid. In embodiments, the amino acid modification at H664 is amino acid modification is a hydrophilic amino acid. In embodiments, the amino acid modification at H664 is a polar and positively charged hydrophilic amino acid. In embodiments, the amino acid modification at H664 is selected from arginine (R) or lysine (K). In embodiments, the amino acid modification at H664 is a polar and neutral charged hydrophilic amino acid. In embodiments, the amino acid modification at H664 is selected from asparagine (N), glutamine (Q), serine (S), threonine (T), proline (P), and cysteine (C). In embodiments, the amino acid modification at H664 is a polar and negatively charged hydrophilic amino acid. In embodiments, the amino acid modification at H664 is selected from aspartate (D) or glutamate (E). In embodiments, the amino acid modification at H664 is an aromatic, polar and positively charged hydrophilic amino acid. In embodiments, the amino acid modification at H664 is a hydrophobic amino acid. In embodiments, the amino acid modification at H664 is a hydrophobic, aliphatic amino acid. In embodiments, the amino acid modification at H664 is selected from glycine (G), alanine (A), leucine (L), isoleucine (I), methionine (M), or valine (V). In embodiments, the amino acid modification at H664 is a hydrophobic, aromatic amino acid. In embodiments, the amino acid modification at H664 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 82 and an amino acid modification at position R234. In embodiments, the amino acid modifications are selected from substitutions and deletions. In embodiments, the amino acid modification to SEQ ID NO: 82 at R234 is an essential or non-essential amino acid. In embodiments, the amino acid modification at R234 is a hydrophilic or hydrophobic amino acid. In embodiments, the amino acid modification at R234 is amino acid modification is a hydrophilic amino acid. In embodiments, the amino acid modification at R234 is a polar and positively charged hydrophilic amino acid. In embodiments, the amino acid modification at R234 is lysine (K). In embodiments, the amino acid modification at R234 is a polar and neutral charged hydrophilic amino acid. In embodiments, the amino acid modification at R234 is selected from asparagine (N), glutamine (Q), serine (S), threonine (T), proline (P), and cysteine (C). In embodiments, the amino acid modification at R234 is a polar and negatively charged hydrophilic amino acid. In embodiments, the amino acid modification at R234 is selected from aspartate (D) or glutamate (E). In embodiments, the amino acid modification at R234 is an aromatic, polar and positively charged hydrophilic amino acid. In embodiments, the amino acid modification at R234 is histidine (H). In embodiments, the amino acid modification at R234 is a hydrophobic amino acid. In embodiments, the amino acid modification at R234 is a hydrophobic, aliphatic amino acid. In embodiments, the amino acid modification at R234 is selected from glycine (G), alanine (A), leucine (L), isoleucine (I), methionine (M), or valine (V). In embodiments, the amino acid modification at R234 is a hydrophobic, aromatic amino acid. In embodiments, the amino acid modification at R234 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 82 and an amino acid modification at position H239. In embodiments, the amino acid modifications are selected from substitutions and deletions. In embodiments, the amino acid modification to SEQ ID NO: 82 at H239 is an essential or non-essential amino acid. In embodiments, the amino acid modification at H239 is a hydrophilic or hydrophobic amino acid. In embodiments, the amino acid modification at H239 is amino acid modification is a hydrophilic amino acid. In embodiments, the amino acid modification at H239 is a polar and positively charged hydrophilic amino acid. In embodiments, the amino acid modification at H239 is selected from arginine (R) or lysine (K). In embodiments, the amino acid modification at H239 is a polar and neutral charged hydrophilic amino acid. In embodiments, the amino acid modification at H239 is selected from asparagine (N), glutamine (Q), serine (S), threonine (T), proline (P), and cysteine (C). In embodiments, the amino acid modification at H239 is a polar and negatively charged hydrophilic amino acid. In embodiments, the amino acid modification at H239 is selected from aspartate (D) or glutamate (E). In embodiments, the amino acid modification at H239 is an aromatic, polar and positively charged hydrophilic amino acid. In embodiments, the amino acid modification at H239 is a hydrophobic amino acid. In embodiments, the amino acid modification at H239 is a hydrophobic, aliphatic amino acid. In embodiments, the amino acid modification at H239 is selected from glycine (G), alanine (A), leucine (L), isoleucine (I), methionine (M), or valine (V). In embodiments, the amino acid modification at H239 is a hydrophobic, aromatic amino acid. In embodiments, the amino acid modification at H239 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 82 and an amino acid modification at position R659. In embodiments, the amino acid modifications are selected from substitutions and deletions. In embodiments, the amino acid modification to SEQ ID NO: 82 at R659 is an essential or non-essential amino acid. In embodiments, the amino acid modification at R659 is a hydrophilic or hydrophobic amino acid. In embodiments, the amino acid modification at R659 is amino acid modification is a hydrophilic amino acid. In embodiments, the amino acid modification at R659 is a polar and positively charged hydrophilic amino acid. In embodiments, the amino acid modification at R659 is lysine (K). In embodiments, the amino acid modification at R659 is a polar and neutral charged hydrophilic amino acid. In embodiments, the amino acid modification at R659 is selected from asparagine (N), glutamine (Q), serine (S), threonine (T), proline (P), and cysteine (C). In embodiments, the amino acid modification at R659 is a polar and negatively charged hydrophilic amino acid. In embodiments, the amino acid modification at R659 is selected from aspartate (D) or glutamate (E). In embodiments, the amino acid modification at R659 is an aromatic, polar and positively charged hydrophilic amino acid. In embodiments, the amino acid modification at R659 is histidine (H). In embodiments, the amino acid modification at R659 is a hydrophobic amino acid. In embodiments, the amino acid modification at R659 is a hydrophobic, aliphatic amino acid. In embodiments, the amino acid modification at R659 is selected from glycine (G), alanine (A), leucine (L), isoleucine (I), methionine (M), or valine (V). In embodiments, the amino acid modification at R659 is a hydrophobic, aromatic amino acid. In embodiments, the amino acid modification at R659 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 82 and an amino acid modification at position H664. In embodiments, the amino acid modifications are selected from substitutions and deletions. In embodiments, the amino acid modification to SEQ ID NO: 82 at H664 is an essential or non-essential amino acid. In embodiments, the amino acid modification at H664 is a hydrophilic or hydrophobic amino acid. In embodiments, the amino acid modification at H664 is amino acid modification is a hydrophilic amino acid. In embodiments, the amino acid modification at H664 is a polar and positively charged hydrophilic amino acid. In embodiments, the amino acid modification at H664 is selected from arginine (R) or lysine (K). In embodiments, the amino acid modification at H664 is a polar and neutral charged hydrophilic amino acid. In embodiments, the amino acid modification at H664 is selected from asparagine (N), glutamine (Q), serine (S), threonine (T), proline (P), and cysteine (C). In embodiments, the amino acid modification at H664 is a polar and negatively charged hydrophilic amino acid. In embodiments, the amino acid modification at H664 is selected from aspartate (D) or glutamate (E). In embodiments, the amino acid modification at H664 is an aromatic, polar and positively charged hydrophilic amino acid. In embodiments, the amino acid modification at H664 is a hydrophobic amino acid. In embodiments, the amino acid modification at H664 is a hydrophobic, aliphatic amino acid. In embodiments, the amino acid modification at H664 is selected from glycine (G), alanine (A), leucine (L), isoleucine (I), methionine (M), or valine (V). In embodiments, the amino acid modification at H664 is a hydrophobic, aromatic amino acid. In embodiments, the amino acid modification at H664 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 83 and an amino acid modification at position R239. In embodiments, the amino acid modifications are selected from substitutions and deletions. In embodiments, the amino acid modification to SEQ ID NO: 83 at R239 is an essential or non-essential amino acid. In embodiments, the amino acid modification at R239 is a hydrophilic or hydrophobic amino acid. In embodiments, the amino acid modification at R239 is amino acid modification is a hydrophilic amino acid. In embodiments, the amino acid modification at R239 is a polar and positively charged hydrophilic amino acid. In embodiments, the amino acid modification at R239 is lysine (K). In embodiments, the amino acid modification at R239 is a polar and neutral charged hydrophilic amino acid. In embodiments, the amino acid modification at R239 is selected from asparagine (N), glutamine (Q), serine (S), threonine (T), proline (P), and cysteine (C). In embodiments, the amino acid modification at R239 is a polar and negatively charged hydrophilic amino acid. In embodiments, the amino acid modification at R239 is selected from aspartate (D) or glutamate (E). In embodiments, the amino acid modification at R239 is an aromatic, polar and positively charged hydrophilic amino acid. In embodiments, the amino acid modification at R239 is histidine (H). In embodiments, the amino acid modification at R239 is a hydrophobic amino acid. In embodiments, the amino acid modification at R239 is a hydrophobic, aliphatic amino acid. In embodiments, the amino acid modification at R239 is selected from glycine (G), alanine (A), leucine (L), isoleucine (I), methionine (M), or valine (V). In embodiments, the amino acid modification at R239 is a hydrophobic, aromatic amino acid. In embodiments, the amino acid modification at R239 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 83 and an amino acid modification at position H244. In embodiments, the amino acid modifications are selected from substitutions and deletions. In embodiments, the amino acid modification to SEQ ID NO: 83 at H244 is an essential or non-essential amino acid. In embodiments, the amino acid modification at H244 is a hydrophilic or hydrophobic amino acid. In embodiments, the amino acid modification at H244 is amino acid modification is a hydrophilic amino acid. In embodiments, the amino acid modification at H244 is a polar and positively charged hydrophilic amino acid. In embodiments, the amino acid modification at H244 is selected from arginine (R) or lysine (K). In embodiments, the amino acid modification at H244 is a polar and neutral charged hydrophilic amino acid. In embodiments, the amino acid modification at H244 is selected from asparagine (N), glutamine (Q), serine (S), threonine (T), proline (P), and cysteine (C). In embodiments, the amino acid modification at H244 is a polar and negatively charged hydrophilic amino acid. In embodiments, the amino acid modification at H244 is selected from aspartate (D) or glutamate (E). In embodiments, the amino acid modification at H244 is an aromatic, polar and positively charged hydrophilic amino acid. In embodiments, the amino acid modification at H244 is a hydrophobic amino acid. In embodiments, the amino acid modification at H244 is a hydrophobic, aliphatic amino acid. In embodiments, the amino acid modification at H244 is selected from glycine (G), alanine (A), leucine (L), isoleucine (I), methionine (M), or valine (V). In embodiments, the amino acid modification at H244 is a hydrophobic, aromatic amino acid. In embodiments, the amino acid modification at H244 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 83 and an amino acid modification at position R677. In embodiments, the amino acid modifications are selected from substitutions and deletions. In embodiments, the amino acid modification to SEQ ID NO: 83 at R677 is an essential or non-essential amino acid. In embodiments, the amino acid modification at R677 is a hydrophilic or hydrophobic amino acid. In embodiments, the amino acid modification at R677 is amino acid modification is a hydrophilic amino acid. In embodiments, the amino acid modification at R677 is a polar and positively charged hydrophilic amino acid. In embodiments, the amino acid modification at R677 is lysine (K). In embodiments, the amino acid modification at R677 is a polar and neutral charged hydrophilic amino acid. In embodiments, the amino acid modification at R677 is selected from asparagine (N), glutamine (Q), serine (S), threonine (T), proline (P), and cysteine (C). In embodiments, the amino acid modification at R677 is a polar and negatively charged hydrophilic amino acid. In embodiments, the amino acid modification at R677 is selected from aspartate (D) or glutamate (E). In embodiments, the amino acid modification at R677 is an aromatic, polar and positively charged hydrophilic amino acid. In embodiments, the amino acid modification at R677 is histidine (H). In embodiments, the amino acid modification at R677 is a hydrophobic amino acid. In embodiments, the amino acid modification at R677 is a hydrophobic, aliphatic amino acid. In embodiments, the amino acid modification at R677 is selected from glycine (G), alanine (A), leucine (L), isoleucine (I), methionine (M), or valine (V). In embodiments, the amino acid modification at R677 is a hydrophobic, aromatic amino acid. In embodiments, the amino acid modification at R677 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 83 and an amino acid modification at position H682. In embodiments, the amino acid modifications are selected from substitutions and deletions. In embodiments, the amino acid modification to SEQ ID NO: 83 at H682 is an essential or non-essential amino acid. In embodiments, the amino acid modification at H682 is a hydrophilic or hydrophobic amino acid. In embodiments, the amino acid modification at H682 is amino acid modification is a hydrophilic amino acid. In embodiments, the amino acid modification at H682 is a polar and positively charged hydrophilic amino acid. In embodiments, the amino acid modification at H682 is selected from arginine (R) or lysine (K). In embodiments, the amino acid modification at H682 is a polar and neutral charged hydrophilic amino acid. In embodiments, the amino acid modification at H682 is selected from asparagine (N), glutamine (Q), serine (S), threonine (T), proline (P), and cysteine (C). In embodiments, the amino acid modification at H682 is a polar and negatively charged hydrophilic amino acid. In embodiments, the amino acid modification at H682 is selected from aspartate (D) or glutamate (E). In embodiments, the amino acid modification at H682 is an aromatic, polar and positively charged hydrophilic amino acid. In embodiments, the amino acid modification at H682 is a hydrophobic amino acid. In embodiments, the amino acid modification at H682 is a hydrophobic, aliphatic amino acid. In embodiments, the amino acid modification at H682 is selected from glycine (G), alanine (A), leucine (L), isoleucine (I), methionine (M), or valine (V). In embodiments, the amino acid modification at H682 is a hydrophobic, aromatic amino acid. In embodiments, the amino acid modification at H682 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 84 and an amino acid modification at position R241. In embodiments, the amino acid modifications are selected from substitutions and deletions. In embodiments, the amino acid modification to SEQ ID NO: 84 at R241 is an essential or non-essential amino acid. In embodiments, the amino acid modification at R241 is a hydrophilic or hydrophobic amino acid. In embodiments, the amino acid modification at R241 is amino acid modification is a hydrophilic amino acid. In embodiments, the amino acid modification at R241 is a polar and positively charged hydrophilic amino acid. In embodiments, the amino acid modification at R241 is lysine (K). In embodiments, the amino acid modification at R241 is a polar and neutral charged hydrophilic amino acid. In embodiments, the amino acid modification at R241 is selected from asparagine (N), glutamine (Q), serine (S), threonine (T), proline (P), and cysteine (C). In embodiments, the amino acid modification at R241 is a polar and negatively charged hydrophilic amino acid. In embodiments, the amino acid modification at R241 is selected from aspartate (D) or glutamate (E). In embodiments, the amino acid modification at R241 is an aromatic, polar and positively charged hydrophilic amino acid. In embodiments, the amino acid modification at R241 is histidine (H). In embodiments, the amino acid modification at R241 is a hydrophobic amino acid. In embodiments, the amino acid modification at R241 is a hydrophobic, aliphatic amino acid. In embodiments, the amino acid modification at R241 is selected from glycine (G), alanine (A), leucine (L), isoleucine (I), methionine (M), or valine (V). In embodiments, the amino acid modification at R241 is a hydrophobic, aromatic amino acid. In embodiments, the amino acid modification at R241 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 84 and an amino acid modification at position H246. In embodiments, the amino acid modifications are selected from substitutions and deletions. In embodiments, the amino acid modification to SEQ ID NO: 84 at H246 is an essential or non-essential amino acid. In embodiments, the amino acid modification at H246 is a hydrophilic or hydrophobic amino acid. In embodiments, the amino acid modification at H246 is amino acid modification is a hydrophilic amino acid. In embodiments, the amino acid modification at H246 is a polar and positively charged hydrophilic amino acid. In embodiments, the amino acid modification at H246 is selected from arginine (R) or lysine (K). In embodiments, the amino acid modification at H246 is a polar and neutral charged hydrophilic amino acid. In embodiments, the amino acid modification at H246 is selected from asparagine (N), glutamine (Q), serine (S), threonine (T), proline (P), and cysteine (C). In embodiments, the amino acid modification at H246 is a polar and negatively charged hydrophilic amino acid. In embodiments, the amino acid modification at H246 is selected from aspartate (D) or glutamate (E). In embodiments, the amino acid modification at H246 is an aromatic, polar and positively charged hydrophilic amino acid. In embodiments, the amino acid modification at H246 is a hydrophobic amino acid. In embodiments, the amino acid modification at H246 is a hydrophobic, aliphatic amino acid. In embodiments, the amino acid modification at H246 is selected from glycine (G), alanine (A), leucine (L), isoleucine (I), methionine (M), or valine (V). In embodiments, the amino acid modification at H246 is a hydrophobic, aromatic amino acid. In embodiments, the amino acid modification at H246 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 84 and an amino acid modification at position R681. In embodiments, the amino acid modifications are selected from substitutions and deletions. In embodiments, the amino acid modification to SEQ ID NO: 84 at R681 is an essential or non-essential amino acid. In embodiments, the amino acid modification at R681 is a hydrophilic or hydrophobic amino acid. In embodiments, the amino acid modification at R681 is amino acid modification is a hydrophilic amino acid. In embodiments, the amino acid modification at R681 is a polar and positively charged hydrophilic amino acid. In embodiments, the amino acid modification at R681 is lysine (K). In embodiments, the amino acid modification at R681 is a polar and neutral charged hydrophilic amino acid. In embodiments, the amino acid modification at R681 is selected from asparagine (N), glutamine (Q), serine (S), threonine (T), proline (P), and cysteine (C). In embodiments, the amino acid modification at R681 is a polar and negatively charged hydrophilic amino acid. In embodiments, the amino acid modification at R681 is selected from aspartate (D) or glutamate (E). In embodiments, the amino acid modification at R681 is an aromatic, polar and positively charged hydrophilic amino acid. In embodiments, the amino acid modification at R681 is histidine (H). In embodiments, the amino acid modification at R681 is a hydrophobic amino acid. In embodiments, the amino acid modification at R681 is a hydrophobic, aliphatic amino acid. In embodiments, the amino acid modification at R681 is selected from glycine (G), alanine (A), leucine (L), isoleucine (I), methionine (M), or valine (V). In embodiments, the amino acid modification at R681 is a hydrophobic, aromatic amino acid. In embodiments, the amino acid modification at R681 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 84 and an amino acid modification at position H686. In embodiments, the amino acid modifications are selected from substitutions and deletions. In embodiments, the amino acid modification to SEQ ID NO: 84 at H686 is an essential or non-essential amino acid. In embodiments, the amino acid modification at H686 is a hydrophilic or hydrophobic amino acid. In embodiments, the amino acid modification at H686 is amino acid modification is a hydrophilic amino acid. In embodiments, the amino acid modification at H686 is a polar and positively charged hydrophilic amino acid. In embodiments, the amino acid modification at H686 is selected from arginine (R) or lysine (K). In embodiments, the amino acid modification at H686 is a polar and neutral charged hydrophilic amino acid. In embodiments, the amino acid modification at H686 is selected from asparagine (N), glutamine (Q), serine (S), threonine (T), proline (P), and cysteine (C). In embodiments, the amino acid modification at H686 is a polar and negatively charged hydrophilic amino acid. In embodiments, the amino acid modification at H686 is selected from aspartate (D) or glutamate (E). In embodiments, the amino acid modification at H686 is an aromatic, polar and positively charged hydrophilic amino acid. In embodiments, the amino acid modification at H686 is a hydrophobic amino acid. In embodiments, the amino acid modification at H686 is a hydrophobic, aliphatic amino acid. In embodiments, the amino acid modification at H686 is selected from glycine (G), alanine (A), leucine (L), isoleucine (I), methionine (M), or valine (V). In embodiments, the amino acid modification at H686 is a hydrophobic, aromatic amino acid. In embodiments, the amino acid modification at H686 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 85 and an amino acid modification at position R244. In embodiments, the amino acid modifications are selected from substitutions and deletions. In embodiments, the amino acid modification to SEQ ID NO: 85 at R244 is an essential or non-essential amino acid. In embodiments, the amino acid modification at R244 is a hydrophilic or hydrophobic amino acid. In embodiments, the amino acid modification at R244 is amino acid modification is a hydrophilic amino acid. In embodiments, the amino acid modification at R244 is a polar and positively charged hydrophilic amino acid. In embodiments, the amino acid modification at R244 is lysine (K). In embodiments, the amino acid modification at R244 is a polar and neutral charged hydrophilic amino acid. In embodiments, the amino acid modification at R244 is selected from asparagine (N), glutamine (Q), serine (S), threonine (T), proline (P), and cysteine (C). In embodiments, the amino acid modification at R244 is a polar and negatively charged hydrophilic amino acid. In embodiments, the amino acid modification at R244 is selected from aspartate (D) or glutamate (E). In embodiments, the amino acid modification at R244 is an aromatic, polar and positively charged hydrophilic amino acid. In embodiments, the amino acid modification at R244 is histidine (H). In embodiments, the amino acid modification at R244 is a hydrophobic amino acid. In embodiments, the amino acid modification at R244 is a hydrophobic, aliphatic amino acid. In embodiments, the amino acid modification at R244 is selected from glycine (G), alanine (A), leucine (L), isoleucine (I), methionine (M), or valine (V). In embodiments, the amino acid modification at R244 is a hydrophobic, aromatic amino acid. In embodiments, the amino acid modification at R244 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 85 and an amino acid modification at position H249. In embodiments, the amino acid modifications are selected from substitutions and deletions. In embodiments, the amino acid modification to SEQ ID NO: 85 at H249 is an essential or non-essential amino acid. In embodiments, the amino acid modification at H249 is a hydrophilic or hydrophobic amino acid. In embodiments, the amino acid modification at H249 is amino acid modification is a hydrophilic amino acid. In embodiments, the amino acid modification at H249 is a polar and positively charged hydrophilic amino acid. In embodiments, the amino acid modification at H249 is selected from arginine (R) or lysine (K). In embodiments, the amino acid modification at H249 is a polar and neutral charged hydrophilic amino acid. In embodiments, the amino acid modification at H249 is selected from asparagine (N), glutamine (Q), serine (S), threonine (T), proline (P), and cysteine (C). In embodiments, the amino acid modification at H249 is a polar and negatively charged hydrophilic amino acid. In embodiments, the amino acid modification at H249 is selected from aspartate (D) or glutamate (E). In embodiments, the amino acid modification at H249 is an aromatic, polar and positively charged hydrophilic amino acid. In embodiments, the amino acid modification at H249 is a hydrophobic amino acid. In embodiments, the amino acid modification at H249 is a hydrophobic, aliphatic amino acid. In embodiments, the amino acid modification at H249 is selected from glycine (G), alanine (A), leucine (L), isoleucine (I), methionine (M), or valine (V). In embodiments, the amino acid modification at H249 is a hydrophobic, aromatic amino acid. In embodiments, the amino acid modification at H249 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 85 and an amino acid modification at position R701. In embodiments, the amino acid modifications are selected from substitutions and deletions. In embodiments, the amino acid modification to SEQ ID NO: 85 at R701 is an essential or non-essential amino acid. In embodiments, the amino acid modification at R701 is a hydrophilic or hydrophobic amino acid. In embodiments, the amino acid modification at R701 is amino acid modification is a hydrophilic amino acid. In embodiments, the amino acid modification at R701 is a polar and positively charged hydrophilic amino acid. In embodiments, the amino acid modification at R701 is lysine (K). In embodiments, the amino acid modification at R701 is a polar and neutral charged hydrophilic amino acid. In embodiments, the amino acid modification at R701 is selected from asparagine (N), glutamine (Q), serine (S), threonine (T), proline (P), and cysteine (C). In embodiments, the amino acid modification at R701 is a polar and negatively charged hydrophilic amino acid. In embodiments, the amino acid modification at R701 is selected from aspartate (D) or glutamate (E). In embodiments, the amino acid modification at R701 is an aromatic, polar and positively charged hydrophilic amino acid. In embodiments, the amino acid modification at R701 is histidine (H). In embodiments, the amino acid modification at R701 is a hydrophobic amino acid. In embodiments, the amino acid modification at R701 is a hydrophobic, aliphatic amino acid. In embodiments, the amino acid modification at R701 is selected from glycine (G), alanine (A), leucine (L), isoleucine (I), methionine (M), or valine (V). In embodiments, the amino acid modification at R701 is a hydrophobic, aromatic amino acid. In embodiments, the amino acid modification at R701 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 85 and an amino acid modification at position H706. In embodiments, the amino acid modifications are selected from substitutions and deletions. In embodiments, the amino acid modification to SEQ ID NO: 85 at H706 is an essential or non-essential amino acid. In embodiments, the amino acid modification at H706 is a hydrophilic or hydrophobic amino acid. In embodiments, the amino acid modification at H706 is amino acid modification is a hydrophilic amino acid. In embodiments, the amino acid modification at H706 is a polar and positively charged hydrophilic amino acid. In embodiments, the amino acid modification at H706 is selected from arginine (R) or lysine (K). In embodiments, the amino acid modification at H706 is a polar and neutral charged hydrophilic amino acid. In embodiments, the amino acid modification at H706 is selected from asparagine (N), glutamine (Q), serine (S), threonine (T), proline (P), and cysteine (C). In embodiments, the amino acid modification at H706 is a polar and negatively charged hydrophilic amino acid. In embodiments, the amino acid modification at H706 is selected from aspartate (D) or glutamate (E). In embodiments, the amino acid modification at H706 is an aromatic, polar and positively charged hydrophilic amino acid. In embodiments, the amino acid modification at H706 is a hydrophobic amino acid. In embodiments, the amino acid modification at H706 is a hydrophobic, aliphatic amino acid. In embodiments, the amino acid modification at H706 is selected from glycine (G), alanine (A), leucine (L), isoleucine (I), methionine (M), or valine (V). In embodiments, the amino acid modification at H706 is a hydrophobic, aromatic amino acid. In embodiments, the amino acid modification at H706 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 86 and an amino acid modification at position R247. In embodiments, the amino acid modifications are selected from substitutions and deletions. In embodiments, the amino acid modification to SEQ ID NO: 86 at R247 is an essential or non-essential amino acid. In embodiments, the amino acid modification at R247 is a hydrophilic or hydrophobic amino acid. In embodiments, the amino acid modification at R247 is amino acid modification is a hydrophilic amino acid. In embodiments, the amino acid modification at R247 is a polar and positively charged hydrophilic amino acid. In embodiments, the amino acid modification at R247 is lysine (K). In embodiments, the amino acid modification at R247 is a polar and neutral charged hydrophilic amino acid. In embodiments, the amino acid modification at R247 is selected from asparagine (N), glutamine (Q), serine (S), threonine (T), proline (P), and cysteine (C). In embodiments, the amino acid modification at R247 is a polar and negatively charged hydrophilic amino acid. In embodiments, the amino acid modification at R247 is selected from aspartate (D) or glutamate (E). In embodiments, the amino acid modification at R247 is an aromatic, polar and positively charged hydrophilic amino acid. In embodiments, the amino acid modification at R247 is histidine (H). In embodiments, the amino acid modification at R247 is a hydrophobic amino acid. In embodiments, the amino acid modification at R247 is a hydrophobic, aliphatic amino acid. In embodiments, the amino acid modification at R247 is selected from glycine (G), alanine (A), leucine (L), isoleucine (I), methionine (M), or valine (V). In embodiments, the amino acid modification at R247 is a hydrophobic, aromatic amino acid. In embodiments, the amino acid modification at R247 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 86 and an amino acid modification at position H252. In embodiments, the amino acid modifications are selected from substitutions and deletions. In embodiments, the amino acid modification to SEQ ID NO: 86 at H252 is an essential or non-essential amino acid. In embodiments, the amino acid modification at H252 is a hydrophilic or hydrophobic amino acid. In embodiments, the amino acid modification at H252 is amino acid modification is a hydrophilic amino acid. In embodiments, the amino acid modification at H252 is a polar and positively charged hydrophilic amino acid. In embodiments, the amino acid modification at H252 is selected from arginine (R) or lysine (K). In embodiments, the amino acid modification at H252 is a polar and neutral charged hydrophilic amino acid. In embodiments, the amino acid modification at H252 is selected from asparagine (N), glutamine (Q), serine (S), threonine (T), proline (P), and cysteine (C). In embodiments, the amino acid modification at H252 is a polar and negatively charged hydrophilic amino acid. In embodiments, the amino acid modification at H252 is selected from aspartate (D) or glutamate (E). In embodiments, the amino acid modification at H252 is an aromatic, polar and positively charged hydrophilic amino acid. In embodiments, the amino acid modification at H252 is a hydrophobic amino acid. In embodiments, the amino acid modification at H252 is a hydrophobic, aliphatic amino acid. In embodiments, the amino acid modification at H252 is selected from glycine (G), alanine (A), leucine (L), isoleucine (I), methionine (M), or valine (V). In embodiments, the amino acid modification at H252 is a hydrophobic, aromatic amino acid. In embodiments, the amino acid modification at H252 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 86 and an amino acid modification at position R711. In embodiments, the amino acid modifications are selected from substitutions and deletions. In embodiments, the amino acid modification to SEQ ID NO: 86 at R711 is an essential or non-essential amino acid. In embodiments, the amino acid modification at R711 is a hydrophilic or hydrophobic amino acid. In embodiments, the amino acid modification at R711 is amino acid modification is a hydrophilic amino acid. In embodiments, the amino acid modification at R711 is a polar and positively charged hydrophilic amino acid. In embodiments, the amino acid modification at R711 is lysine (K). In embodiments, the amino acid modification at R711 is a polar and neutral charged hydrophilic amino acid. In embodiments, the amino acid modification at R711 is selected from asparagine (N), glutamine (Q), serine (S), threonine (T), proline (P), and cysteine (C). In embodiments, the amino acid modification at R711 is a polar and negatively charged hydrophilic amino acid. In embodiments, the amino acid modification at R711 is selected from aspartate (D) or glutamate (E). In embodiments, the amino acid modification at R711 is an aromatic, polar and positively charged hydrophilic amino acid. In embodiments, the amino acid modification at R711 is histidine (H). In embodiments, the amino acid modification at R711 is a hydrophobic amino acid. In embodiments, the amino acid modification at R711 is a hydrophobic, aliphatic amino acid. In embodiments, the amino acid modification at R711 is selected from glycine (G), alanine (A), leucine (L), isoleucine (I), methionine (M), or valine (V). In embodiments, the amino acid modification at R711 is a hydrophobic, aromatic amino acid. In embodiments, the amino acid modification at R711 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 86 and an amino acid modification at position H716. In embodiments, the amino acid modifications are selected from substitutions and deletions. In embodiments, the amino acid modification to SEQ ID NO: 86 at H716 is an essential or non-essential amino acid. In embodiments, the amino acid modification at H716 is a hydrophilic or hydrophobic amino acid. In embodiments, the amino acid modification at H716 is amino acid modification is a hydrophilic amino acid. In embodiments, the amino acid modification at H716 is a polar and positively charged hydrophilic amino acid. In embodiments, the amino acid modification at H716 is selected from arginine (R) or lysine (K). In embodiments, the amino acid modification at H716 is a polar and neutral charged hydrophilic amino acid. In embodiments, the amino acid modification at H716 is selected from asparagine (N), glutamine (Q), serine (S), threonine (T), proline (P), and cysteine (C). In embodiments, the amino acid modification at H716 is a polar and negatively charged hydrophilic amino acid. In embodiments, the amino acid modification at H716 is selected from aspartate (D) or glutamate (E). In embodiments, the amino acid modification at H716 is an aromatic, polar and positively charged hydrophilic amino acid. In embodiments, the amino acid modification at H716 is a hydrophobic amino acid. In embodiments, the amino acid modification at H716 is a hydrophobic, aliphatic amino acid. In embodiments, the amino acid modification at H716 is selected from glycine (G), alanine (A), leucine (L), isoleucine (I), methionine (M), or valine (V). In embodiments, the amino acid modification at H716 is a hydrophobic, aromatic amino acid. In embodiments, the amino acid modification at H716 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 87 and an amino acid modification at position R316. In embodiments, the amino acid modifications are selected from substitutions and deletions. In embodiments, the amino acid modification to SEQ ID NO: 87 at R316 is an essential or non-essential amino acid. In embodiments, the amino acid modification at R316 is a hydrophilic or hydrophobic amino acid. In embodiments, the amino acid modification at R316 is amino acid modification is a hydrophilic amino acid. In embodiments, the amino acid modification at R316 is a polar and positively charged hydrophilic amino acid. In embodiments, the amino acid modification at R316 is lysine (K). In embodiments, the amino acid modification at R316 is a polar and neutral charged hydrophilic amino acid. In embodiments, the amino acid modification at R316 is selected from asparagine (N), glutamine (Q), serine (S), threonine (T), proline (P), and cysteine (C). In embodiments, the amino acid modification at R316 is a polar and negatively charged hydrophilic amino acid. In embodiments, the amino acid modification at R316 is selected from aspartate (D) or glutamate (E). In embodiments, the amino acid modification at R316 is an aromatic, polar and positively charged hydrophilic amino acid. In embodiments, the amino acid modification at R316 is histidine (H). In embodiments, the amino acid modification at R316 is a hydrophobic amino acid. In embodiments, the amino acid modification at R316 is a hydrophobic, aliphatic amino acid. In embodiments, the amino acid modification at R316 is selected from glycine (G), alanine (A), leucine (L), isoleucine (I), methionine (M), or valine (V). In embodiments, the amino acid modification at R316 is a hydrophobic, aromatic amino acid. In embodiments, the amino acid modification at R316 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 87 and an amino acid modification at position H321. In embodiments, the amino acid modifications are selected from substitutions and deletions. In embodiments, the amino acid modification to SEQ ID NO: 87 at H321 is an essential or non-essential amino acid. In embodiments, the amino acid modification at H321 is a hydrophilic or hydrophobic amino acid. In embodiments, the amino acid modification at H321 is amino acid modification is a hydrophilic amino acid. In embodiments, the amino acid modification at H321 is a polar and positively charged hydrophilic amino acid. In embodiments, the amino acid modification at H321 is selected from arginine (R) or lysine (K). In embodiments, the amino acid modification at H321 is a polar and neutral charged hydrophilic amino acid. In embodiments, the amino acid modification at H321 is selected from asparagine (N), glutamine (Q), serine (S), threonine (T), proline (P), and cysteine (C). In embodiments, the amino acid modification at H321 is a polar and negatively charged hydrophilic amino acid. In embodiments, the amino acid modification at H321 is selected from aspartate (D) or glutamate (E). In embodiments, the amino acid modification at H321 is an aromatic, polar and positively charged hydrophilic amino acid. In embodiments, the amino acid modification at H321 is a hydrophobic amino acid. In embodiments, the amino acid modification at H321 is a hydrophobic, aliphatic amino acid. In embodiments, the amino acid modification at H321 is selected from glycine (G), alanine (A), leucine (L), isoleucine (I), methionine (M), or valine (V). In embodiments, the amino acid modification at H321 is a hydrophobic, aromatic amino acid. In embodiments, the amino acid modification at H321 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 87 and an amino acid modification at position R781. In embodiments, the amino acid modifications are selected from substitutions and deletions. In embodiments, the amino acid modification to SEQ ID NO: 87 at R781 is an essential or non-essential amino acid. In embodiments, the amino acid modification at R781 is a hydrophilic or hydrophobic amino acid. In embodiments, the amino acid modification at R781 is amino acid modification is a hydrophilic amino acid. In embodiments, the amino acid modification at R781 is a polar and positively charged hydrophilic amino acid. In embodiments, the amino acid modification at R781 is lysine (K). In embodiments, the amino acid modification at R781 is a polar and neutral charged hydrophilic amino acid. In embodiments, the amino acid modification at R781 is selected from asparagine (N), glutamine (Q), serine (S), threonine (T), proline (P), and cysteine (C). In embodiments, the amino acid modification at R781 is a polar and negatively charged hydrophilic amino acid. In embodiments, the amino acid modification at R781 is selected from aspartate (D) or glutamate (E). In embodiments, the amino acid modification at R781 is an aromatic, polar and positively charged hydrophilic amino acid. In embodiments, the amino acid modification at R781 is histidine (H). In embodiments, the amino acid modification at R781 is a hydrophobic amino acid. In embodiments, the amino acid modification at R781 is a hydrophobic, aliphatic amino acid. In embodiments, the amino acid modification at R781 is selected from glycine (G), alanine (A), leucine (L), isoleucine (I), methionine (M), or valine (V). In embodiments, the amino acid modification at R781 is a hydrophobic, aromatic amino acid. In embodiments, the amino acid modification at R781 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 87 and an amino acid modification at position H786. In embodiments, the amino acid modifications are selected from substitutions and deletions. In embodiments, the amino acid modification to SEQ ID NO: 87 at H786 is an essential or non-essential amino acid. In embodiments, the amino acid modification at H786 is a hydrophilic or hydrophobic amino acid. In embodiments, the amino acid modification at H786 is amino acid modification is a hydrophilic amino acid. In embodiments, the amino acid modification at H786 is a polar and positively charged hydrophilic amino acid. In embodiments, the amino acid modification at H786 is selected from arginine (R) or lysine (K). In embodiments, the amino acid modification at H786 is a polar and neutral charged hydrophilic amino acid. In embodiments, the amino acid modification at H786 is selected from asparagine (N), glutamine (Q), serine (S), threonine (T), proline (P), and cysteine (C). In embodiments, the amino acid modification at H786 is a polar and negatively charged hydrophilic amino acid. In embodiments, the amino acid modification at H786 is selected from aspartate (D) or glutamate (E). In embodiments, the amino acid modification at H786 is an aromatic, polar and positively charged hydrophilic amino acid. In embodiments, the amino acid modification at H786 is a hydrophobic amino acid. In embodiments, the amino acid modification at H786 is a hydrophobic, aliphatic amino acid. In embodiments, the amino acid modification at H786 is selected from glycine (G), alanine (A), leucine (L), isoleucine (I), methionine (M), or valine (V). In embodiments, the amino acid modification at H786 is a hydrophobic, aromatic amino acid. In embodiments, the amino acid modification at H786 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 88 and an amino acid modification at position R274. In embodiments, the amino acid modifications are selected from substitutions and deletions. In embodiments, the amino acid modification to SEQ ID NO: 88 at R274 is an essential or non-essential amino acid. In embodiments, the amino acid modification at R274 is a hydrophilic or hydrophobic amino acid. In embodiments, the amino acid modification at R274 is amino acid modification is a hydrophilic amino acid. In embodiments, the amino acid modification at R274 is a polar and positively charged hydrophilic amino acid. In embodiments, the amino acid modification at R274 is lysine (K). In embodiments, the amino acid modification at R274 is a polar and neutral charged hydrophilic amino acid. In embodiments, the amino acid modification at R274 is selected from asparagine (N), glutamine (Q), serine (S), threonine (T), proline (P), and cysteine (C). In embodiments, the amino acid modification at R274 is a polar and negatively charged hydrophilic amino acid. In embodiments, the amino acid modification at R274 is selected from aspartate (D) or glutamate (E). In embodiments, the amino acid modification at R274 is an aromatic, polar and positively charged hydrophilic amino acid. In embodiments, the amino acid modification at R274 is histidine (H). In embodiments, the amino acid modification at R274 is a hydrophobic amino acid. In embodiments, the amino acid modification at R274 is a hydrophobic, aliphatic amino acid. In embodiments, the amino acid modification at R274 is selected from glycine (G), alanine (A), leucine (L), isoleucine (I), methionine (M), or valine (V). In embodiments, the amino acid modification at R274 is a hydrophobic, aromatic amino acid. In embodiments, the amino acid modification at R274 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 88 and an amino acid modification at position H279. In embodiments, the amino acid modifications are selected from substitutions and deletions. In embodiments, the amino acid modification to SEQ ID NO: 88 at H279 is an essential or non-essential amino acid. In embodiments, the amino acid modification at H279 is a hydrophilic or hydrophobic amino acid. In embodiments, the amino acid modification at H279 is amino acid modification is a hydrophilic amino acid. In embodiments, the amino acid modification at H279 is a polar and positively charged hydrophilic amino acid. In embodiments, the amino acid modification at H279 is selected from arginine (R) or lysine (K). In embodiments, the amino acid modification at H279 is a polar and neutral charged hydrophilic amino acid. In embodiments, the amino acid modification at H279 is selected from asparagine (N), glutamine (Q), serine (S), threonine (T), proline (P), and cysteine (C). In embodiments, the amino acid modification at H279 is a polar and negatively charged hydrophilic amino acid. In embodiments, the amino acid modification at H279 is selected from aspartate (D) or glutamate (E). In embodiments, the amino acid modification at H279 is an aromatic, polar and positively charged hydrophilic amino acid. In embodiments, the amino acid modification at H279 is a hydrophobic amino acid. In embodiments, the amino acid modification at H279 is a hydrophobic, aliphatic amino acid. In embodiments, the amino acid modification at H279 is selected from glycine (G), alanine (A), leucine (L), isoleucine (I), methionine (M), or valine (V). In embodiments, the amino acid modification at H279 is a hydrophobic, aromatic amino acid. In embodiments, the amino acid modification at H279 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 88 and an amino acid modification at position R723. In embodiments, the amino acid modifications are selected from substitutions and deletions. In embodiments, the amino acid modification to SEQ ID NO: 88 at R723 is an essential or non-essential amino acid. In embodiments, the amino acid modification at R723 is a hydrophilic or hydrophobic amino acid. In embodiments, the amino acid modification at R723 is amino acid modification is a hydrophilic amino acid. In embodiments, the amino acid modification at R723 is a polar and positively charged hydrophilic amino acid. In embodiments, the amino acid modification at R723 is lysine (K). In embodiments, the amino acid modification at R723 is a polar and neutral charged hydrophilic amino acid. In embodiments, the amino acid modification at R723 is selected from asparagine (N), glutamine (Q), serine (S), threonine (T), proline (P), and cysteine (C). In embodiments, the amino acid modification at R723 is a polar and negatively charged hydrophilic amino acid. In embodiments, the amino acid modification at R723 is selected from aspartate (D) or glutamate (E). In embodiments, the amino acid modification at R723 is an aromatic, polar and positively charged hydrophilic amino acid. In embodiments, the amino acid modification at R723 is histidine (H). In embodiments, the amino acid modification at R723 is a hydrophobic amino acid. In embodiments, the amino acid modification at R723 is a hydrophobic, aliphatic amino acid. In embodiments, the amino acid modification at R723 is selected from glycine (G), alanine (A), leucine (L), isoleucine (I), methionine (M), or valine (V). In embodiments, the amino acid modification at R723 is a hydrophobic, aromatic amino acid. In embodiments, the amino acid modification at R723 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 88 and an amino acid modification at position H728. In embodiments, the amino acid modifications are selected from substitutions and deletions. In embodiments, the amino acid modification to SEQ ID NO: 88 at H728 is an essential or non-essential amino acid. In embodiments, the amino acid modification at H728 is a hydrophilic or hydrophobic amino acid. In embodiments, the amino acid modification at H728 is amino acid modification is a hydrophilic amino acid. In embodiments, the amino acid modification at H728 is a polar and positively charged hydrophilic amino acid. In embodiments, the amino acid modification at H728 is selected from arginine (R) or lysine (K). In embodiments, the amino acid modification at H728 is a polar and neutral charged hydrophilic amino acid. In embodiments, the amino acid modification at H728 is selected from asparagine (N), glutamine (Q), serine (S), threonine (T), proline (P), and cysteine (C). In embodiments, the amino acid modification at H728 is a polar and negatively charged hydrophilic amino acid. In embodiments, the amino acid modification at H728 is selected from aspartate (D) or glutamate (E). In embodiments, the amino acid modification at H728 is an aromatic, polar and positively charged hydrophilic amino acid. In embodiments, the amino acid modification at H728 is a hydrophobic amino acid. In embodiments, the amino acid modification at H728 is a hydrophobic, aliphatic amino acid. In embodiments, the amino acid modification at H728 is selected from glycine (G), alanine (A), leucine (L), isoleucine (I), methionine (M), or valine (V). In embodiments, the amino acid modification at H728 is a hydrophobic, aromatic amino acid. In embodiments, the amino acid modification at H728 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 89 and an amino acid modification at position R237. In embodiments, the amino acid modifications are selected from substitutions and deletions. In embodiments, the amino acid modification to SEQ ID NO: 89 at R237 is an essential or non-essential amino acid. In embodiments, the amino acid modification at R237 is a hydrophilic or hydrophobic amino acid. In embodiments, the amino acid modification at R237 is amino acid modification is a hydrophilic amino acid. In embodiments, the amino acid modification at R237 is a polar and positively charged hydrophilic amino acid. In embodiments, the amino acid modification at R237 is lysine (K). In embodiments, the amino acid modification at R237 is a polar and neutral charged hydrophilic amino acid. In embodiments, the amino acid modification at R237 is selected from asparagine (N), glutamine (Q), serine (S), threonine (T), proline (P), and cysteine (C). In embodiments, the amino acid modification at R237 is a polar and negatively charged hydrophilic amino acid. In embodiments, the amino acid modification at R237 is selected from aspartate (D) or glutamate (E). In embodiments, the amino acid modification at R237 is an aromatic, polar and positively charged hydrophilic amino acid. In embodiments, the amino acid modification at R237 is histidine (H). In embodiments, the amino acid modification at R237 is a hydrophobic amino acid. In embodiments, the amino acid modification at R237 is a hydrophobic, aliphatic amino acid. In embodiments, the amino acid modification at R237 is selected from glycine (G), alanine (A), leucine (L), isoleucine (I), methionine (M), or valine (V). In embodiments, the amino acid modification at R237 is a hydrophobic, aromatic amino acid. In embodiments, the amino acid modification at R237 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 89 and an amino acid modification at position H242. In embodiments, the amino acid modifications are selected from substitutions and deletions. In embodiments, the amino acid modification to SEQ ID NO: 89 at H242 is an essential or non-essential amino acid. In embodiments, the amino acid modification at H242 is a hydrophilic or hydrophobic amino acid. In embodiments, the amino acid modification at H242 is amino acid modification is a hydrophilic amino acid. In embodiments, the amino acid modification at H242 is a polar and positively charged hydrophilic amino acid. In embodiments, the amino acid modification at H242 is selected from arginine (R) or lysine (K). In embodiments, the amino acid modification at H242 is a polar and neutral charged hydrophilic amino acid. In embodiments, the amino acid modification at H242 is selected from asparagine (N), glutamine (Q), serine (S), threonine (T), proline (P), and cysteine (C). In embodiments, the amino acid modification at H242 is a polar and negatively charged hydrophilic amino acid. In embodiments, the amino acid modification at H242 is selected from aspartate (D) or glutamate (E). In embodiments, the amino acid modification at H242 is an aromatic, polar and positively charged hydrophilic amino acid. In embodiments, the amino acid modification at H242 is a hydrophobic amino acid. In embodiments, the amino acid modification at H242 is a hydrophobic, aliphatic amino acid. In embodiments, the amino acid modification at H242 is selected from glycine (G), alanine (A), leucine (L), isoleucine (I), methionine (M), or valine (V). In embodiments, the amino acid modification at H242 is a hydrophobic, aromatic amino acid. In embodiments, the amino acid modification at H242 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 89 and an amino acid modification at position R694. In embodiments, the amino acid modifications are selected from substitutions and deletions. In embodiments, the amino acid modification to SEQ ID NO: 89 at R694 is an essential or non-essential amino acid. In embodiments, the amino acid modification at R694 is a hydrophilic or hydrophobic amino acid. In embodiments, the amino acid modification at R694 is amino acid modification is a hydrophilic amino acid. In embodiments, the amino acid modification at R694 is a polar and positively charged hydrophilic amino acid. In embodiments, the amino acid modification at R694 is lysine (K). In embodiments, the amino acid modification at R694 is a polar and neutral charged hydrophilic amino acid. In embodiments, the amino acid modification at R694 is selected from asparagine (N), glutamine (Q), serine (S), threonine (T), proline (P), and cysteine (C). In embodiments, the amino acid modification at R694 is a polar and negatively charged hydrophilic amino acid. In embodiments, the amino acid modification at R694 is selected from aspartate (D) or glutamate (E). In embodiments, the amino acid modification at R694 is an aromatic, polar and positively charged hydrophilic amino acid. In embodiments, the amino acid modification at R694 is histidine (H). In embodiments, the amino acid modification at R694 is a hydrophobic amino acid. In embodiments, the amino acid modification at R694 is a hydrophobic, aliphatic amino acid. In embodiments, the amino acid modification at R694 is selected from glycine (G), alanine (A), leucine (L), isoleucine (I), methionine (M), or valine (V). In embodiments, the amino acid modification at R694 is a hydrophobic, aromatic amino acid. In embodiments, the amino acid modification at R694 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 89 and an amino acid modification at position H699. In embodiments, the amino acid modifications are selected from substitutions and deletions. In embodiments, the amino acid modification to SEQ ID NO: 89 at H699 is an essential or non-essential amino acid. In embodiments, the amino acid modification at H699 is a hydrophilic or hydrophobic amino acid. In embodiments, the amino acid modification at H699 is amino acid modification is a hydrophilic amino acid. In embodiments, the amino acid modification at H699 is a polar and positively charged hydrophilic amino acid. In embodiments, the amino acid modification at H699 is selected from arginine (R) or lysine (K). In embodiments, the amino acid modification at H699 is a polar and neutral charged hydrophilic amino acid. In embodiments, the amino acid modification at H699 is selected from asparagine (N), glutamine (Q), serine (S), threonine (T), proline (P), and cysteine (C). In embodiments, the amino acid modification at H699 is a polar and negatively charged hydrophilic amino acid. In embodiments, the amino acid modification at H699 is selected from aspartate (D) or glutamate (E). In embodiments, the amino acid modification at H699 is an aromatic, polar and positively charged hydrophilic amino acid. In embodiments, the amino acid modification at H699 is a hydrophobic amino acid. In embodiments, the amino acid modification at H699 is a hydrophobic, aliphatic amino acid. In embodiments, the amino acid modification at H699 is selected from glycine (G), alanine (A), leucine (L), isoleucine (I), methionine (M), or valine (V). In embodiments, the amino acid modification at H699 is a hydrophobic, aromatic amino acid. In embodiments, the amino acid modification at H699 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 89 and an amino acid modification at position R237. In embodiments, the amino acid modifications are selected from substitutions and deletions. In embodiments, the amino acid modification to SEQ ID NO: 89 at R237 is an essential or non-essential amino acid. In embodiments, the amino acid modification at R237 is a hydrophilic or hydrophobic amino acid. In embodiments, the amino acid modification at R237 is amino acid modification is a hydrophilic amino acid. In embodiments, the amino acid modification at R237 is a polar and positively charged hydrophilic amino acid. In embodiments, the amino acid modification at R237 is lysine (K). In embodiments, the amino acid modification at R237 is a polar and neutral charged hydrophilic amino acid. In embodiments, the amino acid modification at R237 is selected from asparagine (N), glutamine (Q), serine (S), threonine (T), proline (P), and cysteine (C). In embodiments, the amino acid modification at R237 is a polar and negatively charged hydrophilic amino acid. In embodiments, the amino acid modification at R237 is selected from aspartate (D) or glutamate (E). In embodiments, the amino acid modification at R237 is an aromatic, polar and positively charged hydrophilic amino acid. In embodiments, the amino acid modification at R237 is histidine (H). In embodiments, the amino acid modification at R237 is a hydrophobic amino acid. In embodiments, the amino acid modification at R237 is a hydrophobic, aliphatic amino acid. In embodiments, the amino acid modification at R237 is selected from glycine (G), alanine (A), leucine (L), isoleucine (I), methionine (M), or valine (V). In embodiments, the amino acid modification at R237 is a hydrophobic, aromatic amino acid. In embodiments, the amino acid modification at R237 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 89 and an amino acid modification at position H242. In embodiments, the amino acid modifications are selected from substitutions and deletions. In embodiments, the amino acid modification to SEQ ID NO: 89 at H242 is an essential or non-essential amino acid. In embodiments, the amino acid modification at H242 is a hydrophilic or hydrophobic amino acid. In embodiments, the amino acid modification at H242 is amino acid modification is a hydrophilic amino acid. In embodiments, the amino acid modification at H242 is a polar and positively charged hydrophilic amino acid. In embodiments, the amino acid modification at H242 is selected from arginine (R) or lysine (K). In embodiments, the amino acid modification at H242 is a polar and neutral charged hydrophilic amino acid. In embodiments, the amino acid modification at H242 is selected from asparagine (N), glutamine (Q), serine (S), threonine (T), proline (P), and cysteine (C). In embodiments, the amino acid modification at H242 is a polar and negatively charged hydrophilic amino acid. In embodiments, the amino acid modification at H242 is selected from aspartate (D) or glutamate (E). In embodiments, the amino acid modification at H242 is an aromatic, polar and positively charged hydrophilic amino acid. In embodiments, the amino acid modification at H242 is a hydrophobic amino acid. In embodiments, the amino acid modification at H242 is a hydrophobic, aliphatic amino acid. In embodiments, the amino acid modification at H242 is selected from glycine (G), alanine (A), leucine (L), isoleucine (I), methionine (M), or valine (V). In embodiments, the amino acid modification at H242 is a hydrophobic, aromatic amino acid. In embodiments, the amino acid modification at H242 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 89 and an amino acid modification at position R694. In embodiments, the amino acid modifications are selected from substitutions and deletions. In embodiments, the amino acid modification to SEQ ID NO: 89 at R694 is an essential or non-essential amino acid. In embodiments, the amino acid modification at R694 is a hydrophilic or hydrophobic amino acid. In embodiments, the amino acid modification at R694 is amino acid modification is a hydrophilic amino acid. In embodiments, the amino acid modification at R694 is a polar and positively charged hydrophilic amino acid. In embodiments, the amino acid modification at R694 is lysine (K). In embodiments, the amino acid modification at R694 is a polar and neutral charged hydrophilic amino acid. In embodiments, the amino acid modification at R694 is selected from asparagine (N), glutamine (Q), serine (S), threonine (T), proline (P), and cysteine (C). In embodiments, the amino acid modification at R694 is a polar and negatively charged hydrophilic amino acid. In embodiments, the amino acid modification at R694 is selected from aspartate (D) or glutamate (E). In embodiments, the amino acid modification at R694 is an aromatic, polar and positively charged hydrophilic amino acid. In embodiments, the amino acid modification at R694 is histidine (H). In embodiments, the amino acid modification at R694 is a hydrophobic amino acid. In embodiments, the amino acid modification at R694 is a hydrophobic, aliphatic amino acid. In embodiments, the amino acid modification at R694 is selected from glycine (G), alanine (A), leucine (L), isoleucine (I), methionine (M), or valine (V). In embodiments, the amino acid modification at R694 is a hydrophobic, aromatic amino acid. In embodiments, the amino acid modification at R694 is selected from phenylalanine (F), tryptophan (W), or tyrosine (Y).

In embodiments, the endonuclease has at least about 70%, at least about 80%, at least about 85%, at least about 90%, at least about 91%, at least about 91%, at least about 92%, at least about 93%, at least about 94%, at least about 95%, at least about 96%, at least about 97%, at least about 98%, or at least about 99% identity to SEQ ID NO: 89 and an amino acid modification at position H699. In embodiments, the amino acid modifications are selected from substitutions and deletions. In embodiments, the amino acid modification to SEQ ID NO: 89 at H699 is an essential or non-essential amino acid. In embodiments, the amino acid modification at H699 is a hydrophilic or hydrophobic amino acid. In embodiments, the amino acid modification at H699 is amino acid modification is a hydrophilic amino acid. In embodiments, the amino acid modification at H699 is a polar and positively charged hydrophilic amino acid. In embodiments, the amino acid modification at H699 is selected from arginine (R) or lysine (K). In embodiments, the amino acid modification at H699 is a polar and neutral charged hydrophilic amino acid. In embodiments, the amino acid modification at H699 is selected from asparagine (N), glutamine (Q), serine (S), threonine (T), proline (P), and cysteine (C). In embodiments, the amino acid modification at H699 is a polar and negatively charged hydrophilic amino acid. In embodiments, the amino acid modification at H699 is selected from aspartate (D) or glutamate (E). In embodiments, the amino acid modification at H699 is an aromatic, polar and positively charged hydrophilic amino acid. In embodiments, the amino acid modification at H699 is a hydrophobic amino acid. In embodiments, the amino acid modification at H699 is a hydrophobic, aliphatic amino acid. In embodiments, the amino acid modification at H699 is selected from glycine (G), alanine (A), leucine (L), isoleucine (I), methionine (M), or valine (V). In embodiments, the amino acid modification at H699 is a hydrophobic, aromatic amino acid. In embodiments, the amino acid modification at H699 is selected from phenylalanine (F), tryptophan (W), or tyrosine (Y).

In embodiments, disclosed herein is a composition comprising an endonuclease and having an amino acid sequence of least 90%, or at least 95%, or at least 98%, or at least 99% identity to SEQ ID NO: 3. In embodiments, substitutions are made to SEQ ID NO: 3:

Met Asp Lys His Pro Ser Asn Arg Tyr Ala Leu Pro Lys Val Ile Ile Ser Glu Val
Asp His Glu Arg Ile Leu Glu Phe Lys Val Lys Tyr Glu Lys Leu Ala Arg Leu
Asp Arg Phe Glu Val Lys Ala Met His Tyr Asp Gly Ala Glu Ile Val Phe Asp
Glu Val Val Ala Asn Gly Gly Leu Ile Glu Val Glu Tyr Gln Asp Asn Asn Lys
Thr Ile Thr Ile Asn Leu Asn Gly Lys Lys Tyr Thr Ile Asn Gly Arg Lys Val Gly
Gly Lys Arg Arg Leu Leu Glu Asp Arg Ile Ser Arg Gly Lys Val Cys Leu Glu
Leu His Asp Lys Ile Pro Asp Glu Lys Gly Asn Leu Arg Ser Ser Arg Thr Glu
Arg Glu Leu Ile Thr Phe Asp Ser Thr Lys Leu Tyr Ser Gln Ile Ile Gly Arg Asp
Val Ala Ser Thr Lys Glu Ile Tyr Leu Ile Lys Arg Phe Leu Ala Tyr Arg Ser Asp
Leu Leu Phe Tyr Tyr Gly Phe Ile Asp Asn Phe Phe Lys Val Ala Gly Asn Lys
Arg Glu Leu Trp Lys Ile Asp Phe Ser Gly Asp Lys Asn Gln Glu Leu Ile Lys
Tyr Phe Asn Phe Thr Ile Asn Asp Lys Leu Lys Asn Asp Lys Gly Tyr Leu Lys
Glu Tyr Thr Ala Asn Asp Glu Gln Ile Lys Lys Asp Leu Gln Asn Thr Lys Glu
Val Phe Thr Ala Leu Arg His Ala Leu Met His Phe Glu Tyr Asp Phe Phe Glu
Lys Leu Phe Asn Asn Glu Glu Ile Glu Thr Leu Ser Lys Ile His Asp Ile Glu Leu
Leu Asn Thr Met Ile Asn Lys Leu Asp Lys Leu Asn Ile Asp Thr Arg Lys Glu
Tyr Ile Asp Asp Glu Lys Ile Thr Val Phe Gly Glu Glu Ile Ser Leu Lys Thr Leu
Tyr Gly Leu Tyr Ala His Thr Ala Ile Asn Arg Val Ala Phe Asn Lys Leu Ile Asn
Arg Phe Met Val Glu Asn Gly Thr Glu Asn Glu Ala Leu Lys Lys Tyr Phe Asn
Ser Lys Ala Glu Gly Gly Ile Ala Tyr Glu Ile Asp Ile His Gln Asn Ser Glu Tyr
Lys Gln Leu Tyr Ile Gln His Lys Asp Leu Val Ser Lys Leu Ser Ala Leu Ser Asp
Gly Asp Glu Ile Ala Asp Thr Asn Lys Lys Ile Ser Glu Leu Lys Val Lys Met
Lys Ala Ile Thr Lys Ala Asn Ser Leu Lys Arg Leu Glu His Lys Leu Arg Leu
Thr Phe Gly Phe Ile Tyr Thr Glu Tyr Gln Asp Tyr Asn Ala Phe Lys Asn Asn
Phe Asp Thr Asp Ile Lys Ser Gly Arg Phe Ile Pro Lys Asp Ser Glu Gly Lys Arg
Arg Gly Phe Asp His Arg Glu Leu Asp Gln Leu Lys Arg Tyr Tyr Asp Ala Thr
Phe Ala Asp Lys Lys Pro Gln Thr Lys Glu Thr Phe Asp Glu Ile Asp Lys Gln
Ile Asp Gln Leu Ser Leu Lys Asn Leu Ile Gly Asp Asp Thr Leu Leu Lys Val
Ile Leu Leu Ile Tyr Ile Phe Leu Pro Arg Glu Ile Lys Gly Glu Phe Leu Gly Phe
Val Lys Lys Tyr Tyr His Asp Thr Lys His Ile Glu Glu Asp Thr Lys Asp Lys
Asp Glu Gly Phe Asp Asp Thr Phe Pro Val Gly Leu Lys Leu Lys Val Leu Asp
Lys Asn Ile Arg Ala Leu Ser Val Leu Lys His Ser Leu Ser Tyr Gln Ala Lys Tyr
Asn Lys Lys Glu Glu Lys Lys Glu Gln Phe Tyr Glu Ala Gly Asn Arg His Gly
Arg Phe Tyr Lys Lys Leu Gly Ile Ser His Asn Gln Glu Glu Phe Asp Lys Ser
Val Tyr Ala Pro Leu Leu Arg Tyr His Ala Ala Leu Phe Lys Leu Leu Asn Asp
Phe Glu Ile Tyr Ser Leu Ala Gln His Ile Glu Gly Lys Glu Thr Leu Ala Gln Gln
Ile Glu Lys Pro Gln Phe Ser Gln Tyr Glu His Tyr Asn Phe Arg Lys Met Leu
Ser Lys Thr Tyr Pro Lys Ser Ala Glu Arg Gly Ala Leu Asp Asn Asp Ala Phe
Asp Thr Val Ile Asn Met Arg Asn Asp Ile Ala His Leu Ser His Glu Pro Leu Phe
Glu Cys Pro Leu Asp Gly Lys Lys Ser Tyr Lys Leu Lys Gln Gly Lys Arg Thr
Asn Thr Ile Asn Val Lys Pro Leu Pro Ile Ser Arg Lys Met Ile Val Asp Phe Ile
Ser Ser Gln Ser Asp Met Lys Lys Thr Leu Gly Tyr Asp Ala Val Asn Asp Leu
Thr Met Lys Ile Ile Gln Leu Arg Thr Arg Leu Lys Val Tyr Ala Asp Lys Ser Glu
Thr Ile Lys Thr Leu Val Asp Ala Ala Lys Thr Pro Asn Asp Phe Tyr His Ile Tyr
Lys Val Lys Gly Val Glu Ala Ile Asn Arg His Leu Leu Glu Val Ile Gly Glu Thr
Lys Asp Glu Lys Arg Ile Arg Lys Arg Ile Glu Ser Gly Asn Ala Ile Ala Gly Arg
Thr Pro Ala Asp Ser Gln Glu Asn

As described herein, substitutions may be made to this sequence to generate the inventive endonuclease (including, taking into account degeneracy of the genetic code).

In some embodiments, the endonuclease has one or more substitutions at positions corresponding to D38X, A59X, G172X, T236X, T319X, H375X, H419X, T424X, E529X, T541X, G562X, K564X, D569X, A586X, N641X, D642X, S647X, D721X, R779X, K13X, K566X, G554X, A35X, E110X, G314X, K114X, D498X, I86X, V57X, H249X, R704X of SEQ ID NO: 3, wherein the substitution is defined by X and wherein X is any amino acid. In some embodiments, X is an essential or non-essential amino acid.

In some embodiments, X is a hydrophilic or hydrophobic amino acid.

In some embodiments, X is a hydrophilic amino acid.

In some embodiments, X is a polar and positively charged hydrophilic amino acid. In some embodiments, X is selected from arginine I or lysine (K).

In some embodiments, X is a polar and neutral charged hydrophilic amino acid. In some embodiments, X is selected from asparagine (N), glutamine (Q), serine (S), threonine (T), proline (P), and cysteiI(C).

In some embodiments, X is a polar and negatively charged hydrophilic amino acid. In some embodiments, X is selected from aspartate (D) or glutIte (E).

In some embodiments, wherein X is an aromatic, polar and positively charged hydrophilic amino acid. In some embodiments, wherein X is histidine (H).

In some embodiments, X is a hydrophobic amino acid.

In some embodiments, X is a hydrophobic, aliphatic amino acid. In some embodiments, X is selected from glycine (G), alanine (A), leucine (L), isoleucine (I), methionine (M), or valine (V).

In some embodiments, X is a hydrophobic, aromatic amino acid. In some embodiments, wherein X is selected from phenylalanine (F), tryptophan (W), or tyrosine (Y).

In some embodiments, the endonuclease of SEQ ID NO: 3 comprises one or more of the following substitutions:
a hydrophilic residue other than aspartate (D) at a position corresponding to 38;
a hydrophobic residue other than alanine (A) at a position corresponding to 59;
a hydrophobic residue other than glycine (G) at a position corresponding to 172;
a hydrophilic residue other than threonine (T) at a position corresponding to 236;
a hydrophilic residue other than threonine (T) at a position corresponding to 319;
a hydrophilic residue other than histidine (H) at a position corresponding to 375;
a hydrophilic residue other than histidine (H) at a position corresponding to 419;
a hydrophilic residue other than threonine (T) at a position corresponding to 424;
a hydrophilic residue other than gIamate (E) at a position corresponding to 529;
a hydrophilic residue other than threonine (T) at a position corresponding to 541;
a hydrophobic residue other than glycine (G) at a position corresponding to 562;
a hydrophilic residue other than lysine (K) at a position corresponding to 564;
a hydrophilic residue other than aspartate (D) at a position corresponding to 569;
a hydrophobic residue other than alanine (A) at a position corresponding to 586;
a hydrophilic residue other than asparagine (N) at a position corresponding to 641;
a hydrophilic residue other than aspartate (D) at a position corresponding to 642;
a hydrophilic residue other than serine (S) at a position corresponding to 647;
a hydrophilic residue other than aspartate (D) at a position corresponding to 721;
a hydrophilic residue other thIarginine (R) at a position corresponding to 779;
a hydrophilic residue other than lysine (K) at a position corresponding to 13;
a hydrophilic residue other than lysine (K) at a position corresponding to 566;
a hydrophobic residue other than glycine (G) at a position corresponding to 554;
a hydrophobic residue other than alanine (A) at a position corresponding to 35;
a hydrophilic residue other In glutamate (E) at a position corresponding to 110;
a hydrophobic residue other than glycine (G) at a position corresponding to 314;
a hydrophilic residue other than lysine (K) at a position corresponding to 114;
a hydrophilic residue other than aspartate (D) at a position corresponding to 498;
a hydrophobic residue other than isoleucine (I) at a position corresponding to 86;
a hydrophobic residue other than valine (V) at a position corresponding to 57;
a hydrophilic residue other than histidine (H) at a position corresponding to 249; and
a hydrophilic residue otI than arginine (R) at a position corresponding to 704.

In some embodiments, the endonuclease of SEQ ID NO: 3 comprises D38F. In some embodiments, the endonuclease of SEQ ID NO: 3 comprises A59V. In some embodiments, the endonuclease of SEQ ID NO: 3 comprises G172L. In some embodiments, the endonuclease of SEQ ID NO: 3 comprises T236L. In some embodiments, the endonuclease of SEQ ID NO: 3 comprises T319I. In some embodiments, the endonuclease of SEQ ID NO: 3 comprises H375L. In some embodiments, the endonuclease of SEQ ID NO: 3 comprises H419Y. In some embodiments, the endonuclease of SEQ ID NO: 3 comprises T424F. In some embodiments, the endonuclease of SEQ ID NO: 3 comprises E529L. In some embodiments, the endonuclease of SEQ ID NO: 3 comprises T541L. In some embodiments, the endonuclease of SEQ ID NO: 3 comprises G562Y. In some embodiments, the endonuclease of SEQ ID NO: 3 comprises K564M. In some embodiments, the endonuclease of SEQ ID NO: 3 comprises D569L. In some embodiments, the endonuclease of SEQ ID NO: 3 comprises A586I. In some embodiments, the endonuclease of SEQ ID NO: 3 comprises N641F. In some embodiments, the endonuclease of SEQ ID NO: 3 comprises D642L. In some embodiments, the endonuclease of SEQ ID NO: 3 comprises S647L. In some embodiments, the endonuclease of SEQ ID NO: 3 comprises D721L. In some embodiments, the endonuclease of SEQ ID NO: 3 comprises R779I. In some embodiments, the endonuclease of SEQ ID NO: 3 comprises K13R. In some embodiments, the endonuclease of SEQ ID NO: 3 comprises K566R. In some embodiments, the endonuclease of SEQ ID NO: 3 comprises G554H. In some embodiments, the endonuclease of SEQ ID NO: 3 comprises A35N. In some embodiments, the endonuclease of SEQ ID NO: 3 comprises E110T. In some embodiments, the endonuclease of SEQ ID NO: 3 comprises G314Q. In some embodiments, the endonuclease of SEQ ID NO: 3 comprises K114P. In some embodiments, the endonuclease of SEQ ID NO: 3 comprises D498P. In some embodiments, the endonuclease of SEQ ID NO: 3 comprises I86P. In some embodiments, the endonuclease of SEQ ID NO: 3 comprises V57E. In some embodiments, the endonuclease of SEQ ID NO: 3 comprises H249W. In some embodiments, the endonuclease of SEQ ID NO: 3 comprises R704F.

In some embodiments, the endonuclease of SEQ ID NO: 3 comprises D38F and A59V. In some embodiments, the endonuclease of SEQ ID NO: 3 comprises D38F, A59V, and G172L. In some embodiments, the endonuclease of SEQ ID NO: 3 comprises D38F, A59V, G172L, and T236L. In some embodiments, the endonuclease of SEQ ID NO: 3 comprises D38F, A59V, G172L, T236L, and T319I. In some embodiments, the endonuclease of SEQ ID NO: 3 comprises D38F, A59V, G172L, T236L, T319I, and H375L. In some embodiments, the endonuclease of SEQ ID NO: 3 comprises D38F, A59V, G172L, T236L, T319I, H375L, and H419Y. In some embodiments, the endonuclease of SEQ ID NO: 3 comprises D38F, A59V, G172L, T236L, T319I, H375L, H419Y, and T424F. In some embodiments, the endonuclease of SEQ ID NO: 3 comprises D38F, A59V, G172L, T236L, T319I, H375L, H419Y, T424F, and E529L. In some embodiments, the endonuclease of SEQ ID NO: 3 comprises D38F, A59V, G172L, T236L, T319I, H375L, H419Y, T424F, E529L, and T541L. In some embodiments, the endonuclease of SEQ ID NO: 3 comprises D38F, A59V, G172L, T236L, T319I, H375L, H419Y, T424F, E529L, T541L, and G562X. In some embodiments, the endonuclease of SEQ ID NO: 3 comprises D38F, A59V, G172L, T236L, T319I, H375L, H419Y, T424F, E529L, T541I, G562X, and K564M. In some embodiments, thI endonuclease of SEQ ID NO: 3 comprises D38F, A59V, G172L, T236L, T319I, H375L, H419Y, T424F, E529L, T541L, G562X, K564M, and D569L. In some embodiments, the endonuclease of SEQ ID NO: 3 comprises D38F, A59V, G172L, T236L, T319I, H375L, H419Y, T424F, E529L, T541L, G562X, K564M, D569L, and A586I. In some embodiments, the endonuclease of SEQ ID NO: 3 comprises D38F, A59V, G172L, T236L, T319I, H375L, H419Y, T424F, E529L, T541L, G562X, K564M, D569L, A586I, and N641F. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, and D642L. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, and S647L. In some embodiments, the endonuclease of SEQ ID NO: 3 comprises D38F, A59V, G172L, T236L, T319I, H375L, H419Y, T424F, E529L, T541L, G562X, aK564M, D569L, A586I, N641F, D642L, S647L, and D721L. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, and R779I. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, and K13R. In some embodiments, the endonuclease of SEQ ID NO: 3 comprises D38F, A59V, G172L, T236L, T319I, H375L, H419Y, T424F, E529L, T541L, G562X, aK564M, D569L, A586I, N641F, D642L, S647L, D721L, R779I, K13R, and K566R. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, K13R, K566R, and G554H. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, K13R, K566R, G554H, and A35N. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, K13R, K566R, G554H, and A35N. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, K13R, K566R, G554H, A35N, and E110T. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, K13R, K566R, G554H, A35N, E110T, and G314Q. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, K13R, K566R, G554H, A35N, E110T, G314Q, and K114P. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, K13R, K566R, G554H, A35N, E110T, G314Q, K114P, and D498P. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, K13R, K566R, G554H, A35N, E110T, G314Q, K114P, D498P, and 186P. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, K13R, K566R, G554H, A35N, E110T, G314Q, K114P, D498P, I86P, and V57E. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, K13R, K566R, G554H, A35N, E110T, G314Q, K114P, D498P, I86P, V57E, and H249W. In some embodiments, the endonuclease of SEQ ID NO: 3 comprises D38F, A59V, G172L, T236L, T319I, H375L, H419Y, T424F, E529L, T541L, G562X, K564M, D569L, A586I, N641F, D642L, S647L, D721L, R779I, K13R, K566R, G554H, A35N, E110T, G314Q, K114P, D498P, I86P, V57E, H249W, and R704F.

In some embodiments, the disclosed herein are one or more (e.g. about 1, or about 2, or about 3, or about 4, or about 5, or about 6, or about 7, or about 8, or about 9, or about 10, or about 15, or about 20, or about 30) substitutions to SEQ ID NO: 3 or a sequence with at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, 99.8, 99.9% identity to SEQ ID NO: 3 (or about 70%, or about 75%, or about 80%, or about 85%, or about 90, or about 95%, or about 96%, or about 97%, or about 98%, or about 99% identity to SEQ ID NO: 3). In various embodiments, one or more amino acids of SEQ ID NO: 3 is substituted with a naturally occurring amino acid, such as a hydrophilic amino acid (e.g. a polar and positively charged hydrophilic amino Id, such as arginine (R) or lysine (K); a polar and neutral of charge hydrophilic amino acid, such as asparagine (N), glutamine (Q), serine (S), threonine (T), Iline (P), and cysteine (C), a polar and negatively charged hydrophilic amino acid, such alspartate (D) or glutamate (E), or an aromatic, polar and positively charged hydrophilic amino acid, such as histidine (H)) or a hydrophobic amino acid (e.g. a hydrophobic, aliphatic amino acid such as glycine (G), alanine (A), leucine (L), isoleucine (I), methionine (M), or valine (V), a hydrophobic, aromatic amino acid, such as phenylalanine (F), tryptophan (W), or tyrosine (Y) or a non-classical amino acid (e.g. selenocysteine, pyrrolysine, N-formylmethionine β-alanine, GABA and δ-Aminolevulinic acid. 4-Aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

In illustrative embodiments, inventive substitutions include, but are not limited to one or more (e.g. about 1, or about 2, or about 3, or about 4, or about 5, or about 6, or about 7, or about 8, or about 9, or about 10, or about 15, or about 20, or about 30) substitutions to SEQ ID NO: 3, or a sequence with at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, 99.8, 99.9% identity to SEQ ID NO: 3: D38F, A59V, G172L, T236L, T319I, H375L, H419Y, T424F, E529L, T541L, G562X, K564M, D569L, A586I, N641F, D642L, S647L, D721L, R779I, K13R, K566R, G554H, A35N, E110T, G314Q, K114P, D498P, I86P, V57E, H249W, and R704F.

In embodiments, the endonuclease disclosed herein (e.g., SEQ ID NO: 1, SEQ ID NO: 2, SEQ ID NO: 3, and SEQ ID NO: 4, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, or fragments or variants thereof), comprises a bi-lobed structure. In embodiments, the endonuclease comprises two HEPN domains in the Nuclease (NUC) lobe for cleavage of RNA targets, and a Recognition (REC) lobe comprising α-helical domains and an N-terminal domain (NTD). In embodiments, the REC lobe binds the CRISPR repeat of the gRNA. In embodiments, the NTD comprises one or more R sheets.

In embodiments, the endonuclease comprises a C-terminal domain (CTD) in an NUC lobe.

In embodiments, the endonuclease and/or system for targeting a nucleic acid for trans-splicing comprises an inactive endonuclease (dCas). In embodiments, the dCas is a variant of Cas13e3. In embodiments, the Cas13e3 comprises R244A/H249A/R704A/H709A mutations in the HEPN domains or mutations corresponding thereto relative to SEQ ID NO: 3.

In embodiments, disclosed herein is a miniature splice editor. In embodiments, the miniature splice editor is based on dCas13e3. In embodiments, the miniature splice editor is at least about 5%, at least about 10%, at least about 15%, at least about 20%, or at least about 25% shorter in sequence length compared to SE1 (SE1 is fused, linked or associated with dPspCas13b-MCP).

In embodiments, the endonuclease (or chimeric protein) comprises a domain from a different endonuclease. In embodiments, the different endonuclease is a Cas endonuclease. In embodiments, the domain is one or more of a PAM-interacting domain. In embodiments, the domain is derived from one or more of Cas9, Cas12a (Cpf1), Cas12e (CasX), Cas12d (CasY), Cas12b (C2c1), Cas13a (C2c2), Cas13b, Cas13c, Cas13d, Cas13X/Cas13bt, Cas13Y, Cas12c (C2c3), GeoCas9, CjCas9, NmeCas9, Cas12J (CasPhi), Cas12L (CasLambda), Cas12f (Cas14), Cas12g, Cas12h, Cas12i, Cas12k, NmeCas9, Nme2Cas9, CjCas9, GeoCas9, BlatCas9, PpCas9, and Cas14. In embodiments, the domain is derived from a Cas from one or more of Streptococcus pyogenes, Staphylococcus aureus, Neisseria meningitis, Streptococcus thermophilis, or Treponema denticola.

In embodiments, the composition further comprises one or more donor polynucleotides. In embodiments, the endonuclease is suitable for introducing one or more donor polynucleotides into a target nucleic acid molecule. In embodiments, the donor polynucleotide comprises a transgene. In embodiments, the donor polynucleotide comprises a sequence which corrects a mutation. In embodiments, a donor polynucleotide is of any length, for example between about 2 and about 10000 nucleotides in length (or any integer value therebetween or thereabove), for instance, between about 100 and about 1000 nucleotides in length (or any integer therebetween), or between about 200 and about 500 nucleotides in length.

In embodiments, the endonuclease (or chimeric protein) comprises a nuclear localization signal (NLS). Examples of NLSs are provided in Kosugi et al. (J. Biol. Chem. (2009) 284:478-485; hereby incorporated by reference herein). In embodiments, the NLS comprises the consensus sequence K(K/R)X(K/R) (SEQ ID NO: 32). In embodiments, the NLS comprises the consensus sequence (K/R)(K/R)X₁₀₋₁₂(K/R)_{3/5} (SEQ ID NO: 33), where (K/R)_{3/5} represents at least three of the five amino acids is either lysine or arginine. In embodiments, the NLS comprises the c-myc NLS. In embodiments, the c-myc NLS comprises the sequInce PAAKRVKLD (SEQ ID NO: 34). In embodiments, tIe NLS is the nucleoplasmin NLS. In embodiments, the nucleoplasmin NLS comprises the sequence KRPAATKKAGQAKKKK (SEQ ID NO: 35). In embodiments, the NLS comprises the SV40 Large T-antigen NLS. In embodiments, the SV40 Large T-antigen NLS comprises the sequence PKKKRKV (SEQ ID NO: 36). In a particular embodiment, the NLS comprises three SV40 Large T-antigen NLSs (e.g., DPKKKRKVDPKKKRKVDPKKKRKV (SEQ ID NO: 37)). In embodiments, the NLS is or comprises SEQ ID NO: 72. In embodiments, the NLS comprises mutations/variations in the above sequences such that they contain 1 or more substitutions, additions, or deletions (e.g., about 1, or about 2, or about 3, or about 4, or about 5, or about 10 substitutions, additions, or deletions).

In embodiments, the endonuclease (or chimeric protein) comprises a polypeptide permeant domain to promote uptake by a cell. In embodiments, the permeant domain is a peptide, peptidomimetic, or non-peptide carrier. In embodiments, the permeant peptide is derived from the third alpha helix of Drosophila melanogaster transcription factor Antennapaedia, referred to as penetratin, which comprises the amino acid sequence RQIKIWFQNRRMKWKK (SEQ ID NO: 38). In embodiments, the permeant peptide comprises the HIV-1 tat basic region amino acid sequence, which may include, for example, amino acids 49-57 of naturally occurring tat protein. In embodiments, the permeant peptide is a poly-arginine motif, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-9 and 446; and Wender et al. (2000) Proc. Natl. Acad. Sci. U.S.A 2000 Nov. 21; 97(24):13003-8; U.S. Patent Pub. 2003/0220334; 2003/0083256; 2003/0032593; and 2003/0022831, herein incorporated by reference in their entireties).

In embodiments, the endonuclease (or chimeric protein) comprises a polypeptide that promotes or is suitable for VLP delivery, including, without limitation, a retroviral gag polyprotein comprising a matrix polypeptide, a capsid polypeptide, and a nucleocapsid polypeptide (optionally with one or more heterologous protease cleavage sites (e.g. TEV cleavage site, a PreScission (fusion protein of glutathione S-transferase (GST) and human rhinovirus (HRV) type 14 3C protease) cleavage site, a human rhinovirus 3C protease cleavage site, an enterokinase cleavage site, an Epstein-Barr virus protease cleavage site, a cathepsin D cleavage site, and/or a thrombin cleavage site) between one or both of: the matrix polypeptide and the capsid polypeptide; and the capsid polypeptide and the nucleocapsid polypeptide, e.g., a lentiviral gag polyprotein, e.g., a bovine immunodeficiency virus gag polyprotein, a murine leukemia virus (MLV) a gag protein, a simian immunodeficiency virus gag polyprotein, a feline immunodeficiency virus gag polyprotein, a human immunodeficiency virus gag polyprotein, an equine infection anemia virus gag polyprotein, and a caprine arthritis encephalitis virus gag polyprotein or a gag polyprotein of an alpha retrovirus, a beta retrovirus, a gamma retrovirus, a delta retrovirus, an epsilon retrovirus, or a spumavirus. In embodiments, the polypeptide that promotes or is suitable for VLP delivery is co-delivered with a protease to promote cleavage of the chimeric protein. In embodiments, the cleavage of the chimeric protein occurs between the endonuclease and the polypeptide that promotes or is suitable for VLP delivery. In embodiments, the protease is fused to a polypeptide that promotes or is suitable for VLP delivery.

In embodiments, the endonuclease (or chimeric protein) or a delivery vehicle, e.g. one or more lipids, associated therewith comprises a polypeptide or other moiety that interacts with an targeting moiety, e.g. an antibody or antibody-like molecule, a ligand to bind a receptor or a receptor (or fragment thereof) to bind a ligand, aptamer, and the like, for targeted delivery. In embodiments, the endonuclease (or chimeric protein) or a delivery vehicle, e.g. one or more lipids, associated therewith comprises a targeting moiety, e.g., an antibody or antibody-like molecule, a ligand to bind a receptor or a receptor (or fragment thereof) to bind a ligand, aptamer, and the like, for targeted delivery.

In embodiments, the endonuclease is suitable for introducing one or more excisions into a target nucleic acid molecule. In embodiments, the excision is a double-strand DNA break in two strands of the target nucleic acid molecule. In embodiments, the excision is a nick in one or more strands of the target nucleic acid molecule.

### Chimeric Constructs/Nucleic Acid Modulating/Modifying Domains

In aspects, the present disclosure provides a composition comprising a chimeric protein comprising: an endonuclease comprising a sequence, optionally comprising a HEPN domain, or a fragment or variant thereof, and having at least about 70% (or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%) to one or more of SEQ ID NOs: 1-4, and/or SEQ ID NOs: 80-89, or having about 1 to about 20 amino acid modifications (e.g. about 1, or about 2, or about 3, or about 4, or about 5, or about 6, or about 7, or about 8, or about 9, or about 10, or about 11, or about 12, or about 13, or about 14, or about 15, or about 16, or about 17, or about 18, or about 19, or about 20 modifications); and a nucleic acid-modulating domain or a nucleic acid-modifying domain comprising a sequence comprising a catalytic domain, or a fragment or variant thereof, wherein (a) and (b) do not naturally occur together in a same reading frame.

In embodiments, the endonuclease reduces or enhances non-specific degradation of transcripts. In embodiments, the endonuclease reduces or enhances collateral activity, e.g., for use in nucleic acid detection. In embodiments, the endonuclease reduces or enhances collateral activity, e.g. for use in nucleic acid detection using an electrochemical method.

In embodiments, the nucleic acid-modulating domain or the nucleic acid-modifying domain has one or more of nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, debranching activity, transesterification activity, photolyase activity and glycosylase activity. In embodiments, the nucleic acid-modulating domain or the nucleic acid-modifying domain is a METTL3 methyltransferase domain, a METTL3: METTL1 fusion domain, or a fragment or variant thereof.

In embodiments, the nucleic acid-modulating domain or the nucleic acid-modifying domain is a nucleic acid interacting/binding domain. Non-limiting examples of nucleic acid interacting/binding domains are MCP, lambdaN, PP7, QBeta, SLBP, and TBP/TAR.

In embodiments, the nucleic acid-modulating domain or the nucleic acid-modifying domain is a splice-modulating domain. In embodiments, the splice-modulating domain is the RS-rich domain of SRSF1, the Gly-rich domain of hnRNP A1, the alanine-rich motif of RBM4, or the proline-rich motif of DAZAP1. In embodiments, the endonuclease (or chimeric protein) induces exon skipping. In embodiments, the endonuclease (or chimeric protein) induces exon inclusion.

In embodiments, the nucleic acid-modulating domain or the nucleic acid-modifying domain is a degradation domain. In embodiments, the degradation domain is a E2 ubiquitin or ubiquitin-like domain. In embodiments, the degradation domain comprises a ubiquitin core catalytic (UBC) domain. In embodiments, the degradation domain is SUMO, NEDD8, ATG8, ATG12, ISG15, UFM1, FAT10, URM1, or FUB1, or a fragment or variant thereof. In embodiments, the degradation domain is UBE2A (hHR6A), UBE2B (hHR6B), UBE2C (UbcH10), UBE2D1 (UbcH5A), UBE2D2 (UbcH5B), UBE2D3 (UbcH5C), UBE2D4 (HBUCEl), UBE2E1 (UbcH6), UBE2E2, UBE2E3 (UbcH9), UBE2F (NCE2), UBE2G1 (UBE2G), UBE2G2 (UBC7), UBE2H (UBCH), UBE2I (Ubc9), UBE2J1 (NCUBE1), UBE2J2 (NCUBE2), UBE2K (HIP2), UBE2L3 (UbcH7), UBE2L6 (UbcH8), UBE2M (Ubcl2), UBE2N (Ubcl3), UBE2NL, UBE20 (E2-230K), UBE2Q1 (NICE-5), UBE2Q2, UBE2QL, UBE2R1 (CDC34), UBE2R2 (CDC34B), UBE2S (E2-EPF), UBE2T (HSPC150), UBE2U, UBE2V1 (UEV-1A), UBE2V2 (MMS2), UBE2W, UBE2Z (Use1), UVELD (UEV3), BIRC6 (apollon), FTS (AKTIP), TSG101, or UFC1, or a fragment or variant thereof. In embodiments, the degradation domain is cereblon (CRBN) E3 ligase. In embodiments, the degradation domain is modulated by a proteolysis-targeting chimeras (PROTAC).

In embodiments, the degradation domain is a protease, e.g. a protease that is conditionally modulated by another molecule, e.g. a protease inhibitor, e.g. a matrix metalloprotease (MMP) and TIMP-1, TIMP-2, TIMP-3, or TIMP-4.

In embodiments, the degradation domain is modulated by a small molecule. In embodiments, the degradation domain is active in the presence of a small molecule. In embodiments, the degradation domain is inactive in the presence of a small molecule. In embodiments the small molecule is an antiviral drug. In embodiments the small molecule is one or more of abscisic acid (ABA), rapamycin (or rapalog), FK506, Cyclosporine A, FK1012, Gibberellin3-AM, FKCsA, AP1903/AP20187, and auxin.

In embodiments, the nucleic acid-modulating domain or the nucleic acid-modifying domain has nuclease activity, such as that provided by a restriction enzyme (e.g., FokI nuclease).

In embodiments, the nucleic acid-modulating domain or the nucleic acid-modifying domain has methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (e.g. plants), ZMET2, CMT1, CMT2 (e.g. plants), and the like).

In embodiments, the nucleic acid-modulating domain or the nucleic acid-modifying domain has demethylase activity such as that provided by a demethylase (e.g., Ten-Eleven Translocation (TET) dioxygenase 1 (TET1CD), TET1, DME, DML1, DML2, ROS1, and the like).

In embodiments, the nucleic acid-modulating domain or the nucleic acid-modifying domain has deamination activity such as that provided by a deaminase (e.g., a cytosine deaminase enzyme such as rat APOBEC1).

In embodiments, the nucleic acid-modulating domain or the nucleic acid-modifying domain has integrase and/or resolvase activity (e.g., Gin invertase such as the hyperactive mutant of the Gin invertase, GinH106Y; human immunodeficiency virus type 1 integrase (IN); Tn3 resolvase; and the like).

In embodiments, the nucleic acid-modulating domain or the nucleic acid-modifying domain has recombinase activity such as that provided by a recombinase (e.g., catalytic domain of Gin recombinase).

In embodiments, the nucleic acid-modulating domain or the nucleic acid-modifying domain is selected from a deaminase, reverse transcriptase, transposase, integrase, and recombinase. In embodiments, the deaminase is a cytidine or cytosine deaminase, or a fragment or variant thereof. In embodiments, the cytidine or cytosine deaminase is selected from AID, CDA1, and APOBEC, or a fragment or variant thereof. In embodiments, the APOBEC is selected from A3A, AB33, APOBEC1, APOBEC3C, APOBEC31D, APOBEC31F, APOBEC3G, and APOBEC3H, or a fragment or variant thereof. In embodiments, the APOBEC has an amino acid sequence of one of SEQ ID NO: 39 [A3A], SEQ ID NO: 40 [AB3], SEQ ID NO: 41 [APOBEC1], SEQ ID NO: 42 [APOBEC3C], SEQ ID NO: 43 [APOBEC3D], SEQ ID NO: 44 [APOBEC3F], SEQ ID NO: 45 [APOBEC3G], and SEQ ID NO: 46 [APOBEC3H], or a fragment or variant thereof, or an amino acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto. In embodiments, the APOBEC has an amino acid sequence selected from one of SEQ ID NOs: 32-39 as shown in Table 5 below.

TABLE 5
APOBEC amino acid sequences

| | SEQ |
|---|---|
| Sequence | ID NO: |
| MDPHIFTSNFNNGIGRHKTYLCYEVERLDNGTSVKMDQHRG | 39 |
| FLHNQAKNLLCGFYGRHAELRFLDLVPSLQLDPAQIYRVTW | |
| FISWSPCFSWGCAGEVRAFLQENTHVRLRIFAARIYDYDPL | |
| YKEALQMLRDAGAQVSIMTYDEFKHCWDTFVDHQGCPFQPW | |
| DGLDEHSQALSGRLRAILQNQGN | |
| MDPDTFTFNFNNDPLVLRRRQTYLCYEVERLDNGTWVLMDQ | 40 |
| HMGFLCNEAKNLLCGFYGRHAELRFLDLVPSLQLDPAQIYR | |
| VTWFISWSPCFSWGCAGEVRAFLQENTHVRLRIFAARIYDY | |
| DPLYKEALQMLRDAGAQVSIMTYDEFEYCWDTFVYRQGCPF | |
| QPWDGLEEHSQALSGRLRAILQNQGN | |
| MTSEKGPSTGDPTLRRRIEPWEFDVFYDPRELRKEACLLYE | 41 |
| IKWGMSRKIWRSSGKNTTNHVEVNFIKKFTSERDFHPSMSC | |
| SITWFLSWSPCWECSQAIREFLSRHPGVTLVIYVARLFWHM | |
| DQQNRQGLRDLVNSGVTIQIMRASEYYHCWRNFVNYPPGDE | |
| AHWPQYPPLWMMLYALELHCIILSLPPCLKISRRWQNHLTF | |
| FRLHLQNCHYQTIPPHILLATGLIHPSVAWR | |
| MNPQIRNPMKAMYPGTFYFQFKNLWEANDRNETWLCFTVEG | 42 |
| IKRRSVVSWKTGVFRNQVDSETHCHAERCFLSWFCDDILSP | |
| NTKYQVTWYTSWSPCPDCAGEVAEFLARHSNVNLTIFTARL | |
| YYFQYPCYQEGLRSLSQEGVAVEIMDYEDFKYCWENFVYND | |
| NEPFKPWKGLKTNFRLLKRRLRESLQ | |
| MNPQIRNPMERMYRDTFYDNFENEPILYGRSYTWLCYEVKI | 43 |
| KRGRSNLLWDTGVFRGPVLPKRQSNHRQEVYFRFENHAEMC | |
| FLSWFCGNRLPANRRFQITWFVSWNPCLPCVVKVTKFLAEH | |
| PNVTLTISAARLYYYRDRDWRWVLLRLHKAGARVKIMDYED | |
| FAYCWENFVCNEGQPFMPWYKFDDNYASLHRTLKEILRNPM | |
| EAMYPHIFYFHFKNLLKACGRNESWLCFTMEVTKHHSAVFR | |
| KRGVFRNQVDPETHCHAERCFLSWFCDDILSPNTNYEVTWY | |
| TSWSPCPECAGEVAEFLARHSNVNLTIFTARLCYFWDTDYQ | |
| EGLCSLSQEGASVKIMGYKDFVSCWKNFVYSDDEPFKPWKG | |
| LQTNFRLLKRRLREILQ | |
| MKPHFRNTVERMYRDTFSYNFYNRPILSRRNTVWLCYEVKT | 44 |
| KGPSRPRLDAKIFRGQVYSQPEHHAEMCFLSWFCGNQLPAY | |
| KCFQITWFVSWTPCPDCVAKLAEFLAEHPNVTLTISAARLY | |
| YYWERDYRRALCRLSQAGARVKIMDDEEFAYCWENFVYSEG | |
| QPFMPWYKFDDNYAFLHRTLKEILRNPMEAMYPHIFYFHFK | |
| NLRKAYGRNESWLCFTMEVVKHHSPVSWKRGVFRNQVDPET | |
| HCHAERCFLSWFCDDILSPNTNYEVTWYTSWSPCPECAGEV | |
| AEFLARHSNVNLTIFTARLYYFWDTDYQEGLRSLSQEGASV | |
| EIMGYKDFKYCWENFVYNDDEPFKPWKGLKYNFLFLDSKLQ | |
| EILE | |
| MKPHFRNTVERMYRDTFSYNFYNRPILSRRNTVWLCYEVKT | 45 |
| KGPSRPPLDAKIFRGQVYSELKYHPEMRFFHWFSKWRKLHR | |
| DQEYEVTWYISWSPCTKCTRDMATFLAEDPKVTLTIFVARL | |
| YYFWDPDYQEALRSLCQKRDGPRATMKIMNYDEFQHCWSKF | |
| VYSQRELFEPWNNLPKYYILLHIMLGEILRHSMDPPTFTFN | |
| FNNEPWVRGRHETYLCYEVERMHNDTWVLLNQRRGFLCNQA | |
| PHKHGFLEGRHAELCFLDVIPFWKLDLDQDYRVTCFTSWSP | |
| CFSCAQEMAKFISKNKHVSLCIFTARIYDDQGRCQEGLRTL | |
| AEAGAKISIMTYSEFKHCWDTFVDHQGCPFQPWDGLDEHSQ | |
| DLSGRLRAILQNQEN | |
| MALLTAETFRLQFNNKRRLRRPYYPRKALLCYQLTPQNGST | 46 |
| PTRGYFENKKKCHAEICFINEIKSMGLDETQCYQVTCYLTW | |
| SPCSSCAWELVDFIKAHDHLNLGIFASRLYYHWCKPQQKGL | |
| RLLCGSQVPVEVMGFPEFADCWENFVDHEKPLSFNPYKMLE | |
| ELDKNSRAIKRRLERIKQS | |

In embodiments, the deaminase is a DNA-specific adenine or adenosine deaminase or adenosine base editor (ABE), or a fragment or variant thereof.

In embodiments, the ABE is generated by replacing APOBEC1 component of BE3 with natural or engineered E. coli TadA, human ADAR2, mouse ADA, or human ADAT2. In embodiments, ABE comprises an evolved TadA variant. In embodiments, the ABE is ABE 1.2 (TadA*-XTEN-nCas9-NLS). In some embodiments, TadA* comprises A106V and D108N mutations. In embodiments, the ABE is a second-generation ABE. In embodiments, the ABE is ABE2.1, which comprises additional mutations D147Y and E155V in TadA* (TadA*2.1). In embodiments, the ABE is ABE2.2, ABE2.1 fused to catalytically inactivated version of human alkyl adenine DNA glycosylase (AAG with E125Q mutation). In embodiments, the ABE is ABE2.3, ABE2.1 fused to catalytically inactivated version of E. coli Endo V (inactivated with D35A mutation). In embodiments, the ABE is ABE2.6 which has a linker twice as long (32 amino acids, (SGGS)₂-XTEN-(SGGS)₂ (“(SGGS)₂” disclosed as SEQ ID NO: 47)) as the linker in ABE2.1. In embodiments, the ABE is ABE2.7, which is ABE2.1 tethered with an additional wild-type TadA monomer. In embodiments, the ABE is ABE2.8, which is ABE2.1 tethered with an additional TadA*2.1 monomer. In embodiments, the ABE is ABE2.9, which is a direct fusion of evolved TadA (TadA*2.1) to the N-terminus of ABE2.1. In embodiments, the ABE is ABE2.10, which is a direct fusion of wild type TadA to the N-terminus of ABE2.1. In embodiments, the ABE is ABE2.11, which is ABE2.9 with an inactivating E59A mutation at the N-terminus of TadA* monomer. In embodiments, the ABE is ABE2.12, which is ABE2.9 with an inactivating E59A mutation in the internal TadA* monomer. In embodiments, the ABE is a third generation ABE. In embodiments, the ABE is ABE3.1, which is ABE2.3 with three additional TadA mutations (L84F, H123Y, and 1156F). In embodiments, the ABE is a fourth generation ABE. In embodiments, the ABE is ABE4.3, which is ABE3.1 with an additional TadA mutation A142N (TadA*4.3). In embodiments, the ABE is a fifth generation ABE. In embodiments, the ABE is ABE5.1, which is generated by importing a consensus set of mutations from surviving clones (H36L, R51L, S146C, and K157N) into ABE3.1. In embodiments, the ABE is ABE5.3, which has a heterodimeric construct containing wild-type E. coli TadA fused to an internal evolved TadA*. In embodiments, the ABE is ABE5.2, ABE5.4, ABE5.5, ABE5.6, ABE5.7, ABE5.8, ABE5.9, ABE5.10, ABE5.11, ABE5.12, ABE5.13, or ABE5.14, as shown in Table 6 of U.S. Pat. No. 11,142,760 (herein incorporated herein by reference). In embodiments, the ABE is a sixth generation ABE. In embodiments, the ABE is ABE6.1, ABE6.2, ABE6.3, ABI6.4, ABE6.5, or ABE6.6, as shown in below Table 6 of U.S. Pat. No. 11,142,760 (herein incorporated herein by reference). In embodiments, the ABE is a seventh generation ABE. In embodiments, the ABE is ABE7.1, ABE7.2, ABE7.3, ABE7.4, ABE7.5, ABE7.6, ABE7.7, ABE7.8, ABE 7.9, or ABE7.10, as shown in Table 6 of U.S. Pat. No. 11,142,760 (herein incorporated herein by reference). In embodiments, the base editor is an eighth generation ABE (ABE8). In s embodiments, the ABE8 contains a TadA*8 variant. In embodiments, the ABE8 has a monomeric construct containing a TadA*8 variant (ABE8.x-m).

In embodiments, the DNA-specific adenine or adenosine deaminase is selected from tRNA-specific adenosine deaminase 7.10 (TadA 7.10), tRNA-specific adenosine deaminase 6.3 (TadA 6.3), tRNA-specific adenosine deaminase 7.8 (TadA 7.8), tRNA-specific adenosine deaminase 7.9 (TadA 7.9), and tRNA-specific adenosine deaminase 8e (TadA8e (TadA-8e V106W)) or a fragment or variant thereof. In embodiments, the TadA has an amino acid sequence of one of SEQ ID NO: 48 [TadA 7.10], SEQ ID NO: 49 [TadA 6.3], SEQ ID NO: 50 [TadA 7.8], SEQ ID NO: 51 [TadA 7.9], and SEQ ID NO: 52 [TadA 8e], or a fragment or variant thereof, or an amino acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto. In embodiments, the TadA has an amino acid sequence selected from one of SEQ ID NOs: 48-52 as shown in Table 6 below.

TABLE 6
TadA amino acid sequences

| | SEQ |
|---|---|
| Sequence | ID NO: |
| SEVEFSHEYWMRHALTLAKRARDEREVPVGAVLVLNNRVIG | 48 |
| EGWNRAIGLHDPTAHAEIMALRQGGLVMQNYRLIDATLYVT | |
| FEPCVMCAGAMIHSRIGRVVFGVRNAKTGAAGSLMDVLHYP | |
| GMNHRVEITEGILADECAALLCYFFRMPRQVFNAQKKAQSS | |
| TD | |
| MSEVEFSHEYWMRHALTLAKRAWDEREVPVGAVLVLNNRVI | 49 |
| GEGWNRSIGLHDPTAHAEIMALRQGGLVMQNYRLIDATLYV | |
| TFEPCVMCAGAMIHSRIGRVVFGVRNAKTGAAGSLMDVLHY | |
| PGMNHRVEITEGILADECAALLCYFFRMRRQVFNAQKKAQS | |
| STD | |
| MSEVEFSHEYWMRHALTLAKRALDEREVPVGAVLVLNNRVI | 50 |
| GEGWNRAIGLHDPTAHAEIMALRQGGLVMQNYRLIDATLYV | |
| TFEPCVMCAGAMIHSRIGRVVFGVRNAKTGAAGSLMDVLHY | |
| PGMNHRVEITEGILADECNALLCYFFRMRRQVFNAQKKAQS | |
| STD | |
| MSEVEFSHEYWMRHALTLAKRALDEREVPVGAVLVLNNRVI | 51 |
| GEGWNRAIGLHDPTAHAEIMALRQGGLVMQNYRLIDATLYV | |
| TFEPCVMCAGAMIHSRIGRVVFGVRNAKTGAAGSLMDVLHY | |
| PGMNHRVEITEGILADECNALLCYFFRMPRQVFNAQKKAQS | |
| STD | |
| MSEVEFSHEYWMRHALTLAKRARDEREVPVGAVLVLNNRVI | 52 |
| GEGWNRAIGLHDPTAHAEIMALRQGGLVMQNYRLIDATLYV | |
| TFEPCVMCAGAMIHSRIGRVVFGVRNSKRGAAGSLMNVLNY | |
| PGMNHRVEITEGILADECAALLCDFYRMPRQVFNAQKKAQS | |
| SIN | |

In embodiments, the deaminase is a RNA-specific adenine or adenosine deaminase, or a fragment or variant thereof. In embodiments, the RNA-specific adenine or adenosine deaminase is an adenosine deaminases acting on RNA (ADAR) enzyme, or a fragment or variant thereof. In embodiments, the ADAR is selected from ADAR1, ADAR2, and ADAR3, or a fragment or variant thereof. In embodiments, the ADAR has an amino acid sequence of one of SEQ ID NO: 53 [ADAR1] and SEQ ID NO: 54 [ADAR2], or a fragment or variant thereof, or an amino acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto. In embodiments, the ADAR has an amino acid sequence of any one of SEQ ID NOs 53-54 as shown in Table 7. In embodiments, and as a non-limiting example, the catalytic deaminase domain of ADAR1 comprises amino acids 833-1226 of SEQ ID NO: 53. As another non-limiting example the catalytic deaminase domain of ADAR2 comprises amino acids 299-701 of SEQ ID NO: 54.

TABLE 7
ADAR amino acid sequences

| | SEQ |
|---|---|
| Sequence | ID NO: |
| MNPRQGYSLSGYYTHPFQGYEHRQLRYQQPGPGSSPSSFLLK | 53 |
| QIEFLKGQLPEAPVIGKQTPSLPPSLPGLRPRFPVLLASSTR | |
| GRQVDIRGVPRGVHLRSQGLQRGFQHPSPRGRSLPQRGVDCL | |
| SSHFQELSIYQDQEQRILKFLEELGEGKATTAHDLSGKLGTP | |
| KKEINRVLYSLAKKGKLQKEAGTPPLWKIAVSTQAWNQHSGV | |
| VRPDGHSQGAPNSDPSLEPEDRNSTSVSEDLLEPFIAVSAQA | |
| WNQHSGVVRPDSHSQGSPNSDPGLEPEDSNSTSALEDPLEFL | |
| DMAEIKEKICDYLFNVSDSSALNLAKNIGLTKARDINAVLID | |
| MERQGDVYRQGTTPPIWHLTDKKRERMQIKRNTNSVPETAPA | |
| AIPETKRNAEFLTCNIPTSNASNNMVTTEKVENGQEPVIKLE | |
| NRQEARPEPARLKPPVHYNGPSKAGYVDFENGQWATDDIPDD | |
| LNSIRAAPGEFRAIMEMPSFYSHGLPRCSPYKKLTECQLKNP | |
| ISGLLEYAQFASQTCEFNMIEQSGPPHEPRFKFQVVINGREF | |
| PPAEAGSKKVAKQDAAMKAMTILLEEAKAKDSGKSEESSHYS | |
| TEKESEKTAESQTPTPSATSFFSGKSPVTTLLECMHKLGNSC | |
| EFRLLSKEGPAHEPKFQYCVAVGAQTFPSVSAPSKKVAKQMA | |
| AEEAMKALHGEATNSMASDNQPEGMISESLDNLESMMPNKVR | |
| KIGELVRYLNTNPVGGLLEYARSHGFAAEFKLVDQSGPPHEP | |
| KFVYQAKVGGRWFPAVCAHSKKQGKQEAADAALRVLIGENEK | |
| AERMGFTEVTPVTGASLRRTMLLLSRSPEAQPKTLPLTGSTF | |
| HDQIAMLSHRCFNTLTNSFQPSLLGRKILAAIIMKKDSEDMG | |
| VVVSLGTGNRCVKGDSLSLKGETVNDCHAEIISRRGFIRFLY | |
| SELMKYNSQTAKDSIFEPAKGGEKLQIKKTVSFHLYISTAPC | |
| GDGALFDKSCSDRAMESTESRHYPVFENPKQGKLRTKVENGE | |
| GTIPVESSDIVPTWDGIRLGERLRTMSCSDKILRWNVLGLQG | |
| ALLTHFLQPIYLKSVTLGYLFSQGHLTRAICCRVTRDGSAFE | |
| DGLRHPFIVNHPKVGRVSIYDSKRQSGKTKETSVNWCLADGY | |
| DLEILDGTRGTVDGPRNELSRVSKKNIFLLFKKLCSFRYRRD | |
| LLRLSYGEAKKAARDYETAKNYFKKGLKDMGYGNWISKPQEE | |
| KNFYLCPV | |
| MDIEDEENMSSSSTDVKENRNLDNVSPKDGSTPGPGEGSQLS | 54 |
| NGGGGGPGRKRPLEEGSNGHSKYRLKKRRKTPGPVLPKNALM | |
| QLNEIKPGLQYTLLSQTGPVHAPLFVMSVEVNGQVFEGSGPT | |
| KKKAKLHAAEKALRSFVQFPNASEAHLAMGRTLSVNTDFTSD | |
| QADFPDTLFNGFETPDKAEPPFYVGSNGDDSFSSSGDLSLSA | |
| SPVPASLAQPPLPVLPPFPPPSGKNPVMILNELRPGLKYDFL | |
| SESGESHAKSFVMSVVVDGQFFEGSGRNKKLAKARAAQSALA | |
| AIFNLHLDQTPSRQPIPSEGLQLHLPQVLADAVSRLVLGKFG | |
| DLTDNFSSPHARRKVLAGVVMTTGTDVKDAKVISVSTGTKCI | |
| NGEYMSDRGLALNDCHAEIISRRSLLRFLYTQLELYLNNKDD | |
| QKRSIFQKSERGGFRLKENVQFHLYISTSPCGDARIFSPHEP | |
| ILEEPADRHPNRKARGQLRTKIESGEGTIPVRSNASIQTWDG | |
| VLQGERLLTMSCSDKIARWNVVGIQGSLLSIFVEPIYFSSII | |
| LGSLYHGDHLSRAMYQRISNIEDLPPLYTLNKPLLSGISNAE | |
| ARQPGKAPNFSVNWTVGDSAIEVINATTGKDELGRASRLCKH | |
| ALYCRWMRVHGKVPSHLLRSKITKPNVYHESKLAAKEYQAAK | |
| ARLFTAFIKAGLGAWVEKPTEQDQFSLTP | |

In embodiments, the deaminase further comprises a nuclear localization signal (NLS). Examples of NLSs are provided in Kosugi et al. (J. Biol. Chem. (2009) 284:478-485; herein incorporated by reference herein in its entirety). In embodiments, the NLS comprises the consensus sequence K(K/R)X(K/R) (SEQ ID NO: 32). In embodiments, the NLS comprises the consensus sequence (K/R)(K/R)X₁₀₋₁₂(K/R)_{3/5} (SEQ ID NO: 33), where (K/R)_{3/5} represents at least three of the five amino acids is either lysine or arginine. In embodiments, the NLS comprises the c-myc NLS. In embodiments, the c-myc NLS comprises the sequence PAAKRVKLD (SEQ ID NO: 34). In embodiments, the NLS is the nucleoplasmin NLS. In embodiments, the nucleoplasmin NLS comprises the sequence KRPAATKKAGQAKKKK (SEQ ID NO: 35). In embodiments, the NLS comprises the SV40 Large T-antigen NLS. In embodiments, the SV40 Large T-antigen NLS comprises the sequence PKiKKRKV (SEQ ID NO: 36). In a particular embodiment, the NLS comprises three SV40 Large T-antigen NLSs (e.g., DPKKKRKVDPKiKKRKVDPKiKKRKV (SEQ ID NO: 37)). In embodiments, the NLS may comprise mutations/variations in the above sequences such that they contain 1 or more substitutions, additions or deletions (e.g., about 1, or about 2, or about 3, or about 4, or about 5, or about 10 substitutions, additions, or deletions).

In embodiments, the endonuclease further comprises a UGI, or a fragment or variant thereof. In embodiments, the endonuclease (or chimeric protein) comprises 1, or 2, or 3, or UGIs. In embodiments, the endonuclease (or chimeric protein) comprises a UGI at the N- and/or C-terminus. Illustrative UGI proteins for use in the present disclosure include, for example, those published in Wang et al., J Biol. Chem. 264:1163-1171(1989); Lundquist et al., J Biol. Chem. 272:21408-21419(1997); Ravishankar et al., Nucleic Acids Res. 26:4880-4887(1998); and Putnam et al., J. Mol. Biol. 287:331-346(1999), each of which is incorporated herein by reference in their entirety.

In embodiments, the RNA molecule is a gRNA. In embodiments, the gRNA comprises a sequence that interacts with the endonuclease. In embodiments, the endonuclease forms a complex with the gRNA.

In embodiments, the composition is suitable for base editing, see, e.g., Rees, H. A., and Liu, D. R. (2018). Base editing: precision chemistry on the genome and transcriptome of living cells. Nat. Rev. Genet. 19, 770-788. doi: 10.1038/s41576-018-0059-1, hereby incorporated by reference in its entirety. In embodiments, the composition is suitable for DNA base editing. In embodiments, the composition is suitable for RNA base editing. In embodiments, the composition is suitable for catalyzing C>T nucleotide conversions or A>G nucleotide conversions in a target nucleic acid.

In embodiments, the composition comprises both an adenosine deaminase and a cytidine deaminase.

In embodiments, the composition is suitable for dual base editing, see, e.g., Nature Biotechnology volume 38, 856-860 (2020), hereby incorporated by reference in its entirety.

In embodiments, the reverse transcriptase is Moloney murine leukemia virus reverse transcriptase (M-MLV RT) or M-MLV RT(D200N/L603W/T330P/T306K/W313F), or a fragment or variant thereof. In embodiments, the M-MLV RT has an amino acid sequence of SEQ ID NO: 55 [M-MLV RT] or SEQ ID NO 56 [M-MLV RT(D200N/L603W/T330P/T306K/W313F)] or a fragment or variant thereof, or an amino acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto. In embodiments, the Moloney murine leukemia virus reverse transcriptase (M-MLV RT) or M-MLV RT(D200N/L603W/T330P/T306K/W313F) is selected from SEQ ID NO: 55 or SEQ ID NO: 56, as shown in Table 8 below.

TABLE 8
Moloney murine leukemia virus reverse
transcriptase sequences

| | SEQ |
|---|---|
| Sequence | ID NO: |
| TLNIEDEYRLHETSKEPDVSLGSTWLSDFPQAWAETGGMGLA | 55 |
| VRQAPLIIPLKATSTPVSIKQYPMSQEARLGIKPHIQRLLDQ | |
| GILVPCQSPWNTPLLPVKKPGTNDYRPVQDLREVNKRVEDIH | |
| PTVPNPYNLLSGLPPSHQWYTVLDLKDAFFCLRLHPTSQPLF | |
| AFEWRDPEMGISGQLTWTRLPQGFKNSPTLFDEALHRDLADF | |
| RIQHPDLILLQYVDDLLLAATSELDCQQGTRALLQTLGNLGY | |
| RASAKKAQICQKQVKYLGYLLKEGQRWLTEARKETVMGQPTP | |
| KTPRQLREFLGTAGFCRLWIPGFAEMAAPLYPLTKTGTLFNW | |
| GPDQQKAYQEIKQALLTAPALGLPDLTKPFELFVDEKQGYAK | |
| GVLTQKLGPWRRPVAYLSKKLDPVAAGWPPCLRMVAAIAVLT | |
| KDAGKLTMGQPLVILAPHAVEALVKQPPDRWLSNARMTHYQA | |
| LLLDTDRVQFGPVVALNPATLLPLPEEGLQHNCLDILAEAHG | |
| TRPDLTDQPLPDADHTWYTDGSSLLQEGQRKAGAAVTTETEV | |
| IWAKALPAGTSAQRAELIALTQALKMAEGKKLNVYTDSRYAF | |
| ATAHIHGEIYRRRGLLTSEGKEIKNKDEILALLKALFLPKRL | |
| SIIHCPGHQKGHSAEARGNRMADQAARKAAITETPDTSTLLI | |
| ENSSP | |
| AFPLERPDWDYTTQAGRNHLVHYRQLLLAGLQNAGRSPTNLA | |
| KVKGITQGPNESPSAFLERLKEAYRRYTPYDPEDPGQETNVS | |
| MSFIWQSAPDIGRKLGRLEDLKSKTLGDLVREAEKIFNKRET | |
| PEEREERIRRETEEKEERRRTVDEQKEKERDRRRHREMSKLL | |
| ATVVIGQEQDRQEGERKRPQLDKDQCAYCKEKGHWAKDCPKK | |
| PRGPRGPRPQTSLLTLGDXGGQGQDPPPEPRITLKVGGQPVT | |
| FLVDTGAQHSVLTQNPGPLSDKSAWVQGATGGKRYRWTTDRK | |
| VHLATGKVTHSFLHVPDCPYPLLGRDLLTKLKAQIHFEGSGA | 56 |
| QVVGPMGQPLQVLTLNIEDEYRLHETSKEPDVSLGFTWLSDF | |
| PQAWAESGGMGLAVRQAPLIIPLKATSTPVSIKQYPMSQEAR | |
| LGIKPHIQRLLDQGILVPCQSPWNTPLLPVKKPGTNDYRPVQ | |
| DLREVNKRVEDIHPTVPNPYNLLSGLPPSHQWYTVLDLKDAF | |
| FCLRLHPTSQPLFAFEWRDPEMGISGQLTWTRLPQGFKNSPT | |
| LFDEALHRDLADFR | |

In embodiments, the composition further comprises a dominant negative human MutL homolog (MLH1). In embodiments, the composition is suitable for use with a dominant negative MLH1.

In embodiments, the RNA molecule is or comprises a prime editing guide RNA (pegRNA). In embodiments, the endonuclease forms a complex with the pegRNA. In embodiments, the pegRNA serves as a template for transcription of a new DNA sequence. In embodiments, the pegRNA binds to a DNA strand opposite from a typical gRNA binding site. In embodiments, the pegRNA comprises a gRNA containing a primer binding site (PBS) and a reverse transcriptase (RT) template sequence. In embodiments, the RNA molecule is or comprises a gRNA. In embodiments, the gRNA comprises a sequence that interacts with the endonuclease. In embodiments, the endonuclease forms a complex with the gRNA. In embodiments, the composition comprises both a gRNA and a pegRNA.

In embodiments, the composition is suitable for prime editing see, e.g., Nature 576, 149-157 (2019), hereby incorporated by reference in its entirety.

In embodiments, the transposase is selected from Tn1, Tn2, Tn3, Tn5, Tn7, Tn9, Tn10, Tn552, Tn903, Tn1000/Gamma-delta, Tn/O, tnsA, tnsB, tnsC, tniQ, IS10, ISS, IS911, Minos, Sleeping beauty, piggyBac, Tol2, Mos1, Himarl, Hermes, Tol2, Minos, Tel, P-element, MuA, Ty1, Chapaev, transib, Tc1/mariner, and Tc3 donor DNA system.

In embodiments, the transposase is a transposon 7-like (Tn7-like) transposon system, or a fragment or variant thereof. In embodiments, the transposase is one or more of transposon 7 protein A (TnsA), transposon 7 protein B (Tns B), transposon 7 protein C (Tns C), and transposition of integron protein Q (TniQ), or a fragment or variant thereof. In embodiments, the Tn7-like transposon system is derived from Vibrio cholerae Tn6677.

In embodiments, the transposase has an amino acid sequence of one or more of SEQ ID NO: 57 [TnsA], SEQ ID NO: 58 [TnsB], SEQ ID NO: 59 [TnsC], and SEQ ID NO: 60 [TniQ], or a fragment or variant thereof, or an amino acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto. In embodiments, the transposase has an amino acid sequence of one or more of SEQ ID NOs 57-60, as shown in Table 9 below.

TABLE 9
Transposase sequences

| | SEQ |
|---|---|
| Sequence | ID NO: |
| MYRRHLKHSRVKNLFKFVSAKMNTVFTVESALEFDTCFHLE | 57 |
| YSPSVKFYEAQPEGFYYEFAGRQCPYTPDFRLVDQNDSVSF | |
| LEIKPSDKVADPDFLHRFPLKQQRAIELSSPLKLVTEKQIR | |
| IDPILGNLKLLHRYSGFQSFTPLHMQLLGLVQKLGRVSLLR | |
| LSDSIDAPPEEVLASALSLIARGIMQSDLTVQKIGISSFVW | |
| AGGHSGIDHG | |
| MDKHNGGLFEDEFVIPQPSTSTSPIDAIQAVLPATVDSFPY | 58 |
| VLKVEALHRRDYILWVEKNLAGGWTEKNLTPLLADAALVLP | |
| PPTPNWRTLARWRKIYIQHGRKLVSLIPKHQAKGNARSRLP | |
| PSDELFFEQAVHRYLVGEQPSIASAFQLYSDSIRIENLGVV | |
| ENSIKTISYMAFYNRIKKLPAYQVMKSRKGSYIADVEFKAI | |
| ASHKPPSRIMERVEIDHTPLDLLLLDDDLLVPLGRPSLTLL | |
| IDAYSHCVVGFNLNFNQPSYESVRNALLSSISKKDYVKNKY | |
| PSIEHEWPCYGKPETLVVDNGVEFWSASLAQSCLELGINIQ | |
| YNPVRKPWLKPMIERMFGIINRKLLEPIPGKTFSNIQEKGD | |
| YDPQKDAVMRFSTFLEIFHHWVIDVYHYEPDSRYRYIPIIS | |
| WQHGNKDAPPAPIIGDDLTKLEVILSLSLHCTHRRGGIQRY | |
| HLRYDSDELASYRMNYPDQTRGKRKVLVKLNPRDISYVYVF | |
| LEDLGSYIRVPCIDPIGYTKGLSLQEHQINVKLHRDFINEQ | |
| MDVVSLSKARIYLNDRIKNELIEVRRNIRQRNVKGVNKIAK | |
| YRNVGSHAETSIVHELNHPATNEVISKMESASQPEHCDDWD | |
| NFTSGLEPY | |
| MDLSCHDADKLRSFIECYVETPLLRAIQEDFDRLRFNKQFA | 59 |
| GEPQCMLLTGDTGTGKSSLIRHYAAKHPEQVRHGFIHKPLL | |
| VSRIPSRPTLESTMVELLKDLGQFGSSDRIHKSSAESLTEA | |
| LIKCLKRCETELIIIDAFQELIENKTREKRNQIANRLKYIS | |
| ETAKIPIVLVGMPWATKIAEEPQWSSRLLIRRSIPYFKLSD | |
| DRENFIRLIMGLANRMPFETQARLETKHTIYALFAACYGSL | |
| RALKQLLDESVKQALAAHAETLKHEHIAVAYALFYPDQVNP | |
| FLQPIDEIKACEVKQYSRYEIDAAGKEEVLNPLQFTDKIPI | |
| SQLLKKR | |
| MIEAPDVKPWLFLIKPYEGESLSHFLGRFRRANHLSASGLG | 60 |
| TLAGIGAIVARWERFHFNPRPSQQELEAIASVVEVDAQRLA | |
| QMLPPAGVGMQHEPIRLCGACYAESPCHRIEWQYKSVWKCD | |
| RHQLKILAKCPNCQAPFKMPALWEDGCCHRCRMPFAEMAKL | |
| QKV | |

In embodiments, the integrase is a serine-recombinase, or a fragment or variant thereof.

In embodiments, the serine-recombinase is Bxb1, or a fragment or variant thereof. In embodiments, the recombinase is a Gin invertase or Tn3 resolvase, or a fragment or variant thereof.

In embodiments, the nucleic acid-modulating domain or the nucleic acid-modifying domain can increase transcription, e.g. transcriptional activators such as VP16, VP64, VP48, VP160, p65 subdomain (e.g., from NFkB), and the activation domain of EDLL and/or TAL activation domain (e.g., for activity in plants); histone lysine methyltransferases such as SET1A, SET1B, MLL1 to 5, ASH1, SYMD2, NSD1, and the like; histone lysine demethylases such as JHDM2a/b, UTX, JMJD3, and the like; histone acetyltransferases such as GCN5, PCAF, CBP, p300, TAF1, TIP60/PLIP, MOZ/MYST3, MORF/MYST4, SRC1, ACTR, P160, CLOCK, and the like; and DNA demethylases such as Ten-Eleven Translocation (TET) dioxygenase 1 (TET1CD), TET1, DME, DML1, DML2, ROS1, and the like.

In embodiments, the nucleic acid-modulating domain or the nucleic acid-modifying domain can decrease transcription, e.g. transcriptional repressors such as the Krüppel associated box (KRAB or SKD); KOX1 repression domain; the Mad mSIN3 interaction domain (SID); the ERF repressor domain (ERD), the SRDX repression domain (e.g., for repression in plants), and the like; histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARIDlD/SMCY, and the like; histone lysine deacetylases such as HDAC1, HDAC2, HDAC3, HDAC8, HDAC4, HDAC5, HDAC7, HDAC9, SIRT1, SIRT2, HDAC11, and the like; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like; and periphery recruitment elements such as Lamin A, Lamin B, and the like.

In embodiments, the nucleic acid-modulating domain or the nucleic acid-modifying domain has enzymatic activity that modifies a target nucleic acid (e.g., ssRNA, dsRNA, ssDNA, dsDNA). Examples of enzymatic activity that can be provided include but are not limited to: nuclease activity such as that provided by a restriction enzyme (e.g., FokI nuclease), methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (e.g. plants), ZMET2, CMT1, CMT2 (e.g. plants), and the like); demethylase activity such as that provided by a demethylase (e.g., Ten-Eleven Translocation (TET) dioxygenase 1 (TET1CD), TET1, DME, DML1, DML2, ROS1, and the like), DNA repair activity, DNA damage activity, deamination activity such as that provided by a deaminase (e.g., a cytosine deaminase enzyme such as rat APOBEC1), dismutase activity, alkylation activity, depurination activity, oxidation activity, pyrimidine dimer forming activity, integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase; and the like), transposase activity, recombinase activity such as that provided by a recombinase (e.g., catalytic domain of Gin recombinase), polymerase activity, ligase activity, helicase activity, photolyase activity, and glycosylase activity).

In embodiments, the nucleic acid-modulating domain or the nucleic acid-modifying domain has debranching activity, including, for example, lariat debranching enzyme or DBR1.

In embodiments, the nucleic acid-modulating domain or the nucleic acid-modifying domain has transesterification activity, including, for example, a relaxase or Spoil.

In embodiments, the nucleic acid-modulating domain or the nucleic acid-modifying domain has activity that modifies a protein associated with a target nucleic acid (e.g., ssRNA, dsRNA, ssDNA, dsDNA) (e.g., a histone, an RNA binding protein, a DNA binding protein, and the like). Examples of enzymatic activity (that modifies a protein associated with a target nucleic acid) that can be provided by the fusion partner include but are not limited to: methyltransferase activity such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB1, and the like, SET1A, SET1B, MLL1 to 5, ASH1, SYMD2, NSD1, DOT1L, Pr-SET7/8, SUV4-20H1, EZH2, RIZ1), demethylase activity such as that provided by ahistone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARIDlD/SMCY, UTX, JMJD3, and the like), acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK, and the like), deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11, and the like), kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity. In embodiments, the nucleic acid-modulating domain or the nucleic acid-modifying domain is one or more effector domains, including proteins and/or enzymes, such that those can cleave RNA (e.g., a PIN endonuclease domain, an NYN domain, an SMR domain from SOT1, or an RNase domain from Staphylococcal nuclease), those that can affect RNA stability (e.g., tristetraprolin (TTP) or domains from UPF1, EXOSC5, and STAU1), those that can edit a nucleotide or ribonucleotide (e.g., a cytidine deaminase, PPR protein, adenosine deaminase, ADAR family protein, or APOBEC family protein), those that can activate translation (e.g., eIF4E and other translation initiation factors, a domain of the yeast poly(A)-binding protein or GLD2), those that can repress translation (e.g., Pumilio or FBF PUF proteins, deadenylases, CAF1, Argonaute proteins), those that can methylate RNA (e.g., domains from m6A methyltransferase factors such as METTL14, METTL3, or WTAP), those that can demethylate RNA (e.g., human alkylation repair homolog 5 or Alkbh5), those that can affect splicing (e.g., the RS-rich domain of SRSF1, the Gly-rich domain of hnRNP A1, the alanine-rich motif of RBM4, or the proline-rich motif of DAZAP1), those that can enable affinity purification or immunoprecipitation (e.g., FLAG, HA, biotin, or HALO tags), and those that can enable proximity-based protein labeling and identification (e.g., a biotin ligase (such as BirA) or a peroxidase (such as APEX2) in order to biotinylate proteins that interact with the target RNA). In embodiments, the endonuclease (or chimeric protein) induces exon skipping. In embodiments, the endonuclease (or chimeric protein) induces exon inclusion.

In embodiments, the nucleic acid-modulating domain or the nucleic acid-modifying domain comprises one or more mutations to reduce activity relative to an unmutated form.

In embodiments, the nucleic acid-modulating domain or the nucleic acid-modifying domain comprises one or more mutations to increase activity relative to an unmutated form.

### Chimeric Protein Design/Linkers

In embodiments, the sequence of (a) is disposed at the N-terminus of the chimeric protein and the sequence of (b) is disposed at the C-terminus of the chimeric protein.

In embodiments, the sequence of (a) is disposed at the C-terminus of the chimeric protein and the sequence of (b) is disposed at the N-terminus of the chimeric protein.

In embodiments, the composition further comprises a linker that joins the sequence of (a) and the sequence of (b). In embodiments, the linker is between about 4 and about 40 amino acids, or about 10 and about 40 amino acids, or about 20 and about 40 amino acids, or about 30 and about 40 amino acids, or about 4 and about 30 amino acids, or about 4 and about 20 amino acids, or about 4 and about 10 amino acids, or about 5 amino acids, or about 10 amino acids, or about 15 amino acids, or about 20 amino acids, or about 25 amino acids, or about 30 amino acids, or about 35 amino acids, or about 40 amino acids. In embodiments, the linker is substantially comprised of glycine and serine residues. In embodiments, the linker is (GGS)ₙ, wherein ₙ is 1, or 2, or 3, or 4, or 5. In embodiments, the linker is GGSGGSGGSG (SEQ ID NO: 61), GGSGGSGGGGSGGGGS (SEQ ID NO: 62), GGGGS (SEQ ID NO: 63), GGS (SEQ ID NO: 64), (GGGGS)ₙ(n=1-4) (SEQ ID NO: 65), (Gly)₈ (SEQ ID NO: 66), (Gly)₆ (SEQ ID NO: 67), (EAAAK)ₙ(n=1-3) (SEQ ID NO: 68), A(EAAAK)ₙA (n=2-5) (SEQ ID NO: 69), AEAAAKEAAAKA (SEQ ID NO: 70), A(EAAAK)₄ALEA(EAAAK)₄A (SEQ ID NO: 71), PAPAP (SEQ ID NO: 72), KESGSVSSEQLAQFRSLD (SEQ ID NO: 73), EGKSSGSGSESKST (SEQ ID NO: 74), and GSAGSAAGSGEF (SEQ ID NO: 75), or a variant thereof, wherein the variant comprises about 1, or about 2, or about 3, or about 4, or about 5 mutations, the mutations selected from substitutions or deletions.

### Target Nucleic Acid

In embodiments, the target nucleic acid is referred to as a target sequence. In embodiments, the target nucleic acid is or comprises a sequence of contiguous nucleotides present in a target RNA or target DNA. In embodiments, a stretch of contiguous nucleotides refers to a string of nucleotides that are covalently linked and immediately adjacent to one another. In embodiments, the target nucleic acid is or comprises at least about 4, 5, 6, 7, 8, 9, 10, 11, 12, 13, 14, 15, 16, 17, 18, 19, or 20 nucleotides in length. In embodiments, the target nucleic acid is or comprises less than about 300, 250, 200, 100, 150, or 50 nucleotides in length. In embodiments, the target nucleic acid is or comprises about 5-10, about 5-15, about 5-20, about 10-20, about 10-30, about 10-40, about 10-50, about 10-60, about 10-70, about 10-80, about 10-90, about 10-100, about 50-100, about 50-150, about 50-200, about 50-250, about 50-300, about 100-200, about 100-300, or about 200-300 nucleotides in length.

In embodiments, the target nucleic acid is 10-50 nucleotides in length, e.g., 10-45, 10-40, 10-35, 10-30, 10-20, 11-45, 11-40, 11-35, 11-30, 11-20, 12-45, 12-40, 12-35, 12-30, 12-25, 12-20, 13-45, 13-40, 13-35, 13-30, 13-25, 13-20, 14-45, 14-40, 14-35, 14-30, 14-25, 14-20, 15-45, 15-40, 15-35, 15-30, 15-25, 15-20, 16-45, 16-40, 16-35, 16-30, 16-25, 16-20, 17-45, 17-40, 17-35, 17-30, 17-25, 17-20, 18-45, 18-40, 18-35, 18-30, 18-25, 18-20, 19-45, 19-40, 19-35, 19-30, 19-25, 19-20, e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In embodiments, the target nucleic acid is or comprises ssRNA. In embodiments, the target nucleic acid is or comprises dsRNA. In embodiments, the target nucleic acid is or comprises ssDNA. In embodiments, the target nucleic acid is or comprises dsDNA. In embodiments, the target nucleic acid is about 2 to about 6 nucleotides upstream of a PAM sequence.

In embodiments, the target nucleic acid is proximal to an exon. In embodiments, the target nucleic acid is upstream of an exon. In embodiments, the target nucleic acid is downstream of an exon. In embodiments, the target nucleic acid overlaps with an exon.

In embodiments, the target nucleic acid is proximal to an intron. In embodiments, the target nucleic acid is upstream of an intron. In embodiments, the target nucleic acid is downstream of an intron. In embodiments, the target nucleic acid overlaps with an intron.

### General Features of the Compositions

In embodiments, the composition further comprises a viral vector. In embodiments, the viral vector is or comprises an AAV. In embodiments, the AAV is or comprises one or more of AAV1, AAV2, AAV3, AAV4, AAV5, AAV6, AAV7, AAV8, AAV9, AAV10, AAV11, AAV12, AAV13, AAV2/1, AAV2/5, AAV2/8, AAV2/9, AAV3/1, AAV3/5, AAV3/8, and AAV3/9. In embodiments, the composition further comprises a non-viral vector.

In embodiments, the present compositions take the form of, or delivery of the present compositions is effected using, a nanoparticle, e.g., any particle having a diameter of less than about 1000 nm. In embodiments, nanoparticles suitable for use in delivering the present compositions to a target cell have a diameter of about 500 nm or less, e.g., from about 25 nm to about 35 nm, from about 35 nm to about 50 nm, from about 50 nm to about 75 nm, from about 75 nm to about 100 nm, from about 100 nm to about 150 nm, from about 150 nm to about 200 nm, from about 200 nm to about 300 nm, from about 300 nm to about 400 nm, or from about 400 nm to about 500 nm. In embodiments, nanoparticles suitable for use in delivering the present compositions to a target cell have a diameter of from about 25 nm to about 200 nm. In embodiments, nanoparticles suitable for use in delivery have a diameter of about 100 nm or less. In embodiments, nanoparticles suitable for use in delivery have a diameter of from about 35 nm to about 60 nm.

In embodiments, the composition further comprises a lipid nanoparticle (LNP) liposomes, lipoplexes or polymeric nanoparticle. In embodiments, the LNP comprises one or more of ionizable lipids, amino lipids, anionic lipids, neutral lipids, amphipathic lipids, helper lipids, structural lipids, PEG lipids, and lipoids.

In embodiments there is provided a particle, e.g., a delivery particle comprising lipid or lipidoid and hydrophilic polymer, e.g., a cationic lipid and a hydrophilic polymer, for instance where the cationic lipid comprises 1,2-dioleoyl-3-trimethylammonium-propane (DOTAP) or 1,2-ditetradecanoyl-sn-glycero-3-phosphocholine (DMPC) and/or where the hydrophilic polymer comprises ethylene glycol or polyethylene glycol (PEG); and/or where the particle further comprises cholesterol (e.g., particle from formulation 1=DOTAP 100, DMPC 0, PEG 0, Cholesterol 0; formulation number 2=DOTAP 90, DMPC 0, PEG 10, Cholesterol 0; formulation number 3=DOTAP 90, DMPC 0, PEG 5, Cholesterol 5).

In embodiments, a liposome is used to deliver a composition of the present disclosure to a target cell. Liposomes are spherical vesicle structures composed of a uni- or multilamellar lipid bilayer surrounding internal aqueous compartments and a relatively impermeable outer lipophilic phospholipid bilayer. Liposomes can be made from several different types of lipids; however, phospholipids are most commonly used to generate liposomes. Although liposome formation is spontaneous when a lipid film is mixed with an aqueous solution, it can also be expedited by applying force in the form of shaking by using a homogenizer, sonicator, or an extrusion apparatus. Several other additives may be added to liposomes in order to modify their structure and properties. For instance, either cholesterol or sphingomyelin may be added to the liposomal mixture in order to help stabilize the liposomal structure and to prevent the leakage of the liposomal inner cargo. A liposome formulation may be mainly comprised of natural phospholipids and lipids such as 1,2-distearoryl-sn-glycero-3-phosphatidyl choline (DSPC), sphingomyelin, egg phosphatidylcholines and monosialoganglioside.

In embodiments, the composition is in the form of a lipoplex. Lipoplexes that utilize cationic lipids have proven utility for gene transfer. Cationic lipids, due to their positive charge, naturally complex with the negatively charged DNA. Also, as a result of their charge, they interact with the cell membrane. Endocytosis of the lipoplex then occurs, and the DNA is released into the cytoplasm. The cationic lipids also protect against degradation of the DNA by the cell.

In embodiments, the composition is in the form of a polyplex. Most polyplexes consist of cationic polymers and their production is regulated by ionic interactions. One large difference between the methods of action of polyplexes and lipoplexes is that polyplexes cannot release their DNA load into the cytoplasm, so to this end, co-transfection with endosome-lytic agents (to lyse the endosome that is made during endocytosis) such as inactivated adenovirus must occur. However, this is not always the case; polymers such as polyethylenimine have their own method of endosome disruption as does chitosan and trimethylchitosan.

In embodiments, the composition is in the form of a dendrimer, a highly branched macromolecule with a spherical shape may be also be used to genetically modify stem cells. The surface of the dendrimer particle may be functionalized to alter its properties. In particular, it is possible to construct a cationic dendrimer (e.g., one with a positive surface charge). When in the presence of genetic material such as a DNA plasmid, charge complementarity leads to a temporary association of the nucleic acid with the cationic dendrimer. On reaching its destination, the dendrimer-nucleic acid complex can be taken up into a cell by endocytosis.

In aspects, the present disclosure provides a nucleic acid encoding the endonuclease or chimeric protein of any one of the embodiments and/or aspects disclosed herein. In embodiments, the nucleic acid is or comprises a DNA molecule or an RNA molecule. In embodiments, the RNA is or comprises mRNA or modified mRNA (mmRNA). In embodiments, the DNA is or comprises a vector or plasmid. In embodiments, the nucleic acid comprises a codon optimized sequence. In embodiments, the nucleic acid comprises one or more modifications. In embodiments, the modifications are one or more of base modifications and backbone modifications.

In embodiments, sugar-based particles may be used, for example GalNAc, can be used to deliver a composition of the present disclosure to a target cell.

In aspects, the present disclosure provides a viral vector comprising the nucleic acid of any one of the embodiments and/or aspects disclosed herein. In aspects, the present disclosure provides an expression vector comprising the nucleic acid of any one of the embodiments and/or aspects disclosed herein.

In embodiments, the expression vector is selected from viral expression vectors (e.g., viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (AAV) (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In embodiments, a recombinant expression vector of the present disclosure is a recombinant adeno-associated virus (AAV) vector. In embodiments, a recombinant expression vector of the present disclosure is a recombinant lentivirus vector. In embodiments, a recombinant expression vector of the present disclosure is a recombinant retroviral vector.

In embodiments, the viral vector is or comprises an AAV. In embodiments, the AAV is or comprises one or more of AAV1, AAV2, AAV3, AAV4, AAV5, AAV6, AAV7, AAV8, AAV9, AAV10, AAV11, AAV12, AAV13, AAV2/1, AAV2/5, AAV2/8, AAV2/9, AAV3/1, AAV3/5, AAV3/8, and AAV3/9.

In embodiments, the composition further comprises a VLP, e.g. a structure that in at least one attribute resembles a virus, but which has not been demonstrated to be infectious. In embodiments, the VLP is a nonreplicating, noninfectious viral shell that contains a viral capsid but lacks all or part of the viral genome, in particular, the replicative components of the viral genome. In embodiments, the VLP is composed of one or more viral proteins, such as, but not limited to those proteins referred to as capsid, coat, shell, surface, and structural proteins (e.g., VP1, VP2). In embodiments, the VLP resembles the structure of a bacteriophage, being non-replicative and noninfectious, and lacking at least the gene or genes coding for the replication machinery of the bacteriophage, and also lacking the gene or genes encoding the protein or proteins responsible for viral attachment to or entry into the host. In embodiments, the VLP comprises a polypeptide that promotes or is suitable for VLP delivery, including, without limitation, a retroviral gag polyprotein comprising a matrix polypeptide, a capsid polypeptide, and a nucleocapsid polypeptide (optionally with one or more heterologous protease cleavage sites (e.g. TEV cleavage site, a PreScission (fusion protein of glutathione S-transferase (GST) and human rhinovirus (HRV) type 14 3C protease) cleavage site, a human rhinovirus 3C protease cleavage site, an enterokinase cleavage site, an Epstein-Barr virus protease cleavage site, a cathepsin D cleavage site, and/or a thrombin cleavage site) between one or both of: the matrix polypeptide and the capsid polypeptide; and the capsid polypeptide and the nucleocapsid polypeptide, e.g., a lentiviral gag polyprotein, e.g., a bovine immunodeficiency virus gag polyprotein, a murine leukemia virus (MLV) a gag protein, a simian immunodeficiency virus gag polyprotein, a feline immunodeficiency virus gag polyprotein, a human immunodeficiency virus gag polyprotein, an equine infection anemia virus gag polyprotein, and a caprine arthritis encephalitis virus gag polyprotein or a gag polyprotein of an alpha retrovirus, a beta retrovirus, a gamma retrovirus, a delta retrovirus, an epsilon retrovirus, or a spumavirus. In embodiments, the polypeptide that promotes or is suitable for VLP delivery is co-delivered with a protease to promote cleavage of the chimeric protein. In embodiments, the cleavage of the chimeric protein occurs between the endonuclease and the polypeptide that promotes or is suitable for VLP delivery. In embodiments, the protease is fused to a polypeptide that promotes or is suitable for VLP delivery.

Depending on the host/vector system utilized, in embodiments, any of a number of transcription and/or translation control elements, including constitutive and inducible promoters, transcription enhancer elements, transcription terminators, and the like may be used in the expression vector. In embodiments, a nucleotide sequence encoding a present RNA is operably linked to a control element, e.g., a transcriptional control element, such as a promoter. In embodiments, a nucleotide sequence encoding a present protein, or a present fusion polypeptide is operably linked to a control element, e.g., a transcriptional control element, such as a promoter. In embodiments, the transcriptional control element is a promoter. In embodiments, the promoter is a constitutively active promoter. In embodiments, the promoter is a regulatable promoter. In embodiments, the promoter is an inducible promoter. In embodiments, the promoter is a tissue-specific promoter. In embodiments, the promoter is a cell type-specific promoter. In embodiments, the transcriptional control element (e.g., the promoter) is functional in a targeted cell type or targeted cell population. For example, in embodiments, the transcriptional control element is functional in eukaryotic cells, e.g., hematopoietic stem cells (e.g., mobilized peripheral blood (mPB) CD34(+) cell, bone marrow (BM) CD34(+) cell, and the like). In embodiments, eukaryotic promoters (promoters functional in a eukaryotic cell) include EF1α, those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art.

In embodiments, a nucleotide sequence encoding a present RNA and/or a present fusion polypeptide is operably linked to an inducible promoter. In embodiments, a nucleotide sequence encoding a present RNA and/or a present chimeric protein is operably linked to a constitutive promoter.

In embodiments, the promoter is derived from viruses and can therefore be referred to as viral promoters, or they are derived from any organism, including prokaryotic or eukaryotic organisms. In embodiments, the promoter is used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter (e.g. is or comprising SEQ ID NO: 76, or a variant thereof) such as the CMV immediate early promoter region (CMVIE), a rous sarcoma virus (RSV) promoter, a human U6 small nuclear promoter (U6) (Miyagishi et-al., Nature Biotechnology 20, 497-500 (2002)), an enhanced U6 promoter (e.g., Xia et al., Nucleic Acids Res. 2003 Sep. 1; 31 (17)), a human H1 promoter (H1), and the like.

In embodiments, a nucleotide sequence encoding a present RNA is operably linked to (under the control of) a promoter operable in a eukaryotic cell (e.g., a U6 promoter, an enhanced U6 promoter, an H1 promoter, and the like). As would be understood by one of ordinary skill in the art, when expressing an RNA (e.g., a guide RNA) from a nucleic acid (e.g., an expression vector) using a U6 promoter (e.g., in a eukaryotic cell), or another PolIII promoter, the RNA may need to be mutated if there are several Ts in a row (coding for Us in the RNA). This is because a string of Ts (e.g., about 5 Ts) in DNA can act as a terminator for polymerase III (PolIII). Thus, in order to ensure transcription of an RNA in a eukaryotic cell it may be desirable to modify the sequence encoding the RNA to eliminate runs of Ts. In embodiments, a nucleotide sequence encoding a present protein (e.g., a disclosed protein or chimeric protein) is operably linked to a promoter operable in a eukaryotic cell (e.g., a CMV promoter, an EF1α promoter, an estrogen receptor-regulated promoter, and the like).

In embodiments, the inducible promoter includes, but are not limited to, one of T7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter, Steroid-regulated promoter, Metal-regulated promoter, estrogen receptor-regulated promoter, and the like. Inducible promoters can therefore be regulated by molecules including, but not limited to, doxycycline; estrogen and/or an estrogen analog; IPTG; and the like. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In embodiments, the promoter is a spatially restricted promoter (e.g., cell type specific promoter, tissue specific promoter, and the like) such that in a multi-cellular organism, the promoter is active (e.g., “ON”) in a subset of specific cells. Spatially restricted promoters may also be referred to as enhancers, transcriptional control elements, control sequences, etc. Any convenient spatially restricted promoter may be used as long as the promoter is functional in the targeted host cell (e.g., eukaryotic cell; prokaryotic cell).

In embodiments, the promoter is a reversible promoter. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), and the like), tetracycline regulated promoters, (e.g., promoter systems including TetActivators, TetON, TetOFF, and the like), steroid regulated promoters (e.g., rat glucocorticoid receptor promoter systems, human estrogen receptor promoter systems, retinoid promoter systems, thyroid promoter systems, ecdysone promoter systems, mifepristone promoter systems, and the like), metal regulated promoters (e.g., metallothionein promoter systems, and the like), pathogenesis-related regulated promoters (e.g., salicylic acid regulated promoters, ethylene regulated promoters, benzothiadiazole regulated promoters, and the like), temperature regulated promoters (e.g., heat shock inducible promoters (e.g., HSP-70, HSP-90, soybean heat shock promoter, and the like), light regulated promoters, synthetic inducible promoters, and the like.

In embodiments, the vector contains a ribosome binding site for translation initiation and a transcription terminator. In embodiments, the vector includes appropriate sequences for amplifying expression. In embodiments, the vector includes nucleotide sequences encoding protein tags (e.g., 6xHis tag, hemagglutinin tag (e.g., GSGPKKKRKVAAAYPYDVPDYA (SEQ ID NO: 77)), fluorescent protein, and the like) that are fused to the present protein, e.g., at the N- or C-terminus or between the N- or C-terminus, thus resulting in a fusion present polypeptide.

In embodiments, methods of introducing a nucleic acid (e.g., a nucleic acid comprising a donor polynucleotide sequence, one or more nucleic acids encoding a present protein and/or a present RNA, and the like) into a host cell are known in the art, and any convenient method is used to introduce a nucleic acid (e.g., an expression construct) into a cell. Suitable methods include e.g., viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like.

In embodiments, introducing the recombinant expression vector into cells can occur in any culture media and under any culture conditions that promote the survival of the cells. In embodiments, introducing the recombinant expression vector into a target cell is carried out in vivo or ex vivo. In embodiments, introducing the recombinant expression vector into a target cell is carried out in vitro.

In embodiments, the present protein (e.g., endonuclease, chimeric protein) is provided as a nucleic acid. In embodiments, the present protein (e.g., endonuclease, chimeric protein) is provided as RNA. In embodiments, the present protein (e.g., endonuclease, chimeric protein) is provided as DNA. In embodiments, the RNA is generated by direct chemical synthesis or may be transcribed in vitro from a DNA (e.g., encoding the present protein). Once synthesized, the RNA may be introduced into a cell by any of the well-known techniques for introducing nucleic acids into cells (e.g., microinjection, electroporation, transfection, and the like).

Methods of introducing a nucleic acid into a host cell are known in the art, and any convenient method can be used to introduce a present composition into a target cell (e.g., prokaryotic cell, eukaryotic cell, plant cell, animal cell, mammalian cell, human cell, and the like). Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like.

In embodiments, nucleic acids may be provided to the cells using well-developed transfection techniques; see, e.g., Angel and Yanik (2010) PLoS ONE 5(7): e11756, and the commercially available TRANSMESSENGER reagents from Qiagen, STEMFECT RNA Transfection Kit from Stemgent, and TRANSIT-mRNA Transfection Kit from Mirus Bio LLC. See also Beumer et al. (2008) PNAS 105(50):19821-19826.

In aspects, the present disclosure provides a lipid nanoparticle comprising the nucleic acid of any one of the embodiments and/or aspects disclosed herein.

In aspects, the present disclosure provides a cell comprising a nucleic acid of any one of the embodiments and/or aspects disclosed herein, the viral vector of any one of the embodiments and/or aspects disclosed herein, or the lipid nanoparticle of any one of the embodiments and/or aspects disclosed herein.

In embodiments, the cell is a prokaryotic cell. In embodiments, the cell is a eukaryotic cell. In embodiments, the cell is a mammalian cell. In embodiments, the cell is a human cell. In embodiments, the cell is an immortalized cell. In embodiments, the cell is harvested from a subject.

In embodiments, the cell derived from the subject is derived from a biological sample. In embodiments, the biological sample comprises a biopsy, tissue or bodily fluid. In embodiments, the biological sample comprises one or more of tumor cells, cultured cells, stem cells, and differentiated cells. In embodiments, biological sample refers to a sample obtained or derived from a source of interest (e.g., a cell), as described herein. In embodiments, a source of interest comprises an organism, such as an animal or human. In embodiments, a biological sample is a biological tissue or fluid. Non-limiting examples of biological samples include bone marrow, blood, blood cells, ascites, (tissue or fine needle) biopsy samples, cell-containing body fluids, free floating nucleic acids, sputum, saliva, urine, cerebrospinal fluid, peritoneal fluid, pleural fluid, feces, lymph, gynecological fluids, swabs (e.g., skin swabs, vaginal swabs, oral swabs, and nasal swabs), washings or lavages such as a ductal lavages or broncheoalveolar lavages, aspirates, scrapings, specimens (e.g., bone marrow specimens, tissue biopsy specimens, and surgical specimens), feces, other body fluids, secretions, and/or excretions, and cells therefrom, and the like.

In embodiments, the present disclosure provides a modified cell comprising a composition of the present disclosure. In embodiments, the present disclosure provides a modified cell comprising a composition of the present disclosure, where the modified cell is a cell that does not normally comprise a composition of the present disclosure. In embodiments, the present disclosure provides a modified cell (e.g., a genetically modified cell) comprising nucleic acid comprising a nucleotide sequence encoding a composition of the present disclosure. In embodiments, there is provided a genetically modified cell that is genetically modified with an mRNA comprising a nucleotide sequence encoding a composition of the present disclosure. In embodiments, there is provided a genetically modified cell that is genetically modified with a recombinant expression vector comprising a composition of the present disclosure.

In embodiments, the cells are primary cells; cancer cells; animal cells; plant cells; algal cells; fungal cells; and the like. In embodiments, a cell that serves as a recipient for a composition of the present disclosure is referred to as a “host cell” or a “target cell.” In embodiments, the host cell or a target cell can be a recipient of a composition or system of the present disclosure. A host cell or a target cell can be a recipient of a RNP of the present disclosure. A host cell or a target cell can be a recipient of a single component of a k system of the present disclosure.

Non-limiting examples of cells (target cells) include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, angiosperms, ferns, clubmosses, hornworts, liverworts, mosses, dicotyledons, monocotyledons, and the like), an algal cell, (e.g., Botryococcus braunii, Chlamydomonas reinhardtii, Nannochloropsis gaditana, Chlorella pyrenoidosa, Sargassum patens, C. agardh, and the like), seaweeds (e.g. kelp) a fungal cell (e.g., a yeast cell, a cell from a mushroom), an animal cell, a cell from an invertebrate animal (e.g., fruit fly, cnidarian, echinoderm, nematode, and the like), a cell from a vertebrate animal (e.g., fish, amphibian, reptile, bird, mammal), a cell from a mammal (e.g., an ungulate (e.g., a pig, a cow, a goat, a sheep); a rodent (e.g., a rat, a mouse); a non-human primate; a human; a feline (e.g., a cat); a canine (e.g., a dog); and the like), and the like. In embodiments, the cell is a cell that does not originate from a natural organism (e.g., the cell can be a synthetically made cell; also referred to as an artificial cell).

In embodiments, the cell is an in vitro cell (e.g., established cultured cell line). A cell can be an ex vivo cell (cultured cell from an individual). In embodiments, the cell is in vivo cell (e.g., a cell in an individual). In embodiments, the cell is an isolated cell. In embodiments, the cell is a cell inside of an organism. In embodiments, the cell is an organism. In embodiments, the cell is a cell in a cell culture (e.g., in vitro cell culture). In embodiments, the cell is cell in a collection of cells. In embodiments, the cell is a prokaryotic cell or derived from a prokaryotic cell. cell culture (e.g., in vitro cell culture). In embodiments, the cell is cell in a collection of cells. In embodiments, the cell is a bacterial cell or can be derived from a bacterial cell. In embodiments, the cell is an archaeal cell or derived from an archaeal cell. In embodiments, the cell is an eukaryotic cell or derived from a eukaryotic cell. In embodiments, the cell is a plant cell or derived from a plant cell. In embodiments, the cell is an animal cell or derived from an animal cell. In embodiments, the cell is an invertebrate cell or derived from an invertebrate cell. In embodiments, the cell is a vertebrate cell or derived from a vertebrate cell. In embodiments, the cell is a mammalian cell or derived from a mammalian cell. In embodiments, the cell is a rodent cell or derived from a rodent cell. In embodiments, the cell is a human cell or derived from a human cell. In embodiments, the cell is a microbe cell or derived from a microbe cell. In embodiments, the cell is a fungal cell or derived from a fungal cell. In embodiments, the cell is an insect cell. In embodiments, the cell is an arthropod cell. In embodiments, the cell is a protozoan cell.

In embodiments, the suitable cells include a stem cell (e.g., an embryonic stem (ES) cell, an induced pluripotent stem (iPS) cell; a germ cell (e.g., an oocyte, a sperm, an oogonia, a spermatogonia, and the like); a somatic cell, e.g., a fibroblast, an oligodendrocyte, a glial cell, a hematopoietic cell, a neuron, a muscle cell, a bone cell, a hepatocyte, a pancreatic cell, and the like.

In embodiments, the suitable cells include human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In embodiments, the cell is an immune cell, a neuron, an epithelial cell, and endothelial cell, or a stem cell. In embodiments, the immune cell is a T cell, a B cell, a monocyte, a natural killer cell, a dendritic cell, or a macrophage. In embodiments, the immune cell is a cytotoxic T cell. In embodiments, the immune cell is a helper T cell. In embodiments, the immune cell is a regulatory T cell (Treg).

In embodiments, the cell is a stem cell. Stem cells include adult stem cells. Adult stem cells are also referred to as somatic stem cells. Adult stem cells are resident in differentiated tissue but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like. Stem cells of interest include mammalian stem cells, where the term “mammalian” refers to any animal classified as a mammal, including humans; non-human primates; domestic and farm animals; and zoo, laboratory, sports, or pet animals, such as dogs, horses, cats, cows, mice, rats, rabbits, etc. In embodiments, the stem cell is a human stem cell. In embodiments, the stem cell is a rodent (e.g., a mouse; a rat) stem cell. In embodiments, the stem cell is a non-human primate stem cell.

In embodiments, the stem cell is a hematopoietic stem cell (HSC). HSCs are mesoderm-derived cells that can be isolated from bone marrow, blood, cord blood, fetal liver and yolk sac. HSCs are characterized as CD34⁺ and CD3⁻. HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

In embodiments, the stem cell is a neural stem cell (NSC). Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

In embodiments, the stem cell is a mesenchymal stem cell (MSC). MSCs originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC. See, e.g., U.S. Pat. No. 5,736,396, which describes isolation of human MSC.

In embodiments, the cell is a plant cell. For example, the cell can be a cell of a major agricultural plant, e.g., Barley, Beans (Dry Edible), Canola, Corn, Cotton (Pima), Cotton (Upland), Flaxseed, Hay (Alfalfa), Hay (Non-Alfalfa), Oats, Peanuts, Rice, Sorghum, Soybeans, Sugarbeets, Sugarcane, Sunflowers (Oil), Sunflowers (Non-Oil), Sweet Potatoes, Tobacco (Burley), Tobacco (Flue-cured), Tomatoes, Wheat (Durum), Wheat (Spring), Wheat (Winter), and the like. As another example, the cell is a cell of a vegetable crops which include but are not limited to, e.g., alfalfa sprouts, aloe leaves, arrow root, arrowhead, artichokes, asparagus, bamboo shoots, banana flowers, bean sprouts, beans, beet tops, beets, bittermelon, bok choy, broccoli, broccoli rabe (rappini), brussels sprouts, cabbage, cabbage sprouts, cactus leaf (nopales), calabaza, cardoon, carrots, cauliflower, celery, chayote, Chinese artichoke (crosnes), Chinese cabbage, Chinese celery, Chinese chives, choy sum, chrysanthemum leaves (tung ho), collard greens, corn stalks, corn-sweet, cucumbers, daikon, dandelion greens, dasheen, dau mue (pea tips), donqua (winter melon), eggplant, endive, escarole, fiddle head ferns, field cress, frisee, gai choy (Chinese mustard), gailon, galanga (siam, Thai ginger), garlic, ginger root, gobo, greens, Hanover salad greens, huauzontle, Jerusalem artichokes, jicama, kale greens, kohlrabi, lamb's quarters (quilete), lettuce (bibb), lettuce (Boston), lettuce (Boston red), lettuce (green leaf), lettuce (iceberg), lettuc—(lolla rossa), lettuce (oa—leaf-green), lettuce (oak leaf-red), lettuce (processed), lettuce (red leaf), lettuce (romaine), lettuce (ruby romaine), lettuce (Russian red mustard), linkok, lo bok, long beans, lotus root, mache, maguey (agave) leaves, malanga, mesculin mix, mizuna, moap (smooth luffa), moo, moqua (fuzzy squash), mushrooms, mustard, nagaimo, okra, ong choy, onions green, opo (long squash), ornamental corn, ornamental gourds, parsley, parsnips, peas, peppers (bell type), peppers, pumpkins, radicchio, radish sprouts, radishes, rape greens, rape greens, rhubarb, romaine (baby red), rutabagas, salicomia (sea bean), sinqua (angled/ridged luffa), spinach, squash, straw bales, sugarcane, sweet potatoes, Swiss chard, tamarindo, taro, taro leaf, taro shoots, tatsoi, tepeguaje (guaje), tindora, tomatillos, tomatoes, tomatoes (cherry), tomatoes (grape type), tomatoes (plum type), tumeric, turnip tops greens, turnips, water chestnuts, yampi, yams (names), yu choy, yuca (cassava), and the like.

In embodiments, the cell is an arthropod cell. For example, the cell can be a cell of a sub-order, a family, a sub-family, a group, a sub-group, or a species of, e.g., Chelicerata, Myriapodia, Hexipodia, Arachnida, Insecta, Archaeognatha, Thysanura, Palaeoptera, Ephemeroptera, Odonata, Anisoptera, Zygoptera, Neoptera, Exopterygota, Plecoptera, Embioptera, Orthoptera, Zoraptera, Dermaptera, Dictyoptera, Notoptera, Grylloblattidae, Mantophasmatidae, Phasmatodea, Blattaria, Isoptera, Mantodea, Parapneuroptera, Psocoptera, Thysanoptera, Phthiraptera, Hemiptera, Endopterygota or Holometabola, Hymenoptera, Coleoptera, Strepsiptera, Raphidioptera, Megaloptera, Neuroptera, Mecoptera, Siphonaptera, Diptera, Trichoptera, or Lepidoptera.

In embodiments, the cell is an insect cell. For example, in embodiments, the cell is a cell of a mosquito, a grasshopper, a true bug, a fly, a flea, a bee, a wasp, an ant, a louse, a moth, or a beetle.

In aspects, the present disclosure provides a pharmaceutical composition comprising the composition of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, or the cell of any one of the embodiments and/or aspects disclosed herein, and a pharmaceutically acceptable carrier.

The compositions described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety. Pharmaceutically acceptable salts include, by way of non-limiting example, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts.

The term “pharmaceutically acceptable salt” also refers to a salt of the compositions of the present invention having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

In embodiments, the compositions described herein are in the form of a pharmaceutically acceptable salt.

In various embodiments, the present invention pertains to pharmaceutical compositions comprising the compositions described herein and a pharmaceutically acceptable carrier or excipient. Any pharmaceutical compositions described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

In various embodiments, pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents. Other examples of suitable pharmaceutical excipients are described in Remington's Pharmaceutical Sciences 144⁷-1676 (Alfonso R. Gennaro eds., 19th ed. 1995), incorporated herein by reference. The present ⁱⁿvention includes the described pharmaceutical compositions (and/or additional therapeutic agents) in various formulations. Any inventive pharmaceutical composition (and/or additional therapeutic agents) described herein can take the form of solutions, suspensions, emulsion, drops, tablets, pills, pellets, capsules, capsules containing liquids, gelatin capsules, powders, sustained-release formulations, suppositories, emulsions, aerosols, sprays, suspensions, lyophilized powder, frozen suspension, desiccated powder, or any other form suitable for use. In embodiments, the composition is in the form of a capsule. In another embodiment, the composition is in the form of a tablet. In embodiments, the pharmaceutical composition is formulated in the form of a soft-gel capsule. In a further embodiment, the pharmaceutical composition is formulated in the form of a gelatin capsule. In embodiments, the pharmaceutical composition is formulated as a liquid.

Where necessary, the inventive pharmaceutical compositions (and/or additional agents) can also include a solubilizing agent. Also, the agents can be delivered with a suitable vehicle or delivery device as known in the art. Combination therapies outlined herein can be co-delivered in a single delivery vehicle or delivery device.

The formulations comprising the inventive pharmaceutical compositions (and/or additional agents) of the present invention may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In various embodiments, any pharmaceutical compositions (and/or additional agents) described herein is formulated in accordance with routine procedures as a composition adapted for a mode of administration described herein.

Routes of administration include, for example: oral, intradermal, intramuscular, intraperitoneal, intravenous, subcutaneous, intranasal, epidural, sublingual, intranasal, intracerebral, intravaginal, transdermal, rectally, by inhalation, or topically. Administration can be local or systemic. In embodiments, the administration is by parenteral injection. The mode of administration can be left to the discretion of the practitioner and depends in-part upon the site of the medical condition. In most instances, administration results in the release of any agent described herein into the bloodstream.

In embodiments, the compositions described herein is formulated in accordance with routine procedures as a composition adapted for oral administration. Compositions for oral delivery can be in the form of tablets, lozenges, aqueous or oily suspensions, granules, powders, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving any compositions described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In embodiments, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

Dosage forms suitable for parenteral administration (e.g., intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art. Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage and should be preserved against microorganisms. The carrier can be a solvent or dispersion medium containing, for example, water, ethanol, polyol (for example, glycerol, propylene glycol, and liquid polyetheylene glycol), and suitable mixtures thereof.

The compositions provided herein, alone or in combination with other suitable components, can be made into aerosol formulations (e.g., “nebulized”) to be administered via inhalation. Aerosol formulations can be placed into pressurized acceptable propellants, such as dichlorodifluoromethane, propane, nitrogen, and the like.

Any inventive pharmaceutical compositions (and/or additional agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropyl cellulose, hydropropylmethyl cellulose, polyvinylpyrrolidone, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the agents described herein. The invention thus provides single unit dosage forms suitable for oral administration such as, but not limited to, tablets, capsules, gelcaps, and caplets that are adapted for controlled- or sustained-release.

Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

Trans-Splicing System

In embodiments, there is provided a trans-splicing system comprising the compositions or systems of the present disclosure.

In embodiments, the trans-splicing system comprises a splice donor and a splice acceptor. In embodiments, the trans-splicing system comprises a splice donor, a splice acceptor, and the trans-splicing system is suitable for replacing an internal exon in a gene.

In embodiments, the trans-splicing system comprises a repair template comprising a splice donor and/or a splice acceptor. In embodiments, the trans-splicing system comprises a repair template comprising a splice donor and a splice acceptor. In embodiments, the trans-splicing system comprises a repair template comprising a splice donor and a splice acceptor, and the trans-splicing system and/or repair template is suitable for replacing an internal exon in a gene. In embodiments, there is provided a method of trans-splicing an exon in a target nucleic acid, e.g. a pre-mRNA in a cell, the method comprising contacting the cell with the trans-splicing system disclosed herein, wherein the trans-splicing system comprises a repair template comprising a splice donor and a splice acceptor. In embodiments, the present disclosure provides a method of trans-splicing an exon in a target nucleic acid, e.g. a pre-mRNA in a cell in a subject in need thereof, comprising administering an effective amount of the trans-splicing system disclosed herein, wherein the trans-splicing system comprises a repair template comprising a splice donor and a splice acceptor.

In embodiments, the trans-splicing system further comprises a repair template lacking a splice donor and/or a splice acceptor. In embodiments, the trans-splicing system is suitable for splicing of a target nucleic acid comprising a splice donor or a splice acceptor site. In embodiments, the trans-splicing system comprises an RNA molecule, e.g. gRNA that targets a splice acceptor site. In embodiments, the trans-splicing system comprises an RNA molecule, e.g. gRNA that targets a splice donor site. In embodiments, the trans-splicing system comprises is regulated or regulatable by a small molecule. In variols embodiments, the small molecule is selected from abscisic acid (ABA), rapamycin (or rapalog), FK506, Cyclosporine A, FK1012, Gibberellin3-AM, FKCsA, AP1903/AP20187, and auxin. In embodiments, the pre-mRNA is at an intron-exon junction or exon-intron junctions.

In embodiments, the trans-splicing system comprises a pre-trans-splicing (PTS) molecule, wherein the PTS molecule comprises: i) one or more guideRNAs (gRNAs) that target a pre-mRNA; ii) an intronic sequence having a splice signal; and iii) a donor sequence encoding a gene product of a gene of interest, or portion thereof. In embodiments, the gRNA or gRNAs are within the PTS and processed by the endonuclease. In embodiments, the gRNA or gRNAs are within the PTS and are not processed by the endonuclease. In embodiments, the gRNA or gRNAs are not within the PTS. In embodiments, the PTS is the repRNA.

In embodiments, the trans-splicing system comprises a repair RNA (repRNA) sequence, comprising: (a) one or more exons and/or introns; (b) a splice donor and/or splice acceptor, wherein the repRNA is suitable for trans-splicing. In embodiments, the trans-splicing system comprises a system for trans-splicing a target nucleic acid comprising a repRNA, the repRNA comprising: (a) one or more exons and/or introns; and (b) a splice donor and/or splice acceptor. In embodiments, the repRNA comprises one or more binding motifs that direct, and/or hybridizes, the repRNA to a target nucleic acid molecule. In embodiments, the repRNA further comprises a guide RNA (gRNA). In embodiments, the gRNA hybridizes to the target nucleic acid molecule. In embodiments, the gRNA directs the repRNA to a target nucleic acid molecule.

In embodiments, the repRNA is operably linked to one or more sequences that are antisense to the target nucleic acid molecule. In embodiments, the repRNA is provided in cis to one or more sequences that bind to, and/or hybridize to, the target nucleic acid molecule. In embodiments, the repRNA is not operably linked to one or more sequences that bind to, and/or hybridize to, the target nucleic acid molecule. In embodiments, the repRNA is provided in trans to one or more sequences that bind to, and/or hybridize to, the target nucleic acid molecule. In embodiments, the repRNA is operably linked to one or more sequences that bind to, and/or hybridize to, an RNA-binding polypeptide. In embodiments, the repRNA is provided in cis to one or more sequences that bind to, and/or hybridize to, a RNA-binding polypeptide.

In embodiments, the repRNA is not operably to one or more sequences that bind to, and/or hybridize to, a RNA-binding polypeptide, which is herein described as a “grepRNA”.

In embodiments, the grepRNA comprises a repair RNA and a gRNA. In embodiments, “grepRNA” is used interchangeably with “PTS”.

In embodiments, there is provided a method of trans-splicing a target nucleic acid, e.g. a pre-mRNA in a cell, the method comprising contacting the cell with the trans-splicing system disclosed herein, wherein the trans-splicing system is substantially devoid of an RNA binding protein. In embodiments the present disclosure provides a method of trans-splicing a target nucleic acid, e.g. a pre-mRNA in a cell in a subject in need thereof, comprising administering an effective amount of the trans-splicing system disclosed herein, wherein the trans-splicing system is substantially devoid of an RNA binding protein.

In embodiments, there is provided a method of trans-splicing a target nucleic acid, e.g. a pre-mRNA in a cell, the method comprising contacting the cell with the trans-splicing system disclosed herein, wherein the trans-splicing system comprises a repair template operably linked to a guideRNA. In embodiments the present disclosure provides a method of trans-splicing a target nucleic acid, e.g. a pre-mRNA in a cell in a subject in need thereof, comprising administering an effective amount of the trans-splicing system disclosed herein, wherein the trans-splicing system comprises a repair template operably linked to a guideRNA and the trans-splicing system is substantially devoid of an RNA binding protein.

In embodiments, there is provided a method of trans-splicing a target nucleic acid, e.g. a pre-mRNA in a cell, the method comprising contacting the cell with the trans-splicing system disclosed herein, wherein the trans-splicing system comprises a repair template operably linked to a guideRNA. In embodiments the present disclosure provides a method of trans-splicing a target nucleic acid, e.g. a pre-mRNA in a cell in a subject in need thereof, comprising administering an effective amount of the trans-splicing system disclosed herein, wherein the trans-splicing system comprises a repair template operably linked to a guideRNA and the trans-splicing system is substantially devoid of an RNA binding protein.

In embodiments, the endonuclease is fused to a nucleic acid-interacting domain (e.g. MCP) and the PTS is tethered by the nuclease by interacting with the nucleic acid-interacting domain. In embodiments, one or more gRNAs hybridize to one or more pre-mRNAs. In embodiments, the endonuclease is catalytically active. In embodiments the endonuclease is catalytically inactive. In embodiments the endonuclease (or chimera) is delivered to a cell with viral methods. In embodiments the endonuclease (or chimera), and gRNA(s) or PTS are delivered or deliverable with viral methods. In embodiments the endonuclease (or chimera), gRNA(s), and PTS are delivered or deliverable with viral methods. In embodiments the endonuclease (or chimera) is delivered or deliverable to a cell with non-viral methods. In embodiments the endonuclease (or chimera), and gRNA(s) or PTS are delivered or deliverable with non-viral methods. In embodiments the endonuclease (or chimera), gRNA(s), and PTS are delivered or deliverable with non-viral methods. In embodiments, the endonuclease (or chimera) is delivered or deliverable to a cell implicated in disease. In embodiments, the endonuclease (or chimera) mediates trans-splicing of a pre-mRNA implicated in disease.

In embodiments, the system for targeting a nucleic acid for trans-splicing comprises an endonuclease disclosed herein (e.g., SEQ ID NO: 1, SEQ ID NO: 2, SEQ ID NO: 3, and SEQ ID NO: 4, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, or fragments or variants thereof), linked, associated, and/or fused to an RNA binding protein (RBP) selected from an MS binding protein, MS2 (MCP),PP7 coat protein, PRR1, HgaII, a Qbeta coat protein, the IN protein, or M protein.

In embodiments, the system for targeting a nucleic acid for trans-splicing comprises dPspCas13b linked, associated, and/or fused to an RBP. In embodiments, the system for targeting a nucleic acid for trans-splicing comprises dPspCas13b fused to MS2 (“dPspCas13b-MS2”). In embodiments, the system for targeting a nucleic acid for trans-splicing comprises dPspCas13b fused to PP7 (“dPspCas13b-PP7”). In embodiments, the system for targeting a nucleic acid for trans-splicing comprises dPspCas13b linked, associated, and/or fused to one or more of MS2, PRR1, M protein, HgaII, or a Qbeta hairpin and Qbeta coat protein (“QB”). In embodiments, the system for targeting a nucleic acid for trans-splicing comprises an inactive Cas13K2F endonuclease fused to an RBP. In embodiments, the system for targeting a nucleic acid for trans-splicing comprises an inactive Cas13K2F endonuclease fused to MS2 (“dCas13K2F-MS2”). In embodiments, the system for targeting a nucleic acid for trans-splicing comprises an inactive Cas13K2F endonuclease fused to PP7 (“dCas13K2F-PP7”).

In embodiments, the system for targeting a nucleic acid for trans-splicing comprises a second target comprising a splice donor sequence, an intron sequence, a splice acceptor sequence, and an exon sequence. In embodiments, the system for targeting a nucleic acid for trans-splicing comprises a trans-splicing template comprising a splice donor and/or splice acceptor, and an RNA sequence that binds to the specific RNA-binding polypeptide. In embodiments, the trans-splicing template comprises two MS2 stem loops.

In embodiments, disclosed herein is a system for targeting a nucleic acid for trans-splicing, the system comprising: (a) an endonuclease of any one the embodiments disclosed herein, and optionally an RNA molecule comprising a sequence complementary to one strand of a target nucleic acid molecule; (b) an RNA-binding polypeptide that associates with the endonuclease; and (c) a trans-splicing nucleic acid template comprising: (i) a splice donor and/or splice acceptor, and (ii) an RNA sequence that binds to the RNA-binding polypeptide. In embodiments, the RNA molecule is a gRNA.

In embodiments, the endonuclease is linked, associated, and/or fused with the RNA binding protein. In embodiments, the RNA binding protein is a viral protein. In embodiments, the RNA binding protein is an MS binding protein. In embodiments, the RNA binding protein is a PP7 coat protein. In embodiments, the RNA binding protein is PRR1. In embodiments, the RNA binding protein is HgaII. In embodiments, the RNA binding protein is a Qbeta coat protein. In embodiments, the RNA binding protein is the IN protein. In embodiments, the RNA binding protein is the M protein.

In embodiments, disclosed herein is a method for targeted trans-splicing of a pre-mRNA in a cell, comprising contacting the cell with the system of any one of the embodiments disclosed herein.

In embodiments, disclosed herein is a system for targeting a nucleic acid for trans-splicing, the system comprising: (a) dPspCas13b and optionally an RNA molecule comprising a sequence complementary to one strand of a target nucleic acid molecule; (b) an RNA-binding polypeptide that associates with the endonuclease, the RNA-binding polypeptide being selected from one or more of PP7, M, PRR1, HgaII, or Qbeta coat protein; and (c) a trans-splicing nucleic acid template comprising: (i) a splice donor and/or splice acceptor, and (ii) an RNA sequence that binds to the RNA-binding polypeptide.

In embodiments, the RNA molecule is a gRNA.

In embodiments, the endonuclease is linked, associated, and/or fused with the RNA binding protein.

In embodiments, disclosed herein is a method for targeted trans-splicing of a pre-mRNA in a cell, comprising contacting the cell with the system of any one of the embodiments disclosed herein.

### RNA Molecules

In embodiments, the RNA molecule binds to or interacts with the present endonuclease.

In embodiments, the RNA molecule binds to or interacts with the present chimeric protein.

In embodiments, the RNA molecule binds to or interacts with the present target nucleic acid.

In embodiments, the RNA molecule is or comprises a guide ribonucleic structure configured to form a complex with the endonuclease.

In embodiments, the guide ribonucleic structure (i) comprises (a) a CRISPR RNA (crRNA) suitable for hybridizing to a target nucleic acid molecule and/or (b) a transactivating CRISPR RNA (tracrRNA) suitable for interacting with the endonuclease or (ii) lacks a (a) a crRNA suitable for hybridizing to a target nucleic acid molecule and/or (b) a tracrRNA suitable for interacting with the endonuclease.

In embodiments, the RNA molecule is or comprises a gRNA. In embodiments, the gRNA comprises a sequence that interacts with the endonuclease. In embodiments, the endonuclease forms a complex with the gRNA.

In embodiments, the RNA molecule contains a repeat and spacer. In embodiments, the RNA has a repeat before a spacer. In embodiments the RNA has a spacer before a repeat. In embodiments the RNA has a repeat before a spacer before a repeat. In embodiments, the RNA has multiple spacers separated by multiple repeats. In embodiments, the RNA has multiple gRNAs. In embodiments, the RNA has multiple gRNAs and is cleaved or processed to separate the gRNAs. In embodiments, the RNA has spacers that target different nucleic acid targets. In embodiments, the RNA has spacers that target the same nucleic acid target.

In embodiments, the endonuclease repeat sequences to formulate the guide RNA are selected from Table 3 below.

TABLE 3
Endonuclease repeat sequences to
formulate the guide RNA

| | SEQ |
|---|---|
| Sequence | ID NO: |
| GTTAGAATATAACCCTGTTTGTAGGGGTAATAAAAC | 28 |
| GTTAGAATATAACCCTGTTTGTAGGGGTAATAAAAC | 29 |
| GTTGGAATATAACCCCGTTTGTAGGGGTAATAAAAC | 30 |
| GTTAGAATATAACCCTGTTTGTAGGGGTAATAAAAC | 31 |
| GTTAGAATATACCCCATTTTGTATGGGGATTAAAAC | 90 |
| GTTGGAATATAGCCCTGTTTGTAGGGGTAATAAAAC | 91 |
| GATGGAATATAACCCCGTTTGTAGGGGTAATAAAAC | 92 |
| AGTAGAATATAACCCTGATATTAGTAGGGGTAATAAAAC | 93 |
| AGTAGAATATAACCCTGTTAGTAGGGGTAATAAAAC | 94 |
| TATTGACTATACCCCTGATTTGTAGGGGTAAAAGAGA | 95 |
| TATTGACTATACCCCTGATTTGTAGGGGTAAAAAAAC | 96 |
| GTCAGACTATACCCTCGTTTGTAGGGGGAATAAAAC | 97 |

In embodiments, the guide RNA is or comprises a sequence of SEQ ID NOs: 28-31, and/or SEQ ID NOs: 90-97, or a fragment or variant thereof, or a nucleic acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto. In embodiments, the guide RNA is or comprises a sequence of SEQ ID NO: 28 or a fragment or variant thereof, or a nucleic acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto. In embodiments, the guide RNA is or comprises a sequence of SEQ ID NO: 29 or a fragment or variant thereof, or a nucleic acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto. In embodiments, the guide RNA is or comprises a sequence of SEQ ID NO: 30 or a fragment or variant thereof, or a nucleic acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto. In embodiments, the guide RNA is or comprises a sequence of SEQ ID NO: 31 or a fragment or variant thereof, or a nucleic acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto. In embodiments, the guide RNA is or comprises a sequence of SEQ ID NO: 90 or a fragment or variant thereof, or a nucleic acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto. In embodiments, the guide RNA is or comprises a sequence of SEQ ID NO: 91 or a fragment or variant thereof, or a nucleic acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto. In embodiments, the guide RNA is or comprises a sequence of SEQ ID NO: 92 or a fragment or variant thereof, or a nucleic acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto. In embodiments, the guide RNA is or comprises a sequence of SEQ ID NO: 93 or a fragment or variant thereof, or a nucleic acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto. In embodiments, the guide RNA is or comprises a sequence of SEQ ID NO: 94 or a fragment or variant thereof, or a nucleic acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto. In embodiments, the guide RNA is or comprises a sequence of SEQ ID NO: 95 or a fragment or variant thereof, or a nucleic acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto. In embodiments, the guide RNA is or comprises a sequence of SEQ ID NO: 96 or a fragment or variant thereof, or a nucleic acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto. In embodiments, the guide RNA is or comprises a sequence of SEQ ID NO: 97 or a fragment or variant thereof, or a nucleic acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% identity thereto.

In embodiments, the endonuclease is a polypeptide of one of SEQ ID NOs: 1-4, and/or SEQ ID NOs: 80-89, or a fragment or a variant thereof, or a nucleic acid polypeptide of one of SEQ ID NOs: 1-4, and/or SEQ ID NOs: 80-89, or a fragment or variant thereof. In embodiments, the endonuclease is guided to a target nucleic acid using a guide RNA selected from SEQ ID NOs: 28-31, and/or SEQ ID NOs: 90-97, or a fragment or variant thereof, and/or the endonuclease is associated with a guide RNA selected from SEQ ID NOs: 28-31, and/or SEQ ID NOs: 90-97, or a fragment or variant thereof.

In embodiments, the endonuclease is SEQ ID NO: 1, or a fragment or variant thereof, and the guide RNA is selected from SEQ ID NO: 28, SEQ ID NO: 29, SEQ ID NO: 30, or SEQ ID NO: 31, SEQ ID NO: 90, SEQ ID NO: 91, SEQ ID NO: 92, SEQ ID NO: 93, SEQ ID NO: 94, SEQ ID NO: 95, SEQ ID NO: 96, or SEQ ID NO: 97, or a fragment or variant thereof.

In embodiments, the endonuclease is SEQ ID NO: 2, or a fragment or variant thereof, and the guide RNA is selected from SEQ ID NO: 28, SEQ ID NO: 29, SEQ ID NO: 30, or SEQ ID NO: 31, SEQ ID NO: 90, SEQ ID NO: 91, SEQ ID NO: 92, SEQ ID NO: 93, SEQ ID NO: 94, SEQ ID NO: 95, SEQ ID NO: 96, or SEQ ID NO: 97, or a fragment or variant thereof.

In embodiments, the endonuclease is SEQ ID NO: 3, or a fragment or variant thereof, and the guide RNA is selected from SEQ ID NO: 28, SEQ ID NO: 29, SEQ ID NO: 30, or SEQ ID NO: 31, SEQ ID NO: 90, SEQ ID NO: 91, SEQ ID NO: 92, SEQ ID NO: 93, SEQ ID NO: 94, SEQ ID NO: 95, SEQ ID NO: 96, or SEQ ID NO: 97, or a fragment or variant thereof.

In embodiments, the endonuclease is SEQ ID NO: 4, or a fragment or variant thereof, and the guide RNA is selected from SEQ ID NO: 28, SEQ ID NO: 29, SEQ ID NO: 30, or SEQ ID NO: 31, SEQ ID NO: 90, SEQ ID NO: 91, SEQ ID NO: 92, SEQ ID NO: 93, SEQ ID NO: 94, SEQ ID NO: 95, SEQ ID NO: 96, or SEQ ID NO: 97, or a fragment or variant thereof.

In embodiments, the endonuclease is SEQ ID NO: 80, or a fragment or variant thereof, and the guide RNA is selected from SEQ ID NO: 28, SEQ ID NO: 29, SEQ ID NO: 30, or SEQ ID NO: 31, SEQ ID NO: 90, SEQ ID NO: 91, SEQ ID NO: 92, SEQ ID NO: 93, SEQ ID NO: 94, SEQ ID NO: 95, SEQ ID NO: 96, or SEQ ID NO: 97, or a fragment or variant thereof.

In embodiments, the endonuclease is SEQ ID NO: 81, or a fragment or variant thereof, and the guide RNA is selected from SEQ ID NO: 28, SEQ ID NO: 29, SEQ ID NO: 30, or SEQ ID NO: 31, SEQ ID NO: 90, SEQ ID NO: 91, SEQ ID NO: 92, SEQ ID NO: 93, SEQ ID NO: 94, SEQ ID NO: 95, SEQ ID NO: 96, or SEQ ID NO: 97, or a fragment or variant thereof.

In embodiments, the endonuclease is SEQ ID NO: 82, or a fragment or variant thereof, and the guide RNA is selected from SEQ ID NO: 28, SEQ ID NO: 29, SEQ ID NO: 30, or SEQ ID NO: 31, SEQ ID NO: 90, SEQ ID NO: 91, SEQ ID NO: 92, SEQ ID NO: 93, SEQ ID NO: 94, SEQ ID NO: 95, SEQ ID NO: 96, or SEQ ID NO: 97, or a fragment or variant thereof.

In embodiments, the endonuclease is SEQ ID NO: 83, or a fragment or variant thereof, and the guide RNA is selected from SEQ ID NO: 28, SEQ ID NO: 29, SEQ ID NO: 30, or SEQ ID NO: 31, SEQ ID NO: 90, SEQ ID NO: 91, SEQ ID NO: 92, SEQ ID NO: 93, SEQ ID NO: 94, SEQ ID NO: 95, SEQ ID NO: 96, or SEQ ID NO: 97, or a fragment or variant thereof.

In embodiments, the endonuclease is SEQ ID NO: 84, or a fragment or variant thereof, and the guide RNA is selected from SEQ ID NO: 28, SEQ ID NO: 29, SEQ ID NO: 30, or SEQ ID NO: 31, SEQ ID NO: 90, SEQ ID NO: 91, SEQ ID NO: 92, SEQ ID NO: 93, SEQ ID NO: 94, SEQ ID NO: 95, SEQ ID NO: 96, or SEQ ID NO: 97, or a fragment or variant thereof.

In embodiments, the endonuclease is SEQ ID NO: 85, or a fragment or variant thereof, and the guide RNA is selected from SEQ ID NO: 28, SEQ ID NO: 29, SEQ ID NO: 30, or SEQ ID NO: 31, SEQ ID NO: 90, SEQ ID NO: 91, SEQ ID NO: 92, SEQ ID NO: 93, SEQ ID NO: 94, SEQ ID NO: 95, SEQ ID NO: 96, or SEQ ID NO: 97, or a fragment or variant thereof.

In embodiments, the endonuclease is SEQ ID NO: 86, or a fragment or variant thereof, and the guide RNA is selected from SEQ ID NO: 28, SEQ ID NO: 29, SEQ ID NO: 30, or SEQ ID NO: 31, SEQ ID NO: 90, SEQ ID NO: 91, SEQ ID NO: 92, SEQ ID NO: 93, SEQ ID NO: 94, SEQ ID NO: 95, SEQ ID NO: 96, or SEQ ID NO: 97, or a fragment or variant thereof.

In embodiments, the endonuclease is SEQ ID NO: 87, or a fragment or variant thereof, and the guide RNA is selected from SEQ ID NO: 28, SEQ ID NO: 29, SEQ ID NO: 30, or SEQ ID NO: 31, SEQ ID NO: 90, SEQ ID NO: 91, SEQ ID NO: 92, SEQ ID NO: 93, SEQ ID NO: 94, SEQ ID NO: 95, SEQ ID NO: 96, or SEQ ID NO: 97, or a fragment or variant thereof.

In embodiments, the endonuclease is SEQ ID NO: 88, or a fragment or variant thereof, and the guide RNA is selected from SEQ ID NO: 28, SEQ ID NO: 29, SEQ ID NO: 30, or SEQ ID NO: 31, SEQ ID NO: 90, SEQ ID NO: 91, SEQ ID NO: 92, SEQ ID NO: 93, SEQ ID NO: 94, SEQ ID NO: 95, SEQ ID NO: 96, or SEQ ID NO: 97, or a fragment or variant thereof.

In embodiments, the endonuclease is SEQ ID NO: 89, or a fragment or variant thereof, and the guide RNA is selected from SEQ ID NO: 28, SEQ ID NO: 29, SEQ ID NO: 30, or SEQ ID NO: 31, SEQ ID NO: 90, SEQ ID NO: 91, SEQ ID NO: 92, SEQ ID NO: 93, SEQ ID NO: 94, SEQ ID NO: 95, SEQ ID NO: 96, or SEQ ID NO: 97, or a fragment or variant thereof.

In embodiments, the endonuclease is SEQ ID NO: 1, or a fragment or variant thereof, and the guide RNA is SEQ ID NO: 28.

In embodiments, the endonuclease is SEQ ID NO: 2, or a fragment or variant thereof, and the guide RNA is SEQ ID NO: 29.

In embodiments, the endonuclease is SEQ ID NO: 3, or a fragment or variant thereof, and the guide RNA is SEQ ID NO: 30.

In embodiments, the endonuclease is SEQ ID NO: 4, or a fragment or variant thereof, and the guide RNA is SEQ ID NO: 31.

In embodiments, the endonuclease is SEQ ID NO: 80, or a fragment or variant thereof, and the guide RNA is SEQ ID NO: 90.

In embodiments, the endonuclease is SEQ ID NO: 81, or a fragment or variant thereof, and the guide RNA is SEQ ID NO: 90

In embodiments, the endonuclease is SEQ ID NO: 82, or a fragment or variant thereof, and the guide RNA is SEQ ID NO: 90.

In embodiments, the endonuclease is SEQ ID NO: 83, or a fragment or variant thereof, and the guide RNA is SEQ ID NO: 91.

In embodiments, the endonuclease is SEQ ID NO: 84, or a fragment or variant thereof, and the guide RNA is SEQ ID NO: 92.

In embodiments, the endonuclease is SEQ ID NO: 85, or a fragment or variant thereof, and the guide RNA is SEQ ID NO: 93, or SEQ ID NO: 94.

In embodiments, the endonuclease is SEQ ID NO: 86, or a fragment or variant thereof, and the guide RNA is SEQ ID NO: 28.

In embodiments, the endonuclease is SEQ ID NO: 87, or a fragment or variant thereof, and the guide RNA is SEQ ID NO: 28.

In embodiments, the endonuclease is SEQ ID NO: 88, or a fragment or variant thereof, and the guide RNA is SEQ ID NO: 95, or SEQ ID NO: 96.

In embodiments, the endonuclease is SEQ ID NO: 89, or a fragment or variant thereof, and the guide RNA is SEQ ID NO: 97.

In embodiments, the RNA molecule is or comprises at least about 4, 5, 6, 7, 8, 9, 10, 11, 12, 13, 14, 15, 16, 17, 18, 19, or 20 nucleotides in length. In embodiments, the RNA molecule is or comprises less than about 300, 250, 200, 100, 150, or 50 nucleotides in length. In embodiments, the RNA molecule is or comprises about 5-10, about 5-15, about 5-20, about 10-20, about 10-30, about 10-40, about 10-50, about 10-60, about 10-70, about 10-80, about 10-90, about 10-100, about 50-100, about 50-150, about 50-200, about 50-250, about 50-300, about 100-200, about 100-300, or about 200-300 nucleotides in length.

In embodiments, the RNA molecule is 10-50 nucleotides in length, e.g., 10-45, 10-40, 10-35, 10-30,10-20, 11-45, 11-40, 11-35, 11-30,11-20, 12-45, 12-40,12-35, 12-30, 12-25, 12-20, 13-45, 13-40, 13-35, 13-30, 13-25, 13-20, 14-45, 14-40, 14-35, 14-30, 14-25, 14-20, 15-45, 15-40, 15-35, 15-30, 15-25, 15-20, 16-45, 16-40, 16-35, 16-30, 16-25, 16-20, 17-45, 17-40, 17-35, 17-30, 17-25, 17-20, 18-45, 18-40, 18-35, 18-30, 18-25, 18-20, 19-45, 19-40, 19-35, 19-30, 19-25, 19-20, e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In embodiments, the present disclosure provides a composition comprising a nucleic acid encoding an endonuclease comprising a sequence, optionally comprising a HEPN domain, or a fragment or variant thereof, in conjunction with an RNA containing a repeat having at least about 70% identity to one or more of SEQ ID NOs: 28-31, and/or SEQ ID NOs: 90-97. In embodiments, the composition has least about 75%, or at least about 80%, or at least about 85%, or at least about 90%, or at least about 91%, or at least about 92%, or at least about 93%, or at least about 94%, or at least about 95%, or at least about 96%%, or at least about 97%, or at least about 98%, or at least about 99%) identity to SEQ ID NOs: 28-31, and/or SEQ ID NOs: 90-97, or has about 1 to about 20 nucleotide modifications (e.g. about 1, or about 2, or about 3, or about 4, or about 5, or about 6, or about 7, or about 8, or about 9, or about 10, or about 11, or about 12, or about 13, or about 14, or about 15, or about 16, or about 17, or about 18, or about 19, or about 20 modifications). In embodiments, the sequence comprises at least one HEPN domain, or fragments or variants thereof. In embodiments, the sequence comprises at least two HEPN domains, or fragments or variants thereof.

### Nucleic Acid Modifications

In embodiments, a nucleic acid disclosed herein has one or more modifications, e.g., a base modification, a backbone modification, and the like, to provide the nucleic acid with a new or enhanced feature (e.g., improved stability). A nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the ph′sphate ‘roup can b’ linked to the 2′, the 3′, or the 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are suitable. In addition, linear compounds may have internal nucleotide base complementarity and may therefore fold in a manner as to produce a fully or partially double-stranded compound. Within oligonucleotides, the phosphate groups are commonly referred to as forming the intemucleoside backbone of the oligonucleotide. The normal linka′e or′ackbone of RNA and DNA is a 3′ to 5′ phosphodiester linkage.

In embodiments, a suitable nucleic acid modificatio′ include, but are not limited to′ 2O methyl modified nucleotides, 2′ Fluoro modified nucleotides, locked nucleic acid (LNA) modified nucleotides, peptide nucleic acid (PNA) modified nucleotides, nucleotides wit′ phosphorothioate linkages, and a 5′ cap (e.g., a 7-methylguanylate cap (m7G)). A 2′-O-Methyl modified nucleotide (also referred to as 2′-O-Methyl RNA) is a naturally occurring modification of RNA found in tRNA and other small RNAs that arises as a post-transcriptional modification. Oligonucleotides can be′directly synthesized that contain 2′-O-Methyl RNA. Without wishing to be bound by theory, this modification increases Tm of RNA:RNA duplexes but results in only small changes in RNA:DNA stability. It is stabile with respect to attack by single-stranded ribonucleases and is typically about 5 to about 10-fold le's susceptible to DNases than DNA. 2′ ′luoro modified nucleotides (e.g., 2′ Fluoro bases) have a fluorine modified ribolhich increases binding affinity (Tm) and also confers some relative nuclease resistance when compared to native RNA. LNA bases have a modification to the ribos' backbone that locks the base in the C3′-endo position, which favors RNA A-type helix duplex geometry. Without wishing to be bound by theory, this modification significantly increases Tm and is also very nuclease resistant. Multiple LNA insertions can be placed ′n an oligo at any position except the 3′-end. Applications have been described ranging from antisense oligos to hybridization probes to SNP detection and allele specific PCR. Due to the large increase in Tm conferred by LNAs, they also can cause an increase in primer dimer formation as well as self-hairpin formation. In embodiments, the number of LNAs incorporated into a single oligo is 10 bases or less. The phosphorothioate (PS) bond (e.g., a phosphorothioate linkage) substitutes a sulfur atom for a non-bridging oxygen in the phosphate backbone of a nucleic acid (e.g., an oligo). Without wishing to be bound by theory, this modification renders the internucleotide linkage resistant to nuclease degradation. Phosphorothioate bonds can be introduced b′tween′he last 3-5 nucleotides at the 5′- or 3′-end of the oligo to inhibit exonuclease degradation. Including phosphorothioate bonds within the oligo (e.g., throughout the entire oligo) can help reduce attack by endonucleases as well.

### Modified Backbones and Modified Internucleoside Linkages

In embodiments, the present nucleic acids, optionally containing modifications, include nucleic acids containing modified backbones or non-natural internucleoside linkages. In embodiments, nucleic acids having modified backbones include those that retain a phosphorus atom in the backbone and those that do not have a phosphorus atom in the backbone.

In embodiments, oligonucleotide backbones containing a phosphorus atom therein include, for example, phosphorothioates, chiral phosphorothioates, phosphorodithioates, phosphotriesters, aminoalkylphosphotriesters, methyl ′nd other alkyl phosphonat's including 3′-alkylene phosphonates, 5′-alkylene phosphonates and chiral phosphonates, ph′sphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranoph′sp′ates having normal 3′-5′ linkages, 2′-5′ linked analogs of these, and those having inverted polarity wherein one′or mo′e i′ternu′leoti′e lin′ages is a 3′ to 3′, 5′ to 5′ or 2′ to 2′ linkage. Suitable oligonucleotides havi′g inv′rted polarity com′rise a single 3′ to 3′ linkage at the 3′-most intemucleotide linkage i.e a single inverted nucleoside residue which may be a basic (the nucleobase is missing or has a hydroxyl group in place thereof). Various salts (such as, for example, potassium or sodium), mixed salts and free acid forms are also included.

In embodiments, the present nucleic acids comprise one or more phosphorothioate and/or heteroatom internucleoside linkages, in particular —CH₂—NH—O—CH₂—, —CH₂—N(CH₃)—O—CH₂-(known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). MMI type internucleoside linkages are disclosed in the above referenced U.S. Pat. No. 5,489,677, the disclosure of which is incorporated herein by reference in its entirety. Suitable amide intemucleoside linkages are disclosed in U.SIIt. No. 5,602,240, the disclosure of which is incorporated herein by reference in its entirety.

Suitable modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

### Mimetics

In embodiments, the present nucleic acid is in the form of or comprises a nucleic acid mimetic. In embodiments, the term “mimetic” as it is applied to polynucleotides is intended to include polynucleotides where only the furanose ring or both the furanose ring and the internucleotide linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone.

In embodiments, the present nucleic acid is or comprises a mimetic based on morpholino units (morpholino nucleic acid), having heterocyclic bases attached to the morpholino ring. A number of linking groups have been reported that link the morpholino monomeric units in a morpholino nucleic acid. One class of linking groups has been selected to give a non-ionic oligomeric compound. The non-ionic morpholino-based oligomeric compounds are less likely to have undesired interactions with cellular proteins. Morpholino-based polynucleotides are non-ionic mimics of oligonucleotides which are less likely to form undesired interactions with cellular proteins (Dwaine A. Braasch and David R. Corey, Biochemistry, 2002, 41(14), 4503-4510). Morpholino-based polynucleotides are disclosed in U.S. Pat. No. 5,034,506, the disclosure of which is incorporated herein by reference in its entirety. A variety of compounds within the morpholino class of polynucleotides have been prepared, having a variety of different linking groups joining the monomeric subunits.

In embodiments, the present nucleic acid is a mimetic is in the form of or comprises cyclohexenyl nucleic acids (CeNA). The furanose ring normally present in a DNA/RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J Am. Chem. Soc., 2000, 122, 8595-8602, the disclosure of which is incorporated herein by reference in its entirety). In general, the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation.

In embodiments, the present nucleic acid compris's Locked Nucleic Acids (LNAs) in w′ich the 2′-hydroxyl group is linked to the 4′ carb′n ato′ of the sugar ring thereby forming a 2′-C, 4′-C-oxymethylene linkage thereby forming a bicyclic sugar moiety. The linkage can′be a methylene (—CH₂—)′ group bridging the 2′ oxygen atom and the 4′ carbon atom wherein n is 1 or 2 (Singh et al., Chem. Commun., 1998, 4, 455-456, the disclosure of which is incorporated herein by reference in its entirety). LNA and LNA analogs display very high duplex thermal stabilities with complementary DNA a′d RNA (Tm=+3 to +10° C.), stability towards 3′-exonucleolytic degradation and good solubility properties. Potent and nontoxic antisense oligonucleotides containing LNAs have been described (e.g., Wahlestedt et al., Proc. Natl. Acad. Sci. U.S.A., 2000, 97, 5633-5638, the disclosure of which is incorporated herein by reference in its entirety).

### Modified Sugar Moieties

In embodiments, the present nucleic acid comprises one or more substituted sugar moieties. Suitable polynucleotides comprise a sugar substituent group selected from: OH; F; O—, S-, or N-alkyl; O—, S-, or N-alkenyl; O—, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar p′operties. A suita′le modification includes 2′-met′oxyethoxy (2′-O—CH₂ CH₂O′H3, also known as 2′-O-(2-methoxyethyl) or 2′-MOE) (Martin et al., Helv. Chim. Acta, 1995, 78, 486-504, the disclosure of which is incorporated herein by reference in its entirety) i.e., an alkoxyalkoxy grou′. A further suitable modification includes 2′-dimethylaminooxyethoxy, i′e., a O(CH₂)₂ON(CH₃)₂ group, also known as 2′-DMAOE′ as described in examples hereinbelow, and 2′-dimethyl′minoethoxyethoxy (also known in the′art as 2′-O-dimet′yl-amino-ethoxy-ethyl or 2′-DMAEOE), i.e., 2′-O—CH₂—O—CH₂—N(CH₃)₂.

Other suitable sugar substituent groups include methoxy (—O—CH₃), aminopropoxy (—O CH—H₂NH₂), allyl (—CH₂—CH═CH₂), ′O-allyl (—O— CH₂—CH═CH₂) and fluoro (F). 2′-sugar substituent groups may be in the arabino (up) p′sition or ribo (down) posi′ion. A suitable 2′-arabino modification is 2′-F. Similar modifications may also be made at other positions o′ the oligomeric compound, parti′ularly the 3′ position of th′ s′gar on the 3′ terminal nucleoside ′r in 2′-5′ lin′ed oligonucleotides and the 5′ position of 5′ terminal nucleotide. Oligomeric compounds may also have sugar mimetics such as cyclobutyl moieties in place of the pentofuranosyl sugar.

### Base Modifications and Substitutions

In embodiments, the present nucleic acid comprises one or more nucleobase (often referred to in the art simply as “base”) modifications or substitutions. In embodiments, as used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)in′ol′2-one), pyridoindole cytidine (H-pyrido(3′,2′:4,5)pyrrolo(2,3-d)pyrimidin-2-one).

In embodiments, heterocyclic base moieties may also include those in which the purine or pyrimidine base is replaced with other heterocycles, for example 7-deaza-adenine, 7-deazaguanosine, 2-aminopyridine and 2-pyridone. Certain nucleobases are, without wishing to be bound by theory, useful for increasing the binding affinity of an oligomeric compound. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability and are suitable b′se substitutions, e.g., when combined with 2′-O-methoxyethyl sugar modifications.

### Conjugates

In embodiments, the present compositions are chemically linked to one or more moieties or conjugates which enhance the activity, cellular distribution or cellular uptake of the oligonucleotide. In embodiments, the moieties or conjugates include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a subject nucleic acid.

In embodiments, the conjugate may include a Protein Transduction Domain or PTD (also known as a CPP—cell penetrating peptide), which may refer to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle (e.g., the nucleus). In emb′diments, a PTD is covalently linked to the 3′ end of an exogenous polynucleotide. In emb′diments, a PTD is covalently linked to the 5′ end of an exogenous polynucleotide.

### Kits

In aspects, the present disclosure provides a kit comprising a container comprising the composition of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, the cell of any one of the embodiments and/or aspects disclosed herein, or the pharmaceutical composition of any one of the embodiments and/or aspects disclosed herein and with instructions for use in modulating and/or modifying a nucleic acid.

In embodiments, the present disclosure provides kits for carrying out the methods described herein. In embodiments, the kit comprises a composition described herein, a recombinant expression vector, a delivery system, and/or a pharmaceutical composition described herein, optionally further with a reagent for reconstitution and/or dilution.

### Methods of Use

In aspects, the present disclosure provides a method of modulating and/or modifying a nucleic acid in a cell, comprising contacting the cell with the composition of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, the cell of any one of the embodiments and/or aspects disclosed herein, or the pharmaceutical composition of any one of the embodiments and/or aspects disclosed herein.

In aspects, the present disclosure provides a method of modulating and/or modifying a nucleic acid in a subject in need thereof, comprising administering an effective amount of the cell with the composition of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, the cell of any one of the embodiments and/or aspects disclosed herein, or the pharmaceutical composition of any one of the embodiments and/or aspects disclosed herein to the subject.

In embodiments, the modulating and/or modifying is selected from one or more of cleaving, nicking, methylating, labeling, and mutating the nucleic acid. In embodiments, the modulating and/or modifying is selected from one or more of cleaving the nucleic acid; inserting a nucleic acid, editing the nucleic acid; modulating transcription from the nucleic acid; isolating the nucleic acid, binding the nucleic acid, and imaging the nucleic acid.

In aspects, the present disclosure provides a method of disrupting, correcting, and/or replacing a gene in a cell, comprising contacting the cell with the composition of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, the cell of any one of the embodiments and/or aspects disclosed herein, or the pharmaceutical composition of any one of the embodiments and/or aspects disclosed herein.

In aspects, the present disclosure provides a method of disrupting, correcting, and/or replacing a gene in a subject in need thereof, comprising administering an effective amount of the composition of any one of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, the cell of any one of the embodiments and/or aspects disclosed herein, or the pharmaceutical composition of any one of the embodiments and/or aspects disclosed herein to the subject.

In aspects, the present disclosure provides a method of trans-splicing a nucleic acid, e.g. a pre-mRNA in a cell, the method comprising contacting the cell with the composition of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, the cell of any one of the embodiments and/or aspects disclosed herein, or the pharmaceutical composition of any one of the embodiments and/or aspects disclosed herein.

In aspects, the present disclosure provides a method of trans-splicing a nucleic acid, e.g. a pre-mRNA in a cell, in a subject in need thereof, comprising administering an effective amount of the composition of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, the cell of any one of the embodiments and/or aspects disclosed herein, or the pharmaceutical composition of any one of the embodiments and/or aspects disclosed herein.

In embodiments, the trans-splicing method further comprises use of a repair template comprising a splice donor and/or a splice acceptor. In embodiments, the trans-splicing method further comprises use of a repair template lacking a splice donor and/or a splice acceptor. In embodiments, the trans-splicing method provides splicing of a target nucleic acid comprising a splice donor or a splice acceptor site. In embodiments, the trans-splicing method uses an RNA molecule, e.g. gRNA that targets a splice acceptor site. In embodiments, the trans-splicing method uses an RNA molecule, e.g. gRNA that targets a splice donor site. In embodiments, the activity of at least one portion of the system used in the trans-spliciIg methods is regulated by a small molecule. In various embodiments, the small molecule is selected from abscisic acid (ABA), rapamycin (or rapalog), FK506, Cyclosporine A, FK1012, Gibberellin3-AM, FKCsA, AP1903/AP20187, and auxin. In embodiments, the pre-mRNA is at an intron-exon junction or exon-intron junctions. In embodiments, the trans-splicing method further comprises use of a pre-trans-splicing (PTS) molecule, wherein the PTS molecule comprises: i) one or more guideRNAs (gRNAs) that target a pre-mRNA; ii) an intronic sequence having a splice signal; and iii) a donor sequence encoding a gene product of a gene of interest, or portion thereof. In embodiments, the gRNA or gRNAs are within the PTS and processed by the endonuclease. In embodiments, the gRNA or gRNAs are within the PTS and are not processed by the endonuclease. In embodiments, the gRNA or gRNAs are not within the PTS. In embodiments, the endonuclease is fused to a nucleic acid-interacting domain (e.g. MCP) and the PTS is tethered by the nuclease by interacting with the nucleic acid-interacting domain. In embodiments, one or more gRNAs hybridize to one or more pre-mRNAs. In embodiments, the endonuclease is catalytically active. In embodiments the endonuclease is catalytically inactive. In embodiments the endonuclease (or chimera) is delivered to a cell with viral methods. In embodiments the endonuclease (or chimera), and gRNA(s) or PTS are delivered with viral methods. In embodiments the endonuclease (or chimera), gRNA(s), and PTS are delivered with viral methods. In embodiments the endonuclease (or chimera) is delivered to a cell with non-viral methods. In embodiments the endonuclease (or chimera), and gRNA(s) or PTS are delivered with non-viral methods. In embodiments the endonuclease (or chimera), gRNA(s), and PTS are delivered with non-viral methods. In embodiments, the endonuclease (or chimera) is delivered to a cell implicated in disease. In embodiments, the endonuclease (or chimera) mediates trans-splicing of a pre-mRNA implicated in disease.

In aspects, the present disclosure provides a method of modifying a nucleic acid in a cell using a method of exon skipping, comprising contacting the cell with the composition of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, the cell of any one of the embodiments and/or aspects disclosed herein, or the pharmaceutical composition of any one of the embodiments and/or aspects disclosed herein.

In aspects, the present disclosure provides a method of modifying a nucleic acid in a cell in a subject in need thereof, using a method of exon skipping, comprising administering an effective amount of the composition of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, the cell of any one of the embodiments and/or aspects disclosed herein, or the pharmaceutical composition of any one of the embodiments and/or aspects disclosed herein.

In embodiments, the cell is induced to skip over faulty sections of pre-mRNA molecules by interfering with mRNA splicing. In embodiments, the methods produce a truncated, but functional, protein despite the presence of a mutation. In embodiments, the method of exon skipping involves binding an oligonucleotide (e.g. without limitation the present RNA molecule) to a splice site in a pre-mRNA molecule. In embodiments, when the pre-mRNA that is bound by the oligonucleotide is processed into a mature mRNA, the corresponding exon is skipped over, which, for example, restores a disrupted reading frame caused by the mutation. In embodiments, the exon skipping allows translation of an internally-deleted, but substantially functional protein.

In embodiments, the present exon skipping methods comprise creating a single-strand or double-strand break in a gene. In embodiments, the single-strand or double-strand break causes persistent altered splicing of the gene. In embodiments, the altered splicing results in expression of a truncated protein which lacks at least the polypeptide sequence corresponding to an exon containing the mutation. In embodiments, the single-strand or double-strand break removes a splice acceptor site or produces a non-functional splice acceptor site in or near an exon of the gene or removes a splice donor site or produces a non-functional splice donor site in or near an exon of the gene.

In aspects, the present disclosure provides a method of treating, ameliorating or preventing a disease or disorder in a subject, comprising (a) contacting a cell with the composition of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, the cell of any one of the embodiments and/or aspects disclosed herein, or the pharmaceutical composition of any one of the embodiments and/or aspects disclosed herein, and (b) administering an effective amount of the cell to the subject.

In aspects, the present disclosure provides a method of treating, ameliorating or preventing a disease or disorder in a subject, comprising administering an effective amount of the composition of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, the cell of any one of the embodiments and/or aspects disclosed herein, or the pharmaceutical composition of any one of the embodiments and/or aspects disclosed herein to the subject.

In embodiments, composition of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, the cell of any one of the embodiments and/or aspects disclosed herein, or the pharmaceutical composition of any one of the embodiments and/or aspects disclosed herein for use in treating, ameliorating or preventing a patient with a disease or disorder.

In aspects, the present disclosure provides use of the composition of any one of the embodiments and/or aspects disclosed herein, the nucleic acid of any one of the embodiments and/or aspects disclosed herein, the viral vector of any one of the embodiments and/or aspects disclosed herein, the lipid nanoparticle of any one of the embodiments and/or aspects disclosed herein, the cell of any one of the embodiments and/or aspects disclosed herein, or the pharmaceutical composition of any one of the embodiments and/or aspects disclosed herein in the manufacture of a medicament for the treating, ameliorating or preventing of a disease or disorder.

In aspects, the present disclosure provides a method of detecting and/or quantifying a nucleic acid in a sample, comprising contacting the sample with a composition of any one of the embodiments and/or aspects disclosed herein.

In embodiments, the nucleic acid is a target and/or reporter nucleic acid. In embodiments, the method comprises detection of a reporter signal, the reporter signal being generated upon endonuclease cleavage. In embodiments, the reporter signal is a fluorescent signal. In embodiments, the endonuclease has collateral cleavage activity.

In various embodiments, the composition disclosed herein, or the trans-splicing system disclosed herein, further comprises a repair RNA (repRNA) sequence, comprising: (a) one or more exons and/or introns; (b) a splice donor and/or splice acceptor, wherein the repRNA is suitable for trans-splicing. In embodiments, the trans-splicing system comprises a splice donor, a splice acceptor, and replaces an internal exon. In embodiments, repRNA is operably linked to the RNA molecule comprising a sequence complementary to one strand of a target nucleic acid molecule or the gRNA.

In aspects, the present disclosure provides a system for targeting a nucleic acid for trans-splicing, the system comprising: (a) an endonuclease of any one of the embodiments disclosed herein, and optionally an RNA molecule comprising a sequence complementary to one strand of a target nucleic acid molecule; (b) an RNA-binding polypeptide that associates with the endonuclease; and (c) a repair RNA (repRNA) sequence, comprising: (i) one or more exons and/or introns; (ii) a splice donor and/or splice acceptor.

In embodiments, the RNA molecule is a gRNA.

In embodiments, the endonuclease is not linked, associated, and/or fused with an RNA binding protein.

In embodiments, the repRNA is not operably linked to one or more gRNAs. In embodiments, the repRNA is provided in trans to one or more gRNAs.

In embodiments, the repRNA further comprises a ribozyme site. In embodiments, the ribozyme site is a hairpin, hammerhead, hepatitis delta virus (HDV), Varkud satellite (VS), or glmS ribozyme site, or a variant thereof. In embodiments, the ribozyme site is a HDV ribozyme site. In embodiments, the ribozyme site is upstream of the one or more exons and/or introns of the repRNA.

In aspects, the present disclosure provides a system for targeting a nucleic acid for trans-splicing, the system comprising: (a) an endonuclease of any one of claims 1-108 and an RNA molecule comprising a sequence complementary to one strand of a target nucleic acid molecule; and (b) a repair RNA (repRNA) sequence, comprising: (i) one or more exons and/or introns; (ii) a splice donor and/or splice acceptor.

In embodiments, the RNA molecule is a gRNA. In embodiments, the endonuclease is not linked, associated, and/or fused with an RNA binding protein. In embodiments, the repRNA is operably linked to one or more gRNAs.

In aspects, the present disclosure provides a variety of methods (e.g., with a composition of any one of the embodiments and/or aspects disclosed herein). For example, a composition of any one of the embodiments and/or aspects disclosed herein can be used to (i) modify (e.g., cleave, e.g., nick; methylate; and the like) target nucleic acid (DNA or RNA; single stranded or double stranded); (ii) modulate transcription of a target nucleic acid; (iii) label a target nucleic acid; (iv) bind a target nucleic acid e.g., for purposes of isolation, labeling, imaging, tracking, and the like); (v) modify a polypeptide (e.g., a histone) associated with a target nucleic acid; and the like.

In embodiments, the present disclosure provides a method of modifying a target nucleic acid. In embodiments, a method of the present disclosure for modifying a target nucleic acid comprises contacting the target nucleic acid with (a) a composition of the present disclosure (e.g. an endonuclease and/or a chimeric protein of any one of the embodiments and/or aspects disclosed herein); and (b) one or more (e.g., two) RNAs of any one of the embodiments and/or aspects disclosed herein. In embodiments, a method of the present disclosure for modifying a target nucleic acid comprises contacting the target nucleic acid with: a) a composition of the present disclosure (e.g. an endonuclease and/or a chimeric protein of any one of the embodiments and/or aspects disclosed herein); and b) one or more (e.g., two) RNAs of any one of the embodiments and/or aspects disclosed herein; and c) a donor nucleic acid (e.g., a donor template). In embodiments, the contacting step is carried out in a cell in vitro. In embodiments, the contacting step is carried out in a cell in vivo. In embodiments, the contacting step is carried out in a cell ex vivo.

It is to be understood that, in emboIiments, while a method of binding may result in nothing more than binding of the target nucleic acid, in other embodiments, the method can have different final results (e.g., the method can result in modification of the target nucleic acid, e.g., cleavage/methylation/and the like, modulation of transcription from the target nucleic acid; modulation of translation of the target nucleic acid; genome editing; modulation of a protein associated with the target nucleic acid; isolation of the target nucleic acid; and the like).

For examples of suitable methods, see, for example, Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 Sep.; 31(9):839-43; Qi et al, Cell. 2013 Feb. 28; 152(5):1173-83; Wang et al., Cell. 2013 May 9; 153(4):910-8; Auer et al., Genome Res. 2013 Oct. 31; Chen et al., Nucleic Acids Res. 2013 Nov. 1; 41 (20):e19; Cheng et al., Cell Res. 2013 Oct.; 23(10):1163-71; Cho et al., Genetics. 2013 Nov.; 195(3):1177-80; DiCarlo et al., Nucleic Acids Res. 2013 April; 41(7):4336-43; Dickinson et al., Nat Methods. 2013 Oct.; 10(10):1028-34; Ebina et al., Sci Rep. 2013; 3:2510; Fujii et al, Nucleic Acids Res. 2013 Nov. 1; 41 (20):e187; Hu et al., Cell Res. 2013 Nov.; 23(11):1322-5; Jiang et al., Nucleic Acids Res. 2013 Nov. 1; 41 (20):e188; Larson et al., Nat Protoc. 2013 Nov.; 8(11):2180-96; Mali et. at., Nat Methods. 2013 Oct.; 10(10):957-63; Nakayama et al., Genesis. 2013 Dec.; 51(12):835-43; Ran et al., Nat Protoc. 2013 Nov.; 8(11):2281-308; Ran et al., Cell. 2013 Sep. 12; 154(6):1380-9; Upadhyay et al., G3 (Bethesda). 2013 Dec. 9; 3(12):2233-8; Walsh et al., Proc Natl Acad Sci USA. 2013 Sep. 24; 110(39):15514-5; Xie et al., Mol Plant. 2013 Oct. 9; Yang et al., Cell. 2013 Sep. 12; 154(6):1370-9; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; each of which is hereby incorporated by reference in its entirety.

In embodiments, the present disclosure provides (but is not limited to) methods of cleaving a target nucleic acid; methods of editing a target nucleic acid; methods of modulating transcription from a target nucleic acid; methods of isolating a target nucleic acid, methods of binding a target nucleic acid, methods of imaging a target nucleic acid, methods of modifying a target nucleic acid, and the like.

In embodiments, the terms “contact a target nucleic acid” and “contacting a target nucleic acid”, for example, encompass all methods for contacting the target nucleic acid. For example, a present polypeptide (e.g. of the present endonuclease and/or chimeric protein) is provided to a cell as protein, RNA (encoding the present polypeptide), or DNA (encoding the present polypeptide); while a present RNA can be provided as a RNA or as a nucleic acid encoding the RNA. As such, when, for example, performing a method in a cell (e.g., inside of a cell in vitro, inside of a cell in vivo, inside of a cell ex vivo), a method that includes contacting the target nucleic acid encompasses the introduction into the cell of any or all of the components in their active/final state (e.g., in the form of a protein(s) for a polypeptide; in the form of a protein for a chimeric polypeptide; in the form of an RNA in embodiments for the present RNA), and also encompasses the introduction into the cell of one or more nucleic acids encoding one or more of the components (e.g., nucleic acid(s) comprising nucleotide sequence(s) encoding a present polypeptide or a present chimeric polypeptide, nucleic acid(s) comprising nucleotide sequence(s) encoding guide RNA(s), nucleic acid comprising a nucleotide sequence encoding a donor template, and the like). Because the methods can also be performed in vitro outside of a cell, a method that includes contacting a target nucleic acid, (unless otherwise specified) encompasses contacting outside of a cell in vitro, inside of a cell in vitro, inside of a cell in vivo, or inside of a cell ex vivo.

In embodiments, the method of the present disclosure for modifying a target nucleic acid comprises introducing into a target cell a present locus, e.g., a nucleic acid comprising a nucleotide sequence encoding a present polypeptide as well as nucleotide sequences of about 1 kilobase (kb) to 5 kb in length surrounding the present-encoding nucleotide sequence from a cell (e.g., in embodiments, a cell that in its natural state (the state in which it occurs in nature) comprises a present locus) comprising a present locus, where the target cell does not normally (in its natural state) comprise a present locus. However, one or more spacer sequences, encoding guide sequences for the encoded crRNA(s), can be modified such that one or more target sequences of interest are targeted. Thus, for example, in embodiments, a method of the present disclosure for modifying a target nucleic acid comprises introducing into a target cell a present locus, e.g., a nucleic acid obtained from a source cell (e.g., in embodiments, a cell that in its natural state (the state in which it occurs in nature) comprises a present locus), where the nucleic acid has a length of from 100 nucleotides (nt) to 5 kb in length (e.g., from 100 nt to 500 nt, from 500 nt to 1 kb, from 1 kb to 1.5 kb, from 1.5 kb to 2 kb, from 2 kb to 2.5 kb, from 2.5 kb to 3 kb, from 3 kb to 3.5 kb, from 3.5 kb to 4 kb, or from 4 kb to 5 kb in length) and comprises a nucleotide sequence encoding a present polypeptide. As noted above, in some such embodiments, one or more spacer sequences, encoding guide sequences for the encoded crRNA(s), can be modified such that one or more target sequences of interest are targeted. In embodiments, the method comprises introducing into a target cell: i) a present locus; and ii) a donor DNA template. In embodiments, the target nucleic acid is in a cell-free composition in vitro. In embodiments, the target nucleic acid is present in a target cell. In embodiments, the target nucleic acid is present in a target cell, where the target cell is a prokaryotic cell. In embodiments, the target nucleic acid is present in a target cell, where the target cell is a eukaryotic cell. In embodiments, the target nucleic acid is present in a target cell, where the target cell is a mammalian cell. In embodiments, the target nucleic acid is present in a target cell, where the target cell is a plant cell.

In embodiments, a method of the present disclosure for modifying a target nucleic acid comprises contacting a target nucleic acid with a present polypeptide of the present disclosure, or with a present chimeric polypeptide of the present disclosure. In embodiments, a method of the present disclosure for modifying a target nucleic acid comprises contacting a target nucleic acid with a present polypeptide and a present guide RNA. In embodiments, a method of the present disclosure for modifying a target nucleic acid comprises contacting a target nucleic acid with a present polypeptide, a first present guide RNA, and a second present guide RNA In embodiments, a method of the present disclosure for modifying a target nucleic acid comprises contacting a target nucleic acid with a present polypeptide of the present disclosure and a present guide RNA and a donor DNA template.

### Usher Syndrome

Usher syndrome is inherited as an autosomal recessive condition characterized by hearing loss or deafness and progressive vision loss. The loss of vision is caused by retinitis pigmentosa (RP), which affects the layer of light-sensitive tissue at the retina. Without wishing to be bound by theory, vision loss occurs as the light-sensing cells of the retina gradually deteriorate.

Three major types of Usher syndrome, designated as types I (subtypes IA through IG), II (subtypes IIA, IIB, and IIC), and III, have been identified. These types are distinguished by their severity and the age when signs and symptoms appear.

Subjects with Usher Syndrome type I are born profoundly deaf and begin to lose their vision in the first decade of life. They also exhibit balance difficulties and learn to walk slowly as children, due to problems in their vestibular system.

Usher Syndrome type II is a heterogeneous autosomal recessive disorder characterized by progressive retinitis pigmentosa and sensorineural hearing deficiencies, resulting in deaf-blindness in patients. Subjects with Usher II are generally have a reduced ability to hear rather than deaf, and their hearing does not degrade over time. They do not seem to have noticeable problems with balance. They typically begin to lose their vision in the second decade of life, and may preserve some vision into middle age.

Subjects with Usher syndrome III experience a progressive loss of hearing, and roughly half have balance difficulties. Mutations in CLRN1, have been linked to Usher syndrome type III. CLRN1 encodes clarin-1, a protein involved in the development and maintenance of the inner ear and retina.

Several genes have been implicated or associated with Usher syndrome, and mutations in the genese: CDH23, CLRN1, GPR98, MY07A, PCDH15, USH1C, USH1G, USH2A, and ADGRV1, WHRN. Mutations in any one of these genes alters gene expression or protein function and can lead to Usher syndrome. The genes play roles in the development and maintenance of hair cells, which have sensory functionalities in the inner ear that help transmit sound and motion signals to the brain. In the retina, these genes are also involved in determining the structure and function of light-sensing cells called rods and cones. In some cases, the exact role of these genes in hearing and vision is unknown. Most of the mutations responsible for Usher syndrome lead to a loss or reduction of hair cells in the inner ear and a gradual loss or reduction of rods and cones in the retina. Degeneration of these sensory cells causes hearing loss, balance problems, and vision loss characteristic of this condition.

Usher syndrome type II can be caused by mutations in, e.g., USH2A, ADGRV1, WHRN, GPR98 (also called VLGR1) gene, and DFNB31. Usher syndrome type III can be caused by mutations in e.g., CLRN1.

The protein encoded by the USH2A gene, usherin, is located in the supportive tissue in the inner ear and retina. Usherin is involved in the development and maintenance of these structures. WHRN mutations are associated with Usher syndrome type 2D or non-syndromic deafness (DFNB31). The associated phenotypes are dependent on the mutation location within the two predominantly expressed variants (long and short). Variants in ADGRV1 are a cause of Usher syndrome type 2, and the associated phenotype is less known.

In embodiments, the system for targeting a nucleic acid for trans-splicing is for use in treating a disease, such as Usher Syndrome type II.

In embodiments, disclosed herein is a method of treating, ameliorating or preventing Usher syndrome or a symptom thereof in a subject in need thereof, comprising: administering an effective amount of the composition of any one of the embodiments disclosed herein, the nucleic acid of any one of the embodiments disclosed herein, the viral vector of any one of the embodiments disclosed herein, the lipid nanoparticle of any one of the embodiments disclosed herein, the cell of any one of the embodiments disclosed herein, the pharmaceutical composition of any one of the embodiments disclosed herein, or the trans-splicing system any one of any one of the embodiments disclosed herein to the subject.

In embodiments, disclosed herein is a method of treating, ameliorating or preventing Usher syndrome or a symptom thereof in a subject, in need thereof, comprising: (a) contacting a cell with the composition of any one of the embodiments disclosed herein, the nucleic acid of any one of the embodiments disclosed herein, the viral vector of any one of the embodiments disclosed herein, the lipid nanoparticle of any one of the embodiments disclosed herein, the cell of any one of the embodiments disclosed herein, the pharmaceutical composition of any one of the embodiments disclosed herein, or the trans-splicing system any one of any one of the embodiments disclosed herein and (b) administering an effective amount of the cell to the subject.

In embodiments, the cell is derived from the subject.

In embodiments, the Usher syndrome is selected from Usher syndrome type I, Usher syndrome type II, or Usher syndrome type III.

In embodiments, the Usher syndrome is Usher syndrome type I.

In embodiments, the Usher syndrome is Usher syndrome type II.

In embodiments, the Usher syndrome is Usher syndrome type III.

In embodiments, the method targets one or more Usher syndrome-associated gene.

In embodiments, the method targets one or more genes selected from CDH23, MY07A, PCDH15, USH1C, USH1G, USH2A, ADGRV1, WHRN, GPR98, DFNB31, and CLRN1.

In embodiments, the method targets one or more of USH2A, GPR98, and DFNB31.

In embodiments, the method targets USH2A.

In embodiments, the method corrects a mutation or defect in one or more Usher syndrome-associated gene.

In embodiments, the method corrects a mutation or defect in one or more genes selected from USH2A, CDH23, MY07A, PCDH15, USH1C, USH1G, ADGRV1, WHRN, GPR98, DFNB31, and CLRN1.

In embodiments, the method corrects a mutation or defect in one or more of USH2A, GPR98, and DFNB31.

In embodiments, the method corrects a mutation or defect in USH2A.

In embodiments, the method causes trans-splicing of one or more genes selected from USH2A, CDH23, MY07A, PCDH15, USH1C, USH1G, ADGRV1, WHRN, GPR98, DFNB31, and CLRN1.

In embodiments, the method causes trans-splicing of one or more of USH2A, GPR98, and DFNB31.

In embodiments, the method causes trans-splicing of USH2A.

In embodiments, the method treats, ameliorates or prevents one or more symptoms of retinitis pigmentosa.

In embodiments, the method treats, ameliorates or prevents hearing reduction or loss.

In embodiments, the method treats, ameliorates or prevents vision reduction or loss.

In embodiments, the method treats, ameliorates or prevents one or more of night blindness and loss or reduction of peripheral vision.

In various embodiments, the “subject” refers to any animal (e.g., a mammal), including, but not limited to, humans, and non-human animals (including, but not limited to, non-human primates, dogs, cats, rodents, horses, cows, pigs, mice, rats, hamsters, rabbits, and the like (e.g., which is to be the recipient of a particular treatment, or from whom cells are harvested)). In embodiments, the subject is a human.

It will also be understood that, although the terms first, second, etc., may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients. Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein. All publications, patents, and patent publications cited are incorporated by reference herein in their entirety for all purposes.

This disclosure is further illustrated by the following non-limiting examples.

### EXAMPLES

### Example 1: Guide RNA Design

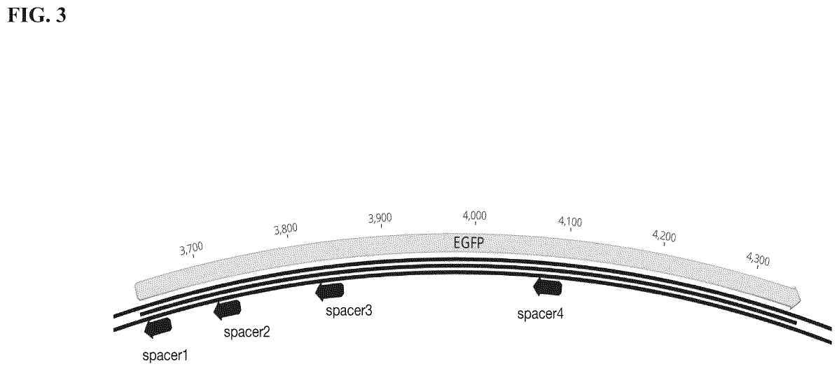

The experiments of this example evaluated the structure of the CRISPR RNA of the Cas13K2F system disclosed herein. In these experiments, gRNAs were designed for the Cas13K2F system disclosed herein to target multiple sites across the coding sequence of eGFP in HEK293T cells (FIG. 3). The gRNAs were tested alongside a scrambled non-targeting guide, demonstrating reprogrammability and specificity of the RNase activity of this CRISPR-Cas system towards different targets.

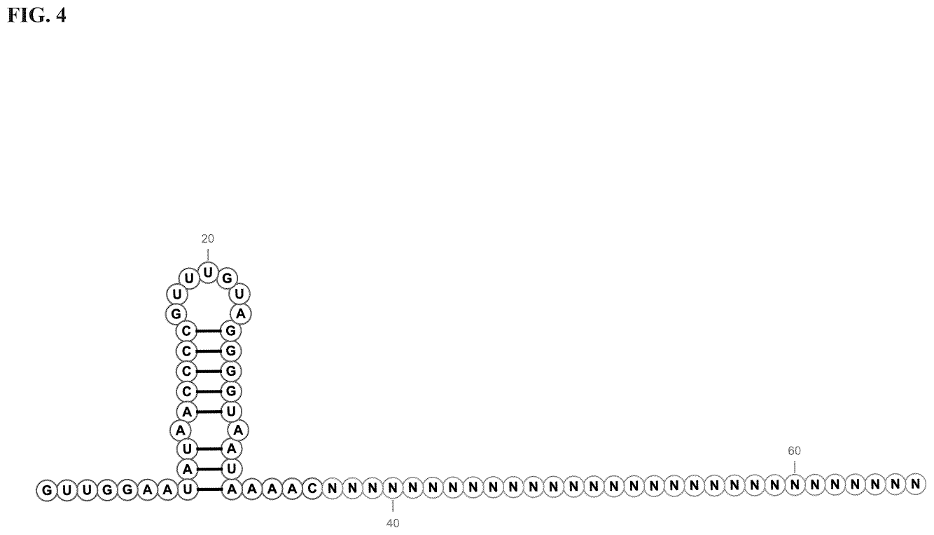

Several gRNA orientations were tested to evaluate the correct structure of the CRISPR RNA of the Cas13K2F system. The gRNA structure for Cas13K2F system was determined to consist of a CRISPR repeat at the 3′ end of the RNA followed by a spacer sequence complementary to the target RNA (see, e.g., FIG. 4).

### Example 2: Mammalian Experiments Demonstrating Knockdown of eGFP RNA

The experiments of this example demonstrate the capability of the Cas13K2F system disclosed herein to target and knockdown eGFP RNA. In these experiments, for each Cas open reading frame (“ORF”), an SV40 NLS sequence was added to the N-terminus (MSPKKKRKVEAS (SEQ ID NO: 78)) and an SV40 NLS with an appended HA tag was added to the C-terminus (GSGPKKKRKVAAAYPYDVPDYA (SEQ ID NO: 77)). The resulting ORFs were then mammalian codon optimized and synthesized into mammalian expression vectors. Transcription was driven by a CMV promoter, and an SV40 polyadenylation signal was used to terminate transcription. The sequence of the CMV promoter is the following:

(SEQ ID NO: 76)
CGCCCCATTGACGCAAATGGGCGGTAGGCGTGTACGGTGGGAGGTCTATA
TAAGCAGAGCTGGTTTAGTGAACCGTCAGATC

The sequence of the SV40 polyadenylation signal is the following:

(SEQ ID NO: 79)
CAGCTCACAGACATGATAAGATACATTGATGAGTTTGGACAAACCACAAC
TAGAATGCAGTGAAAAAAATGCTTTATTTGTGAAATTTGTGATGCTATTG
CTTTATTTGTAACCATTATAAGCTGCAATAAACAAGTTAACAACAACAAT
TGCATTCATTTTATGTTTCAGGTTCAGGGGGAGGTGTGGGAGGTTTTTTA
AAGCAAGTAAAACCTCTACAAATGTGGTATTGGC

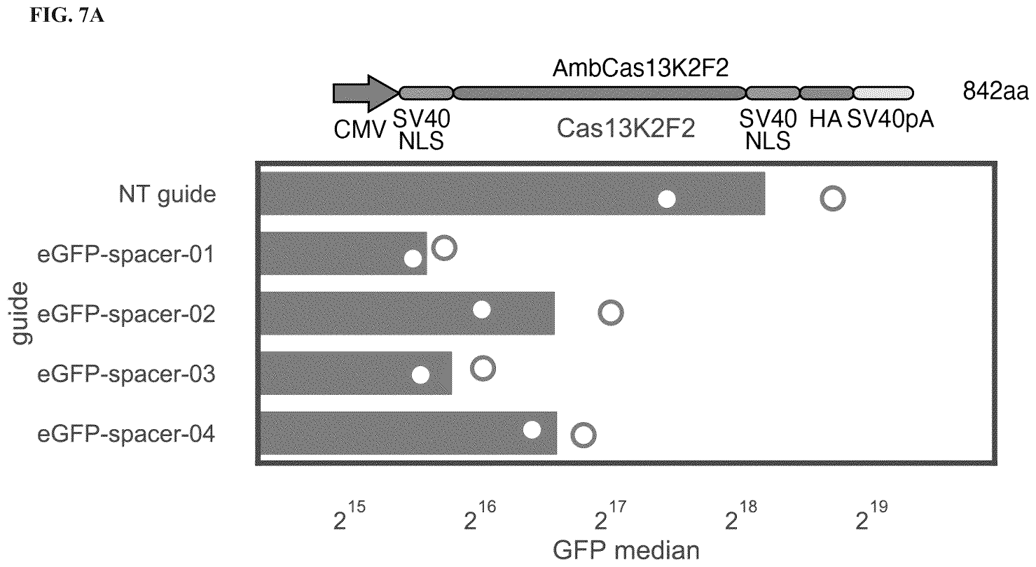

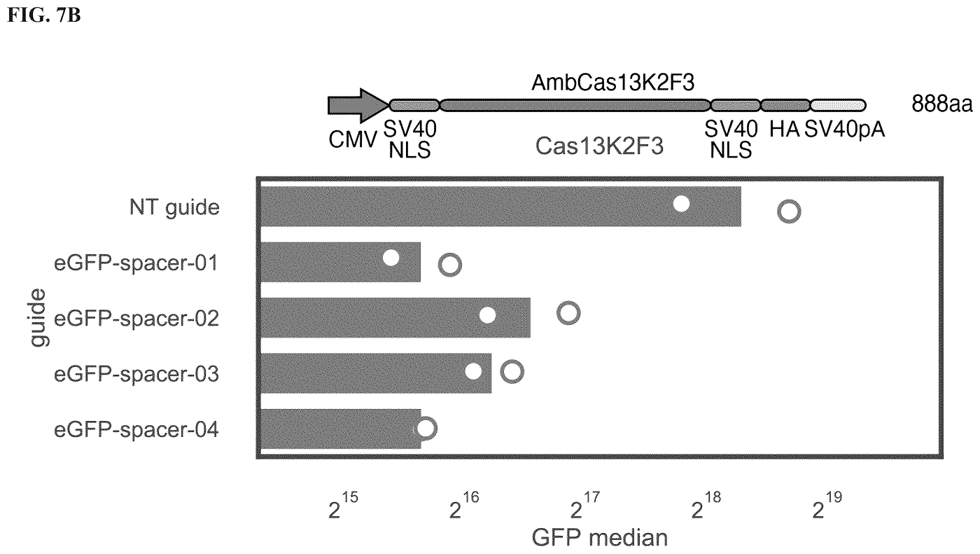

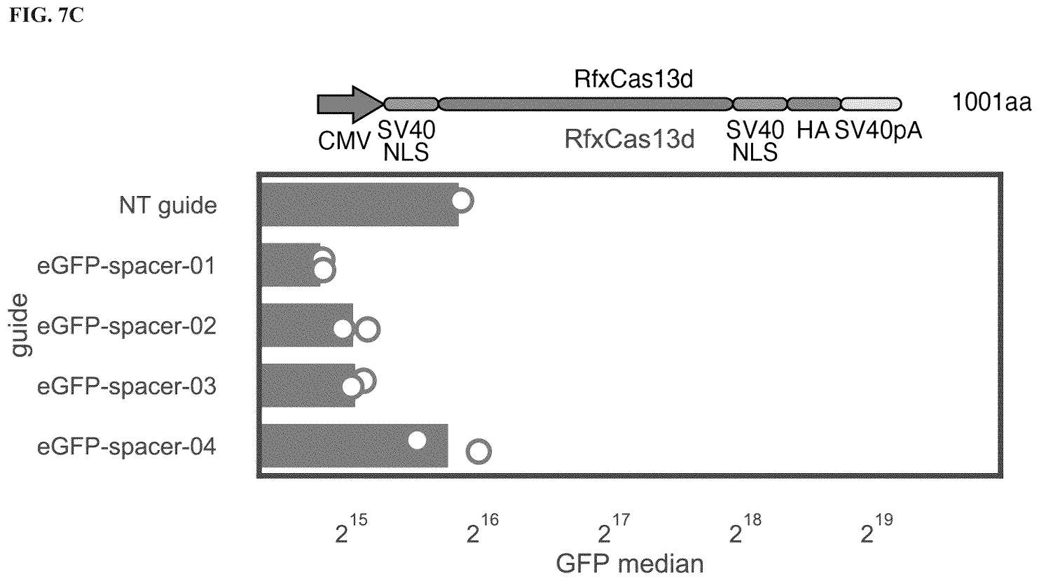

A plasmid expressing eGFP and plasmids expressing Cas13K2F proteins and gRNAs were co-transfected into HEK293T cells (bio reps n=2) using lipofectamine 2000 per manufacturer's instructions. Protein expression was confirmed by observing fluorescence via fluorescent microscopy on a Bio-Rad ZOE Fluorescent Cell Imager, and flow cytometry was performed 48 hours following transfection on a Sartorius iQue3. Cell and singlet gating was performed with Sartorius iQue3 instrument software, and median GFP intensity of singlets was calculated in python using FlowCytometryTools. Results were plotted using Matplotlib (See FIG. 7A, FIG. 7B, and FIG. 7C).

These experiments further compared the Cas13K2F system disclosed herein to other CAS systems by phylogenetic analysis. FIG. 5 and FIG. 6 each show clear distinction in phylogenetic placement as separate protein families for the Cas13K2F system disclosed herein. Compared to other CAS systems, the Cas13K2F system disclosed herein is novel and shows low sequence similarity to other systems. The results in FIG. 5 are reproduced in Table 4 below.

Accordingly, the results of the experiments of this example demonstrate the Cas13K2F systems disclosed herein is novel and is capable of targeting and knocking down eGFP RNA.

TABLE 4
Percent Identity Matrix

| | Cas13X.1 | Cas13bt3 | Cas13bt2 | Cas13bt1 | Cas13bt8 |
|---|---|---|---|---|---|
| Cas13X.1 | | 99.5 | 39.8 | 38.3 | 37.4 |
| Cas13bt3 | 99.5 | | 39.3 | 37.8 | 36.9 |
| Cas13bt2 | 39.8 | 39.3 | | 69.3 | 62.5 |
| Cas13bt1 | 38.3 | 37.8 | 69.3 | | 63 |
| Cas13bt8 | 37.4 | 36.9 | 62.5 | 63 | |
| Cas13X.2 | 37.4 | 36.9 | 62.5 | 63 | 100 |
| Cas13bt9 | 37.8 | 37.4 | 43.8 | 43.2 | 49.2 |
| Cas13bt11 | 38 | 37.5 | 43.3 | 42.7 | 48.7 |
| Cas13bt5 | 31.8 | 31.3 | 36.8 | 35.5 | 36.3 |
| Cas13bt10 | 29 | 28.6 | 28.9 | 29.9 | 29.9 |
| Cas13bt15 | 21.9 | 21.4 | 23.3 | 22.2 | 23.5 |
| Cas13bt7 | 19.9 | 19.5 | 21.6 | 21.4 | 21.7 |
| Cas13bt6 | 13.8 | 13.3 | 14.2 | 14.1 | 14.1 |
| Cas13bt14 | 13.6 | 13.2 | 14.6 | 14.5 | 14.6 |
| Cas13Y.3 | 13.6 | 13.2 | 14.6 | 14.5 | 14.6 |
| Cas13bt12 | 14.4 | 13.9 | 15.3 | 14.6 | 14.7 |
| Cas13Y.1 | 14.4 | 13.9 | 15.3 | 14.6 | 14.7 |
| Cas13bt4 | 14.1 | 13.6 | 14.7 | 14.8 | 14.9 |
| Cas13bt16 | 14.3 | 13.8 | 14.7 | 14.7 | 14.9 |
| Cas13Y.5 | 13.9 | 13.4 | 15.6 | 15.9 | 15.8 |
| Cas13Y.4 | 13 | 12.5 | 14.9 | 15.5 | 14.8 |
| Cas13_K2F1 | 9 | 8.7 | 8.4 | 9.2 | 8.6 |
| Cas13_K2F2 | 9.1 | 8.9 | 8.7 | 9.8 | 8.6 |
| Cas13_K2F5 | 9.5 | 9.3 | 9.6 | 9.9 | 9.5 |
| Cas13_K2F3 | 9.4 | 9.4 | 8.9 | 9.1 | 9.2 |

| | Cas13X.2 | Cas13bt9 | Cas13bt11 | Cas13bt5 | Cas13bt10 | Cas13bt15 |
|---|---|---|---|---|---|---|
| Cas13X.1 | 37.4 | 37.8 | 38 | 31.8 | 29 | 21.9 |
| Cas13bt3 | 36.9 | 37.4 | 37.5 | 31.3 | 28.6 | 21.4 |
| Cas13bt2 | 62.5 | 43.8 | 43.3 | 36.8 | 28.9 | 23.3 |
| Cas13bt1 | 63 | 43.2 | 42.7 | 35.5 | 29.9 | 22.2 |
| Cas13bt8 | 100 | 49.2 | 48.7 | 36.3 | 29.9 | 23.5 |
| Cas13X.2 | | 49.2 | 48.7 | 36.3 | 29.9 | 23.5 |
| Cas13bt9 | 49.2 | | 97.9 | 33.1 | 28.7 | 22.6 |
| Cas13bt11 | 48.7 | 97.9 | | 33.2 | 29.2 | 22.5 |
| Cas13bt5 | 36.3 | 33.1 | 33.2 | | 29.1 | 22.2 |
| Cas13bt10 | 29.9 | 28.7 | 29.2 | 29.1 | | 21.2 |
| Cas13bt15 | 23.5 | 22.6 | 22.5 | 22.2 | 21.2 | |
| Cas13bt7 | 21.7 | 22.7 | 22.5 | 20.6 | 20.5 | 44.8 |
| Cas13bt6 | 14.1 | 12.5 | 12.6 | 12.2 | 13.5 | 12 |
| Cas13bt14 | 14.6 | 13 | 13.1 | 12.1 | 14 | 12.1 |
| Cas13Y.3 | 14.6 | 13 | 13.1 | 12.1 | 14 | 12.1 |
| Cas13bt12 | 14.7 | 12.9 | 12.9 | 12.5 | 14 | 13.3 |
| Cas13Y.1 | 14.7 | 12.9 | 12.9 | 12.5 | 14 | 13.3 |
| Cas13bt4 | 14.9 | 13.3 | 13.5 | 12.2 | 13 | 12.3 |
| Cas13bt16 | 14.9 | 13.7 | 13.8 | 11.7 | 13.9 | 12.2 |
| Cas13Y.5 | 15.8 | 14 | 14.1 | 13.6 | 15 | 11.8 |
| Cas13Y.4 | 14.8 | 13.3 | 13.2 | 12.5 | 13.7 | 12.8 |
| Cas13_K2F1 | 8.6 | 8.4 | 8.3 | 9.4 | 9.1 | 7.9 |
| Cas13_K2F2 | 8.6 | 8.4 | 8.3 | 9.9 | 8.9 | 8.5 |
| Cas13_K2F5 | 9.5 | 9.3 | 9.1 | 10.1 | 8.8 | 8.6 |
| Cas13_K2F3 | 9.2 | 8.6 | 8.4 | 8.3 | 8.4 | 8.1 |

| | Cas13bt7 | Cas13bt6 | Cas13bt14 | Cas13Y.3 | Cas13bt12 |
|---|---|---|---|---|---|
| Cas13X.1 | 19.9 | 13.8 | 13.6 | 13.6 | 14.4 |
| Cas13bt3 | 19.5 | 13.3 | 13.2 | 13.2 | 13.9 |
| Cas13bt2 | 21.6 | 14.2 | 14.6 | 14.6 | 15.3 |
| Cas13bt1 | 21.4 | 14.1 | 14.5 | 14.5 | 14.6 |
| Cas13bt8 | 21.7 | 14.1 | 14.6 | 14.6 | 14.7 |
| Cas13X.2 | 21.7 | 14.1 | 14.6 | 14.6 | 14.7 |
| Cas13bt9 | 22.7 | 12.5 | 13 | 13 | 12.9 |
| Cas13bt11 | 22.5 | 12.6 | 13.1 | 13.1 | 12.9 |
| Cas13bt5 | 20.6 | 12.2 | 12.1 | 12.1 | 12.5 |
| Cas13bt10 | 20.5 | 13.5 | 14 | 14 | 14 |
| Cas13bt15 | 44.8 | 12 | 12.1 | 12.1 | 13.3 |
| Cas13bt7 | | 12.1 | 12.2 | 12.2 | 13.3 |
| Cas13bt6 | 12.1 | | 87.2 | 87.2 | 78.1 |
| Cas13bt14 | 12.2 | 87.2 | | 100 | 78.1 |
| Cas13Y.3 | 12.2 | 87.2 | 100 | | 78.1 |
| Cas13bt12 | 13.3 | 78.1 | 78.1 | 78.1 | |
| Cas13Y.1 | 13.3 | 78.1 | 78.1 | 78.1 | 100 |
| Cas13bt4 | 12.4 | 77 | 76.9 | 76.9 | 80.6 |
| Cas13bt16 | 12.7 | 73.2 | 76.8 | 76.8 | 72.7 |
| Cas13Y.5 | 12.3 | 63.8 | 65 | 65 | 64.5 |
| Cas13Y.4 | 13.5 | 62.1 | 62.7 | 62.7 | 62.5 |
| Cas13_K2F1 | 8.3 | 7.5 | 7.4 | 7.4 | 7.7 |
| Cas13_K2F2 | 8.3 | 7.9 | 7.6 | 7.6 | 7.7 |
| Cas13_K2F5 | 8.9 | 7.9 | 7.9 | 7.9 | 7.9 |
| Cas13_K2F3 | 8.3 | 7.9 | 7.4 | 7.4 | 7.7 |
| | Cas13Y.1 | Cas13bt4 | Cas13bt16 | Cas13Y.5 | Cas13Y.4 |
| Cas13X.1 | 14.4 | 14.1 | 14.3 | 13.9 | 13 |
| Cas13bt3 | 13.9 | 13.6 | 13.8 | 13.4 | 12.5 |
| Cas13bt2 | 15.3 | 14.7 | 14.7 | 15.6 | 14.9 |
| Cas13bt1 | 14.6 | 14.8 | 14.7 | 15.9 | 15.5 |
| Cas13bt8 | 14.7 | 14.9 | 14.9 | 15.8 | 14.8 |
| Cas13X.2 | 14.7 | 14.9 | 14.9 | 15.8 | 14.8 |
| Cas13bt9 | 12.9 | 13.3 | 13.7 | 14 | 13.3 |
| Cas13bt11 | 12.9 | 13.5 | 13.8 | 14.1 | 13.2 |
| Cas13bt5 | 12.5 | 12.2 | 11.7 | 13.6 | 12.5 |
| Cas13bt10 | 14 | 13 | 13.9 | 15 | 13.7 |
| Cas13bt15 | 13.3 | 12.3 | 12.2 | 11.8 | 12.8 |
| Cas13bt7 | 13.3 | 12.4 | 12.7 | 12.3 | 13.5 |
| Cas13bt6 | 78.1 | 77 | 73.2 | 63.8 | 62.1 |
| Cas13bt14 | 78.1 | 76.9 | 76.8 | 65 | 62.7 |
| Cas13Y.3 | 78.1 | 76.9 | 76.8 | 65 | 62.7 |
| Cas13bt12 | 100 | 80.6 | 72.7 | 64.5 | 62.5 |
| Cas13Y.1 | | 80.6 | 72.7 | 64.5 | 62.5 |
| Cas13bt4 | 80.6 | | 72.3 | 64.8 | 60.7 |
| Cas13bt16 | 72.7 | 72.3 | | 64.5 | 61.4 |
| Cas13Y.5 | 64.5 | 64.8 | 64.5 | | 64.9 |
| Cas13Y.4 | 62.5 | 60.7 | 61.4 | 64.9 | |
| Cas13_K2F1 | 7.7 | 8 | 7.8 | 8.3 | 8 |
| Cas13_K2F2 | 7.7 | 8.3 | 7.8 | 8.4 | 8.3 |
| Cas13_K2F5 | 7.9 | 8.4 | 7.9 | 7.7 | 8 |
| Cas13_K2F3 | 7.7 | 7.6 | 7.5 | 7.6 | 7.2 |

| | Cas13_K2F1 | Cas13_K2F2 |
|---|---|---|
| Cas13X.1 | 9 | 9.1 |
| Cas13bt3 | 8.7 | 8.9 |
| Cas13bt2 | 8.4 | 8.7 |
| Cas13bt1 | 9.2 | 9.8 |
| Cas13bt8 | 8.6 | 8.6 |
| Cas13X.2 | 8.6 | 8.6 |
| Cas13bt9 | 8.4 | 8.4 |
| Cas13bt11 | 8.3 | 8.3 |
| Cas13bt5 | 9.4 | 9.9 |
| Cas13bt10 | 9.1 | 8.9 |
| Cas13bt15 | 7.9 | 8.5 |
| Cas13bt7 | 8.3 | 8.3 |
| Cas13bt6 | 7.5 | 7.9 |
| Cas13bt14 | 7.4 | 7.6 |
| Cas13Y.3 | 7.4 | 7.6 |
| Cas13bt12 | 7.7 | 7.7 |
| Cas13Y.1 | 7.7 | 7.7 |
| Cas13bt4 | 8 | 8.3 |
| Cas13bt16 | 7.8 | 7.8 |
| Cas13Y.5 | 8.3 | 8.4 |
| A69:B95 | 8 | 8.3 |
| Cas13_K2F1 | | 90.2 |
| Cas13_K2F2 | 90.2 | |
| Cas13_K2F5 | 78 | 83.5 |
| Cas13_K2F3 | 50.2 | 52.4 |
| | Cas13_K2F5 | Cas13_K2F3 |
| Cas13X.1 | 9.5 | 9.4 |
| Cas13bt3 | 9.3 | 9.4 |
| Cas13bt2 | 9.6 | 8.9 |
| Cas13bt1 | 9.9 | 9.1 |
| Cas13bt8 | 9.5 | 9.2 |
| Cas13X.2 | 9.5 | 9.2 |
| Cas13bt9 | 9.3 | 8.6 |
| Cas13bt11 | 9.1 | 8.4 |
| Cas13bt5 | 10.1 | 8.3 |
| Cas13bt10 | 8.8 | 8.4 |
| Cas13bt15 | 8.6 | 8.1 |
| Cas13bt7 | 8.9 | 8.3 |
| Cas13bt6 | 7.9 | 7.9 |
| Cas13bt14 | 7.9 | 7.4 |
| Cas13Y.3 | 7.9 | 7.4 |
| Cas13bt12 | 7.9 | 7.7 |
| Cas13Y.1 | 7.9 | 7.7 |
| Cas13bt4 | 8.4 | 7.6 |
| Cas13bt16 | 7.9 | 7.5 |
| Cas13Y.5 | 7.7 | 7.6 |
| A69:B95 | 8 | 7.2 |
| Cas13_K2F1 | 78 | 50.2 |
| Cas13_K2F2 | 83.5 | 52.4 |
| Cas13_K2F5 | | 52 |
| Cas13_K2F3 | 52 | |

### Example 3: Targeted Trans-Splicing for Cas13K2F Family and dPspCas13b

In the experiments of this example, the Cas13K2F family was evaluated for targeted trans-splicing using a truncated GFP reporter assay. In this assay, the truncated GFP exons (5′ GFP, 3′ GFP) introduced by the splice donor and acceptor reporters were spliced together, generating a transcript that contains a complete GFP mRNA sequence capable of creating a functional GFP protein product, and thereby demonstrating successful trans-splicing.

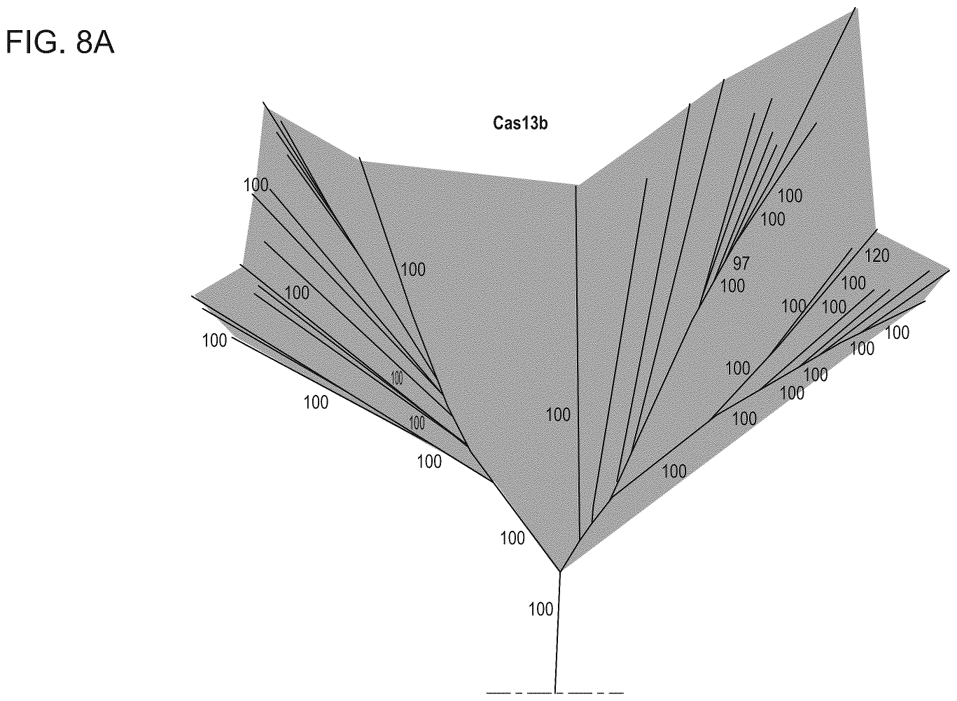

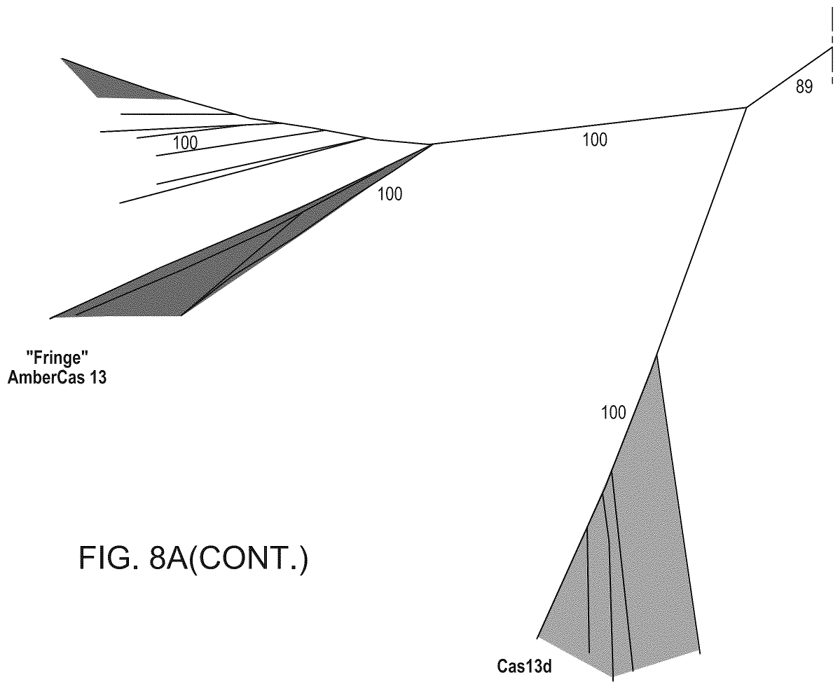

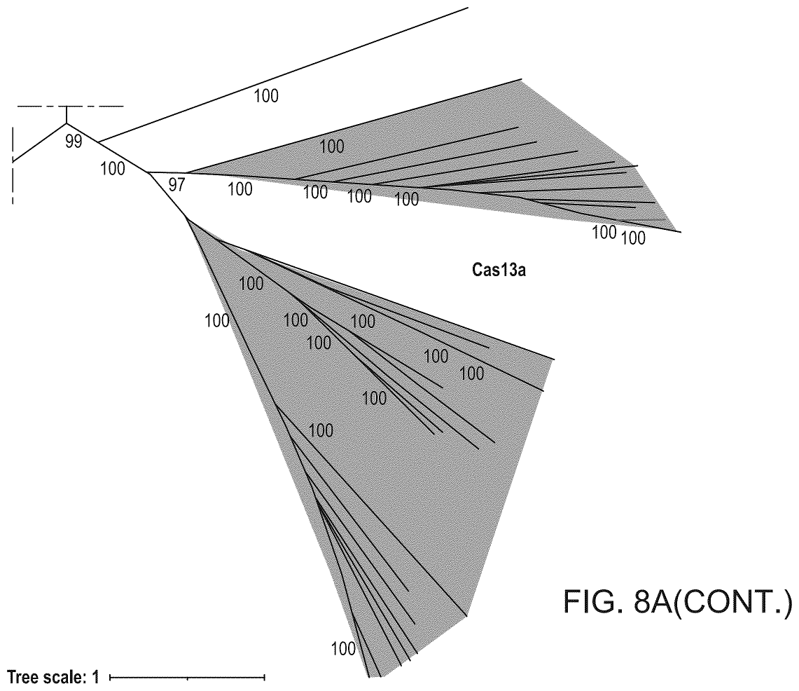

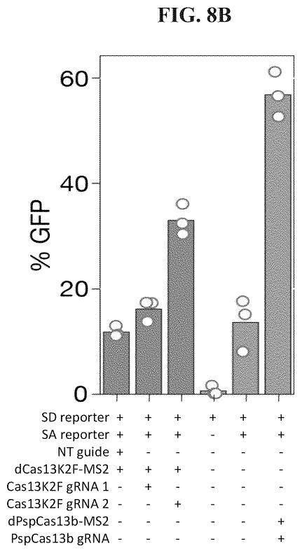

The Cas13 family is highly divergent from Cas13b and Cas13d, as demonstrated by phylogenetic analysis (see FIG. 8A and FIG. 8B). It is also structurally divergent and biochemically distinct from other known Cas13 enzymes.

HEK293 T cells were transfected with either a first or second Cas13K2F gRNA targeting the splice donor (SD) reporter (Cas13K2F gRNA 1 or 2), a fusion of catalytically inactive Cas13K2F orthologue (dCas13K2F) and MS2 (dCas13K2F-MS2), and the splice acceptor (SA) reporter having MS2 stem loops. In these experiments, HEK293FT (Thermo Fisher Scientific Cat. No. R70007) were cultured in a humidified incubator at 5% CO2 at 37° C. using high-glucose DMEM (Invitrogen) complemented with 10% FBS and 1% penicillin/streptomycin. Cell cultures were kept at low passage (<20) and regularly tested for mycoplasma contamination. Cells were plated in 96-well format at 15,000 cells per well with 100 uL of media. Cells were transfected 24 hours later with 25 ng gRNA plasmid (U6 driven expression), 25 ng Cas13-RBP plasmid, 25 ng of the SD reporter, and 25 ng of the SA reporter using 0.32 uL lipofectamine and 10 uL of OptiMEM total. Flow cytometry was performed 48 hours after transfection on a Sartorius iQue3 flow cytometer.

For comparison, HEK293T cells were transfected using a dCas13b variant from Prevotella sp. (“dPspCas13b”), fused to a panel of RBPs sourced from RNA bacteriophage genomes. As shown in FIG. 8B, more than 30% of cells underwent successful trans-splicing using the Cas13K2F system, which was a level of trans-splicing comparable to that achieved using the dPspCas13b system.

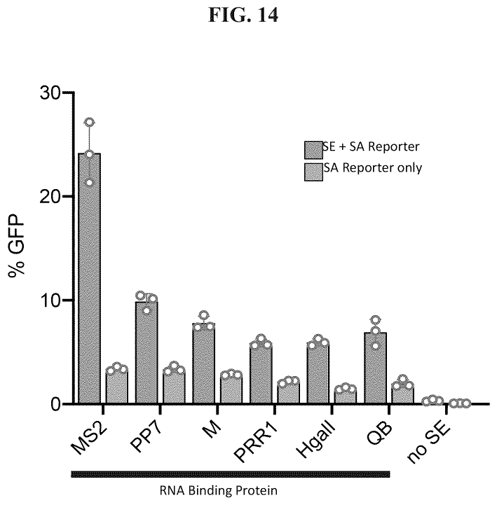

The experiments of this example further evaluated the dPspCas13b systems using different RNA binding proteins. These experiments measured trans-splicing between the SD reporter encoding a 5′GFP upstream of a splice donor, and a SA reporter having two sequence motifs upstream a splice donor and exon encoding 3′GFP. The SA reporters that were tested had sequence motifs recognized by RNA binding proteins (RBPs). The sequence motif and corresponding RBP evaluated included: (i) a MS2 hairpin and a MS2 coat protein (“MS2”); (ii) a PP7 hairpin and a PP7 coat protein (“PP7”); (iii) a M hairpin and a phage M coat protein (“M”); (iv) a PRR1 hairpin and a PRR1 coat protein (“PRR1”); and (v) a Qbeta hairpin and Qbeta coat protein (“QB”). HEK293T cells were transfected with the SD reporter, splice editor (i.e., PspCas13b gRNA and a fusion of dPspCas13b to an RBP), and an SA reporter with the corresponding RBP sequence motif. Control cells were transfected with the SD reporter and SA reporter only. Trans-splicing between the SD reporter and SA reporter yielded a measurable GFP signal. As shown in FIG. 14, each of the splice editors that were evaluated resulted in a level of trans-splicing that was higher than that observed in control cells.

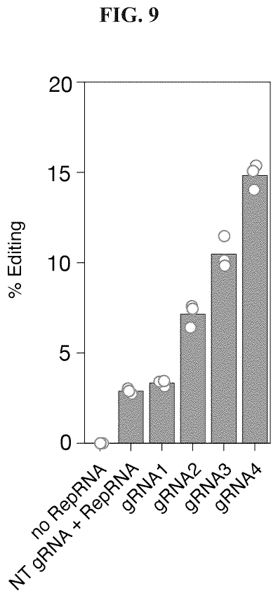

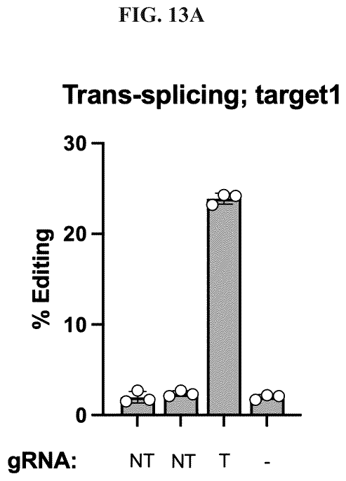

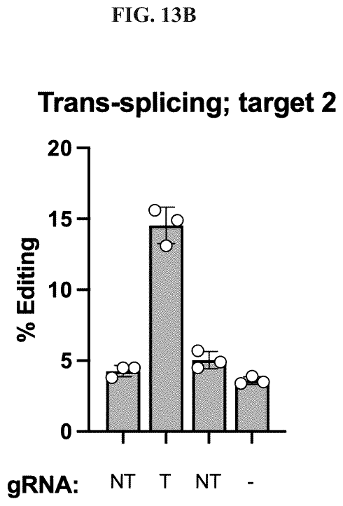

Additionally, the experiments of this example evaluated the Cas13K2F system for trans-splicing using an alternate reporter system having a target nucleic acid containing distinct gene segments from an SD reporter (the target nucleic acid (“target”)). In these experiments, the target contained from 5′ to 3′: the 5′ portion of GFP, a first intron from a human protein-coding gene (“gene A”), a splice acceptor, and an exon from gene A. The trans-splicing template (the “template”) contained from 5′ to 3′: an intron containing two MS2 stem loops, a splice acceptor, and the 3′ portion of GFP. The constructs were designed such that successful trans-splicing between the splice donor of the target and the splice acceptor of the template would yield a GFP signal. HEK293T cells were transfected using lipofectamine 2000 per manufacturer's instructions in 96-well format. Each plasmid (target, template, Cas13K2F-MCP+gRNA) was one-third (33 ng) of the total DNA transfection. As non-coding dummy DNA was included in conditions where less than four components were delivered, such that there would be a total of 100 ng of DNA in each transfection. Media was changed 12 hours after the transfection. Protein expression was confirmed by observing fluorescence via fluorescent microscopy on a Bio-rad ZOE Fluorescent Cell Imager, and flow cytometry was performed 48 hours following transfection on a Sartorius iQue3. Cell and singlet gating was performed with Sartorius iQue3 instrument software, and the percentage of GFP+singlets was calculated in python. HEK293T cells were transfected with constructs encoding dCas13K2F fused to a MS2 coat protein, a gRNA directed to the target (gRNA12, gRNA2, gRNA18, or gRNA19), the target, and the template. Control cells were transfected with a non-targeting gRNA or target only. As shown in FIG. 9, the percentage of GFP-positive cells, and the editing performance (FIG. 13A and FIG. 13B) was dramatically increased using the Cas13K2F system, indicating successful trans-splicing of the target. FIG. 13A and FIG. 13B are graphs showing Cas13K2F trans-splicing across two different RNA targets (MMP9 (FIG. 13A) and USH2A (FIG. 13B); USH2A encodes the 5′end of GFP, a splice donor, a gene A intron, a splice acceptor, and a gene A exon) using PP7 (PCP) as the RNA binding partner (RBP) partner.

### Example 4: Structural Insights of Novel Miniature CRISPR-Cas Effectors

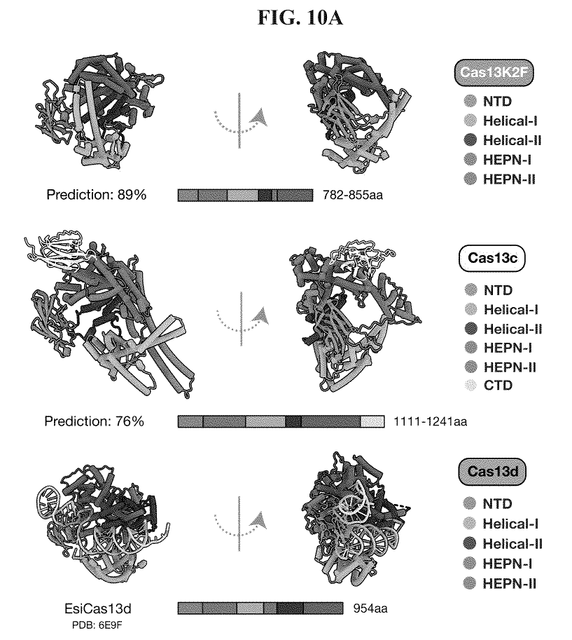

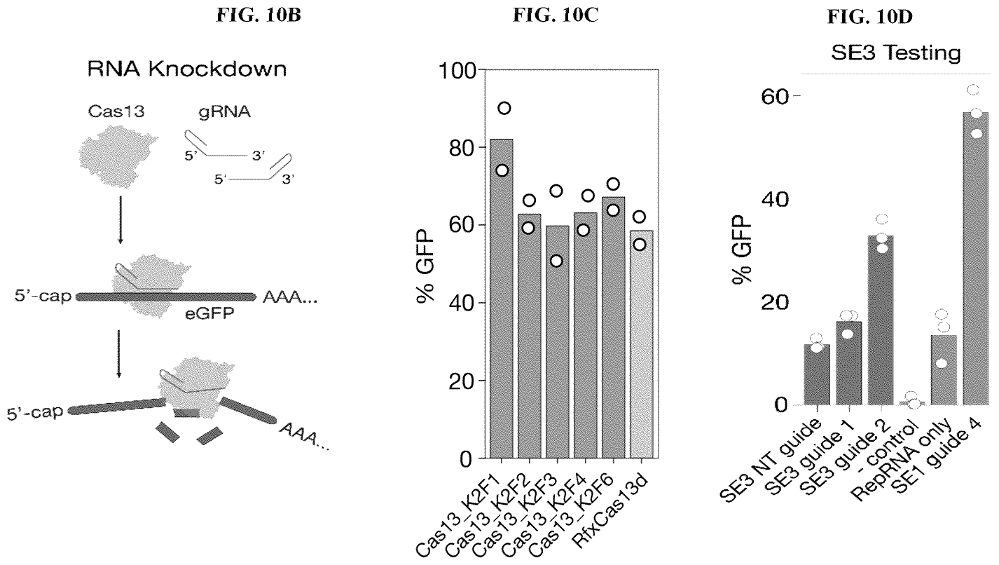

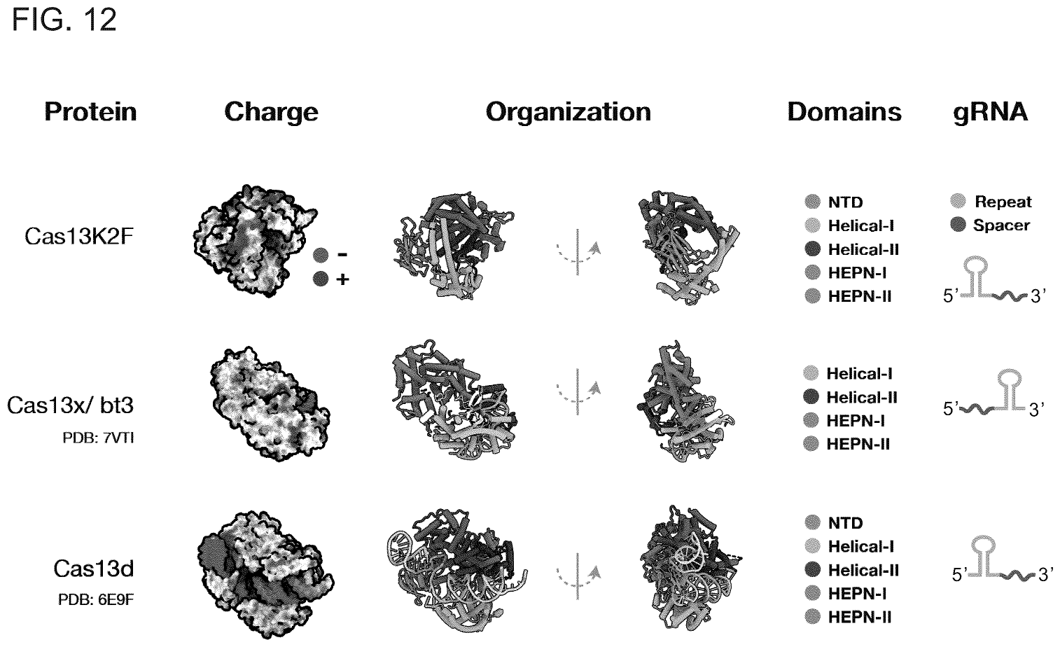

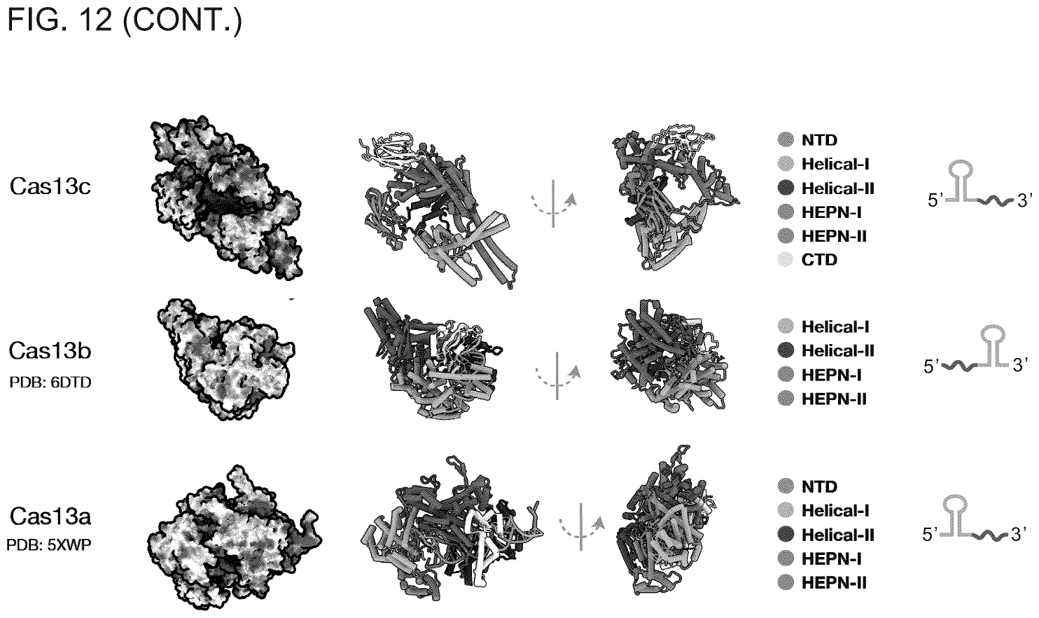

The experiments of this example produced high-confidence predictions of their structures using ColabFold (FIG. 10A, FIG. 12). As has been reported for other single-effector (Class 2) Cas proteins, the Cas13e (also referred to herein as “Cas13K2F”) predicted structure is bi-lobed. Cas13e consists of two HEPN domains in the Nuclease (NUC) lobe for cleavage of RNA targets, and a Recognition (REC) lobe consisting of α-helical domains and an N-terminal domain (NTD) that may be used to bind the CRISPR repeat of the gRNA. As opposed to the NTD comprised of α helices in Cas13a, or short a helices flanking a β sandwich region in Cas13d, the NTD in Cas13e and Cas13c appears to be comprised almost entirely of β sheets. Without wishing to be bound by theory, crRNA and target RNA in the Cas13e RNP would be coordinated within the large, positively charged central channel through the center of the enzyme (FIG. 10A; FIG. 12). Interestingly, the experiments of this example discovered the presence of a novel C-terminal domain (CTD) in the NUC lobe of Cas13c that is not found in previously reported Cas13 systems, and is comprised primarily of R sheets. In addition to the miniaturization of the NTD, HEPN, and helical domains in Cas13e compared to Cas13c, there was no homologous domain structure to the CTD in Cas13e.

### Example 5: Miniature Splice Editors Using CRISPR-Cas13e

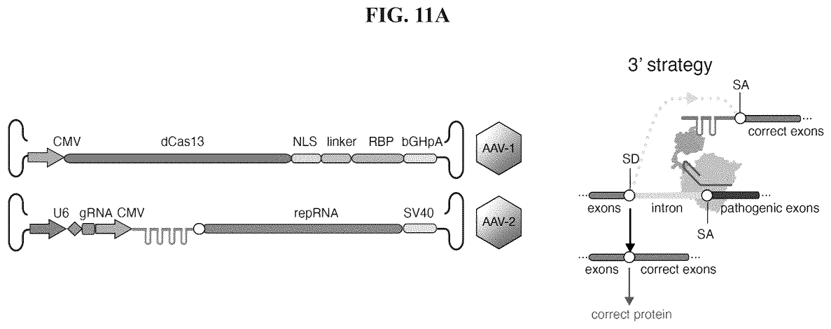

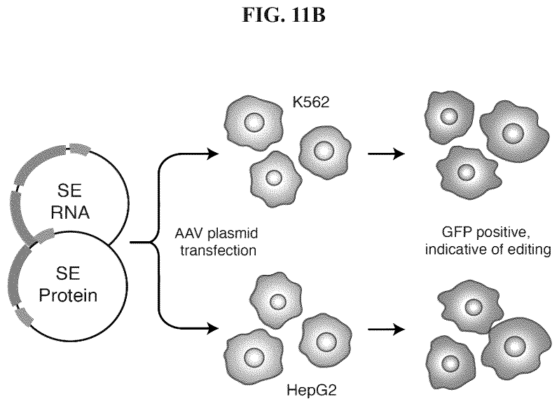

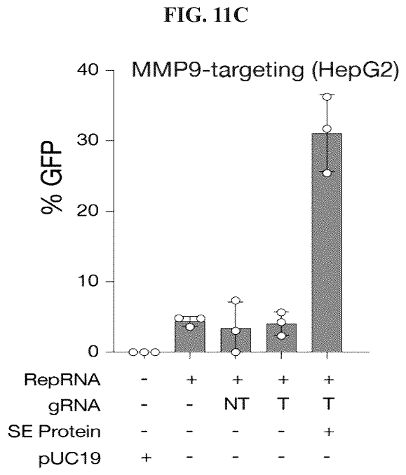

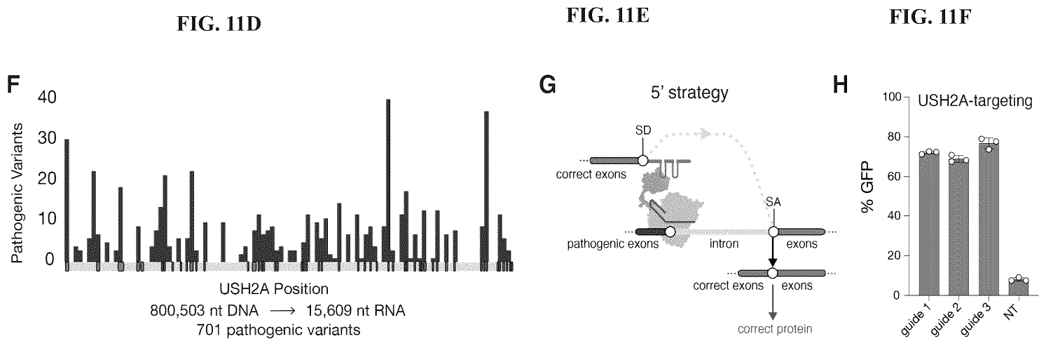

Given the relatively small size of Cas13e and its sequence divergence from Cas13 systems successfully employed in mammalian cells (<7%), the experiments of this example first sought to define its stability and activity in HEK293 cells. The experiments of this example tested the ability of the native Cas13e enzymes (also referred to as the Cas13K2F family of enzymes disclosed herein) to target and cleave GFP transcripts as compared to RfxCas13d using different guide orientations (FIG. 10B). Cas13e3 exhibited the most robust RNA knockdown activity, with a gRNA orientation that was the reverse of that of Cas13x (Cas13bt) (FIG. 10C; FIG. 12). The experiments of this example thus created nuclease-inactive (dCas) variants of Cas13e3 with R244A/H249A/R704A/H709A mutations in the HEPN domains. Using dCas13e3, a splice editor (SE3; fused, linked, or associated with dCas13K2F3-MCP or dCas13e3-MCP) was created which is 22% smaller than SE1, which allowed for the packaging of the full fusion protein into AAVs for delivery to mammalian cells. The experiments of this example first demonstrated the efficacy of the SE3 system by targeting the MMP9 splice acceptor site, leading to multi-kilobase 3′ replacement of RNA, not only via transient delivery (FIG. 10D) but also by packaging the system within AAVs (FIG. 11A). The experiments of this example further demonstrated that Splice Editing is active in a variety of human-derived cell lines beyond HEK293 (FIG. 11B), including in K562 cells (FIG. 11C).

### Example 6: Efficient and Programmable 5′ RNA Replacement in Alternative Gene Targets

USH2A has a gene size of 800 kb and an open reading frame of 15.6 kb (FIG. 11D). USH2A is too heterogeneous to be compatible with existing gene-editing approaches, with many genetic variants spanning missense, nonsense, insertion, deletion, and frame-shift mutations throughout its 72 exons (FIG. 11D). To test splice editing as a corrective approach for USH2A transcripts and demonstrate the possibility of replacement of the 5′ end of an RNA target (FIG. 4G), the experiments of this example designed gRNAs to bind intron 12 within an USH2A reporter, and a repRNA that would enable GFP fluorescence upon transcript correction with 5′ replacement. Via transient transfection into HEK293 cells, the experiments of this example surprisingly observed splice editing efficiencies up to ˜80% (FIG. 11F).

### Example 7: Internal Exon Replacement by Trans-Splicing

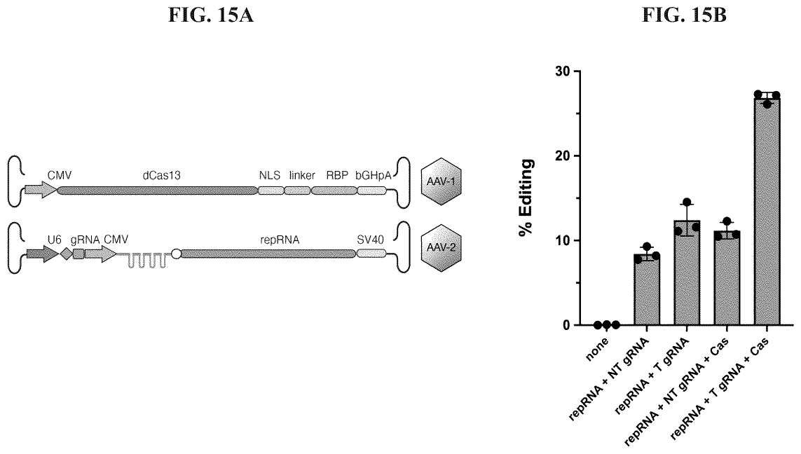

In the experiments of this example, AAV delivery of dCas13K2F3 with a targeting (T) or non-targeting (NT) gRNA and a repRNA was achieved to facilitate trans-splicing in HEK293T cells. (See FIG. 15A and FIG. 15B) In these experiments, splice editing is achieved by delivering splice editor components based on AAV transduction. In these experiments, a two vector system was used: one AAV with the guide RNA and ′epRNA, and one AAV with the Cas-RBP fusion. 5′ trans-splicing of split GFP reporter with USH2A introns and exons, functional GFP was detectable by microscopy and flow cytometry to indicate editing efficiency.

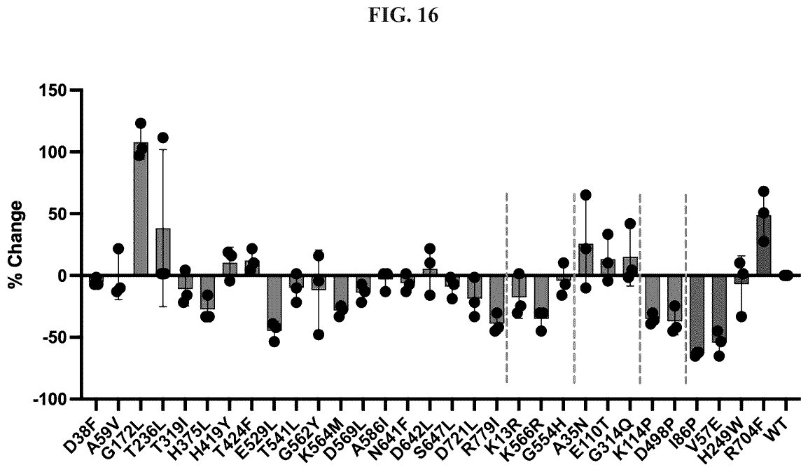

A graph showing amino acid substitutions in dCas13K2F3 that enable either improvement or reduction of trans-splicing efficacy in human cells compared to the original (WT) sequence is shown FIG. 16. In these experiments, 25 ng repRNA, 25 ng gRNA, 50 ng dCas13 variant were transfected into cells with the integrated USH2A reporter and GFP fluorescence measured via flow cytometry to indicate trans-splicing efficiency relative to the wild-type dCas13 sequence.

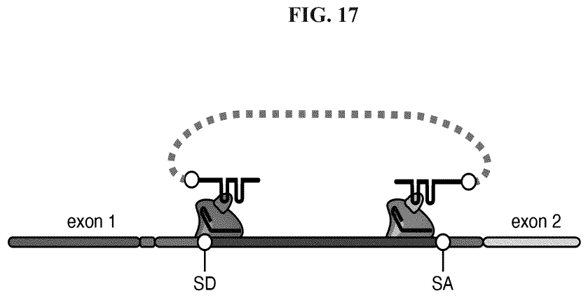

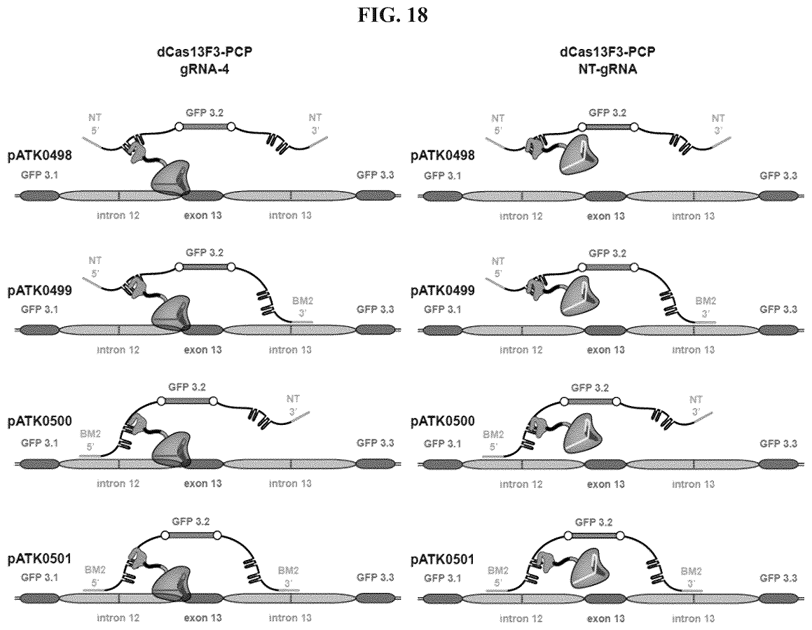

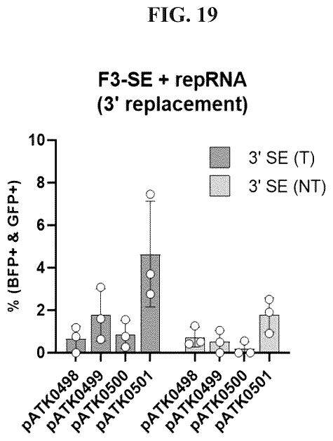

In these experiments, internal exon replacement was demonstrated using 2 nucleases with 2 separate gRNAs and RBPs, with a repRNA shown in green (directly to the left of the splice donor site in the image) (FIG. 17). In these experiments, internal exon replacement can also be achieved with a single gRNA, nuclease and an RBP (in this case, PCP instead of MCP which is used in the experiments above) in combination with a binding motif (BM) in the repRNA, shown in orange (and labeled BM2 in the image) (FIG. 18). In these experiments, cells were transfected with 25 ng reporter (pA0120), 25 ng repRNA, 25 ng gRNA, 25 ng Cas13. The graph in FIG. 19 shows targeting (T) in the first set of four bars on the left, or non-targeting (NT) in the second set of four bars on the right, of one gRNA, nuclease and RBP (PCP) in combination with a binding motif (BM) (see constructs in FIG. 18) to facilitate internal exon replacement.

### Example 8: RNA-Binding Protein-Free Trans-Splicing and repRNA Design

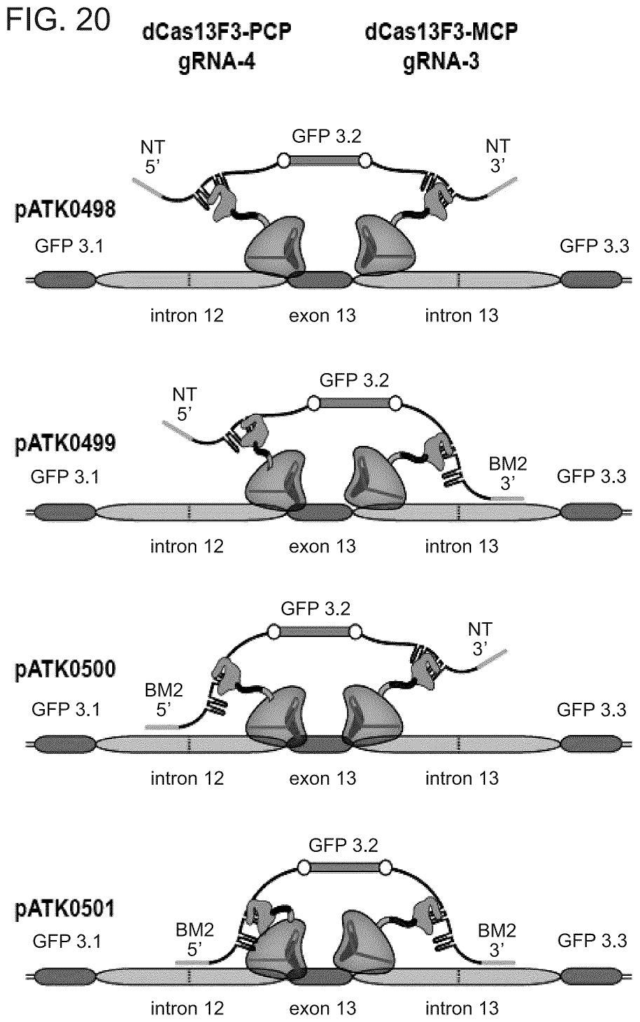

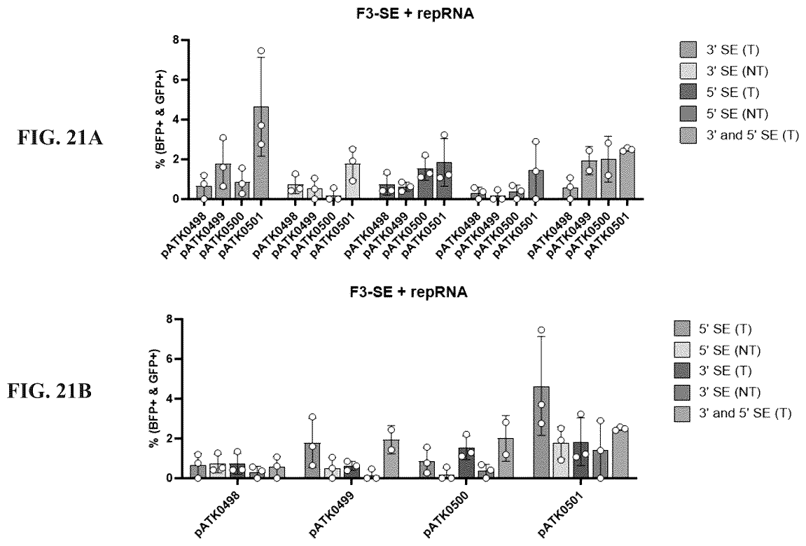

The experiments of this example demonstrate how internal exon replacement is achieved with two gRNAs, two nucleases and RBPs (in this case, PCP and MCP) in combination with, or without, a binding motif (BM) in the repRNA, shown in orange (and labeled BM2 in FIG. 20). The experiments in FIG. 21A and FIG. 21B show targeting (T) or non-targeting (NT) of two gRNAs, two nucleases and RBPs in combination with, or without, a binding motif (BM) in the repRNA (see constructs in FIG. 20) to facilitate internal exon replacement.

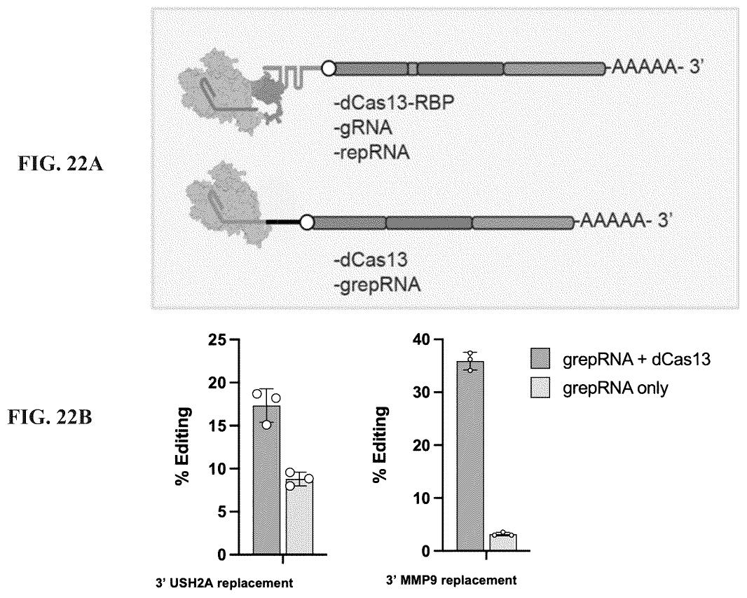

The experiments of this example demonstrate the design of a guide repair RNA (“grepRNA”), which is devoid of an RBP (FIG. 22A, bottom image). dCas13 can be used with or without an RBP fusion. FIG. 22B shows grepRNA only, or grepRNA and dCas13, for internal exon replacement across two different RNA targets (USH2A, left side of FIG. 22B; and MMP9, right side of FIG. 22B). In these experiments, HEK293T cells were transfected with pA0120 reporter plasmid (25 ng), grepRNA (50 ng), dCas13F3-MCP (25 ng).

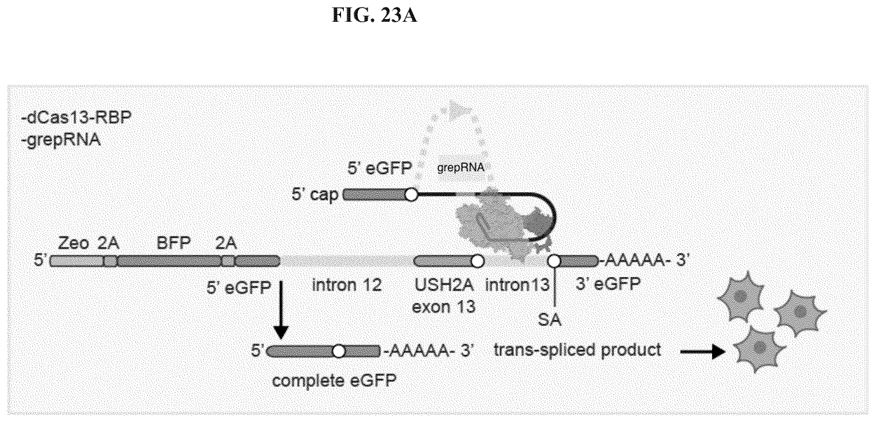

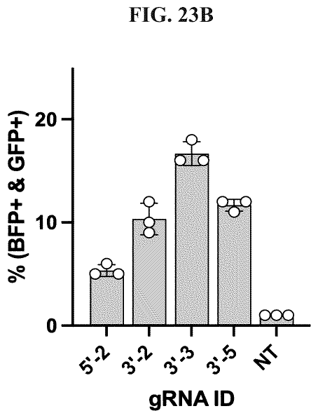

The experiments in FIG. 23A and FIG. 23B show targeting (T) or non-targeting (NT) of an integrated 5′ USH2A target to facilitate internal exon replacement. FIG. 23A is an image showing integrated USH2A target for 5′ replacement. FIG. 23B is a graph showing targeting (T) or non-targeting (NT) of an integrated 5′ USH2A target to facilitate internal exon replacement. Transient transfection of the dCas13F3-MCP, grepRNA (targeting (T) or non-targeting (NT)), and pUC19 in HEK293FT cells with an integrated USH2A target. Each splice editor (SE) component received 25 ng for a total of 100 ng/transfection/well in a 96-well plate.

### Materials and Methods

### Cell Culture

HEK293FT (Thermo Fisher Scientific Cat. No. R70007) were cultured in a humidified incubator at 5% CO2 at 37C using high-glucose DMEM (Invitrogen) complemented with 10% FBS and 1% penicillin/streptomycin. Cell cultures were kept at low passage (<20) and regularly tested for mycoplasma contamination.

### Cloning

Plasmids were synthesized, and genes were synthesized. The RBP sequences were synthesized by IDT and inserted with Golden Gate assembly. gRNAs were ordered as oligos from IDT and were hybridized and phosphorylated for golden gate assembly.

### Transfections

HEK293FT cells were transfected in a 12-well plate format, with a total of 1250 ng of DNA and 4 μL of Lipofectamine 2000 per condition. Each construct was one fourth (312.5 ng) of the total DNA transfection, with the exception of pUC19, which was used as non-coding dummy DNA in conditions where less than four components were delivered, such that there would be a total of 1250 ng of DNA in each transfection. Media was changed 6 hours after the transfection, and the cells were analyzed via flow cytometry (Beckman Coulter CytoFLEX platform) 48 hours after the transfection.

### RNA Knockdown Experiments in Mammalian Cells

For each Cas ORF, an SV40 NLS sequence was added to the N-terminus (MSPKKKRKVEAS (SEQ ID NO: 102)) and an SV40 NLS with an appended HA tag was added to the C-terminus (GSGPKKKRKVAAAYPYDVPDYA (SEQ ID NO: 103)). The resulting ORFs were then mammalian codon-optimized and synthesized into mammalian expression vectors. Transcription was driven by a CMV promoter (CGCCCCATTGACGCAAATGGGCGGTAGGCGTGTACGGTGGGAGGTCTATATAAGCA GAGCTGGTTTAGTGAACCGTCAGATC (SEQ ID NO: 104)) and an SV40 polyadenylation signal was used to terminate transcription (CAGCTCACAGACATGATAAGATACATTGATGAGTTTGGACAAACCACAACTAGAAT GCAGTGAAAAAAATGCTTTATTTGTGAAATTTGTGATGCTATTGCTTTATTTGTAACC ATTATAAGCTGCAATAAACAAGTTAACAACAACAATTGCATTCATTTTATGTTTCAG GTTCAGGGGGAGGTGTGGGAGGTTTTTTAAAGCAAGTAAAACCTCTACAAATGTGG TATTGGC (SEQ ID NO: 105)). A plasmid expressing eGFP and plasmids expressing Cas13e proteins and gRNAs were co-transfected into HEK293T cells (bio reps n=2) using lipofectamine 2000. Protein expression was confirmed by observing fluorescence via fluorescent microscopy, and flow cytometry was performed 48 hours following transfection. Cell and singlet gating was performed, and median GFP intensity of singlets was calculated. Results were then plotted. gRNAs were designed for Cas13e systems to target multiple sites across the coding sequence of eGFP in HEK293T cells, to be tested alongside a scrambled non-targeting guide, demonstrating reprogrammability and specificity of the RNase activity of these CRISPR-Cas systems towards different targets. Several gRNA orientations were tested to evaluate the correct structure of the CRISPR RNA of Cas13e systems. gRNA structure for Cas13e systems was determined to consist of a CRISPR repeat at the 5′ end of the RNA followed by a spacer sequence complementary to the target RNA. In contrast, the gRNA structure for Cas13e systems was determined to consist of a spacer sequence complementary to the target RNA on the 5′ end of the gRNA, followed by a CRISPR repeat at the 3′ end.

### Metagenome-Assembled Genome Construction, Binning, and Phylogenetic Classification of Cas13 Genomes

Briefly, samples were assembled with metaSPAdes v3.15.4. Proteins were identified using Prodigal v2.6.3 and aligned to custom Cas13 profiles. Reference proteins were aligned with Cas13 hits using MAFFT, and phylogenetic trees were built using IQTree. Samples containing one or more significant matches to Cas13 systems were binned into metagenome-assembled genomes (MAGs) using metabat2 v2.16, MaxBin v2.2.7 and CONCOCT v1.1 in parallel. Completeness and contamination were estimated using CheckM v1.2.2 using lineage-specific markers. Bins recovered from all three tools for each sample were refined and merged using DAS Tool v1.1.6. Medium and high-quality MAGs containing Cas13e (≥60% completeness and ≤10% contamination based on essential genes) from all samples were taxonomically classified using GTDB-tk using the “classify_wf” setting. In addition, bin phylogeny was determined by placing these genomes into a phylogenetic tree of existing Epsilonproteobacteria genomes built from a concatenated set of rRNA genes. CRISPR spacers from Cas13 genomes were searched against metagenomes using BLAST, and hits with ≥20nt of matches and ≤4 mismatches were retained for analysis.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A composition comprising an endonuclease comprising a sequence, or a fragment or variant thereof, and having at least 90% identity to SEQ ID NO: 3 or having about 1 to about 20 amino acid modifications relative to SEQ ID NO: 3.

2. A composition comprising a nucleic acid encoding an endonuclease comprising an endonuclease comprising a sequence, or a fragment or variant thereof, and having at least 90% identity to SEQ ID NO: 3 or having about 1 to about 20 amino acid modifications relative to SEQ ID NO: 3.

3. A composition comprising a nuclease system, comprising
(a) an endonuclease comprising a nucleic acid encoding an endonuclease comprising an endonuclease comprising a sequence, or a fragment or variant thereof, and having at least about 90% identity to SEQ ID NO: 3 or having about 1 to about 20 amino acid modifications relative to SEQ ID NO: 3; and
(b) an RNA molecule comprising a sequence complementary to one strand of a target nucleic acid molecule.

4. The composition of claim 1, further comprising one or more donor polynucleotides.

5. (canceled)

6. (canceled)

7. A composition comprising a chimeric protein comprising:
(a) an endonuclease comprising an endonuclease comprising a sequence, or a fragment or variant thereof, and having at least 90% identity to SEQ ID NO: 3 or having about 1 to about 20 amino acid modifications relative to SEQ ID NO: 3; and
(b) a nucleic acid-modulating domain or a nucleic acid-modifying domain comprising a sequence comprising a catalytic domain, or a fragment or variant thereof, wherein (a) and (b) do not naturally occur together in a same reading frame.

8. The composition of claim 7, wherein the nucleic acid-modulating domain or the nucleic acid-modifying domain is selected from MCP, lambdaN, PP7, QBeta, SLBP, and TBP/TAR.

9. (canceled)

10. A composition comprising a complex comprising chimeric protein and an RNA molecule, wherein the chimeric protein comprises
(a) an endonuclease, optionally comprising a sequence, or a fragment or variant thereof, and having at least 90% identity to SEQ ID NO: 3 or having about 1 to about 20 amino acid modifications relative to SEQ ID NO: 3; and
(b) a nucleic acid-modulating domain or a nucleic acid-modifying domain comprising a sequence comprising a catalytic domain, or a fragment or variant thereof,
wherein (a) and (b) do not naturally occur together in a same reading frame and
the RNA molecule comprises a sequence complementary to one strand of a target nucleic acid molecule.

11.-301. (canceled)

302. The composition of claim 1, comprising a higher eukaryotes and prokaryotes nucleotide-binding domain (HEPN) domain.

303. The composition of claim 2, comprising a higher eukaryotes and prokaryotes nucleotide-binding domain (HEPN) domain.

304. The composition of claim 3, comprising a higher eukaryotes and prokaryotes nucleotide-binding domain (HEPN) domain.

305. The composition of claim 7, comprising a higher eukaryotes and prokaryotes nucleotide-binding domain (HEPN) domain.

306. The composition of claim 10, comprising a higher eukaryotes and prokaryotes nucleotide-binding domain (HEPN) domain.

307. The composition of claim 2, further comprising one or more donor polynucleotides.

308. The composition of claim 3, further comprising one or more donor polynucleotides.

309. The composition of claim 7, further comprising one or more donor polynucleotides.

310. The composition of claim 10, further comprising one or more donor polynucleotides.
